SEQUENCE LISTING (574 sequences)

SEQ ID NO: 1 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 2-3 - skipped (2 entries)

SEQ ID NO: 4 - RNA, 69 nt, synthetic construct
  source 1..69: mol_type = other RNA
acagcttatttggaagctgaaatgtgaggtttataacactcacaagaatcctgaaaaaggatgccaaac

SEQ ID NO: 5 - RNA, 20 nt, synthetic construct
  source 1..20: mol_type = other RNA
atactaaccttggtttctgt

SEQ ID NO: 6 - RNA, 20 nt, synthetic construct
  source 1..20: mol_type = other RNA
ctgttcatttcagctttaac

SEQ ID NO: 7 - RNA, 20 nt, synthetic construct
  source 1..20: mol_type = other RNA
ccactgcactttagcctggg

SEQ ID NO: 8 - RNA, 20 nt, synthetic construct
  source 1..20: mol_type = other RNA
tcaaatgtaaccagtatttt

SEQ ID NO: 9 - RNA, 20 nt, synthetic construct
  source 1..20: mol_type = other RNA
gcctgggtgacagtgagact

SEQ ID NO: 10 - RNA, 20 nt, synthetic construct
  source 1..20: mol_type = other RNA
aaaaggtatctttgatacta

SEQ ID NO: 11 - RNA, 20 nt, synthetic construct
  source 1..20: mol_type = other RNA
acgtgattttctgttaataa

SEQ ID NO: 12 - RNA, 20 nt, synthetic construct
  source 1..20: mol_type = other RNA
caaagtctactgttcatttc

SEQ ID NO: 13 - RNA, 20 nt, synthetic construct
  source 1..20: mol_type = other RNA
attttatcaaatgtaaccag

SEQ ID NO: 14 - RNA, 20 nt, synthetic construct
  source 1..20: mol_type = other RNA
ttttcttagagacagagtct

SEQ ID NO: 15 - RNA, 20 nt, synthetic construct
  source 1..20: mol_type = other RNA
ccatagattgtaatttaatg

SEQ ID NO: 16 - RNA, 20 nt, synthetic construct
  source 1..20: mol_type = other RNA
tttattttcttagagacaga

SEQ ID NO: 17 - RNA, 20 nt, synthetic construct
  source 1..20: mol_type = other RNA
gctaggatgatgaacaacag

SEQ ID NO: 18 - RNA, 20 nt, synthetic construct
  source 1..20: mol_type = other RNA
gtagtaaatgctagtctgga

SEQ ID NO: 19 - RNA, 20 nt, synthetic construct
  source 1..20: mol_type = other RNA
atggcaaatattagtttctg

SEQ ID NO: 20 - RNA, 20 nt, synthetic construct
  source 1..20: mol_type = other RNA
tagtagtaaatgctagtctg

SEQ ID NO: 21 - RNA, 20 nt, synthetic construct
  source 1..20: mol_type = other RNA
ttatggctaggatgatgaac

SEQ ID NO: 22 - RNA, 20 nt, synthetic construct
  source 1..20: mol_type = other RNA
gaggagacattttaaatgta

SEQ ID NO: 23 - RNA, 20 nt, synthetic construct
  source 1..20: mol_type = other RNA
aatgtaacttccaaacgtta

SEQ ID NO: 24 - RNA, 20 nt, synthetic construct
  source 1..20: mol_type = other RNA
acttccaaacgttatctcac

SEQ ID NO: 25 - RNA, 20 nt, synthetic construct
  source 1..20: mol_type = other RNA
cttttttgatggcaaatatt

SEQ ID NO: 26 - RNA, 20 nt, synthetic construct
  source 1..20: mol_type = other RNA
agataacgtttggaagttac

SEQ ID NO: 27 - RNA, 20 nt, synthetic construct
  source 1..20: mol_type = other RNA
ccatcaaaaaagcaaagaat

SEQ ID NO: 28 - RNA, 20 nt, synthetic construct
  source 1..20: mol_type = other RNA
aataagcaacataaatgtga

SEQ ID NO: 29 - RNA, 20 nt, synthetic construct
  source 1..20: mol_type = other RNA
gttgaaagaattcagaatca

SEQ ID NO: 30 - RNA, 20 nt, synthetic construct
  source 1..20: mol_type = other RNA
gaagttacatttaaaatgtc

SEQ ID NO: 31 - RNA, 20 nt, synthetic construct
  source 1..20: mol_type = other RNA
acagaaactaatatttgcca

SEQ ID NO: 32 - RNA, 20 nt, synthetic construct
  source 1..20: mol_type = other RNA
aaatgtctcctccagactag

SEQ ID NO: 33 - RNA, 20 nt, synthetic construct
  source 1..20: mol_type = other RNA
ttctagttgaaagaattcag

SEQ ID NO: 34 - RNA, 20 nt, synthetic construct
  source 1..20: mol_type = other RNA
ccaacttttatcattttttc

SEQ ID NO: 35 - RNA, 20 nt, synthetic construct
  source 1..20: mol_type = other RNA
tttgctgagagagaaacagt

SEQ ID NO: 36 - RNA, 20 nt, synthetic construct
  source 1..20: mol_type = other RNA
cttaggctgaatagtgagag

SEQ ID NO: 37 - RNA, 20 nt, synthetic construct
  source 1..20: mol_type = other RNA
tcattttttctcataccttc

SEQ ID NO: 38 - RNA, 20 nt, synthetic construct
  source 1..20: mol_type = other RNA
cttgatgatcatctcgttga

SEQ ID NO: 39 - RNA, 20 nt, synthetic construct
  source 1..20: mol_type = other RNA
ctgagagagaaacagttgcc

SEQ ID NO: 40 - RNA, 20 nt, synthetic construct
  source 1..20: mol_type = other RNA
gctacttttgttatttgcat

SEQ ID NO: 41 - RNA, 20 nt, synthetic construct
  source 1..20: mol_type = other RNA
ggagagtaaagtgattggtg

SEQ ID NO: 42 - RNA, 20 nt, synthetic construct
  source 1..20: mol_type = other RNA
tggctacttttgttatttgc

SEQ ID NO: 43 - RNA, 20 nt, synthetic construct
  source 1..20: mol_type = other RNA
aagaaaaacttctgccaact

SEQ ID NO: 44 - RNA, 20 nt, synthetic construct
  source 1..20: mol_type = other RNA
tatccttgattatacttagg

SEQ ID NO: 45 - RNA, 20 nt, synthetic construct
  source 1..20: mol_type = other RNA
tccttgattatacttaggct

SEQ ID NO: 46 - RNA, 20 nt, synthetic construct
  source 1..20: mol_type = other RNA
aaatgaagattttccaccaa

SEQ ID NO: 47 - RNA, 20 nt, synthetic construct
  source 1..20: mol_type = other RNA
ctctcctagaccatttccca

SEQ ID NO: 48 - RNA, 20 nt, synthetic construct
  source 1..20: mol_type = other RNA
agtaggagctaaaatatttt

SEQ ID NO: 49 - RNA, 20 nt, synthetic construct
  source 1..20: mol_type = other RNA
gatactttgtttagcaatac

SEQ ID NO: 50 - RNA, 20 nt, synthetic construct
  source 1..20: mol_type = other RNA
ggtttttgcaaaaaggaaaa

SEQ ID NO: 51 - RNA, 20 nt, synthetic construct
  source 1..20: mol_type = other RNA
tctttttctaacaatgtgga

SEQ ID NO: 52 - RNA, 20 nt, synthetic construct
  source 1..20: mol_type = other RNA
acaatgtggatactttgttt

SEQ ID NO: 53 - RNA, 20 nt, synthetic construct
  source 1..20: mol_type = other RNA
agccaaactcttattcatga

SEQ ID NO: 54 - RNA, 20 nt, synthetic construct
  source 1..20: mol_type = other RNA
attgaagagtaacaatttga

SEQ ID NO: 55 - RNA, 20 nt, synthetic construct
  source 1..20: mol_type = other RNA
gcaatacatggtagaaaatg

SEQ ID NO: 56 - RNA, 20 nt, synthetic construct
  source 1..20: mol_type = other RNA
caaaaaggaaaaaagaagaa

SEQ ID NO: 57 - RNA, 20 nt, synthetic construct
  source 1..20: mol_type = other RNA
tttagcaatacatggtagaa

SEQ ID NO: 58 - RNA, 20 nt, synthetic construct
  source 1..20: mol_type = other RNA
tatctttttctaacaatgtg

SEQ ID NO: 59 - RNA, 20 nt, synthetic construct
  source 1..20: mol_type = other RNA
atgtcatgaataagagtttg

SEQ ID NO: 60 - RNA, 20 nt, synthetic construct
  source 1..20: mol_type = other RNA
gctcctactcagactgttac

SEQ ID NO: 61 - RNA, 20 nt, synthetic construct
  source 1..20: mol_type = other RNA
gcttgtgtttctaatttttc

SEQ ID NO: 62 - RNA, 20 nt, synthetic construct
  source 1..20: mol_type = other RNA
ttgctaaacaaagtatccac

SEQ ID NO: 63 - RNA, 20 nt, synthetic construct
  source 1..20: mol_type = other RNA
taatgtcatgaataagagtt

SEQ ID NO: 64 - RNA, 20 nt, synthetic construct
  source 1..20: mol_type = other RNA
ccatgtattgctaaacaaag

SEQ ID NO: 65 - RNA, 20 nt, synthetic construct
  source 1..20: mol_type = other RNA
gctcaaattgttactcttca

SEQ ID NO: 66 - RNA, 20 nt, synthetic construct
  source 1..20: mol_type = other RNA
agactttttgcacagtcaat

SEQ ID NO: 67 - RNA, 20 nt, synthetic construct
  source 1..20: mol_type = other RNA
cttacaggctccaatagtgg

SEQ ID NO: 68 - RNA, 20 nt, synthetic construct
  source 1..20: mol_type = other RNA
cacagtcaataacacaaagg

SEQ ID NO: 69 - RNA, 20 nt, synthetic construct
  source 1..20: mol_type = other RNA
gaattgaaacaaattttctc

SEQ ID NO: 70 - RNA, 20 nt, synthetic construct
  source 1..20: mol_type = other RNA
tctctatctttagaattgaa

SEQ ID NO: 71 - RNA, 20 nt, synthetic construct
  source 1..20: mol_type = other RNA
aacaaatagctagagccaaa

SEQ ID NO: 72 - RNA, 20 nt, synthetic construct
  source 1..20: mol_type = other RNA
aatcagagtcaatttccaag

SEQ ID NO: 73 - RNA, 20 nt, synthetic construct
  source 1..20: mol_type = other RNA
ctctaagactttttgcacag

SEQ ID NO: 74 - RNA, 20 nt, synthetic construct
  source 1..20: mol_type = other RNA
ttcaaaagtgcaactatgaa

SEQ ID NO: 75 - RNA, 20 nt, synthetic construct
  source 1..20: mol_type = other RNA
gctctagctatttgttcaaa

SEQ ID NO: 76 - RNA, 20 nt, synthetic construct
  source 1..20: mol_type = other RNA
gctatttgttcaaaagtgca

SEQ ID NO: 77 - RNA, 20 nt, synthetic construct
  source 1..20: mol_type = other RNA
agtatactggatcccattct

SEQ ID NO: 78 - RNA, 20 nt, synthetic construct
  source 1..20: mol_type = other RNA
tgttattgactgtgcaaaaa

SEQ ID NO: 79 - RNA, 20 nt, synthetic construct
  source 1..20: mol_type = other RNA
attcaaagtgttgcatgaca

SEQ ID NO: 80 - RNA, 20 nt, synthetic construct
  source 1..20: mol_type = other RNA
tttcaattctaaagatagag

SEQ ID NO: 81 - RNA, 20 nt, synthetic construct
  source 1..20: mol_type = other RNA
aagatagagataaacctttg

SEQ ID NO: 82 - RNA, 20 nt, synthetic construct
  source 1..20: mol_type = other RNA
ttattgactgtgcaaaaagt

SEQ ID NO: 83 - RNA, 20 nt, synthetic construct
  source 1..20: mol_type = other RNA
aagtgatgactgggtgagag

SEQ ID NO: 84 - RNA, 20 nt, synthetic construct
  source 1..20: mol_type = other RNA
ctggatcccattctctttgg

SEQ ID NO: 85 - RNA, 20 nt, synthetic construct
  source 1..20: mol_type = other RNA
caaaaagtcttagagtacat

SEQ ID NO: 86 - RNA, 20 nt, synthetic construct
  source 1..20: mol_type = other RNA
aagataattcatgaacatct

SEQ ID NO: 87 - RNA, 20 nt, synthetic construct
  source 1..20: mol_type = other RNA
attattttagccaaccaccc

SEQ ID NO: 88 - RNA, 20 nt, synthetic construct
  source 1..20: mol_type = other RNA
cttctaaattaactttagtg

SEQ ID NO: 89 - RNA, 20 nt, synthetic construct
  source 1..20: mol_type = other RNA
aattaactttagtgggtaga

SEQ ID NO: 90 - RNA, 20 nt, synthetic construct
  source 1..20: mol_type = other RNA
gccaaccaccctacaaatat

SEQ ID NO: 91 - RNA, 20 nt, synthetic construct
  source 1..20: mol_type = other RNA
gtgggtagaatttcttttaa

SEQ ID NO: 92 - RNA, 20 nt, synthetic construct
  source 1..20: mol_type = other RNA
acagaaaagcatacacatta

SEQ ID NO: 93 - RNA, 20 nt, synthetic construct
  source 1..20: mol_type = other RNA
acttcctctttaacagaaaa

SEQ ID NO: 94 - RNA, 20 nt, synthetic construct
  source 1..20: mol_type = other RNA
aaagaaattctacccactaa

SEQ ID NO: 95 - RNA, 20 nt, synthetic construct
  source 1..20: mol_type = other RNA
tagggtggttggctaaaata

SEQ ID NO: 96 - RNA, 20 nt, synthetic construct
  source 1..20: mol_type = other RNA
tatgcttttctgttaaagag

SEQ ID NO: 97 - RNA, 20 nt, synthetic construct
  source 1..20: mol_type = other RNA
cccactaaagttaatttaga

SEQ ID NO: 98 - RNA, 20 nt, synthetic construct
  source 1..20: mol_type = other RNA
ttaaagaggaagttagaaga

SEQ ID NO: 99 - RNA, 20 nt, synthetic construct
  source 1..20: mol_type = other RNA
tgcttttctgttaaagagga

SEQ ID NO: 100 - RNA, 20 nt, synthetic construct
  source 1..20: mol_type = other RNA
ttcaccaaatggattaagat

SEQ ID NO: 101 - RNA, 20 nt, synthetic construct
  source 1..20: mol_type = other RNA
gggtggttggctaaaataat

SEQ ID NO: 102 - RNA, 20 nt, synthetic construct
  source 1..20: mol_type = other RNA
cttttctgttaaagaggaag

SEQ ID NO: 103 - RNA, 20 nt, synthetic construct
  source 1..20: mol_type = other RNA
ctaaaatgttttcattccta

SEQ ID NO: 104 - RNA, 20 nt, synthetic construct
  source 1..20: mol_type = other RNA
ctgctgttgattaatggttg

SEQ ID NO: 105 - RNA, 20 nt, synthetic construct
  source 1..20: mol_type = other RNA
atctctcatgaaatattctt

SEQ ID NO: 106 - RNA, 20 nt, synthetic construct
  source 1..20: mol_type = other RNA
aagaaagcttaaaaagtctg

SEQ ID NO: 107 - RNA, 20 nt, synthetic construct
  source 1..20: mol_type = other RNA
tcgccctacctcttttttct

SEQ ID NO: 108 - RNA, 20 nt, synthetic construct
  source 1..20: mol_type = other RNA
atgttagtgcctttcaccct

SEQ ID NO: 109 - RNA, 20 nt, synthetic construct
  source 1..20: mol_type = other RNA
agcagggtgaaaggcactaa

SEQ ID NO: 110 - RNA, 20 nt, synthetic construct
  source 1..20: mol_type = other RNA
gaagaatatttcatgagaga

SEQ ID NO: 111 - RNA, 20 nt, synthetic construct
  source 1..20: mol_type = other RNA
agctttctttagaagaatat

SEQ ID NO: 112 - RNA, 20 nt, synthetic construct
  source 1..20: mol_type = other RNA
ctaaaatatatacttgtggc

SEQ ID NO: 113 - RNA, 20 nt, synthetic construct
  source 1..20: mol_type = other RNA
gcagactttttaagctttct

SEQ ID NO: 114 - RNA, 20 nt, synthetic construct
  source 1..20: mol_type = other RNA
aaaatttccctatgaaactg

SEQ ID NO: 115 - RNA, 20 nt, synthetic construct
  source 1..20: mol_type = other RNA
tttgagaaaagattaaacag

SEQ ID NO: 116 - RNA, 20 nt, synthetic construct
  source 1..20: mol_type = other RNA
agaaaagattaaacagtgtg

SEQ ID NO: 117 - RNA, 20 nt, synthetic construct
  source 1..20: mol_type = other RNA
agataccaaaaaggcaaaac

SEQ ID NO: 118 - RNA, 20 nt, synthetic construct
  source 1..20: mol_type = other RNA
ctggcaaagaaagaaataca

SEQ ID NO: 119 - RNA, 20 nt, synthetic construct
  source 1..20: mol_type = other RNA
ctaccacatgcagttgtact

SEQ ID NO: 120 - RNA, 20 nt, synthetic construct
  source 1..20: mol_type = other RNA
aaactgacatgcccatatcc

SEQ ID NO: 121 - RNA, 20 nt, synthetic construct
  source 1..20: mol_type = other RNA
ttttgcctttttggtatctt

SEQ ID NO: 122 - RNA, 20 nt, synthetic construct
  source 1..20: mol_type = other RNA
gtagcacactgtttaatctt

SEQ ID NO: 123 - RNA, 20 nt, synthetic construct
  source 1..20: mol_type = other RNA
tcctttggatatgggcatgt

SEQ ID NO: 124 - RNA, 20 nt, synthetic construct
  source 1..20: mol_type = other RNA
tatttctttctttgccagta

SEQ ID NO: 125 - RNA, 20 nt, synthetic construct
  source 1..20: mol_type = other RNA
tcttgtatcctttggatatg

SEQ ID NO: 126 - RNA, 20 nt, synthetic construct
  source 1..20: mol_type = other RNA
cctttttggtatcttacagg

SEQ ID NO: 127 - RNA, 20 nt, synthetic construct
  source 1..20: mol_type = other RNA
gatatgggcatgtcagtttc

SEQ ID NO: 128 - RNA, 20 nt, synthetic construct
  source 1..20: mol_type = other RNA
gtatcttacaggaactccag

SEQ ID NO: 129 - RNA, 20 nt, synthetic construct
  source 1..20: mol_type = other RNA
tttctttctttgccagtaca

SEQ ID NO: 130 - RNA, 20 nt, synthetic construct
  source 1..20: mol_type = other RNA
ccagtacaactgcatgtggt

SEQ ID NO: 131 - RNA, 20 nt, synthetic construct
  source 1..20: mol_type = other RNA
ggcatgtcagtttcataggg

SEQ ID NO: 132 - RNA, 20 nt, synthetic construct
  source 1..20: mol_type = other RNA
gtaatataatgatgacaaca

SEQ ID NO: 133 - RNA, 20 nt, synthetic construct
  source 1..20: mol_type = other RNA
agaagttaaagagtccagat

SEQ ID NO: 134 - RNA, 20 nt, synthetic construct
  source 1..20: mol_type = other RNA
atgatgacaacaacagtcaa

SEQ ID NO: 135 - RNA, 20 nt, synthetic construct
  source 1..20: mol_type = other RNA
ctgaagataaatacaatttc

SEQ ID NO: 136 - RNA, 20 nt, synthetic construct
  source 1..20: mol_type = other RNA
tttatcttcagcacatctgg

SEQ ID NO: 137 - RNA, 20 nt, synthetic construct
  source 1..20: mol_type = other RNA
aagggtgatggaaattactt

SEQ ID NO: 138 - RNA, 20 nt, synthetic construct
  source 1..20: mol_type = other RNA
actgttgttgtcatcattat

SEQ ID NO: 139 - RNA, 20 nt, synthetic construct
  source 1..20: mol_type = other RNA
acttcttaaagatcaggttc

SEQ ID NO: 140 - RNA, 20 nt, synthetic construct
  source 1..20: mol_type = other RNA
tcttcagcacatctggactc

SEQ ID NO: 141 - RNA, 20 nt, synthetic construct
  source 1..20: mol_type = other RNA
gactttttgtgtcaggatga

SEQ ID NO: 142 - RNA, 20 nt, synthetic construct
  source 1..20: mol_type = other RNA
gactctttaacttcttaaag

SEQ ID NO: 143 - RNA, 20 nt, synthetic construct
  source 1..20: mol_type = other RNA
gttatactgacaaagatatc

SEQ ID NO: 144 - RNA, 20 nt, synthetic construct
  source 1..20: mol_type = other RNA
ttttttggttatactgacaa

SEQ ID NO: 145 - RNA, 20 nt, synthetic construct
  source 1..20: mol_type = other RNA
ctgacaaagatatcactctg

SEQ ID NO: 146 - RNA, 20 nt, synthetic construct
  source 1..20: mol_type = other RNA
gcgtatattttttggttata

SEQ ID NO: 147 - RNA, 20 nt, synthetic construct
  source 1..20: mol_type = other RNA
cagagtttagtttcaagtaa

SEQ ID NO: 148 - RNA, 20 nt, synthetic construct
  source 1..20: mol_type = other RNA
atagtttcctgcatttgcag

SEQ ID NO: 149 - RNA, 20 nt, synthetic construct
  source 1..20: mol_type = other RNA
tcaaatcgcctgcaggtaaa

SEQ ID NO: 150 - RNA, 20 nt, synthetic construct
  source 1..20: mol_type = other RNA
gatcaagaaaaatagatgga

SEQ ID NO: 151 - RNA, 20 nt, synthetic construct
  source 1..20: mol_type = other RNA
cacacctagcatgtacacac

SEQ ID NO: 152 - RNA, 20 nt, synthetic construct
  source 1..20: mol_type = other RNA
gagatatagcgtatattttt

SEQ ID NO: 153 - RNA, 20 nt, synthetic construct
  source 1..20: mol_type = other RNA
cctgcaggcgatttgacaga

SEQ ID NO: 154 - RNA, 20 nt, synthetic construct
  source 1..20: mol_type = other RNA
cgctatatctctataatctg

SEQ ID NO: 155 - RNA, 20 nt, synthetic construct
  source 1..20: mol_type = other RNA
tttttcttgatccatatgct

SEQ ID NO: 156 - RNA, 20 nt, synthetic construct
  source 1..20: mol_type = other RNA
caaatgcaggaaactatcag

SEQ ID NO: 157 - RNA, 20 nt, synthetic construct
  source 1..20: mol_type = other RNA
tttgttacttgaaactaaac

SEQ ID NO: 158 - RNA, 20 nt, synthetic construct
  source 1..20: mol_type = other RNA
tacatgctaggtgtgtatat

SEQ ID NO: 159 - RNA, 20 nt, synthetic construct
  source 1..20: mol_type = other RNA
tctctataatctgttttaca

SEQ ID NO: 160 - RNA, 20 nt, synthetic construct
  source 1..20: mol_type = other RNA
tgtacatgctaggtgtgtat

SEQ ID NO: 161 - RNA, 20 nt, synthetic construct
  source 1..20: mol_type = other RNA
cttttacctgcaggcgattt

SEQ ID NO: 162 - RNA, 20 nt, synthetic construct
  source 1..20: mol_type = other RNA
ttacttgaaactaaactctg

SEQ ID NO: 163 - RNA, 20 nt, synthetic construct
  source 1..20: mol_type = other RNA
acagatctgttgagaaatgg

SEQ ID NO: 164 - RNA, 20 nt, synthetic construct
  source 1..20: mol_type = other RNA
aaatgttgtgtgtacatgct

SEQ ID NO: 165 - RNA, 20 nt, synthetic construct
  source 1..20: mol_type = other RNA
taatctgttttacataatcc

SEQ ID NO: 166 - RNA, 20 nt, synthetic construct
  source 1..20: mol_type = other RNA
catgctaggtgtgtatatta

SEQ ID NO: 167 - RNA, 20 nt, synthetic construct
  source 1..20: mol_type = other RNA
cataatccatctatttttct

SEQ ID NO: 168 - RNA, 20 nt, synthetic construct
  source 1..20: mol_type = other RNA
atctgttttacataatccat

SEQ ID NO: 169 - RNA, 20 nt, synthetic construct
  source 1..20: mol_type = other RNA
accaaaaaatatacgctata

SEQ ID NO: 170 - RNA, 20 nt, synthetic construct
  source 1..20: mol_type = other RNA
attttcttttggattgcatc

SEQ ID NO: 171 - RNA, 20 nt, synthetic construct
  source 1..20: mol_type = other RNA
ttgaatcctttaacatttca

SEQ ID NO: 172 - RNA, 20 nt, synthetic construct
  source 1..20: mol_type = other RNA
gattgcatctactgtatagg

SEQ ID NO: 173 - RNA, 20 nt, synthetic construct
  source 1..20: mol_type = other RNA
acatttcattcaactgttgc

SEQ ID NO: 174 - RNA, 20 nt, synthetic construct
  source 1..20: mol_type = other RNA
tagggaccctccttccatga

SEQ ID NO: 175 - RNA, 20 nt, synthetic construct
  source 1..20: mol_type = other RNA
cttcatcccactgattctga

SEQ ID NO: 176 - RNA, 20 nt, synthetic construct
  source 1..20: mol_type = other RNA
aaggtgttcttgtacttcat

SEQ ID NO: 177 - RNA, 20 nt, synthetic construct
  source 1..20: mol_type = other RNA
tgattttcttttggattgca

SEQ ID NO: 178 - RNA, 20 nt, synthetic construct
  source 1..20: mol_type = other RNA
gggaccctccttccatgact

SEQ ID NO: 179 - RNA, 20 nt, synthetic construct
  source 1..20: mol_type = other RNA
ctgtatagggaccctccttc

SEQ ID NO: 180 - RNA, 20 nt, synthetic construct
  source 1..20: mol_type = other RNA
gcctgtcctaagacctgctc

SEQ ID NO: 181 - RNA, 20 nt, synthetic construct
  source 1..20: mol_type = other RNA
cagtagatgcaatccaaaag

SEQ ID NO: 182 - RNA, 20 nt, synthetic construct
  source 1..20: mol_type = other RNA
tccaagcccggttgaaatct

SEQ ID NO: 183 - RNA, 20 nt, synthetic construct
  source 1..20: mol_type = other RNA
gtttggagatggcagtttcc

SEQ ID NO: 184 - RNA, 20 nt, synthetic construct
  source 1..20: mol_type = other RNA
tcaccagagtaacagtctga

SEQ ID NO: 185 - RNA, 20 nt, synthetic construct
  source 1..20: mol_type = other RNA
attttataacttgatcaagc

SEQ ID NO: 186 - RNA, 20 nt, synthetic construct
  source 1..20: mol_type = other RNA
taacttgatcaagcagagaa

SEQ ID NO: 187 - RNA, 20 nt, synthetic construct
  source 1..20: mol_type = other RNA
acttgatcaagcagagaaag

SEQ ID NO: 188 - RNA, 20 nt, synthetic construct
  source 1..20: mol_type = other RNA
ccagagcaggtacctccaac

SEQ ID NO: 189 - RNA, 20 nt, synthetic construct
  source 1..20: mol_type = other RNA
gagatggcagtttccttagt

SEQ ID NO: 190 - RNA, 20 nt, synthetic construct
  source 1..20: mol_type = other RNA
gttactaaggaaactgccat

SEQ ID NO: 191 - RNA, 20 nt, synthetic construct
  source 1..20: mol_type = other RNA
gcagatttcaaccgggcttg

SEQ ID NO: 192 - RNA, 20 nt, synthetic construct
  source 1..20: mol_type = other RNA
gtgacacaacctgtggttac

SEQ ID NO: 193 - RNA, 20 nt, synthetic construct
  source 1..20: mol_type = other RNA
aaatcacagagggtgatggt

SEQ ID NO: 194 - RNA, 20 nt, synthetic construct
  source 1..20: mol_type = other RNA
cttgatcaagttataaaatc

SEQ ID NO: 195 - RNA, 20 nt, synthetic construct
  source 1..20: mol_type = other RNA
ccgccttccactcagagctc

SEQ ID NO: 196 - RNA, 20 nt, synthetic construct
  source 1..20: mol_type = other RNA
ccctcagctcttgaagtaaa

SEQ ID NO: 197 - RNA, 20 nt, synthetic construct
  source 1..20: mol_type = other RNA
agtggaaggcggtaaaccgt

SEQ ID NO: 198 - RNA, 20 nt, synthetic construct
  source 1..20: mol_type = other RNA
cttcaagagctgagggcaaa

SEQ ID NO: 199 - RNA, 20 nt, synthetic construct
  source 1..20: mol_type = other RNA
agctctgagtggaaggcggt

SEQ ID NO: 200 - RNA, 20 nt, synthetic construct
  source 1..20: mol_type = other RNA
acagctgtttgcagacctcc

SEQ ID NO: 201 - RNA, 20 nt, synthetic construct
  source 1..20: mol_type = other RNA
tttttgaggattgctgaatt

SEQ ID NO: 202 - RNA, 20 nt, synthetic construct
  source 1..20: mol_type = other RNA
cagacctcctgccaccgcag

SEQ ID NO: 203 - RNA, 20 nt, synthetic construct
  source 1..20: mol_type = other RNA
aggattgctgaattatttct

SEQ ID NO: 204 - RNA, 20 nt, synthetic construct
  source 1..20: mol_type = other RNA
gaatactggcatctgttttt

SEQ ID NO: 205 - RNA, 20 nt, synthetic construct
  source 1..20: mol_type = other RNA
tctgacagctgtttgcagac

SEQ ID NO: 206 - RNA, 20 nt, synthetic construct
  source 1..20: mol_type = other RNA
acaacagtttgccgctgccc

SEQ ID NO: 207 - RNA, 20 nt, synthetic construct
  source 1..20: mol_type = other RNA
ccgctgcccaatgccatcct

SEQ ID NO: 208 - RNA, 20 nt, synthetic construct
  source 1..20: mol_type = other RNA
caaacagctgtcagacagaa

SEQ ID NO: 209 - RNA, 20 nt, synthetic construct
  source 1..20: mol_type = other RNA
caggaaaaattgggaagcct

SEQ ID NO: 210 - RNA, 20 nt, synthetic construct
  source 1..20: mol_type = other RNA
cggtggcaggaggtctgcaa

SEQ ID NO: 211 - RNA, 20 nt, synthetic construct
  source 1..20: mol_type = other RNA
gcatgttcccaattctcagg

SEQ ID NO: 212 - RNA, 20 nt, synthetic construct
  source 1..20: mol_type = other RNA
tcataatgaaaacgccgcca

SEQ ID NO: 213 - RNA, 20 nt, synthetic construct
  source 1..20: mol_type = other RNA
tctttctgagaaactgttca

SEQ ID NO: 214 - RNA, 20 nt, synthetic construct
  source 1..20: mol_type = other RNA
ttagccactgattaaatatc

SEQ ID NO: 215 - RNA, 20 nt, synthetic construct
  source 1..20: mol_type = other RNA
agaaactgttcagcttctgt

SEQ ID NO: 216 - RNA, 20 nt, synthetic construct
  source 1..20: mol_type = other RNA
tatcataatgaaaacgccgc

SEQ ID NO: 217 - RNA, 20 nt, synthetic construct
  source 1..20: mol_type = other RNA
tttagcatgttcccaattct

SEQ ID NO: 218 - RNA, 20 nt, synthetic construct
  source 1..20: mol_type = other RNA
tatttagcatgttcccaatt

SEQ ID NO: 219 - RNA, 20 nt, synthetic construct
  source 1..20: mol_type = other RNA
tgtctttctgagaaactgtt

SEQ ID NO: 220 - RNA, 20 nt, synthetic construct
  source 1..20: mol_type = other RNA
atcagtggctaacagaagct

SEQ ID NO: 221 - RNA, 20 nt, synthetic construct
  source 1..20: mol_type = other RNA
ttgagaaatggcggcgtttt

SEQ ID NO: 222 - RNA, 20 nt, synthetic construct
  source 1..20: mol_type = other RNA
aagatatttaatcagtggct

SEQ ID NO: 223 - RNA, 89 nt, synthetic construct
  source 1..89: mol_type = other RNA
acagcttatttggaagctgaaatgtgaggtttataacactcacaagaatcctgaaaaaggatgccaaacatactaaccttggtttctgt

SEQ ID NO: 224 - RNA, 89 nt, synthetic construct
  source 1..89: mol_type = other RNA
acagcttatttggaagctgaaatgtgaggtttataacactcacaagaatcctgaaaaaggatgccaaacctgttcatttcagctttaac

SEQ ID NO: 225 - RNA, 89 nt, synthetic construct
  source 1..89: mol_type = other RNA
acagcttatttggaagctgaaatgtgaggtttataacactcacaagaatcctgaaaaaggatgccaaacccactgcactttagcctggg

SEQ ID NO: 226 - RNA, 89 nt, synthetic construct
  source 1..89: mol_type = other RNA
acagcttatttggaagctgaaatgtgaggtttataacactcacaagaatcctgaaaaaggatgccaaactcaaatgtaaccagtatttt

SEQ ID NO: 227 - RNA, 89 nt, synthetic construct
  source 1..89: mol_type = other RNA
acagcttatttggaagctgaaatgtgaggtttataacactcacaagaatcctgaaaaaggatgccaaacgcctgggtgacagtgagact

SEQ ID NO: 228 - RNA, 89 nt, synthetic construct
  source 1..89: mol_type = other RNA
acagcttatttggaagctgaaatgtgaggtttataacactcacaagaatcctgaaaaaggatgccaaacaaaaggtatctttgatacta

SEQ ID NO: 229 - RNA, 89 nt, synthetic construct
  source 1..89: mol_type = other RNA
acagcttatttggaagctgaaatgtgaggtttataacactcacaagaatcctgaaaaaggatgccaaacacgtgattttctgttaataa

SEQ ID NO: 230 - RNA, 89 nt, synthetic construct
  source 1..89: mol_type = other RNA
acagcttatttggaagctgaaatgtgaggtttataacactcacaagaatcctgaaaaaggatgccaaaccaaagtctactgttcatttc

SEQ ID NO: 231 - RNA, 89 nt, synthetic construct
  source 1..89: mol_type = other RNA
acagcttatttggaagctgaaatgtgaggtttataacactcacaagaatcctgaaaaaggatgccaaacattttatcaaatgtaaccag

SEQ ID NO: 232 - RNA, 89 nt, synthetic construct
  source 1..89: mol_type = other RNA
acagcttatttggaagctgaaatgtgaggtttataacactcacaagaatcctgaaaaaggatgccaaacttttcttagagacagagtct

SEQ ID NO: 233 - RNA, 89 nt, synthetic construct
  source 1..89: mol_type = other RNA
acagcttatttggaagctgaaatgtgaggtttataacactcacaagaatcctgaaaaaggatgccaaacccatagattgtaatttaatg

SEQ ID NO: 234 - RNA, 89 nt, synthetic construct
  source 1..89: mol_type = other RNA
acagcttatttggaagctgaaatgtgaggtttataacactcacaagaatcctgaaaaaggatgccaaactttattttcttagagacaga

SEQ ID NO: 235 - RNA, 89 nt, synthetic construct
  source 1..89: mol_type = other RNA
acagcttatttggaagctgaaatgtgaggtttataacactcacaagaatcctgaaaaaggatgccaaacgctaggatgatgaacaacag

SEQ ID NO: 236 - RNA, 89 nt, synthetic construct
  source 1..89: mol_type = other RNA
acagcttatttggaagctgaaatgtgaggtttataacactcacaagaatcctgaaaaaggatgccaaacgtagtaaatgctagtctgga

SEQ ID NO: 237 - RNA, 89 nt, synthetic construct
  source 1..89: mol_type = other RNA
acagcttatttggaagctgaaatgtgaggtttataacactcacaagaatcctgaaaaaggatgccaaacatggcaaatattagtttctg

SEQ ID NO: 238 - RNA, 89 nt, synthetic construct
  source 1..89: mol_type = other RNA
acagcttatttggaagctgaaatgtgaggtttataacactcacaagaatcctgaaaaaggatgccaaactagtagtaaatgctagtctg

SEQ ID NO: 239 - RNA, 89 nt, synthetic construct
  source 1..89: mol_type = other RNA
acagcttatttggaagctgaaatgtgaggtttataacactcacaagaatcctgaaaaaggatgccaaacttatggctaggatgatgaac

SEQ ID NO: 240 - RNA, 89 nt, synthetic construct
  source 1..89: mol_type = other RNA
acagcttatttggaagctgaaatgtgaggtttataacactcacaagaatcctgaaaaaggatgccaaacgaggagacattttaaatgta

SEQ ID NO: 241 - RNA, 89 nt, synthetic construct
  source 1..89: mol_type = other RNA
acagcttatttggaagctgaaatgtgaggtttataacactcacaagaatcctgaaaaaggatgccaaacaatgtaacttccaaacgtta

SEQ ID NO: 242 - RNA, 89 nt, synthetic construct
  source 1..89: mol_type = other RNA
acagcttatttggaagctgaaatgtgaggtttataacactcacaagaatcctgaaaaaggatgccaaacacttccaaacgttatctcac

SEQ ID NO: 243 - RNA, 89 nt, synthetic construct
  source 1..89: mol_type = other RNA
acagcttatttggaagctgaaatgtgaggtttataacactcacaagaatcctgaaaaaggatgccaaaccttttttgatggcaaatatt

SEQ ID NO: 244 - RNA, 89 nt, synthetic construct
  source 1..89: mol_type = other RNA
acagcttatttggaagctgaaatgtgaggtttataacactcacaagaatcctgaaaaaggatgccaaacagataacgtttggaagttac

SEQ ID NO: 245 - RNA, 89 nt, synthetic construct
  source 1..89: mol_type = other RNA
acagcttatttggaagctgaaatgtgaggtttataacactcacaagaatcctgaaaaaggatgccaaacccatcaaaaaagcaaagaat

SEQ ID NO: 246 - RNA, 89 nt, synthetic construct
  source 1..89: mol_type = other RNA
acagcttatttggaagctgaaatgtgaggtttataacactcacaagaatcctgaaaaaggatgccaaacaataagcaacataaatgtga

SEQ ID NO: 247 - RNA, 89 nt, synthetic construct
  source 1..89: mol_type = other RNA
acagcttatttggaagctgaaatgtgaggtttataacactcacaagaatcctgaaaaaggatgccaaacgttgaaagaattcagaatca

SEQ ID NO: 248 - RNA, 89 nt, synthetic construct
  source 1..89: mol_type = other RNA
acagcttatttggaagctgaaatgtgaggtttataacactcacaagaatcctgaaaaaggatgccaaacgaagttacatttaaaatgtc

SEQ ID NO: 249 - RNA, 89 nt, synthetic construct
  source 1..89: mol_type = other RNA
acagcttatttggaagctgaaatgtgaggtttataacactcacaagaatcctgaaaaaggatgccaaacacagaaactaatatttgcca

SEQ ID NO: 250 - RNA, 89 nt, synthetic construct
  source 1..89: mol_type = other RNA
acagcttatttggaagctgaaatgtgaggtttataacactcacaagaatcctgaaaaaggatgccaaacaaatgtctcctccagactag

SEQ ID NO: 251 - RNA, 89 nt, synthetic construct
  source 1..89: mol_type = other RNA
acagcttatttggaagctgaaatgtgaggtttataacactcacaagaatcctgaaaaaggatgccaaacttctagttgaaagaattcag

SEQ ID NO: 252 - RNA, 89 nt, synthetic construct
  source 1..89: mol_type = other RNA
acagcttatttggaagctgaaatgtgaggtttataacactcacaagaatcctgaaaaaggatgccaaacccaacttttatcattttttc

SEQ ID NO: 253 - RNA, 89 nt, synthetic construct
  source 1..89: mol_type = other RNA
acagcttatttggaagctgaaatgtgaggtttataacactcacaagaatcctgaaaaaggatgccaaactttgctgagagagaaacagt

SEQ ID NO: 254 - RNA, 89 nt, synthetic construct
  source 1..89: mol_type = other RNA
acagcttatttggaagctgaaatgtgaggtttataacactcacaagaatcctgaaaaaggatgccaaaccttaggctgaatagtgagag

SEQ ID NO: 255 - RNA, 89 nt, synthetic construct
  source 1..89: mol_type = other RNA
acagcttatttggaagctgaaatgtgaggtttataacactcacaagaatcctgaaaaaggatgccaaactcattttttctcataccttc

SEQ ID NO: 256 - RNA, 89 nt, synthetic construct
  source 1..89: mol_type = other RNA
acagcttatttggaagctgaaatgtgaggtttataacactcacaagaatcctgaaaaaggatgccaaaccttgatgatcatctcgttga

SEQ ID NO: 257 - RNA, 89 nt, synthetic construct
  source 1..89: mol_type = other RNA
acagcttatttggaagctgaaatgtgaggtttataacactcacaagaatcctgaaaaaggatgccaaacctgagagagaaacagttgcc

SEQ ID NO: 258 - RNA, 89 nt, synthetic construct
  source 1..89: mol_type = other RNA
acagcttatttggaagctgaaatgtgaggtttataacactcacaagaatcctgaaaaaggatgccaaacgctacttttgttatttgcat

SEQ ID NO: 259 - RNA, 89 nt, synthetic construct
  source 1..89: mol_type = other RNA
acagcttatttggaagctgaaatgtgaggtttataacactcacaagaatcctgaaaaaggatgccaaacggagagtaaagtgattggtg

SEQ ID NO: 260 - RNA, 89 nt, synthetic construct
  source 1..89: mol_type = other RNA
acagcttatttggaagctgaaatgtgaggtttataacactcacaagaatcctgaaaaaggatgccaaactggctacttttgttatttgc

SEQ ID NO: 261 - RNA, 89 nt, synthetic construct
  source 1..89: mol_type = other RNA
acagcttatttggaagctgaaatgtgaggtttataacactcacaagaatcctgaaaaaggatgccaaacaagaaaaacttctgccaact

SEQ ID NO: 262 - RNA, 89 nt, synthetic construct
  source 1..89: mol_type = other RNA
acagcttatttggaagctgaaatgtgaggtttataacactcacaagaatcctgaaaaaggatgccaaactatccttgattatacttagg

SEQ ID NO: 263 - RNA, 89 nt, synthetic construct
  source 1..89: mol_type = other RNA
acagcttatttggaagctgaaatgtgaggtttataacactcacaagaatcctgaaaaaggatgccaaactccttgattatacttaggct

SEQ ID NO: 264 - RNA, 89 nt, synthetic construct
  source 1..89: mol_type = other RNA
acagcttatttggaagctgaaatgtgaggtttataacactcacaagaatcctgaaaaaggatgccaaacaaatgaagattttccaccaa

SEQ ID NO: 265 - RNA, 89 nt, synthetic construct
  source 1..89: mol_type = other RNA
acagcttatttggaagctgaaatgtgaggtttataacactcacaagaatcctgaaaaaggatgccaaacctctcctagaccatttccca

SEQ ID NO: 266 - RNA, 89 nt, synthetic construct
  source 1..89: mol_type = other RNA
acagcttatttggaagctgaaatgtgaggtttataacactcacaagaatcctgaaaaaggatgccaaacagtaggagctaaaatatttt

SEQ ID NO: 267 - RNA, 89 nt, synthetic construct
  source 1..89: mol_type = other RNA
acagcttatttggaagctgaaatgtgaggtttataacactcacaagaatcctgaaaaaggatgccaaacgatactttgtttagcaatac

SEQ ID NO: 268 - RNA, 89 nt, synthetic construct
  source 1..89: mol_type = other RNA
acagcttatttggaagctgaaatgtgaggtttataacactcacaagaatcctgaaaaaggatgccaaacggtttttgcaaaaaggaaaa

SEQ ID NO: 269 - RNA, 89 nt, synthetic construct
  source 1..89: mol_type = other RNA
acagcttatttggaagctgaaatgtgaggtttataacactcacaagaatcctgaaaaaggatgccaaactctttttctaacaatgtgga

SEQ ID NO: 270 - RNA, 89 nt, synthetic construct
  source 1..89: mol_type = other RNA
acagcttatttggaagctgaaatgtgaggtttataacactcacaagaatcctgaaaaaggatgccaaacacaatgtggatactttgttt

SEQ ID NO: 271 - RNA, 89 nt, synthetic construct
  source 1..89: mol_type = other RNA
acagcttatttggaagctgaaatgtgaggtttataacactcacaagaatcctgaaaaaggatgccaaacagccaaactcttattcatga

SEQ ID NO: 272 - RNA, 89 nt, synthetic construct
  source 1..89: mol_type = other RNA
acagcttatttggaagctgaaatgtgaggtttataacactcacaagaatcctgaaaaaggatgccaaacattgaagagtaacaatttga

SEQ ID NO: 273 - RNA, 89 nt, synthetic construct
  source 1..89: mol_type = other RNA
acagcttatttggaagctgaaatgtgaggtttataacactcacaagaatcctgaaaaaggatgccaaacgcaatacatggtagaaaatg

SEQ ID NO: 274 - RNA, 89 nt, synthetic construct
  source 1..89: mol_type = other RNA
acagcttatttggaagctgaaatgtgaggtttataacactcacaagaatcctgaaaaaggatgccaaaccaaaaaggaaaaaagaagaa

SEQ ID NO: 275 - RNA, 89 nt, synthetic construct
  source 1..89: mol_type = other RNA
acagcttatttggaagctgaaatgtgaggtttataacactcacaagaatcctgaaaaaggatgccaaactttagcaatacatggtagaa

SEQ ID NO: 276 - RNA, 89 nt, synthetic construct
  source 1..89: mol_type = other RNA
acagcttatttggaagctgaaatgtgaggtttataacactcacaagaatcctgaaaaaggatgccaaactatctttttctaacaatgtg

SEQ ID NO: 277 - RNA, 89 nt, synthetic construct
  source 1..89: mol_type = other RNA
acagcttatttggaagctgaaatgtgaggtttataacactcacaagaatcctgaaaaaggatgccaaacatgtcatgaataagagtttg

SEQ ID NO: 278 - RNA, 89 nt, synthetic construct
  source 1..89: mol_type = other RNA
acagcttatttggaagctgaaatgtgaggtttataacactcacaagaatcctgaaaaaggatgccaaacgctcctactcagactgttac

SEQ ID NO: 279 - RNA, 89 nt, synthetic construct
  source 1..89: mol_type = other RNA
acagcttatttggaagctgaaatgtgaggtttataacactcacaagaatcctgaaaaaggatgccaaacgcttgtgtttctaatttttc

SEQ ID NO: 280 - RNA, 89 nt, synthetic construct
  source 1..89: mol_type = other RNA
acagcttatttggaagctgaaatgtgaggtttataacactcacaagaatcctgaaaaaggatgccaaacttgctaaacaaagtatccac

SEQ ID NO: 281 - RNA, 89 nt, synthetic construct
  source 1..89: mol_type = other RNA
acagcttatttggaagctgaaatgtgaggtttataacactcacaagaatcctgaaaaaggatgccaaactaatgtcatgaataagagtt

SEQ ID NO: 282 - RNA, 89 nt, synthetic construct
  source 1..89: mol_type = other RNA
acagcttatttggaagctgaaatgtgaggtttataacactcacaagaatcctgaaaaaggatgccaaacccatgtattgctaaacaaag

SEQ ID NO: 283 - RNA, 89 nt, synthetic construct
  source 1..89: mol_type = other RNA
acagcttatttggaagctgaaatgtgaggtttataacactcacaagaatcctgaaaaaggatgccaaacgctcaaattgttactcttca

SEQ ID NO: 284 - RNA, 89 nt, synthetic construct
  source 1..89: mol_type = other RNA
acagcttatttggaagctgaaatgtgaggtttataacactcacaagaatcctgaaaaaggatgccaaacagactttttgcacagtcaat

SEQ ID NO: 285 - RNA, 89 nt, synthetic construct
  source 1..89: mol_type = other RNA
acagcttatttggaagctgaaatgtgaggtttataacactcacaagaatcctgaaaaaggatgccaaaccttacaggctccaatagtgg

SEQ ID NO: 286 - RNA, 89 nt, synthetic construct
  source 1..89: mol_type = other RNA
acagcttatttggaagctgaaatgtgaggtttataacactcacaagaatcctgaaaaaggatgccaaaccacagtcaataacacaaagg

SEQ ID NO: 287 - RNA, 89 nt, synthetic construct
  source 1..89: mol_type = other RNA
acagcttatttggaagctgaaatgtgaggtttataacactcacaagaatcctgaaaaaggatgccaaacgaattgaaacaaattttctc

SEQ ID NO: 288 - RNA, 89 nt, synthetic construct
  source 1..89: mol_type = other RNA
acagcttatttggaagctgaaatgtgaggtttataacactcacaagaatcctgaaaaaggatgccaaactctctatctttagaattgaa

SEQ ID NO: 289 - RNA, 89 nt, synthetic construct
  source 1..89: mol_type = other RNA
acagcttatttggaagctgaaatgtgaggtttataacactcacaagaatcctgaaaaaggatgccaaacaacaaatagctagagccaaa

SEQ ID NO: 290 - RNA, 89 nt, synthetic construct
  source 1..89: mol_type = other RNA
acagcttatttggaagctgaaatgtgaggtttataacactcacaagaatcctgaaaaaggatgccaaacaatcagagtcaatttccaag

SEQ ID NO: 291 - RNA, 89 nt, synthetic construct
  source 1..89: mol_type = other RNA
acagcttatttggaagctgaaatgtgaggtttataacactcacaagaatcctgaaaaaggatgccaaacctctaagactttttgcacag

SEQ ID NO: 292 - RNA, 89 nt, synthetic construct
  source 1..89: mol_type = other RNA
acagcttatttggaagctgaaatgtgaggtttataacactcacaagaatcctgaaaaaggatgccaaacttcaaaagtgcaactatgaa

SEQ ID NO: 293 - RNA, 89 nt, synthetic construct
  source 1..89: mol_type = other RNA
acagcttatttggaagctgaaatgtgaggtttataacactcacaagaatcctgaaaaaggatgccaaacgctctagctatttgttcaaa

SEQ ID NO: 294 - RNA, 89 nt, synthetic construct
  source 1..89: mol_type = other RNA
acagcttatttggaagctgaaatgtgaggtttataacactcacaagaatcctgaaaaaggatgccaaacgctatttgttcaaaagtgca

SEQ ID NO: 295 - RNA, 89 nt, synthetic construct
  source 1..89: mol_type = other RNA
acagcttatttggaagctgaaatgtgaggtttataacactcacaagaatcctgaaaaaggatgccaaacagtatactggatcccattct

SEQ ID NO: 296 - RNA, 89 nt, synthetic construct
  source 1..89: mol_type = other RNA
acagcttatttggaagctgaaatgtgaggtttataacactcacaagaatcctgaaaaaggatgccaaactgttattgactgtgcaaaaa

SEQ ID NO: 297 - RNA, 89 nt, synthetic construct
  source 1..89: mol_type = other RNA
acagcttatttggaagctgaaatgtgaggtttataacactcacaagaatcctgaaaaaggatgccaaacattcaaagtgttgcatgaca

SEQ ID NO: 298 - RNA, 89 nt, synthetic construct
  source 1..89: mol_type = other RNA
acagcttatttggaagctgaaatgtgaggtttataacactcacaagaatcctgaaaaaggatgccaaactttcaattctaaagatagag

SEQ ID NO: 299 - RNA, 89 nt, synthetic construct
  source 1..89: mol_type = other RNA
acagcttatttggaagctgaaatgtgaggtttataacactcacaagaatcctgaaaaaggatgccaaacaagatagagataaacctttg

SEQ ID NO: 300 - RNA, 89 nt, synthetic construct
  source 1..89: mol_type = other RNA
acagcttatttggaagctgaaatgtgaggtttataacactcacaagaatcctgaaaaaggatgccaaacttattgactgtgcaaaaagt

SEQ ID NO: 301 - RNA, 89 nt, synthetic construct
  source 1..89: mol_type = other RNA
acagcttatttggaagctgaaatgtgaggtttataacactcacaagaatcctgaaaaaggatgccaaacaagtgatgactgggtgagag

SEQ ID NO: 302 - RNA, 89 nt, synthetic construct
  source 1..89: mol_type = other RNA
acagcttatttggaagctgaaatgtgaggtttataacactcacaagaatcctgaaaaaggatgccaaacctggatcccattctctttgg

SEQ ID NO: 303 - RNA, 89 nt, synthetic construct
  source 1..89: mol_type = other RNA
acagcttatttggaagctgaaatgtgaggtttataacactcacaagaatcctgaaaaaggatgccaaaccaaaaagtcttagagtacat

SEQ ID NO: 304 - RNA, 89 nt, synthetic construct
  source 1..89: mol_type = other RNA
acagcttatttggaagctgaaatgtgaggtttataacactcacaagaatcctgaaaaaggatgccaaacaagataattcatgaacatct

SEQ ID NO: 305 - RNA, 89 nt, synthetic construct
  source 1..89: mol_type = other RNA
acagcttatttggaagctgaaatgtgaggtttataacactcacaagaatcctgaaaaaggatgccaaacattattttagccaaccaccc

SEQ ID NO: 306 - RNA, 89 nt, synthetic construct
  source 1..89: mol_type = other RNA
acagcttatttggaagctgaaatgtgaggtttataacactcacaagaatcctgaaaaaggatgccaaaccttctaaattaactttagtg

SEQ ID NO: 307 - RNA, 89 nt, synthetic construct
  source 1..89: mol_type = other RNA
acagcttatttggaagctgaaatgtgaggtttataacactcacaagaatcctgaaaaaggatgccaaacaattaactttagtgggtaga

SEQ ID NO: 308 - RNA, 89 nt, synthetic construct
  source 1..89: mol_type = other RNA
acagcttatttggaagctgaaatgtgaggtttataacactcacaagaatcctgaaaaaggatgccaaacgccaaccaccctacaaatat

SEQ ID NO: 309 - RNA, 89 nt, synthetic construct
  source 1..89: mol_type = other RNA
acagcttatttggaagctgaaatgtgaggtttataacactcacaagaatcctgaaaaaggatgccaaacgtgggtagaatttcttttaa

SEQ ID NO: 310 - RNA, 89 nt, synthetic construct
  source 1..89: mol_type = other RNA
acagcttatttggaagctgaaatgtgaggtttataacactcacaagaatcctgaaaaaggatgccaaacacagaaaagcatacacatta

SEQ ID NO: 311 - RNA, 89 nt, synthetic construct
  source 1..89: mol_type = other RNA
acagcttatttggaagctgaaatgtgaggtttataacactcacaagaatcctgaaaaaggatgccaaacacttcctctttaacagaaaa

SEQ ID NO: 312 - RNA, 89 nt, synthetic construct
  source 1..89: mol_type = other RNA
acagcttatttggaagctgaaatgtgaggtttataacactcacaagaatcctgaaaaaggatgccaaacaaagaaattctacccactaa

SEQ ID NO: 313 - RNA, 89 nt, synthetic construct
  source 1..89: mol_type = other RNA
acagcttatttggaagctgaaatgtgaggtttataacactcacaagaatcctgaaaaaggatgccaaactagggtggttggctaaaata

SEQ ID NO: 314 - RNA, 89 nt, synthetic construct
  source 1..89: mol_type = other RNA
acagcttatttggaagctgaaatgtgaggtttataacactcacaagaatcctgaaaaaggatgccaaactatgcttttctgttaaagag

SEQ ID NO: 315 - RNA, 89 nt, synthetic construct
  source 1..89: mol_type = other RNA
acagcttatttggaagctgaaatgtgaggtttataacactcacaagaatcctgaaaaaggatgccaaaccccactaaagttaatttaga

SEQ ID NO: 316 - RNA, 89 nt, synthetic construct
  source 1..89: mol_type = other RNA
acagcttatttggaagctgaaatgtgaggtttataacactcacaagaatcctgaaaaaggatgccaaacttaaagaggaagttagaaga

SEQ ID NO: 317 - RNA, 89 nt, synthetic construct
  source 1..89: mol_type = other RNA
acagcttatttggaagctgaaatgtgaggtttataacactcacaagaatcctgaaaaaggatgccaaactgcttttctgttaaagagga

SEQ ID NO: 318 - RNA, 89 nt, synthetic construct
  source 1..89: mol_type = other RNA
acagcttatttggaagctgaaatgtgaggtttataacactcacaagaatcctgaaaaaggatgccaaacttcaccaaatggattaagat

SEQ ID NO: 319 - RNA, 89 nt, synthetic construct
  source 1..89: mol_type = other RNA
acagcttatttggaagctgaaatgtgaggtttataacactcacaagaatcctgaaaaaggatgccaaacgggtggttggctaaaataat

SEQ ID NO: 320 - RNA, 89 nt, synthetic construct
  source 1..89: mol_type = other RNA
acagcttatttggaagctgaaatgtgaggtttataacactcacaagaatcctgaaaaaggatgccaaaccttttctgttaaagaggaag

SEQ ID NO: 321 - RNA, 89 nt, synthetic construct
  source 1..89: mol_type = other RNA
acagcttatttggaagctgaaatgtgaggtttataacactcacaagaatcctgaaaaaggatgccaaacctaaaatgttttcattccta

SEQ ID NO: 322 - RNA, 89 nt, synthetic construct
  source 1..89: mol_type = other RNA
acagcttatttggaagctgaaatgtgaggtttataacactcacaagaatcctgaaaaaggatgccaaacctgctgttgattaatggttg

SEQ ID NO: 323 - RNA, 89 nt, synthetic construct
  source 1..89: mol_type = other RNA
acagcttatttggaagctgaaatgtgaggtttataacactcacaagaatcctgaaaaaggatgccaaacatctctcatgaaatattctt

SEQ ID NO: 324 - RNA, 89 nt, synthetic construct
  source 1..89: mol_type = other RNA
acagcttatttggaagctgaaatgtgaggtttataacactcacaagaatcctgaaaaaggatgccaaacaagaaagcttaaaaagtctg

SEQ ID NO: 325 - RNA, 89 nt, synthetic construct
  source 1..89: mol_type = other RNA
acagcttatttggaagctgaaatgtgaggtttataacactcacaagaatcctgaaaaaggatgccaaactcgccctacctcttttttct

SEQ ID NO: 326 - RNA, 89 nt, synthetic construct
  source 1..89: mol_type = other RNA
acagcttatttggaagctgaaatgtgaggtttataacactcacaagaatcctgaaaaaggatgccaaacatgttagtgcctttcaccct

SEQ ID NO: 327 - RNA, 89 nt, synthetic construct
  source 1..89: mol_type = other RNA
acagcttatttggaagctgaaatgtgaggtttataacactcacaagaatcctgaaaaaggatgccaaacagcagggtgaaaggcactaa

SEQ ID NO: 328 - RNA, 89 nt, synthetic construct
  source 1..89: mol_type = other RNA
acagcttatttggaagctgaaatgtgaggtttataacactcacaagaatcctgaaaaaggatgccaaacgaagaatatttcatgagaga

SEQ ID NO: 329 - RNA, 89 nt, synthetic construct
  source 1..89: mol_type = other RNA
acagcttatttggaagctgaaatgtgaggtttataacactcacaagaatcctgaaaaaggatgccaaacagctttctttagaagaatat

SEQ ID NO: 330 - RNA, 89 nt, synthetic construct
  source 1..89: mol_type = other RNA
acagcttatttggaagctgaaatgtgaggtttataacactcacaagaatcctgaaaaaggatgccaaacctaaaatatatacttgtggc

SEQ ID NO: 331 - RNA, 89 nt, synthetic construct
  source 1..89: mol_type = other RNA
acagcttatttggaagctgaaatgtgaggtttataacactcacaagaatcctgaaaaaggatgccaaacgcagactttttaagctttct

SEQ ID NO: 332 - RNA, 89 nt, synthetic construct
  source 1..89: mol_type = other RNA
acagcttatttggaagctgaaatgtgaggtttataacactcacaagaatcctgaaaaaggatgccaaacaaaatttccctatgaaactg

SEQ ID NO: 333 - RNA, 89 nt, synthetic construct
  source 1..89: mol_type = other RNA
acagcttatttggaagctgaaatgtgaggtttataacactcacaagaatcctgaaaaaggatgccaaactttgagaaaagattaaacag

SEQ ID NO: 334 - RNA, 89 nt, synthetic construct
  source 1..89: mol_type = other RNA
acagcttatttggaagctgaaatgtgaggtttataacactcacaagaatcctgaaaaaggatgccaaacagaaaagattaaacagtgtg

SEQ ID NO: 335 - RNA, 89 nt, synthetic construct
  source 1..89: mol_type = other RNA
acagcttatttggaagctgaaatgtgaggtttataacactcacaagaatcctgaaaaaggatgccaaacagataccaaaaaggcaaaac

SEQ ID NO: 336 - RNA, 89 nt, synthetic construct
  source 1..89: mol_type = other RNA
acagcttatttggaagctgaaatgtgaggtttataacactcacaagaatcctgaaaaaggatgccaaacctggcaaagaaagaaataca

SEQ ID NO: 337 - RNA, 89 nt, synthetic construct
  source 1..89: mol_type = other RNA
acagcttatttggaagctgaaatgtgaggtttataacactcacaagaatcctgaaaaaggatgccaaacctaccacatgcagttgtact

SEQ ID NO: 338 - RNA, 89 nt, synthetic construct
  source 1..89: mol_type = other RNA
acagcttatttggaagctgaaatgtgaggtttataacactcacaagaatcctgaaaaaggatgccaaacaaactgacatgcccatatcc

SEQ ID NO: 339 - RNA, 89 nt, synthetic construct
  source 1..89: mol_type = other RNA
acagcttatttggaagctgaaatgtgaggtttataacactcacaagaatcctgaaaaaggatgccaaacttttgcctttttggtatctt

SEQ ID NO: 340 - RNA, 89 nt, synthetic construct
  source 1..89: mol_type = other RNA
acagcttatttggaagctgaaatgtgaggtttataacactcacaagaatcctgaaaaaggatgccaaacgtagcacactgtttaatctt

SEQ ID NO: 341 - RNA, 89 nt, synthetic construct
  source 1..89: mol_type = other RNA
acagcttatttggaagctgaaatgtgaggtttataacactcacaagaatcctgaaaaaggatgccaaactcctttggatatgggcatgt

SEQ ID NO: 342 - RNA, 89 nt, synthetic construct
  source 1..89: mol_type = other RNA
acagcttatttggaagctgaaatgtgaggtttataacactcacaagaatcctgaaaaaggatgccaaactatttctttctttgccagta

SEQ ID NO: 343 - RNA, 89 nt, synthetic construct
  source 1..89: mol_type = other RNA
acagcttatttggaagctgaaatgtgaggtttataacactcacaagaatcctgaaaaaggatgccaaactcttgtatcctttggatatg

SEQ ID NO: 344 - RNA, 89 nt, synthetic construct
  source 1..89: mol_type = other RNA
acagcttatttggaagctgaaatgtgaggtttataacactcacaagaatcctgaaaaaggatgccaaaccctttttggtatcttacagg

SEQ ID NO: 345 - RNA, 89 nt, synthetic construct
  source 1..89: mol_type = other RNA
acagcttatttggaagctgaaatgtgaggtttataacactcacaagaatcctgaaaaaggatgccaaacgatatgggcatgtcagtttc

SEQ ID NO: 346 - RNA, 89 nt, synthetic construct
  source 1..89: mol_type = other RNA
acagcttatttggaagctgaaatgtgaggtttataacactcacaagaatcctgaaaaaggatgccaaacgtatcttacaggaactccag

SEQ ID NO: 347 - RNA, 89 nt, synthetic construct
  source 1..89: mol_type = other RNA
acagcttatttggaagctgaaatgtgaggtttataacactcacaagaatcctgaaaaaggatgccaaactttctttctttgccagtaca

SEQ ID NO: 348 - RNA, 89 nt, synthetic construct
  source 1..89: mol_type = other RNA
acagcttatttggaagctgaaatgtgaggtttataacactcacaagaatcctgaaaaaggatgccaaacccagtacaactgcatgtggt

SEQ ID NO: 349 - RNA, 89 nt, synthetic construct
  source 1..89: mol_type = other RNA
acagcttatttggaagctgaaatgtgaggtttataacactcacaagaatcctgaaaaaggatgccaaacggcatgtcagtttcataggg

SEQ ID NO: 350 - RNA, 89 nt, synthetic construct
  source 1..89: mol_type = other RNA
acagcttatttggaagctgaaatgtgaggtttataacactcacaagaatcctgaaaaaggatgccaaacgtaatataatgatgacaaca

SEQ ID NO: 351 - RNA, 89 nt, synthetic construct
  source 1..89: mol_type = other RNA
acagcttatttggaagctgaaatgtgaggtttataacactcacaagaatcctgaaaaaggatgccaaacagaagttaaagagtccagat

SEQ ID NO: 352 - RNA, 89 nt, synthetic construct
  source 1..89: mol_type = other RNA
acagcttatttggaagctgaaatgtgaggtttataacactcacaagaatcctgaaaaaggatgccaaacatgatgacaacaacagtcaa

SEQ ID NO: 353 - RNA, 89 nt, synthetic construct
  source 1..89: mol_type = other RNA
acagcttatttggaagctgaaatgtgaggtttataacactcacaagaatcctgaaaaaggatgccaaacctgaagataaatacaatttc

SEQ ID NO: 354 - RNA, 89 nt, synthetic construct
  source 1..89: mol_type = other RNA
acagcttatttggaagctgaaatgtgaggtttataacactcacaagaatcctgaaaaaggatgccaaactttatcttcagcacatctgg

SEQ ID NO: 355 - RNA, 89 nt, synthetic construct
  source 1..89: mol_type = other RNA
acagcttatttggaagctgaaatgtgaggtttataacactcacaagaatcctgaaaaaggatgccaaacaagggtgatggaaattactt

SEQ ID NO: 356 - RNA, 89 nt, synthetic construct
  source 1..89: mol_type = other RNA
acagcttatttggaagctgaaatgtgaggtttataacactcacaagaatcctgaaaaaggatgccaaacactgttgttgtcatcattat

SEQ ID NO: 357 - RNA, 89 nt, synthetic construct
  source 1..89: mol_type = other RNA
acagcttatttggaagctgaaatgtgaggtttataacactcacaagaatcctgaaaaaggatgccaaacacttcttaaagatcaggttc

SEQ ID NO: 358 - RNA, 89 nt, synthetic construct
  source 1..89: mol_type = other RNA
acagcttatttggaagctgaaatgtgaggtttataacactcacaagaatcctgaaaaaggatgccaaactcttcagcacatctggactc

SEQ ID NO: 359 - RNA, 89 nt, synthetic construct
  source 1..89: mol_type = other RNA
acagcttatttggaagctgaaatgtgaggtttataacactcacaagaatcctgaaaaaggatgccaaacgactttttgtgtcaggatga

SEQ ID NO: 360 - RNA, 89 nt, synthetic construct
  source 1..89: mol_type = other RNA
acagcttatttggaagctgaaatgtgaggtttataacactcacaagaatcctgaaaaaggatgccaaacgactctttaacttcttaaag

SEQ ID NO: 361 - RNA, 89 nt, synthetic construct
  source 1..89: mol_type = other RNA
acagcttatttggaagctgaaatgtgaggtttataacactcacaagaatcctgaaaaaggatgccaaacgttatactgacaaagatatc

SEQ ID NO: 362 - RNA, 89 nt, synthetic construct
  source 1..89: mol_type = other RNA
acagcttatttggaagctgaaatgtgaggtttataacactcacaagaatcctgaaaaaggatgccaaacttttttggttatactgacaa

SEQ ID NO: 363 - RNA, 89 nt, synthetic construct
  source 1..89: mol_type = other RNA
acagcttatttggaagctgaaatgtgaggtttataacactcacaagaatcctgaaaaaggatgccaaacctgacaaagatatcactctg

SEQ ID NO: 364 - RNA, 89 nt, synthetic construct
  source 1..89: mol_type = other RNA
acagcttatttggaagctgaaatgtgaggtttataacactcacaagaatcctgaaaaaggatgccaaacgcgtatattttttggttata

SEQ ID NO: 365 - RNA, 89 nt, synthetic construct
  source 1..89: mol_type = other RNA
acagcttatttggaagctgaaatgtgaggtttataacactcacaagaatcctgaaaaaggatgccaaaccagagtttagtttcaagtaa

SEQ ID NO: 366 - RNA, 89 nt, synthetic construct
  source 1..89: mol_type = other RNA
acagcttatttggaagctgaaatgtgaggtttataacactcacaagaatcctgaaaaaggatgccaaacatagtttcctgcatttgcag

SEQ ID NO: 367 - RNA, 89 nt, synthetic construct
  source 1..89: mol_type = other RNA
acagcttatttggaagctgaaatgtgaggtttataacactcacaagaatcctgaaaaaggatgccaaactcaaatcgcctgcaggtaaa

SEQ ID NO: 368 - RNA, 89 nt, synthetic construct
  source 1..89: mol_type = other RNA
acagcttatttggaagctgaaatgtgaggtttataacactcacaagaatcctgaaaaaggatgccaaacgatcaagaaaaatagatgga

SEQ ID NO: 369 - RNA, 89 nt, synthetic construct
  source 1..89: mol_type = other RNA
acagcttatttggaagctgaaatgtgaggtttataacactcacaagaatcctgaaaaaggatgccaaaccacacctagcatgtacacac

SEQ ID NO: 370 - RNA, 89 nt, synthetic construct
  source 1..89: mol_type = other RNA
acagcttatttggaagctgaaatgtgaggtttataacactcacaagaatcctgaaaaaggatgccaaacgagatatagcgtatattttt

SEQ ID NO: 371 - RNA, 89 nt, synthetic construct
  source 1..89: mol_type = other RNA
acagcttatttggaagctgaaatgtgaggtttataacactcacaagaatcctgaaaaaggatgccaaaccctgcaggcgatttgacaga

SEQ ID NO: 372 - RNA, 89 nt, synthetic construct
  source 1..89: mol_type = other RNA
acagcttatttggaagctgaaatgtgaggtttataacactcacaagaatcctgaaaaaggatgccaaaccgctatatctctataatctg

SEQ ID NO: 373 - RNA, 89 nt, synthetic construct
  source 1..89: mol_type = other RNA
acagcttatttggaagctgaaatgtgaggtttataacactcacaagaatcctgaaaaaggatgccaaactttttcttgatccatatgct

SEQ ID NO: 374 - RNA, 89 nt, synthetic construct
  source 1..89: mol_type = other RNA
acagcttatttggaagctgaaatgtgaggtttataacactcacaagaatcctgaaaaaggatgccaaaccaaatgcaggaaactatcag

SEQ ID NO: 375 - RNA, 89 nt, synthetic construct
  source 1..89: mol_type = other RNA
acagcttatttggaagctgaaatgtgaggtttataacactcacaagaatcctgaaaaaggatgccaaactttgttacttgaaactaaac

SEQ ID NO: 376 - RNA, 89 nt, synthetic construct
  source 1..89: mol_type = other RNA
acagcttatttggaagctgaaatgtgaggtttataacactcacaagaatcctgaaaaaggatgccaaactacatgctaggtgtgtatat

SEQ ID NO: 377 - RNA, 89 nt, synthetic construct
  source 1..89: mol_type = other RNA
acagcttatttggaagctgaaatgtgaggtttataacactcacaagaatcctgaaaaaggatgccaaactctctataatctgttttaca

SEQ ID NO: 378 - RNA, 89 nt, synthetic construct
  source 1..89: mol_type = other RNA
acagcttatttggaagctgaaatgtgaggtttataacactcacaagaatcctgaaaaaggatgccaaactgtacatgctaggtgtgtat

SEQ ID NO: 379 - RNA, 89 nt, synthetic construct
  source 1..89: mol_type = other RNA
acagcttatttggaagctgaaatgtgaggtttataacactcacaagaatcctgaaaaaggatgccaaaccttttacctgcaggcgattt

SEQ ID NO: 380 - RNA, 89 nt, synthetic construct
  source 1..89: mol_type = other RNA
acagcttatttggaagctgaaatgtgaggtttataacactcacaagaatcctgaaaaaggatgccaaacttacttgaaactaaactctg

SEQ ID NO: 381 - RNA, 89 nt, synthetic construct
  source 1..89: mol_type = other RNA
acagcttatttggaagctgaaatgtgaggtttataacactcacaagaatcctgaaaaaggatgccaaacacagatctgttgagaaatgg

SEQ ID NO: 382 - RNA, 89 nt, synthetic construct
  source 1..89: mol_type = other RNA
acagcttatttggaagctgaaatgtgaggtttataacactcacaagaatcctgaaaaaggatgccaaacaaatgttgtgtgtacatgct

SEQ ID NO: 383 - RNA, 89 nt, synthetic construct
  source 1..89: mol_type = other RNA
acagcttatttggaagctgaaatgtgaggtttataacactcacaagaatcctgaaaaaggatgccaaactaatctgttttacataatcc

SEQ ID NO: 384 - RNA, 89 nt, synthetic construct
  source 1..89: mol_type = other RNA
acagcttatttggaagctgaaatgtgaggtttataacactcacaagaatcctgaaaaaggatgccaaaccatgctaggtgtgtatatta

SEQ ID NO: 385 - RNA, 89 nt, synthetic construct
  source 1..89: mol_type = other RNA
acagcttatttggaagctgaaatgtgaggtttataacactcacaagaatcctgaaaaaggatgccaaaccataatccatctatttttct

SEQ ID NO: 386 - RNA, 89 nt, synthetic construct
  source 1..89: mol_type = other RNA
acagcttatttggaagctgaaatgtgaggtttataacactcacaagaatcctgaaaaaggatgccaaacatctgttttacataatccat

SEQ ID NO: 387 - RNA, 89 nt, synthetic construct
  source 1..89: mol_type = other RNA
acagcttatttggaagctgaaatgtgaggtttataacactcacaagaatcctgaaaaaggatgccaaacaccaaaaaatatacgctata

SEQ ID NO: 388 - RNA, 89 nt, synthetic construct
  source 1..89: mol_type = other RNA
acagcttatttggaagctgaaatgtgaggtttataacactcacaagaatcctgaaaaaggatgccaaacattttcttttggattgcatc

SEQ ID NO: 389 - RNA, 89 nt, synthetic construct
  source 1..89: mol_type = other RNA
acagcttatttggaagctgaaatgtgaggtttataacactcacaagaatcctgaaaaaggatgccaaacttgaatcctttaacatttca

SEQ ID NO: 390 - RNA, 89 nt, synthetic construct
  source 1..89: mol_type = other RNA
acagcttatttggaagctgaaatgtgaggtttataacactcacaagaatcctgaaaaaggatgccaaacgattgcatctactgtatagg

SEQ ID NO: 391 - RNA, 89 nt, synthetic construct
  source 1..89: mol_type = other RNA
acagcttatttggaagctgaaatgtgaggtttataacactcacaagaatcctgaaaaaggatgccaaacacatttcattcaactgttgc

SEQ ID NO: 392 - RNA, 89 nt, synthetic construct
  source 1..89: mol_type = other RNA
acagcttatttggaagctgaaatgtgaggtttataacactcacaagaatcctgaaaaaggatgccaaactagggaccctccttccatga

SEQ ID NO: 393 - RNA, 89 nt, synthetic construct
  source 1..89: mol_type = other RNA
acagcttatttggaagctgaaatgtgaggtttataacactcacaagaatcctgaaaaaggatgccaaaccttcatcccactgattctga

SEQ ID NO: 394 - RNA, 89 nt, synthetic construct
  source 1..89: mol_type = other RNA
acagcttatttggaagctgaaatgtgaggtttataacactcacaagaatcctgaaaaaggatgccaaacaaggtgttcttgtacttcat

SEQ ID NO: 395 - RNA, 89 nt, synthetic construct
  source 1..89: mol_type = other RNA
acagcttatttggaagctgaaatgtgaggtttataacactcacaagaatcctgaaaaaggatgccaaactgattttcttttggattgca

SEQ ID NO: 396 - RNA, 89 nt, synthetic construct
  source 1..89: mol_type = other RNA
acagcttatttggaagctgaaatgtgaggtttataacactcacaagaatcctgaaaaaggatgccaaacgggaccctccttccatgact

SEQ ID NO: 397 - RNA, 89 nt, synthetic construct
  source 1..89: mol_type = other RNA
acagcttatttggaagctgaaatgtgaggtttataacactcacaagaatcctgaaaaaggatgccaaacctgtatagggaccctccttc

SEQ ID NO: 398 - RNA, 89 nt, synthetic construct
  source 1..89: mol_type = other RNA
acagcttatttggaagctgaaatgtgaggtttataacactcacaagaatcctgaaaaaggatgccaaacgcctgtcctaagacctgctc

SEQ ID NO: 399 - RNA, 89 nt, synthetic construct
  source 1..89: mol_type = other RNA
acagcttatttggaagctgaaatgtgaggtttataacactcacaagaatcctgaaaaaggatgccaaaccagtagatgcaatccaaaag

SEQ ID NO: 400 - RNA, 89 nt, synthetic construct
  source 1..89: mol_type = other RNA
acagcttatttggaagctgaaatgtgaggtttataacactcacaagaatcctgaaaaaggatgccaaactccaagcccggttgaaatct

SEQ ID NO: 401 - RNA, 89 nt, synthetic construct
  source 1..89: mol_type = other RNA
acagcttatttggaagctgaaatgtgaggtttataacactcacaagaatcctgaaaaaggatgccaaacgtttggagatggcagtttcc

SEQ ID NO: 402 - RNA, 89 nt, synthetic construct
  source 1..89: mol_type = other RNA
acagcttatttggaagctgaaatgtgaggtttataacactcacaagaatcctgaaaaaggatgccaaactcaccagagtaacagtctga

SEQ ID NO: 403 - RNA, 89 nt, synthetic construct
  source 1..89: mol_type = other RNA
acagcttatttggaagctgaaatgtgaggtttataacactcacaagaatcctgaaaaaggatgccaaacattttataacttgatcaagc

SEQ ID NO: 404 - RNA, 89 nt, synthetic construct
  source 1..89: mol_type = other RNA
acagcttatttggaagctgaaatgtgaggtttataacactcacaagaatcctgaaaaaggatgccaaactaacttgatcaagcagagaa

SEQ ID NO: 405 - RNA, 89 nt, synthetic construct
  source 1..89: mol_type = other RNA
acagcttatttggaagctgaaatgtgaggtttataacactcacaagaatcctgaaaaaggatgccaaacacttgatcaagcagagaaag

SEQ ID NO: 406 - RNA, 89 nt, synthetic construct
  source 1..89: mol_type = other RNA
acagcttatttggaagctgaaatgtgaggtttataacactcacaagaatcctgaaaaaggatgccaaacccagagcaggtacctccaac

SEQ ID NO: 407 - RNA, 89 nt, synthetic construct
  source 1..89: mol_type = other RNA
acagcttatttggaagctgaaatgtgaggtttataacactcacaagaatcctgaaaaaggatgccaaacgagatggcagtttccttagt

SEQ ID NO: 408 - RNA, 89 nt, synthetic construct
  source 1..89: mol_type = other RNA
acagcttatttggaagctgaaatgtgaggtttataacactcacaagaatcctgaaaaaggatgccaaacgttactaaggaaactgccat

SEQ ID NO: 409 - RNA, 89 nt, synthetic construct
  source 1..89: mol_type = other RNA
acagcttatttggaagctgaaatgtgaggtttataacactcacaagaatcctgaaaaaggatgccaaacgcagatttcaaccgggcttg

SEQ ID NO: 410 - RNA, 89 nt, synthetic construct
  source 1..89: mol_type = other RNA
acagcttatttggaagctgaaatgtgaggtttataacactcacaagaatcctgaaaaaggatgccaaacgtgacacaacctgtggttac

SEQ ID NO: 411 - RNA, 89 nt, synthetic construct
  source 1..89: mol_type = other RNA
acagcttatttggaagctgaaatgtgaggtttataacactcacaagaatcctgaaaaaggatgccaaacaaatcacagagggtgatggt

SEQ ID NO: 412 - RNA, 89 nt, synthetic construct
  source 1..89: mol_type = other RNA
acagcttatttggaagctgaaatgtgaggtttataacactcacaagaatcctgaaaaaggatgccaaaccttgatcaagttataaaatc

SEQ ID NO: 413 - RNA, 89 nt, synthetic construct
  source 1..89: mol_type = other RNA
acagcttatttggaagctgaaatgtgaggtttataacactcacaagaatcctgaaaaaggatgccaaacccgccttccactcagagctc

SEQ ID NO: 414 - RNA, 89 nt, synthetic construct
  source 1..89: mol_type = other RNA
acagcttatttggaagctgaaatgtgaggtttataacactcacaagaatcctgaaaaaggatgccaaacccctcagctcttgaagtaaa

SEQ ID NO: 415 - RNA, 89 nt, synthetic construct
  source 1..89: mol_type = other RNA
acagcttatttggaagctgaaatgtgaggtttataacactcacaagaatcctgaaaaaggatgccaaacagtggaaggcggtaaaccgt

SEQ ID NO: 416 - RNA, 89 nt, synthetic construct
  source 1..89: mol_type = other RNA
acagcttatttggaagctgaaatgtgaggtttataacactcacaagaatcctgaaaaaggatgccaaaccttcaagagctgagggcaaa

SEQ ID NO: 417 - RNA, 89 nt, synthetic construct
  source 1..89: mol_type = other RNA
acagcttatttggaagctgaaatgtgaggtttataacactcacaagaatcctgaaaaaggatgccaaacagctctgagtggaaggcggt

SEQ ID NO: 418 - RNA, 89 nt, synthetic construct
  source 1..89: mol_type = other RNA
acagcttatttggaagctgaaatgtgaggtttataacactcacaagaatcctgaaaaaggatgccaaacacagctgtttgcagacctcc

SEQ ID NO: 419 - RNA, 89 nt, synthetic construct
  source 1..89: mol_type = other RNA
acagcttatttggaagctgaaatgtgaggtttataacactcacaagaatcctgaaaaaggatgccaaactttttgaggattgctgaatt

SEQ ID NO: 420 - RNA, 89 nt, synthetic construct
  source 1..89: mol_type = other RNA
acagcttatttggaagctgaaatgtgaggtttataacactcacaagaatcctgaaaaaggatgccaaaccagacctcctgccaccgcag

SEQ ID NO: 421 - RNA, 89 nt, synthetic construct
  source 1..89: mol_type = other RNA
acagcttatttggaagctgaaatgtgaggtttataacactcacaagaatcctgaaaaaggatgccaaacaggattgctgaattatttct

SEQ ID NO: 422 - RNA, 89 nt, synthetic construct
  source 1..89: mol_type = other RNA
acagcttatttggaagctgaaatgtgaggtttataacactcacaagaatcctgaaaaaggatgccaaacgaatactggcatctgttttt

SEQ ID NO: 423 - RNA, 89 nt, synthetic construct
  source 1..89: mol_type = other RNA
acagcttatttggaagctgaaatgtgaggtttataacactcacaagaatcctgaaaaaggatgccaaactctgacagctgtttgcagac

SEQ ID NO: 424 - RNA, 89 nt, synthetic construct
  source 1..89: mol_type = other RNA
acagcttatttggaagctgaaatgtgaggtttataacactcacaagaatcctgaaaaaggatgccaaacacaacagtttgccgctgccc

SEQ ID NO: 425 - RNA, 89 nt, synthetic construct
  source 1..89: mol_type = other RNA
acagcttatttggaagctgaaatgtgaggtttataacactcacaagaatcctgaaaaaggatgccaaacccgctgcccaatgccatcct

SEQ ID NO: 426 - RNA, 89 nt, synthetic construct
  source 1..89: mol_type = other RNA
acagcttatttggaagctgaaatgtgaggtttataacactcacaagaatcctgaaaaaggatgccaaaccaaacagctgtcagacagaa

SEQ ID NO: 427 - RNA, 89 nt, synthetic construct
  source 1..89: mol_type = other RNA
acagcttatttggaagctgaaatgtgaggtttataacactcacaagaatcctgaaaaaggatgccaaaccaggaaaaattgggaagcct

SEQ ID NO: 428 - RNA, 89 nt, synthetic construct
  source 1..89: mol_type = other RNA
acagcttatttggaagctgaaatgtgaggtttataacactcacaagaatcctgaaaaaggatgccaaaccggtggcaggaggtctgcaa

SEQ ID NO: 429 - RNA, 89 nt, synthetic construct
  source 1..89: mol_type = other RNA
acagcttatttggaagctgaaatgtgaggtttataacactcacaagaatcctgaaaaaggatgccaaacgcatgttcccaattctcagg

SEQ ID NO: 430 - RNA, 89 nt, synthetic construct
  source 1..89: mol_type = other RNA
acagcttatttggaagctgaaatgtgaggtttataacactcacaagaatcctgaaaaaggatgccaaactcataatgaaaacgccgcca

SEQ ID NO: 431 - RNA, 89 nt, synthetic construct
  source 1..89: mol_type = other RNA
acagcttatttggaagctgaaatgtgaggtttataacactcacaagaatcctgaaaaaggatgccaaactctttctgagaaactgttca

SEQ ID NO: 432 - RNA, 89 nt, synthetic construct
  source 1..89: mol_type = other RNA
acagcttatttggaagctgaaatgtgaggtttataacactcacaagaatcctgaaaaaggatgccaaacttagccactgattaaatatc

SEQ ID NO: 433 - RNA, 89 nt, synthetic construct
  source 1..89: mol_type = other RNA
acagcttatttggaagctgaaatgtgaggtttataacactcacaagaatcctgaaaaaggatgccaaacagaaactgttcagcttctgt

SEQ ID NO: 434 - RNA, 89 nt, synthetic construct
  source 1..89: mol_type = other RNA
acagcttatttggaagctgaaatgtgaggtttataacactcacaagaatcctgaaaaaggatgccaaactatcataatgaaaacgccgc

SEQ ID NO: 435 - RNA, 89 nt, synthetic construct
  source 1..89: mol_type = other RNA
acagcttatttggaagctgaaatgtgaggtttataacactcacaagaatcctgaaaaaggatgccaaactttagcatgttcccaattct

SEQ ID NO: 436 - RNA, 89 nt, synthetic construct
  source 1..89: mol_type = other RNA
acagcttatttggaagctgaaatgtgaggtttataacactcacaagaatcctgaaaaaggatgccaaactatttagcatgttcccaatt

SEQ ID NO: 437 - RNA, 89 nt, synthetic construct
  source 1..89: mol_type = other RNA
acagcttatttggaagctgaaatgtgaggtttataacactcacaagaatcctgaaaaaggatgccaaactgtctttctgagaaactgtt

SEQ ID NO: 438 - RNA, 89 nt, synthetic construct
  source 1..89: mol_type = other RNA
acagcttatttggaagctgaaatgtgaggtttataacactcacaagaatcctgaaaaaggatgccaaacatcagtggctaacagaagct

SEQ ID NO: 439 - RNA, 89 nt, synthetic construct
  source 1..89: mol_type = other RNA
acagcttatttggaagctgaaatgtgaggtttataacactcacaagaatcctgaaaaaggatgccaaacttgagaaatggcggcgtttt

SEQ ID NO: 440 - RNA, 89 nt, synthetic construct
  source 1..89: mol_type = other RNA
acagcttatttggaagctgaaatgtgaggtttataacactcacaagaatcctgaaaaaggatgccaaacaagatatttaatcagtggct

SEQ ID NO: 441 - RNA, 52 nt, synthetic construct
  source 1..52: mol_type = other RNA
acagcttatttggaagctgaaatgtgaggtttataacactcacaagaatcct

SEQ ID NO: 442 - skipped

SEQ ID NO: 443 - RNA, 13 nt, synthetic construct
  source 1..13: mol_type = other RNA
aaggatgccaaac

SEQ ID NO: 444 - RNA, 56 nt, synthetic construct
  source 1..56: mol_type = other RNA
gtttgagaaccttatgaaattacaaggatgccaaactattaaatactcgtattgct

SEQ ID NO: 445 - RNA, 56 nt, synthetic construct; identical to SEQ ID NO: 444
  source 1..56: mol_type = other RNA

SEQ ID NO: 446 - RNA, 69 nt, synthetic construct
  source 1..69: mol_type = other RNA
tatatttgataaaaatatacagcttatttggaagctgaaatgtgaggtttataacactcacaagaatcc

SEQ ID NO: 447 - RNA, 89 nt, synthetic construct
  source 1..89: mol_type = other RNA
acagcttatttggaagctgaaatgtgaggtttataacactcacaagaatcctgaaaaaggatgccaaactattaaatactcgtattgct

SEQ ID NO: 448 - protein, 3685 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 449 - DNA, 13992 bp, synthetic construct
  source 1..13992: mol_type = other DNA
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttacaaaaaa

SEQ ID NO: 450 - RNA, 89 nt, synthetic construct; identical to SEQ ID NO: 402
  source 1..89: mol_type = other RNA

SEQ ID NO: 451 - DNA, 148 bp, synthetic construct
  source 1..148: mol_type = other DNA
cttaagataccatttgtatttagcatgttcccaattctcaggaatttgtgtctttctgagaaactgttcagcttctgttagccactgattaaatatctttatatcataatgaaaacgccgccatttctcaacagatctgtcaaatcgc

SEQ ID NO: 452 - DNA, 176 bp, synthetic construct
  source 1..176: mol_type = other DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 453 - DNA, 109 bp, synthetic construct
  source 1..109: mol_type = other DNA
aggaagttagaagatctgagctctgagtggaaggcggtaaaccgtttacttcaagagctgagggcaaagcagcctgacctagctcctggactgaccactattggagcct

SEQ ID NO: 454 - DNA, 233 bp, synthetic construct
  source 1..233: mol_type = other DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 455 - DNA, 212 bp, synthetic construct
  source 1..212: mol_type = other DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 456 - DNA, 60 bp, synthetic construct
  source 1..60: mol_type = other DNA
tcgtcggcagcgtcagatgtgtataagagacagctctgcaaatgcaggaaactatcagag

SEQ ID NO: 457 - DNA, 54 bp, synthetic construct
  source 1..54: mol_type = other DNA
gtctcgtgggctcggagatgtgtataagagacagtaaaccagctccgtccaggc

SEQ ID NO: 458 - DNA, 59 bp, synthetic construct
  source 1..59: mol_type = other DNA
tcgtcggcagcgtcagatgtgtataagagacagctgtcttgtatcctttggatatgggc

SEQ ID NO: 459 - DNA, 62 bp, synthetic construct
  source 1..62: mol_type = other DNA
gtctcgtgggctcggagatgtgtataagagacagctgctgttgattaatggttgataggttc

SEQ ID NO: 460 - DNA, 63 bp, synthetic construct
  source 1..63: mol_type = other DNA
tcgtcggcagcgtcagatgtgtataagagacaggcaattgataaatatttgtagggtggttgg

SEQ ID NO: 461 - DNA, 64 bp, synthetic construct
  source 1..64: mol_type = other DNA
gtctcgtgggctcggagatgtgtataagagacagagtcaatttccaaggaatgtactctaagac

SEQ ID NO: 462 - DNA, 63 bp, synthetic construct
  source 1..63: mol_type = other DNA
tcgtcggcagcgtcagatgtgtataagagacagattgaaattggctctttagcttgtgtttct

SEQ ID NO: 463 - DNA, 57 bp, synthetic construct
  source 1..57: mol_type = other DNA
gtctcgtgggctcggagatgtgtataagagacagcagttgcctaagaactggtggga

SEQ ID NO: 464 - DNA, 61 bp, synthetic construct
  source 1..61: mol_type = other DNA
tcgtcggcagcgtcagatgtgtataagagacagttgttcatcatcctagccataacacaat

SEQ ID NO: 465 - DNA, 62 bp, synthetic construct
  source 1..62: mol_type = other DNA
gtctcgtgggctcggagatgtgtataagagacaggtctactgttcatttcagctttaacgtg

SEQ ID NO: 466-467 - skipped (2 entries)

SEQ ID NO: 468 - protein, 8 aa, synthetic construct
  VARIANT 3: note = Any amino acid
  VARIANT 4: note = W, F or Y
  VARIANT 5: note = Any amino acid
  VARIANT 6: note = Any amino acid
KRXXXXAF

SEQ ID NO: 469 - skipped

SEQ ID NO: 470 - protein, 6 aa, synthetic construct
  VARIANT 5: note = K or R
  VARIANT 6: note = W, F or Y
LGKRXX

SEQ ID NO: 471 - skipped

SEQ ID NO: 472 - protein, 16 aa, synthetic construct
APKKKRKVGIHGVPAA

SEQ ID NO: 473 - protein, 18 aa, synthetic construct
KRPAATKKAGQAKKKKEF

SEQ ID NO: 474-475 - skipped (2 entries)

SEQ ID NO: 476 - protein, 659 aa, synthetic construct
MDYKDHDGDYKDHDIDYKDDDDKMAPKKKRKVGIHGVPAAMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKKEFGSGEGRGSLLTCGDVEENPGPMAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 477 - protein, 23 aa, synthetic construct
MDYKDHDGDYKDHDIDYKDDDDK

SEQ ID NO: 478 - protein, 9 aa, synthetic construct
MAPKKKRKV

SEQ ID NO: 479 - protein, 8 aa, synthetic construct
GIHGVPAA

SEQ ID NO: 480 - protein, 16 aa, synthetic construct
KRPAATKKAGQAKKKK

SEQ ID NO: 481 - protein, 23 aa, synthetic construct
EFGSGEGRGSLLTCGDVEENPGP

SEQ ID NO: 482 - protein, 132 aa, synthetic construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 483 - DNA, 56 bp, synthetic construct
  source 1..56: mol_type = other DNA
  misc_difference 34: note = a, c, g, t, unknown or other
acactctttccctacacgacgctcttccgatctncctttcgtctcgcgcgtttcgg

SEQ ID NO: 484 - DNA, 58 bp, synthetic construct
  source 1..58: mol_type = other DNA
  misc_difference 34: note = a, c, g, t, unknown or other
  misc_difference 35: note = a, c, g, t, unknown or other
  misc_difference 36: note = a, c, g, t, unknown or other
acactctttccctacacgacgctcttccgatctnnncctttcgtctcgcgcgtttcgg

SEQ ID NO: 485 - DNA, 60 bp, synthetic construct
  source 1..60: mol_type = other DNA
  misc_difference 34: note = a, c, g, t, unknown or other
  misc_difference 35: note = a, c, g, t, unknown or other
  misc_difference 36: note = a, c, g, t, unknown or other
  misc_difference 37: note = a, c, g, t, unknown or other
  misc_difference 38: note = a, c, g, t, unknown or other
acactctttccctacacgacgctcttccgatctnnnnncctttcgtctcgcgcgtttcgg

SEQ ID NO: 486 - DNA, 62 bp, synthetic construct
  source 1..62: mol_type = other DNA
  misc_difference 34: note = a, c, g, t, unknown or other
  misc_difference 35: note = a, c, g, t, unknown or other
  misc_difference 36: note = a, c, g, t, unknown or other
  misc_difference 37: note = a, c, g, t, unknown or other
  misc_difference 38: note = a, c, g, t, unknown or other
  misc_difference 39: note = a, c, g, t, unknown or other
  misc_difference 40: note = a, c, g, t, unknown or other
acactctttccctacacgacgctcttccgatctnnnnnnncctttcgtctcgcgcgtttcgg

SEQ ID NO: 487-503 - skipped (17 entries)

SEQ ID NO: 504 - RNA, 36 nt, synthetic construct
  source 1..36: mol_type = other RNA
gtttgagaaccttatgaaattacaaggatgccaaac

SEQ ID NO: 505 - protein, 20 aa, synthetic construct
  VARIANT 4: note = Any amino acid
  VARIANT 8: note = Any amino acid
  VARIANT 11: note = Any amino acid
  VARIANT 15: note = Any amino acid
  VARIANT 19: note = Any amino acid
GLFXALLXLLXSLWXLLLXA

SEQ ID NO: 506 - protein, 20 aa, synthetic construct
GLFHALLHLLHSLWHLLLHA

SEQ ID NO: 507 - protein, 26 aa, synthetic construct
GSGGSPAGSPTSTEEGTSESATPGSG

SEQ ID NO: 508 - protein, 6 aa, synthetic construct
HHHHHH

SEQ ID NO: 509 - protein, 5 aa, synthetic construct
GSGGS

SEQ ID NO: 510 - protein, 6 aa, synthetic construct
GGSGGS

SEQ ID NO: 511 - protein, 4 aa, synthetic construct
GGGS

SEQ ID NO: 512 - protein, 5 aa, synthetic construct
GSGGS

SEQ ID NO: 513 - protein, 6 aa, synthetic construct
GGSGGS

SEQ ID NO: 514 - protein, 4 aa, synthetic construct
GGGS

SEQ ID NO: 515 - protein, 4 aa, synthetic construct
GGSG

SEQ ID NO: 516 - protein, 5 aa, synthetic construct
GGSGG

SEQ ID NO: 517 - protein, 5 aa, synthetic construct
GSGSG

SEQ ID NO: 518 - protein, 5 aa, synthetic construct
GSGGG

SEQ ID NO: 519 - protein, 5 aa, synthetic construct
GGGSG

SEQ ID NO: 520 - protein, 5 aa, synthetic construct
GSSSG

SEQ ID NO: 521 - DNA, 40 bp, synthetic construct
  source 1..40: mol_type = other DNA
agagtaacagtctgagtaggagctaaaatattttgggttt

SEQ ID NO: 522 - DNA, 10 bp, synthetic construct
  source 1..10: mol_type = other DNA
agagtaacag

SEQ ID NO: 523 - DNA, 21 bp, synthetic construct
  source 1..21: mol_type = other DNA
agagtaaaatattttgggttt

SEQ ID NO: 524 - DNA, 28 bp, synthetic construct
  source 1..28: mol_type = other DNA
agagtaacagctaaaatattttgggttt

SEQ ID NO: 525 - DNA, 38 bp, synthetic construct
  source 1..38: mol_type = other DNA
agagtaacagtctgagtaagctaaaatattttgggttt

SEQ ID NO: 526 - DNA, 25 bp, synthetic construct
  source 1..25: mol_type = other DNA
agagtaactaaaatattttgggttt

SEQ ID NO: 527 - DNA, 13 bp, synthetic construct
  source 1..13: mol_type = other DNA
agagtaacagttt

SEQ ID NO: 528 - DNA, 24 bp, synthetic construct
  source 1..24: mol_type = other DNA
agagtaacaaaatattttgggttt

SEQ ID NO: 529 - DNA, 20 bp, synthetic construct
  source 1..20: mol_type = other DNA
agagtaaatattttgggttt

SEQ ID NO: 530 - DNA, 19 bp, synthetic construct
  source 1..19: mol_type = other DNA
agagtaatattttgggttt

SEQ ID NO: 531 - DNA, 26 bp, synthetic construct
  source 1..26: mol_type = other DNA
agagtaacagaaaatattttgggttt

SEQ ID NO: 532 - DNA, 37 bp, synthetic construct
  source 1..37: mol_type = other DNA
agagtaacagtctgagtagctaaaatattttgggttt

SEQ ID NO: 533 - DNA, 18 bp, synthetic construct
  source 1..18: mol_type = other DNA
agagtatattttgggttt

SEQ ID NO: 534 - DNA, 16 bp, synthetic construct
  source 1..16: mol_type = other DNA
agagtattttgggttt

SEQ ID NO: 535 - DNA, 23 bp, synthetic construct
  source 1..23: mol_type = other DNA
agagtaacagatattttgggttt

SEQ ID NO: 536 - DNA, 21 bp, synthetic construct
  source 1..21: mol_type = other DNA
agagtaacatattttgggttt

SEQ ID NO: 537 - DNA, 30 bp, synthetic construct
  source 1..30: mol_type = other DNA
agagtaacagagctaaaatattttgggttt

SEQ ID NO: 538 - DNA, 11 bp, synthetic construct
  source 1..11: mol_type = other DNA
agagtaacagt

SEQ ID NO: 539 - DNA, 16 bp, synthetic construct
  source 1..16: mol_type = other DNA
agagtaacatgggttt

SEQ ID NO: 540 - DNA, 23 bp, synthetic construct
  source 1..23: mol_type = other DNA
agagtaacaaatattttgggttt

SEQ ID NO: 541 - DNA, 11 bp, synthetic construct
  source 1..11: mol_type = other DNA
attttgggttt

SEQ ID NO: 542 - DNA, 22 bp, synthetic construct
  source 1..22: mol_type = other DNA
agagtaaaaatattttgggttt

SEQ ID NO: 543 - DNA, 19 bp, synthetic construct
  source 1..19: mol_type = other DNA
agagtaacattttgggttt

SEQ ID NO: 544 - DNA, 10 bp, synthetic construct
  source 1..10: mol_type = other DNA
agagtaaaag

SEQ ID NO: 545 - DNA, 39 bp, synthetic construct
  source 1..39: mol_type = other DNA
agagtaacagtctgagtagagctaaaatattttgggttt

SEQ ID NO: 546 - DNA, 34 bp, synthetic construct
  source 1..34: mol_type = other DNA
agagtaacagtctgagctaaaatattttgggttt

SEQ ID NO: 547 - DNA, 20 bp, synthetic construct
  source 1..20: mol_type = other DNA
agctaaaatattttgggttt

SEQ ID NO: 548 - DNA, 22 bp, synthetic construct
  source 1..22: mol_type = other DNA
agagctaaaatattttgggttt

SEQ ID NO: 549 - DNA, 15 bp, synthetic construct
  source 1..15: mol_type = other DNA
agatattttgggttt

SEQ ID NO: 550 - DNA, 14 bp, synthetic construct
  source 1..14: mol_type = other DNA
agagtaacaggttt

SEQ ID NO: 551 - DNA, 36 bp, synthetic construct
  source 1..36: mol_type = other DNA
agagtaacagtctgagtactaaaatattttgggttt

SEQ ID NO: 552 - DNA, 11 bp, synthetic construct
  source 1..11: mol_type = other DNA
agagtgggttt

SEQ ID NO: 553 - DNA, 15 bp, synthetic construct
  source 1..15: mol_type = other DNA
agagtaactgggttt

SEQ ID NO: 554 - DNA, 38 bp, synthetic construct
  source 1..38: mol_type = other DNA
agagtaacagtctgagtgagctaaaatattttgggttt

SEQ ID NO: 555 - DNA, 15 bp, synthetic construct
  source 1..15: mol_type = other DNA
agagtaacagggttt

SEQ ID NO: 556 - DNA, 40 bp, synthetic construct
  source 1..40: mol_type = other DNA
agagtaacagtctgagtaggggctaaaatattttgggttt

SEQ ID NO: 557 - DNA, 42 bp, synthetic construct
  source 1..42: mol_type = other DNA
taagtaatgtgtatgcttttctgttaaagaggaagttagaag

SEQ ID NO: 558 - DNA, 40 bp, synthetic construct
  source 1..40: mol_type = other DNA
atcttcaaagtgttaatcgaataagtaatgtgtatgcttt

SEQ ID NO: 559 - DNA, 20 bp, synthetic construct
  source 1..20: mol_type = other DNA
atcttcaaagtgttaatcga

SEQ ID NO: 560 - DNA, 19 bp, synthetic construct
  source 1..19: mol_type = other DNA
atcttcaaagtgttaatcg

SEQ ID NO: 561 - DNA, 18 bp, synthetic construct
  source 1..18: mol_type = other DNA
atcttcaaagtgttaatc

SEQ ID NO: 562 - DNA, 15 bp, synthetic construct
  source 1..15: mol_type = other DNA
atcttcaaagtgtta

SEQ ID NO: 563 - DNA, 13 bp, synthetic construct
  source 1..13: mol_type = other DNA
atcttcaaagtgt

SEQ ID NO: 564 - DNA, 16 bp, synthetic construct
  source 1..16: mol_type = other DNA
atcttcaaagtgttaa

SEQ ID NO: 565 - DNA, 14 bp, synthetic construct
  source 1..14: mol_type = other DNA
atcttcaaagtgtt

SEQ ID NO: 566 - DNA, 39 bp, synthetic construct
  source 1..39: mol_type = other DNA
ctattggagcctgtaagtatactggatcccattctcttt

SEQ ID NO: 567 - DNA, 40 bp, synthetic construct
  source 1..40: mol_type = other DNA
actggatcccattctctttggctctagctatttgttcaaa

SEQ ID NO: 568 - DNA, 19 bp, synthetic construct
  source 1..19: mol_type = other DNA
ctctagctatttgttcaaa

SEQ ID NO: 569 - DNA, 18 bp, synthetic construct
  source 1..18: mol_type = other DNA
tctagctatttgttcaaa

SEQ ID NO: 570 - DNA, 17 bp, synthetic construct
  source 1..17: mol_type = other DNA
ctagctatttgttcaaa

SEQ ID NO: 571 - DNA, 21 bp, synthetic construct
  source 1..21: mol_type = other DNA
ggctctagctatttgttcaaa

SEQ ID NO: 572 - DNA, 20 bp, synthetic construct
  source 1..20: mol_type = other DNA
gctctagctatttgttcaaa

SEQ ID NO: 573 - DNA, 16 bp, synthetic construct
  source 1..16: mol_type = other DNA
tagctatttgttcaaa

SEQ ID NO: 574 - DNA, 22 bp, synthetic construct
  source 1..22: mol_type = other DNA
tggctctagctatttgttcaaa

(19) United States
(12) Patent Application Publication
(10) Pub. No.: US 2025/0144246 A1
(43) Pub. Date: May 8, 2025

(54) COMPOSITIONS AND METHODS FOR MODIFYING A HUMAN DYSTROPHIN GENE

(71) Applicant: Mammoth Biosciences, Inc., Brisbane, CA (US)

(72) Inventors: Lucas Benjamin HARRINGTON, San Francisco, CA (US); Wiputra Jaya HARTONO, San Francisco, CA (US); Pei-Qi LIU, Oakland, CA (US); Darren Bo Yee LO, Pacifica, CA (US); Renan B. SPER, San Francisco, CA (US)

(21) Appl. No.: 18/919,415

(22) Filed: Oct. 17, 2024

Related U.S. Application Data
(63) Continuation of application No. PCT/US2023/066060, filed on Apr. 21, 2023.
(60) Provisional application No. 63/333,817, filed on Apr. 22, 2022.
(60) Provisional application No. 63/334,664, filed on Apr. 25, 2022.
(60) Provisional application No. 63/346,503, filed on May 27, 2022.
(60) Provisional application No. 63/355,004, filed on Jun. 23, 2022.

(51) Int. Cl. A61K 48/00; C12N 9/12; C12N 9/22; C12N 15/11; C12N 15/86

(52) U.S. Cl. CPC A61K 48/0058; C12N 9/1276; C12N 9/22; C12N 15/11; C12N 15/86; C12N 2310/20; C12N 2750/14143

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

This application is a continuation of International Patent Application No. PCT/US2023/066060, filed Apr. 21, 2023, which claims the benefit of priority to U.S. Provisional Application No. 63/333,817, filed on Apr. 22, 2022, U.S. Provisional Application No. 63/334,664, filed on Apr. 25, 2022, U.S. Provisional Application No. 63/346,503, filed on May 27, 2022, and U.S. Provisional Application No. 63/355,004, filed on Jun. 23, 2022, the entire contents of each of which are incorporated herein by reference.

### INCORPORATION BY REFERENCE OF SEQUENCE LISTING

The instant application contains a Sequence Listing, which has been submitted via patent Center. The Sequence Listing titled 203477-754301_US_SL.xml, which was created on Oct. 14, 2024 and is 534,525 bytes in size, is hereby incorporated by reference in its entirety.

### FIELD

The present disclosure relates generally to compositions of effector proteins and guide nucleic acids, and methods and systems of using such compositions, including detecting and editing target nucleic acids, as well as, the treatment of disorders associated with the dystrophin gene (DMD).

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. Native systems contain a CRISPR array, which includes direct repeats flanking short spacer sequences that, in part, guide Cas proteins to their targets. The discovery of CRISPR/Cas systems has revolutionized the field of genomic manipulation and engineering, and therapeutic applications of these systems are being explored. While the programmable nature of these systems has promising implications in the field of genome engineering, there remains a need to explore alternative strategies and components to leverage the CRISPR-Cas system in ways that are efficient for in vitro detection and effective for in vivo genome engineering. Effector proteins, guide nucleic acids, compositions, systems and methods described herein may satisfy this need and provides related advantages.

### SUMMARY

The present disclosure provides for compositions, methods and systems comprising an effector protein, a guide nucleic acid, and uses thereof. Compositions, systems, and methods disclosed herein leverage nucleic acid modifying activities (e.g., cis cleavage activity) of these effector proteins and guide nucleic acids for the modification and detection of target nucleic acids of the DMD gene. Accordingly, in one aspect, provided herein is a composition comprising an effector protein and a guide nucleic acid for the treatment of a disorder associated with the DMD gene.

### I. Certain Embodiments

Provided herein are compositions comprising: an effector protein comprising an amino acid sequence that is at least 70% identical to SEQ ID NO: 1, and a guide nucleic acid that comprises a spacer sequence that binds/hybridizes to a target sequence in a human dystrophin (DMD) locus.

Also provided herein is a composition comprising a guide nucleic acid, wherein the guide nucleic acid is a single guide RNA (sgRNA). In some embodiments, the sgRNA comprises a handle sequence. In some embodiments, the handle sequence comprises the nucleotide sequence of SEQ ID NO: 441. In some embodiments, the sgRNA comprises a linker. In some embodiments, the linker comprises the nucleotide sequence of SEQ ID NO: 442. In some embodiments, the sgRNA comprises a repeat sequence. In some embodiments, the repeat sequence comprises the nucleotide sequence of SEQ ID NO: 443. In some embodiments, the sgRNA comprises a linker and a repeat sequence. In some embodiments, the spacer sequence comprises a nucleotide sequence of any one of SEQ ID NOs: 5-222. In some embodiments, the sgRNA comprises a nucleotide sequence of any one of SEQ ID NOs: 223-440. In some embodiments, the sgRNA comprises a handle sequence that is at least 90% identical to SEQ ID NO: 4. In some embodiments, the sgRNA comprises a handle sequence that is at least 95% identical to SEQ ID NO: 4. In some embodiments, the sgRNA comprises a nucleotide sequence of any one of SEQ ID NOS: 223-440. In some embodiments, the sgRNA comprises a nucleotide sequence that is at least 90% identical to a nucleotide sequence of any one of SEQ ID NOS: 297, 300, and 303. In some embodiments, the sgRNA comprises a nucleotide sequence that is at least 95% identical to a nucleotide sequence of any one of SEQ ID NOS: 297, 300, and 303. In some embodiments, the sgRNA comprises a nucleotide sequence that is identical to a nucleotide sequence of any one of SEQ ID NOS: 297, 300, and 303. In some embodiments, the guide nucleic acid comprises a repeat sequence of SEQ ID NO: 443.

Also provided herein are compositions comprising an effector protein and a guide nucleic acid, wherein the spacer sequence is at least 95% identical to a sequence recited in TABLE 4.

Also provided herein are compositions comprising an effector protein and a guide nucleic acid, wherein the spacer sequence is a sequence recited in TABLE 4.

Also provided herein are compositions comprising an effector protein and a guide nucleic acid, wherein the spacer sequence is at least 90% identical to a nucleotide sequence of any one of SEQ ID NOS: 79, 82 and 85 as recited in TABLE 4.

Also provided herein are compositions comprising an effector protein and a guide nucleic acid, wherein the spacer sequence is at least 95% identical to a nucleotide sequence of any one of SEQ ID NOS: 79, 82 and 85 as recited in TABLE 4.

Also provided herein are compositions comprising an effector protein and a guide nucleic acid, wherein the spacer sequence is identical to a nucleotide sequence of any one of SEQ ID NOS: 79, 82 and 85 as recited in TABLE 4.

Also provided herein are compositions comprising an effector protein and a guide nucleic acid, wherein the spacer sequence is at least 90% identical to a nucleotide sequence of any one of SEQ ID NOS: 79 and 85 as recited in TABLE 4.

Also provided herein are compositions comprising an effector protein and a guide nucleic acid, wherein the spacer sequence is at least 95% identical to a nucleotide sequence of any one of SEQ ID NOS: 79 and 85 as recited in TABLE 4.

Also provided herein are compositions comprising an effector protein and a guide nucleic acid, wherein the spacer sequence is a nucleotide sequence of any one of SEQ ID NOS: 79 and 85 as recited in TABLE 4.

Also provided herein are compositions comprising an effector protein and a guide nucleic acid, wherein the spacer sequence is at least 90% identical to a nucleotide sequence of SEQ ID NO: 79 as recited in TABLE 4.

Also provided herein are compositions comprising an effector protein and a guide nucleic acid, wherein the spacer sequence is at least 95% identical to a nucleotide sequence of SEQ ID NO: 79 as recited in TABLE 4.

Also provided herein are compositions comprising an effector protein and a guide nucleic acid, wherein the spacer sequence is a nucleotide sequence of SEQ ID NO: 79 as recited in TABLE 4.

Also provided herein are compositions comprising an effector protein and a guide nucleic acid, wherein the guide nucleic acid is at least 90% identical to a guide RNA sequence recited in TABLE 6.

Also provided herein are compositions comprising an effector protein and a guide nucleic acid, wherein the guide nucleic acid is at least 95% identical to a guide RNA sequence recited in TABLE 6.

Also provided herein are compositions comprising an effector protein and a guide nucleic acid, wherein the guide nucleic acid is a guide RNA sequence recited in TABLE 6.

Also provided herein are compositions comprising an effector protein and a guide nucleic acid, wherein the guide nucleic acid consists of a nucleotide sequence that is at least 90% identical to a guide RNA sequence recited in TABLE 6.

Also provided herein are compositions comprising an effector protein and a guide nucleic acid, wherein the guide nucleic acid consists of a nucleotide sequence that is at least 95% identical to a guide RNA sequence recited in TABLE 6.

Also provided herein are compositions comprising an effector protein and a guide nucleic acid, wherein the guide nucleic acid consists of a guide RNA sequence recited in TABLE 6.

Also provided herein are compositions comprising an effector protein and a guide nucleic acid, wherein the effector protein comprises an amino acid sequence that is at least 80% identical to SEQ ID NO: 1.

Also provided herein are compositions comprising an effector protein and a guide nucleic acid, wherein the effector protein comprises an amino acid sequence that is at least 85% identical to SEQ ID NO: 1.

Also provided herein are compositions comprising an effector protein and a guide nucleic acid, wherein the effector protein comprises an amino acid sequence that is at least 90% identical to SEQ ID NO: 1.

Also provided herein are compositions comprising an effector protein and a guide nucleic acid, wherein the effector protein comprises an amino acid sequence that is at least 95% identical to SEQ ID NO: 1.

Also provided herein are compositions comprising an effector protein and a guide nucleic acid, wherein the effector protein comprises an amino acid sequence that is at least 98% identical to SEQ ID NO: 1.

Also provided herein are compositions comprising an effector protein and a guide nucleic acid, wherein the effector protein comprises SEQ ID NO: 1.

Also provided herein are compositions comprising an effector protein and a guide nucleic acid, wherein the effector protein recognizes any one of the protospacer adjacent motif (PAM) sequences recited in TABLE 2.

Also provided herein are compositions comprising an effector protein and a guide nucleic acid, wherein the effector protein interacts with a handle sequence that is at least 90% identical to any one of the handle sequences recited in TABLE 5.

Also provided herein are compositions comprising an effector protein and a guide nucleic acid, wherein the effector protein interacts with a handle sequence that is at least 95% identical to any one of the handle sequences recited in TABLE 5.

Also provided herein are compositions comprising an effector protein and a guide nucleic acid, wherein the effector protein interacts with any one of the handle sequences recited in TABLE 5.

Also provided herein are compositions comprising an effector protein and a guide nucleic acid, wherein the guide nucleic acid comprises a handle sequence and a crRNA, wherein the crRNA comprises the spacer sequence.

Also provided herein are compositions comprising an effector protein and a guide nucleic acid, wherein the handle sequence is covalently linked to the 5′ end of the crRNA.

Also provided herein are compositions comprising an effector protein and a guide nucleic acid, wherein the handle sequence is covalently linked to the 3′ end of the crRNA.

Also provided herein are compositions comprising an effector protein and a guide nucleic acid, wherein the effector protein comprises an amino acid sequence that is at least 90% identical to the amino acid sequence of SEQ ID NO: 1, and wherein the guide nucleic acid comprises a handle sequence with at least 90% identical to any one of the handle sequences recited in TABLE 5 and a spacer sequence that is at least 90% identical to any one of the nucleotide sequences recited in TABLE 4.

Also provided herein are compositions comprising an effector protein and a guide nucleic acid, wherein the effector protein comprises an amino acid sequence that is at least 95% identical to the amino acid sequence of SEQ ID NO: 1, and wherein the guide nucleic acid comprises a handle sequence that is at least 95% identical to any one of the handle sequences recited in TABLE 5 and a spacer sequence that is at least 95% identical to any one of the nucleotide sequences recited in TABLE 4.

Also provided herein are compositions comprising an effector protein and a guide nucleic acid, wherein the effector protein comprises the sequence of SEQ ID NO: 1, and wherein the guide nucleic acid comprises any one of the handle sequences recited in TABLE 5 and any one of the spacer sequences recited in TABLE 4.

Also provided herein are compositions comprising an effector protein and a guide nucleic acid, wherein the effector protein comprises an amino acid sequence that is at least 90% identical to an amino acid sequence of SEQ ID NO: 1, and wherein the guide nucleic acid comprises a nucleotide sequence that is at least 90% identical to any one of the guide nucleic acid sequences of TABLE 6.

Also provided herein are compositions comprising an effector protein and a guide nucleic acid, wherein the effector protein comprises an amino acid sequence that is at least 95% identical to an amino acid sequence of SEQ ID NO: 1, and wherein the guide nucleic acid comprises a nucleotide sequence that is at least 95% identical to any one of the guide nucleic acid sequences of TABLE 6.

Also provided herein are compositions comprising an effector protein and a guide nucleic acid, wherein the effector protein comprises an amino acid sequence of SEQ ID NO: 1, and wherein the guide nucleic acid comprises any one of the guide nucleic acid sequences of TABLE 6.

Also provided herein are compositions comprising an effector protein and a guide nucleic acid, wherein the effector protein comprises a nuclear localization signal.

Also provided herein are compositions comprising an effector protein and a guide nucleic acid, wherein the target sequence is located in an intron of the human DMD locus.

Also provided herein are compositions comprising an effector protein and a guide nucleic acid, wherein the target sequence is located in an exon of the human DMD locus.

Also provided herein are compositions comprising an effector protein and a guide nucleic acid, wherein the target sequence spans an exon-intron junction of the human DMD locus.

Also provided herein are compositions comprising an effector protein and a guide nucleic acid, wherein the composition further comprises an additional guide nucleic acid that binds/hybridizes a different portion of the target nucleic acid than the guide nucleic acid.

Also provided herein are compositions comprising an effector protein and a guide nucleic acid, wherein the guide nucleic acid binds/hybridizes a portion of the target nucleic acid that is upstream or downstream of a premature stop codon associated with the human gene, and wherein the additional guide nucleic acid binds/hybridizes to a portion of the target nucleic acid that is upstream or downstream of the premature stop codon associated with the human gene.

Also provided herein are compositions comprising an effector protein and a guide nucleic acid, further comprising a donor nucleic acid.

Also provided herein are compositions comprising an effector protein and a guide nucleic acid, further comprising a fusion partner protein linked to the effector protein.

Also provided herein are compositions comprising an effector protein and a guide nucleic acid, wherein the fusion partner protein is directly fused to the N terminus or C terminus of the effector protein via an amide bond.

Also provided herein are compositions comprising an effector protein and a guide nucleic acid, wherein the fusion partner protein comprises a polypeptide selected from a deaminase, a transcriptional activator, a transcriptional repressor, or a functional domain thereof.

Also provided herein are compositions comprising an effector protein and a guide nucleic acid, wherein the effector protein comprises at least one mutation that reduces its nuclease activity, relative to an otherwise comparable effector protein without the mutation, as measured in a cleavage assay.

Also provided herein are compositions comprising an effector protein and a guide nucleic acid, wherein the effector protein is a catalytically inactive nuclease.

Also provided herein are compositions comprising an effector protein and a guide nucleic acid, wherein the composition modifies the target nucleic acid.

Also provided herein are compositions comprising an effector protein and a guide nucleic acid, wherein the modification of the target nucleic acid comprises cleaving the target nucleic acid, deleting a nucleotide of the target nucleic acid, inserting a nucleotide into the target nucleic acid, substituting a nucleotide of the target nucleic acid with an alternative nucleotide, more than one of the foregoing, or any combination thereof.

Also provided herein are compositions comprising an effector protein and a guide nucleic acid, wherein the composition removes all or a portion of the sequence between the guide nucleic acid and the additional guide nucleic acid.

Also provided herein are compositions comprising an effector protein and a guide nucleic acid, wherein the gene comprises a non-WT (wild-type) reading frame, and wherein upon modification of the target nucleic acid, the WT reading frame is restored.

Also provided herein are nucleic acid expression vectors that encode a guide nucleic acid that comprises a spacer sequence that binds/hybridizes to a target sequence and that is at least 90% identical to any one of the nucleotide sequences recited in TABLE 4.

Also provided herein are nucleic acid expression vectors, wherein the nucleic acid expression vector further encodes a donor nucleic acid.

Also provided herein are nucleic acid expression vectors, wherein the nucleic acid expression vector is a viral vector.

Also provided herein are nucleic acid expression vectors, wherein the viral vector is an adeno associated viral (AAV) vector.

Also provided herein are nucleic acid expression vectors, wherein the nucleic acid expression vector is a viral vector, wherein the viral vector comprises a nucleotide sequence of a first promoter, wherein the first promoter drives transcription of a nucleotide sequence encoding the guide nucleic acid, and wherein the first promoter is selected from a group consisting of CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK.

Also provided herein are nucleic acid expression vectors, wherein the nucleic acid expression vector is a viral vector, wherein the viral vector comprises a nucleic acid sequence encoding an effector protein, and wherein an amino acid sequence of the effector protein that is at least 80% identical to an amino acid sequence recited in SEQ ID NO: 1

Also provided herein are nucleic acid expression vectors, wherein the nucleic acid expression vector is a viral vector, wherein the viral vector comprises a nucleotide sequence of a second promoter, wherein the second promoter drives expression of the effector protein, and wherein the second promoter is a ubiquitous promoter or a site-specific promoter.

Also provided herein are nucleic acid expression vectors, wherein the nucleic acid expression vector is a viral vector, wherein the viral vector comprises a nucleotide sequence of a second promoter, wherein the second promoter is a ubiquitous promoter, and wherein the ubiquitous promoter is selected from a group consisting of MND and CAG.

Also provided herein are nucleic acid expression vectors, wherein the nucleic acid expression vector is a viral vector, wherein the viral vector comprises a nucleotide sequence of a second promoter, wherein the second promoter is a site-specific promoter, and wherein the site-specific promoter is selected from a group consisting of Ck8e, Spc5-12, and Desmin.

Also provided herein are nucleic acid expression vectors, wherein the nucleic acid expression vector is a viral vector, wherein the viral vector comprises an enhancer, wherein the enhancer is a nucleotide sequence having the effect of enhancing promoter activity, and wherein the enhancer is selected from a group consisting of WPRE enhancer, CMV enhancers, the R—U5′ segment in LTR of HTLV-I, SV40 enhancer, the intron sequence between exons 2 and 3 of rabbit β-globin, and the genome region of human growth hormone.

Also provided herein are nucleic acid expression vectors, wherein the nucleic acid expression vector is a viral vector, wherein the viral vector comprises a poly A signal sequence, and wherein the poly A signal sequence is selected from hGH poly A signal sequence and sv40 poly A signal sequence.

Also provided herein are nucleic acid expression vectors, wherein the nucleic acid expression vector is a viral vector, and wherein the viral vector comprises a nucleotide sequence of a first promoter, a nucleotide sequence encoding a guide nucleic acid, a nucleotide sequence of a second promoter, a nucleotide sequence encoding an effector protein, and a poly A signal sequence, in a 5′ to 3′ direction.

Also provided herein are nucleic acid expression vectors, wherein the nucleic acid expression vector is a viral vector, and wherein the viral vector comprises, a nucleotide sequence of a first promoter, a nucleotide sequence encoding a guide nucleic acid, a nucleotide sequence of a second promoter, a nucleotide sequence encoding an effector protein, an enhancer, and a poly A signal sequence, in a 5′ to 3′ direction.

Also provided herein are nucleic acid expression vectors, wherein the nucleic acid expression vector is a viral vector, wherein the nucleic acid expression vector comprises a nucleotide sequence encoding a first guide nucleic acid and a nucleotide sequence encoding a second guide nucleic acid, and wherein the first guide nucleic acid is different from the second guide nucleic acid.

Also provided herein are nucleic acid expression vectors, wherein the nucleic acid expression vector is a viral vector, wherein the viral vector comprises a nucleotide sequence encoding a first guide nucleic acid and a nucleotide sequence encoding a second guide nucleic acid, wherein the viral vector comprises a nucleotide sequence of a first promoter and a nucleotide sequence of a third promoter, wherein the third promoter drives transcription of a nucleotide sequence encoding the second guide nucleic acid, wherein the first promoter and the third promoter are selected from a group consisting of CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK, and wherein the first promoter and the third promoter are different.

Also provided herein are nucleic acid expression vectors, wherein the nucleic acid expression vector is a viral vector, wherein the nucleic acid expression vector comprises a nucleotide sequence encoding a first guide nucleic acid and a nucleotide sequence encoding a second guide nucleic acid, wherein the first guide nucleic acid is different from the second guide nucleic acid, and wherein the viral vector comprises a nucleotide sequence of a first promoter, a nucleotide sequence encoding the first guide nucleic acid, a nucleotide sequence of a second promoter, a nucleotide sequence encoding an effector protein, an enhancer, a poly A signal sequence, a nucleotide sequence of a third promotor, and a nucleotide sequence encoding the second guide nucleic acid, in a 5′ to 3′ direction.

Also provided herein are pharmaceutical compositions comprising compositions described herein or nucleic acid vectors described herein; and a pharmaceutically acceptable excipient.

Also provided herein are systems comprising compositions described herein or nucleic acid vectors described herein.

Also provided herein are systems comprising at least one detection reagent for detecting a target nucleic acid.

Also provided herein are systems, wherein the at least one detection reagent is selected from a reporter nucleic acid, a detection moiety, an additional effector protein, or a combination thereof, optionally wherein the reporter nucleic acid comprises a fluorophore, a quencher, or a combination thereof.

Also provided herein are systems, wherein the detection reagent is operably linked to the effector protein or the guide nucleic acid, such that a detection event occurs upon contacting the system with a target nucleic acid.

Also provided herein are systems, comprising at least one amplification reagent for amplifying a target nucleic acid.

Also provided herein are systems, wherein the at least one amplification reagent is selected from the group consisting of a primer, an activator, a dNTP, an rNTP, and combinations thereof.

Also provided herein is a method of modifying a target nucleic acid within a human dystrophin gene, or associated with expression of a human dystrophin gene, the method comprising contacting the target nucleic acid with a composition described herein, a nucleic acid expression vector described herein, a pharmaceutical composition described herein, or a system described herein, thereby modifying the target nucleic acid.

Also provided herein is a method of modifying a target nucleic acid within a human dystrophin gene, wherein the modifying of the target nucleic acid comprises cleaving the target nucleic acid, deleting a nucleotide of the target nucleic acid, inserting a nucleotide into the target nucleic acid, substituting a nucleotide of the target nucleic acid with an alternative nucleotide, more than one of the foregoing, or any combination thereof.

Also provided herein is a method of modifying a target nucleic acid within a human dystrophin gene, wherein the composition further comprises an additional guide nucleic acid that binds/hybridizes a different portion of the target nucleic acid than the guide nucleic acid

Also provided herein is a method of modifying a target nucleic acid within a human dystrophin gene, wherein at least one of the guide nucleic acids binds/hybridizes a portion of the target nucleic acid that is upstream or downstream of a premature stop codon associated with the human dystrophin gene, and wherein the additional guide nucleic acid binds/hybridizes to a portion of the target nucleic acid that is upstream or downstream of the premature stop codon associated with the human dystrophin gene.

Also provided herein is a method of modifying a target nucleic acid within a human dystrophin gene, wherein the composition removes all or a portion of the sequence between the guide nucleic acid and the additional guide nucleic acid.

Also provided herein is a method of modifying a target nucleic acid within a human dystrophin gene, further comprising contacting the target nucleic acid with a donor nucleic acid.

Also provided herein is a method of modifying a target nucleic acid within a human dystrophin gene, wherein the human dystrophin gene comprises a non-WT (wild-type) reading frame, and wherein upon modification of the target nucleic acid, the WT reading frame is restored.

Also provided herein is a method of modifying a target nucleic acid within a human dystrophin gene, wherein the method is performed in a cell.

Also provided herein is a method of modifying a target nucleic acid within a human dystrophin gene, wherein the method is performed in vivo.

Also provided herein is a method of modifying a target nucleic acid within a human dystrophin gene, wherein the target nucleic acid comprises a mutation associated with a disease.

In some embodiments, the disease is a genetic disorder.

In some embodiments, the genetic disorder is a neurological disorder.

In some embodiments, the neurological disorder is DMD, BMD, or CMD Type 3B.

In some embodiments, the disease is any one of the diseases recited in TABLE 11.

In some embodiments, wherein the disease is DMD.

In some embodiments, a gene associated with the genetic disorder comprises one or more mutations.

In some embodiments, the target nucleic acid is encoded by a gene recited in TABLE 7.

In some embodiments, the gene is DMD.

In some embodiments, the gene comprises one or more mutations.

In some embodiments, the one or more mutations comprise a point mutation, a single nucleotide polymorphism (SNP), a chromosomal mutation, a copy number mutation, or any combination thereof.

Also provided herein is a cell comprising a composition described herein or a nucleic acid expression vector described herein.

Also provided herein is a cell comprising a target nucleic acid modified by a composition described herein or a nucleic acid expression vector described herein.

In some embodiments, the cell is a eukaryotic cell.

In some embodiments, the cell is a mammalian cell.

In some embodiments, the cell is a human cell.

In some embodiments, the cell is a muscle cell.

In some embodiments, the muscle cell is a cardiac muscle cell, a cardiomyocyte, a myocyte, a smooth muscle cell, a skeletal muscle cell, or a visceral muscle cell.

In some embodiments, the muscle cell is a skeletal muscle cell.

In some embodiments, the cell comprises a mutation in the dystrophin gene.

In some embodiments, the cell is a: muscle satellite cell, muscle stem cell, myoblast, muscle progenitor cell, induced pluripotent stem cell (iPSC) or a cell derived from an iPSC.

Also provided herein is a population of cells that comprises at least one cell as described herein.

Also provided herein is a method of treating a disease associated with a mutation or aberrant expression of a human dystrophin gene in a subject in need thereof, the method comprising administering to the subject a composition that comprises: an effector protein, a guide nucleic acid, or at least one expression vector that encodes the effector protein and the guide nucleic acid; wherein the guide nucleic acid comprises a spacer sequence that binds/hybridizes to a target sequence within the human dystrophin gene or associated with the expression of the human dystrophin gene; and wherein the spacer sequence that is at least 90% identical to a nucleotide sequence recited in TABLE 4.

Also provided herein is a method of treating a disease, wherein the disease is a genetic disorder.

In some embodiments, the genetic disorder is a neurological disorder.

In some embodiments, the neurological disorder is DMD, BMD, or CMD Type 3B.

Also provided herein is a method of treating a disease, wherein the disease is anyone of the diseases recited in TABLE 11.

Also provided herein is a method of treating a disease, wherein the disease is DMD.

In some embodiments, the spacer sequence is at least 95% identical to any one of the nucleotide sequences recited in TABLE 4.

In some embodiments, the spacer sequence is any one of the nucleotide sequences recited in TABLE 4.

In some embodiments, the guide nucleic acid is at least 90% identical to any one of the guide RNA sequences recited in TABLE 6.

In some embodiments, the guide nucleic acid is at least 95% identical to any one of the guide RNA sequences recited in TABLE 6.

In some embodiments, the guide nucleic acid is any one of the guide RNA sequences recited in TABLE 6.

The compositions, systems, and methods described herein, wherein the target sequence is within the human dystrophin gene.

The compositions, systems, and methods described herein, wherein the target sequence is at least partially within a targeted exon within the human dystrophin gene.

The compositions, systems, and methods described herein, wherein at least a portion of the target nucleic acid that a guide nucleic acid binds/hybridizes can comprise about 30 nucleotides to about 150 nucleotides adjacent to: the start of the targeted exon, the end of the targeted exon, or both.

The compositions, systems, and methods described herein, wherein one or more of exons 44, 45, 50, 51 and 53 of the human dystrophin gene are targeted. The compositions, systems, and methods described herein, wherein the one of more of exons 44, 45, 50, 51, and 53 of the human dystrophin gene, or portions thereof are removed.

The compositions, systems, and methods described herein, wherein one or more of exons 44 and 50 of the human dystrophin gene are targeted.

The compositions, systems, and methods described herein, wherein exon 50 of the human dystrophin gene is targeted. The compositions, systems, and methods described herein, wherein the exon 50 of the human dystrophin gene, or portions thereof are removed.

Also provided herein is a system comprising: (a) a polypeptide comprising an amino acid sequence at least 90% identical to SEQ ID NO: 1; (b) a first guide nucleic acid comprising a first spacer sequence complementary to a first target sequence of the human DMD locus; (c) a second guide nucleic acid comprising a second spacer sequence complementary to a second target sequence of the human DMD locus, wherein the first spacer sequence and the second spacer sequence are selected from a guide pair of TABLE 17.

Also provided herein is a system for editing human DMD locus gene comprising one or more components, wherein the one or more components individually comprise the following: (a) an effector protein, or a nucleic acid encoding the effector protein, wherein the effector protein comprises an amino acid sequence that is at least 90% identical to the amino acid sequence of SEQ ID NO: 1; and (b) a guide nucleic acid, or DNA molecule encoding a guide nucleic acid, wherein the guide nucleic acid comprises a spacer sequence complementary to a target sequence of the human DMD locus, wherein the spacer sequence is selected from any one of the guide sequences recited in TABLE 17.

The system described herein, wherein at least one of the first guide nucleic acid and the second guide nucleic acid comprise a sequence that is at least 80% identical to the nucleotide sequence of SEQ ID NO: 441.

The system described herein, wherein at least one of the first guide nucleic acid and the second guide nucleic acid comprise a sequence that is at least 80% identical to the nucleotide sequence of SEQ ID NO: 443.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates an exemplary schematic of AAV construct for gene editing according to one or more embodiments of the present disclosure. Included in FIG. 1 are the following abbreviations representing elements of the AAV construct: ITR=Inverted terminal repeat; gRNA=guide RNA; UTR=untranslated region; ssAAV=single-stranded AAV; scAAV=self-complementary AAV; and WPRE=Woodchuck Hepatitis Virus (WHV) posttranscriptional regulatory element.

FIG. 2 illustrates exemplary schematics of ssAAV and scAAV constructs for gene editing according to one or more embodiments of the present disclosure. Construct E), F), G) and H) are ssAAV constructs, whereas construct I) is an scAAV construct. Included in FIG. 2 are the following abbreviations representing elements of the AAV construct: ITR=Inverted terminal repeat; gRNA=guide RNA; UTR=untranslated region; WPRE=Woodchuck Hepatitis Virus (WHV) posttranscriptional regulatory element; and hGH Poly A=human growth hormone polyadenylation signal.

FIG. 3 illustrates exemplary schematics of ssAAV constructs for gene editing according to one or more embodiments of the present disclosure. Included in FIG. 3 are the following abbreviations representing elements of the AAV construct: ITR=Inverted terminal repeat; gRNA=guide RNA; UTR=untranslated region; WPRE=Woodchuck Hepatitis Virus (WHV) posttranscriptional regulatory element; and hGH Poly A=human growth hormone polyadenylation signal.

FIG. 4 shows the nuclease activity of CasM.265466 with flexible PAM sequences, in accordance with an embodiment of the present disclosure.

FIG. 5A shows exemplary indels generated in DMD of human cells that were transfected by lipofection with an mRNA encoding CasM.265466 protein and a guide nucleic acid targeting DMD.

FIG. 5B shows indel generation in DMD by CasM.265466 protein and a guide nucleic acid. Analysis of indels indicates the types of mutations that occur in DMD as a result of the indels. Types of mutations include in frame mutations, splice disruption mutations, +1 frameshift mutations, and +2 frameshift mutations, which are summarized as a proportion of total % indel observed. Other types of effects may also be observed but are not visible within the graphed results.

FIGS. 6A-6B show indels generated in DMD of human cells transfected by lipofection with mRNA encoding CasM.265466 protein and two guide nucleic acids.

FIG. 6C shows indel generation or exon deletion in a target nucleic acid due to contact with CasM.265466 protein and two guide nucleic acids, which are summarized as a proportion of total % indel observed.

FIG. 7A shows deletion of DMD exon 50 in iPSC by contacting the iPSC with CasM.265466 protein and two guide nucleic acids.

FIG. 7B shows deletion of DMD exon 50 in iPSC by contacting iPSC colonies with CasM.265466 protein and two guide nucleic acids.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### II. Definitions

The terms, “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The term, “amplification,” “amplifying,” or grammatical equivalents thereof, as used herein, refers to a process by which a nucleic acid molecule is enzymatically copied to generate a plurality of nucleic acid molecules containing the same sequence as the original nucleic acid molecule or a distinguishable portion thereof.

The term, “base editing enzyme,” as used herein, refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term, “base editor,” as used herein, refers to a fusion protein comprising abase editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The terms, “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein, refers to an assay designed to visualize, quantitate or identify cleavage of a nucleic acid. In some instances, the cleavage activity may be cis-cleavage activity. In some instances, the cleavage activity may be trans-cleavage activity.

The terms, “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The term, “CRISPR RNA” or “crRNA,” as used herein, refers to a type of guide nucleic acid, wherein the nucleic acid is RNA comprising a first sequence, often referred to herein as a spacer sequence, that hybridizes to a target sequence of a target nucleic acid, and a second sequence that is capable of connecting a crRNA to an effector protein by either a) hybridizing to a portion of a tracrRNA or b) being non-covalently bound by an effector protein. In a dual nucleic acid system, where a crRNA and a tracrRNA forms a complex with an effector protein, a crRNA includes the first sequence that hybridizes to the target sequence of the target nucleic acid and the second sequence hybridizes to a portion of the tracrRNA.

The term, “detectable signal,” as used herein, refers to a signal that can be detected using optical, fluorescent, chemiluminescent, electrochemical and other detection methods known in the art.

The term, “donor nucleic acid,” as used herein, refers to a nucleic acid that is incorporated into a target nucleic acid or target sequence.

The term “dual nucleic acid system” as used herein refers to a system that uses a transactivated or transactivating tracrRNA-crRNA duplex complexed with one or more effector proteins described herein, wherein the complex is capable of interacting with a target nucleic acid in a sequence selective manner. Accordingly, in a dual nucleic acid system, a tracrRNA or a tracrRNA-crRNA duplex enables an effector protein to have a binding and/or nuclease activity on a target nucleic acid.

The term, “effector protein,” as used herein, refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity.

The terms, “fusion effector protein,” “fusion protein,” and “fusion polypeptide,” as used herein, refer to a protein comprising at least two heterologous polypeptides. Often a fusion effector protein comprises an effector protein and a fusion partner protein. In general, the fusion partner protein is not an effector protein. Examples of fusion partner proteins are provided herein.

The term, “fusion partner protein” or “fusion partner,” as used herein, refers to a protein, polypeptide or peptide that is fused to an effector protein. The fusion partner generally imparts some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

The term, “genetic disease”, as used herein, refers to a disease caused by one or more mutations in the DNA of an organism. In some instances, a disease is referred to as a “disorder.” Mutations may be due to several different cellular mechanisms, including, but not limited to, an error in DNA replication, recombination, or repair, or due to environmental factors. Mutations may be encoded in the sequence of a target nucleic acid from the germline of an organism. A genetic disease may comprise a single mutation, multiple mutations, a chromosomal aberration, or combinations thereof.

The term, “guide nucleic acid,” as used herein, refers to a nucleic acid comprising: a first nucleotide sequence that hybridizes to a target nucleic acid; and a second nucleotide sequence that is capable of connecting an effector protein to the nucleic acid by either a) hybridizing to a portion of an additional nucleic acid that is bound by an effector protein (e.g., a tracrRNA) or b) being non-covalently bound by an effector protein. The first sequence may be referred to herein as a spacer sequence. In some instances, the second sequence may be referred to herein as a repeat sequence. In some instances, the second sequence may be referred to herein as a handle sequence. In some instances, the handle sequence may comprise a portion of, or all of a repeat sequence. In some instances, the first sequence is located 5′ of the second nucleotide sequence. In some instances, the first sequence is located 3′ of the second nucleotide sequence. In a single guide nucleic acid system, also referred to as a single guide RNA (sgRNA), the second sequence may be a handle sequence.

The term, “handle sequence,” as used herein, in the context of a sgRNA, refers to a portion of the sgRNA that is capable of being non-covalently bound by an effector protein. The nucleotide sequence of a handle sequence may contain or be derived from a tracrRNA sequence. For example, in some aspects, a handle sequence can include a portion of a tracrRNA sequence that is capable of being non-covalently bound by an effector protein, but does not include all or a part of the portion of a tracrRNA sequence that hybridizes to a portion of a crRNA as found in a dual nucleic acid system. In some aspects, a handle sequence can include a portion of a tracrRNA sequence as well as a portion of a repeat sequence, which can optionally be connected by a linker. In some aspects, a handle sequence in the context of a sgRNA can also be described as the portion of the sgRNA that does not hybridize to a target sequence in a target nucleic acid (e.g., a spacer sequence).

The term, “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some instances, the heterologous protein exhibits an activity (e.g., enzymatic activity) when it is fused to the effector protein. In some instances, the heterologous protein exhibits increased or reduced activity (e.g., enzymatic activity) when it is fused to the effector protein, relative to when it is not fused to the effector protein. In some instances, the heterologous protein exhibits an activity (e.g., enzymatic activity) that it does not exhibit when it is fused to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “in vitro,” as used herein, is used to describe an event that takes places contained in a container for holding laboratory reagents such that it is separated from the biological source from which the material is obtained. In vitro assays can encompass cell-based assays in which living or dead cells are employed. In vitro assays can also encompass a cell-free assay in which no intact cells are employed. The term, “in vivo”, is used to describe an event that takes place in a subject's body. The term, “ex vivo”, is used to describe an event that takes place outside of a subject's body. An ex vivo assay is not performed on a subject. Rather, it is performed upon a sample separate from a subject. An example of an ex vivo assay performed on a sample is an “in vitro” assay.

The term, “linked amino acids,” as used herein, refers to at least two amino acids linked by an amide bond.

The term, “linker,” as used herein, refers to a bond or molecule that links a first polypeptide to a second polypeptide or a first nucleic acid to a second nucleic acid. A “peptide linker” comprises at least two amino acids linked by an amide bond.

The term, “modified target nucleic acid,” as used herein, refers to a target nucleic acid, wherein the target nucleic acid has undergone a modification, for example, after contact with an effector protein. In some instances, the modification is an alteration in the sequence of the target nucleic acid. In some instances, the modified target nucleic acid comprises an insertion, deletion, or replacement of one or more nucleotides compared to the unmodified target nucleic acid.

The term, “mutation associated with a disease,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “nucleic acid expression vector,” as used herein, refers to a plasmid that can be used to express a nucleic acid of interest.

The term, “nuclear localization signal,” as used herein, refers to an entity (e.g., peptide) that facilitates localization of a nucleic acid, protein, or small molecule to the nucleus, when present in a cell that contains a nuclear compartment.

The term, “nuclease activity,” as used herein, refers to the enzymatic activity of an enzyme which allows the enzyme to cleave the phosphodiester bonds between the nucleotide subunits of nucleic acids; the term, “endonuclease activity”, refers to the enzymatic activity of an enzyme which allows the enzyme to cleave the phosphodiester bond within a polynucleotide chain. An enzyme with nuclease activity may be referred to as a “nuclease.”

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The term, “protospacer adjacent motif (PAM),” as used herein, refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term, “recombinant,” as used herein, as applied to proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Thus, for example, the term, “recombinant polynucleotide” or “recombinant nucleic acid”, refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. Similarly, the term, “recombinant polypeptide” or “recombinant protein”, refers to a polypeptide which is not naturally occurring, e.g., is made by the artificial combination of two otherwise separated segments of amino sequences through human intervention. Thus, for example, a polypeptide that includes a heterologous amino acid sequence is a recombinant polypeptide.

In some embodiments, the term, “region”, as used herein may be used to describe a portion of or all of a corresponding sequence, for example, a spacer region is understood to comprise a portion of or all of a spacer sequence.

The terms, “reporter,” “reporter nucleic acid,” and “reporter molecule,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein, generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “subject,” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease.

The term, “syndrome”, as used herein, refers to a group of symptoms which, taken together, characterize a condition.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence may bring an effector protein into contact with the target nucleic acid.

The term, “trans cleavage,” is used herein, in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids.

The term, “trans-activating RNA (tracrRNA),” as used herein, refers to a nucleic acid that comprises a first sequence that is capable of being non-covalently bound by an effector protein. TracrRNAs may comprise a second sequence that hybridizes to a portion of a crRNA, which may be referred to as a repeat hybridization sequence.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The terms, “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

### III. Introduction

Programmable nucleases are proteins that bind and cleave nucleic acids in a sequence-specific manner. A programmable nuclease may bind a target region of a nucleic acid and cleave the nucleic acid within the target region or at a position adjacent to the target region. In some embodiments, a programmable nuclease is activated when it binds (e.g., non-covalently interacts (e.g., ionic bonds, hydrogen bonds, van der Waals and hydrophobic interactions)) a target region of a nucleic acid to cleave regions of the nucleic acid that are near, but not adjacent to the target region. A programmable nuclease, such as a CRISPR-associated (Cas) protein, may be coupled to a guide nucleic acid that imparts activity or sequence selectivity to the programmable nuclease. The programmable nuclease and guide nucleic acid may form a complex that recognizes a target region of a nucleic acid and cleaves the nucleic acid within the target region, at a position adjacent to the target region, or at a position near to the target region. In general, guide nucleic acids comprise a CRISPR RNA (crRNA) that is at least partially complementary to a target nucleic acid.

In some embodiments, compositions, systems, and methods comprise a single guide RNA (sgRNA) or uses thereof. In some embodiments, the sgRNA comprises a handle sequence and a spacer sequence. In some embodiments, the handle sequence comprises an intermediary sequence, a repeat sequence or a combination thereof. In some embodiments, a handle sequence or at least a portion thereof interacts with the programmable nuclease. Accordingly, in some embodiments, an intermediary sequence, a repeat sequence, a portion thereof, or a combination thereof, interacts with the programmable nuclease. In some embodiments, an intermediary sequence is not a transactivating nucleic acid in systems, methods, and compositions described herein.

In some embodiments, in a dual nucleic acid system, a composition comprising effector proteins and guide nucleic acids further comprise a trans-activating crRNA (tracrRNA), at least a portion of which interacts with the programmable nuclease. In some embodiments, a tracrRNA or intermediary sequence (e.g., intermediary RNA) is provided separately from the guide nucleic acid, wherein the guide nucleic acid is the crRNA. The tracrRNA may hybridize to a portion of the guide nucleic acid that does not hybridize to the target nucleic acid, wherein the guide nucleic acid is the crRNA.

In some embodiments, hybridizing nucleotide sequences non-covalently interacts with each other, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anticodon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences hybridize. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide RNA (crRNA or sgRNA), wherein cleavage occurs within or directly adjacent to the region of the target nucleic acid that is hybridized to guideRNA. Trans cleavage activity (also referred to as transcollateral cleavage) is cleavage of ssDNA or ssRNA that is near, but not hybridized to the guide RNA. Trans cleavage activity is triggered by the hybridization of guide RNA to the target nucleic acid. Nickase activity is the selective cleavage of one strand of a dsDNA molecule.

Programmable CRISPR-associated (Cas) nucleases, through their ability to cleave DNA at a precise target location in the genome of a wide variety of cells and organisms, allow for precise and efficient editing of DNA sequences of interest. SSBs and DSBs are an effective way to disrupt a gene of interest, generate DNA or RNA modifications, and to treat genetic disease through gene correction.

Duchenne Muscular Dystrophy (DMD) is a severe X-linked recessive neuromuscular disorder effecting approximately 1 in 4,000 live male births. It is caused by mutations in the dystrophin gene (Chromosome X: 31,117,228-33,344,609 (Genome Reference Consortium—GRCh38/hg38)). With a genomic region of over 2.2 megabases in length, dystrophin is the second largest human gene. The dystrophin gene contains 79 exons that are processed into an 11,000 base pair mRNA that is translated into a 427 kDa protein. Functionally, dystrophin acts as a linker between the actin filaments and the extracellular matrix within muscle fibers. The N-terminus of dystrophin is an actin binding domain, while the C-terminus interacts with a transmembrane scaffold that anchors the muscle fiber to the extracellular matrix. Upon muscle contraction, dystrophin provides structural support that allows the muscle tissue to withstand mechanical force. DMD is caused by a wide variety of mutations within the dystrophin gene that result in premature stop codons and therefore a truncated dystrophin protein. Truncated dystrophin proteins do not contain the C-terminus, and therefore cannot provide the structural support necessary to withstand the stress of muscle contraction. As a result, the muscle fibers pull themselves apart, which leads to muscle wasting.

Patients are generally diagnosed by the age of 4, and wheelchair bound by the age of 10. Most patients do not live past the age of 25 due to cardiac and/or respiratory failure. Existing treatments are palliative at best. The most common treatment for DMD is steroids, which are used to slow the loss of muscle strength. However, because most DMD patients start receiving steroids early in life, the treatment delays puberty and further contributes to the patient's diminished quality of life. Thus, there remains a need for compositions, systems and methods for treating disorders associated with the dystrophin gene, such as DMD.

Disclosed herein are non-naturally occurring compositions, methods and systems comprising at least one of an engineered effector protein and an engineered guide nucleic acid (which may simply be referred to herein as an effector protein and a guide nucleic acid, respectively), or a use thereof. In general, an effector protein and a guide nucleic acid refer to an effector protein and a guide nucleic acid, respectively, that are not found in nature. In some embodiments, systems, methods and compositions described herein comprise an engineered effector protein or a use thereof. In some embodiments, systems, methods and compositions described herein comprise at least one non-naturally occurring component. For example, compositions, methods and systems may comprise a guide nucleic acid, wherein the nucleotide sequence of the guide nucleic acid is different or modified from that of a naturally-occurring guide nucleic acid. In some embodiments, compositions, methods and systems comprise at least two components that do not naturally occur together. For example, compositions, methods and systems may comprise a guide nucleic acid comprising a repeat region and a spacer region which do not naturally occur together. Also, by way of example, composition and systems may comprise a guide nucleic acid and an effector protein that do not naturally occur together. Likewise, by way of non-limiting example, disclosed compositions, systems, and methods may comprise a ribonucleotide protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural sequence is a nucleotide sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions, methods and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a nucleotide sequence of a naturally-occurring repeat region and a spacer region that is complementary to a naturally-occurring eukaryotic sequence. The guide nucleic acid may comprise a nucleotide sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, a guide nucleic acid may comprise a naturally occurring crRNA and handle sequence coupled by a linker sequence. In some embodiments, the guide nucleic acid comprises two heterologous sequences arranged in an order or proximity that is not observed in nature. Therefore, compositions described herein are not naturally occurring.

In some embodiments, compositions, methods and systems described herein comprise an effector protein described herein or a nucleotide sequence encoding the effector protein. In some embodiments, compositions, methods and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. In some embodiments, the effector protein may comprise a heterologous effector protein. In some embodiments, the heterologous effector protein comprises an effector protein described herein fused to a heterologous polypeptide described herein. The effector protein may also comprise at least one additional amino acid relative to the naturally-occurring effector protein. For example, the effector protein may comprise an addition of a nuclear localization signal relative to the natural occurring effector protein. In some embodiments compositions, methods and systems described herein may comprise one or more nuclear localization signals (NLS). In some embodiments, compositions, methods and systems described herein may comprise a NLS that is adjacent to the N terminal of the effector protein or that is adjacent to the C terminal of the effector protein, or both. In certain embodiments, the nucleotide sequence encoding the effector protein is codon optimized (e.g., for expression in a eukaryotic cell) relative to the naturally occurring sequence. In some embodiments, the nucleotide sequence encoding the effector protein is codon optimized for expression in a eukaryotic cell.

### IV. Effector Proteins

Provided herein, in certain embodiments, are compositions that comprise one or more effector proteins, or nucleotide sequences encoding the one or more effector proteins. In some embodiments, an effector protein is a protein, polypeptide, or peptide that non-covalently binds to a guide nucleic acid to form a complex that contacts a target nucleic acid, wherein at least a portion of the guide nucleic acid hybridizes to a target region of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. In some embodiments, an interaction between a target nucleic acid and a complex, wherein the complex comprises an effector protein and a guide nucleic acid, comprises one or more of: recognition of a PAM sequence within the target nucleic acid by the effector protein, hybridization of the guide nucleic acid to the target nucleic acid, modification of the target nucleic acid by the effector protein, or a combination thereof. In some embodiments, a PAM sequence is within or adjacent to the target nucleic acid. Accordingly, in some embodiments, the modification activity may occur within 1, 2, 3, 4, 5, 6, 7, 8, 9 or 10 nucleotides of a 5′ or 3′ terminus of a PAM sequence. In some embodiments, a given effector protein may not require a PAM sequence being present in a target nucleic acid for the effector protein to modify the target nucleic acid. Accordingly, in some embodiments, an effector protein may also recognize a sequence that is at least 1, at least 2, at least 3, at least 4, at least 5, at least 6, at least 7, at least 8, at least 9, or at least 10 nucleotides away from 5′ or 3′ terminus of a PAM sequence present in the target nucleic acid, which may direct the modification activity of the effector protein.

Modification activity of an effector protein or an engineered protein described herein may be cleavage activity, binding activity, insertion activity, substitution activity, and the like. In some embodiments, modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or a combination thereof. An effector protein may modify a nucleic acid by cis cleavage or trans cleavage. The modification of the target nucleic acid generated by an effector protein may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization, increasing or decreasing catalytic activity of the translation product, or increasing or decreasing downstream signaling activity by the translation product).

An effector protein may be a CRISPR-associated (“Cas”) protein. An effector protein may function as a single protein, including a single protein that is capable of binding (e.g., non-covalently interacting) to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). An effector protein may be a modified effector protein having reduced modification activity (e.g., a catalytically defective effector protein) or no modification activity (e.g., a catalytically inactive effector protein). Accordingly, an effector protein as used herein encompasses a modified or programmable nuclease that does not have nuclease activity.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some embodiments, the target nucleic acid is single-stranded DNA. In some embodiments, the target nucleic acid is single-stranded RNA. The effector proteins may provide cis cleavage activity, trans cleavage activity, nickase activity, or a combination thereof. Cis cleavage activity is cleavage of a target nucleic acid that is hybridized to a guide RNA (e.g., a dual nucleic acid system or a sgRNA), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide RNA. Trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid. Nickase activity is a selective cleavage of one strand of a dsDNA.

In some embodiments, the effector proteins function as an endonuclease that catalyzes cleavage within a target nucleic acid. In some embodiments, the effector proteins are capable of catalyzing non-sequence-specific cleavage of a single stranded nucleic acid. In some embodiments, the effector proteins (e.g., the effector proteins having the amino acid sequence of SEQ ID NO: 1) are activated to perform trans cleavage activity after binding/hybridizing of a guide nucleic acid with a target nucleic acid. This trans cleavage activity may also be referred to as “collateral” or “transcollateral” cleavage. Trans cleavage activity may be non-specific cleavage of nearby single-stranded nucleic acid by the activated effector protein, such as trans cleavage of detector nucleic acids with a detection moiety.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is at least 400 linked amino acid residues. In some embodiments, the length of the effector protein is less than 500 linked amino acid residues. In some embodiments, the length of the effector protein is about 400 to about 500 linked amino acid residues. In some embodiments, the length of the effector protein is about 450 to about 550, about 400 to about 420, about 420 to about 440, about 440 to about 460, about 460 to about 480, or about 480 to about 500. In some embodiments, compositions, systems, and methods described herein comprise an effector protein, wherein the amino acid sequence of the effector protein comprises at least 200, at least 220, at least 240, at least 260, at least 280, at least 300, at least 320, at least about 340, at least 360, at least 380, at least 400, at least 420, at least 440, or more contiguous amino acids of the amino acid sequence of SEQ ID NO: 1.

TABLE 1 provides an illustrative amino acid sequence of an effector protein. In some embodiments, an effector protein is at least 65%, at least 70%, at least 75%, at least 80%, at least 85%, at least 90%, at least 95%, at least 97%, or at least 98%, at least 99%, or 100% identical to the amino acid sequence as set forth in SEQ ID NO: 1. In some embodiments, percent of residues that are identical between respective positions of two sequences when the two sequences are aligned for maximum sequence identity. In some embodiments, the % identity is calculated by dividing the total number of the aligned residues by the number of the residues that are identical between the respective positions of the at least two sequences and multiplying by 100. In some embodiments, an effector protein provided herein comprises an amino acid sequence that is at least 65% identical to the amino acid sequence as set forth in SEQ ID NO: 1. In some embodiments, an effector protein provided herein comprises an amino acid sequence that is at least 70% identical to the amino acid sequence as set forth in SEQ ID NO: 1. In some embodiments, an effector protein provided herein comprises an amino acid sequence that is at least 75% identical to the amino acid sequence as set forth in SEQ ID NO: 1. In some embodiments, an effector protein provided herein comprises an amino acid sequence that is at least 80% identical to the amino acid sequence as set forth in SEQ ID NO: 1. In some embodiments, an effector protein provided herein comprises an amino acid sequence that is at least 85% identical to the amino acid sequence as set forth in SEQ ID NO: 1. In some embodiments, an effector protein provided herein comprises an amino acid sequence that is at least 90% identical to the amino acid sequence as set forth in SEQ ID NO: 1. In some embodiments, an effector protein provided herein comprises an amino acid sequence that is at least 95% identical to the amino acid sequence as set forth in SEQ ID NO: 1. In some embodiments, an effector protein provided herein comprises an amino acid sequence that is at least 97% identical to the amino acid sequence as set forth in SEQ ID NO: 1. In some embodiments, an effector protein provided herein comprises an amino acid sequence that is at least 98% identical to the amino acid sequence as set forth in SEQ ID NO: 1. In some embodiments, an effector protein provided herein comprises an amino acid sequence that is at least 99% identical to the amino acid sequence as set forth in SEQ ID NO: 1. In some embodiments, an effector protein provided herein comprises an amino acid sequence that is identical to the amino acid sequence of SEQ ID NO: 1.

In certain embodiments, compositions, systems, and methods described herein comprise an effector protein and an engineered guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to the amino acid sequence of SEQ ID NO: 1. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to the amino acid sequence of SEQ ID NO: 1.

In certain embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 98%, at least 99%, or 100% similar to the amino acid sequence of SEQ ID NO: 1. An amino acid sequence of the effector protein is similar to the reference amino acid sequence, when a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1. In some embodiments, an effector protein provided herein comprises an amino acid sequence that is at least 80% similar to the amino acid sequence as set forth in SEQ ID NO: 1. In some embodiments, an effector protein provided herein comprises an amino acid sequence that is at least 85% similar to the amino acid sequence as set forth in SEQ ID NO: 1. In some embodiments, an effector protein provided herein comprises an amino acid sequence that is at least 90% identical to the amino acid sequence as set forth in SEQ ID NO: 1. In some embodiments, an effector protein provided herein comprises an amino acid sequence that is at least 95% similar to the amino acid sequence as set forth in SEQ ID NO: 1. In some embodiments, an effector protein provided herein comprises an amino acid sequence that is at least 97% similar to the amino acid sequence as set forth in SEQ ID NO: 1. In some embodiments, an effector protein provided herein comprises an amino acid sequence that is at least 98% similar to the amino acid sequence as set forth in SEQ ID NO: 1. In some embodiments, an effector protein provided herein comprises an amino acid sequence that is at least 99% similar to the amino acid sequence as set forth in SEQ ID NO: 1. In some embodiments, an effector protein provided herein comprises an amino acid sequence that is 100% similar to the amino acid sequence as set forth in SEQ ID NO: 1.

In certain embodiments, compositions, systems, and methods described herein comprise an effector protein and an engineered guide nucleic acid, wherein the amino acid sequence of the effector protein comprises at least about 200, at least about 220, at least about 240, at least about 260, at least about 280, at least about 300, at least about 320, at least about 340, at least about 360, at least about 380, or at least about 400 contiguous amino acids or more of the amino acid sequence as set forth in SEQ ID NO: 1. In certain instances, compositions, systems, and methods described herein comprise an effector protein and an engineered guide nucleic acid, wherein the amino acid sequence of the effector protein comprises at least about 200 contiguous amino acids or more of the amino acid sequence as set forth in SEQ ID NO: 1. In certain instances, compositions, systems, and methods described herein comprise an effector protein and an engineered guide nucleic acid, wherein the amino acid sequence of the effector protein comprises at least about 300 contiguous amino acids or more of the amino acid sequence as set forth in SEQ ID NO: 1. In certain instances, compositions, systems, and methods described herein comprise an effector protein and an engineered guide nucleic acid, wherein the amino acid sequence of the effector protein comprises at least about 400 contiguous amino acids or more of the amino acid sequence of SEQ ID NO: 1.

In some embodiments, compositions, systems, and methods described herein comprise an effector protein, or a nucleic acid encoding the effector protein, wherein the effector protein comprises one or more amino acid alterations relative to the amino acid sequence of SEQ ID NO: 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to the amino acid sequence of SEQ ID NO: 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more amino acid alterations relative to the amino acid sequence of SEQ ID NO: 1. In some embodiments, the effector protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 amino acid alterations relative to the amino acid sequence of SEQ ID NO: 1. In some embodiments, the effector protein comprises one, two, three, four, five, six, seven, eight, nine, or ten amino acid alterations relative to the amino acid sequence of SEQ ID NO: 1. In some embodiments, the one or more amino acid alterations comprises substitutions (e.g., conservative substitutions or non-conservative substitutions), deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the amino acid sequence of SEQ ID NO: 1.

In some embodiments, compositions, systems, and methods described herein comprise an effector protein, or a nucleic acid encoding the effector protein, wherein the effector protein comprises one or more substitutions (e.g., conservative substations or non-conservative substitutions) relative to the amino acid sequence of SEQ ID NO: 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to the amino acid sequence of SEQ ID NO: 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more substitutions relative to the amino acid sequence of SEQ ID NO: 1. In some embodiments, the one or more substitutions comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid substitutions relative to the amino acid sequence of SEQ ID NO: 1. In some embodiments, the one or more substitutions comprise one, two, three, four, five, six, seven, eight, nine, ten or more substitutions relative to the amino acid sequence of SEQ ID NO: 1.

In some embodiments, the effector proteins described herein comprise one or more amino acid substitutions with a positively charged amino acid residues. In some embodiments, the positively charged amino acid residue is independently selected from Lys (K), Arg (R), and His (H). In some embodiments, the effector protein comprising one or more positively charged substitutions effects change in catalytic activity of the effector protein relative to the effector protein of SEQ ID NO: 1. In some embodiments, the effector protein comprising one or more positively charged substitutions effects increase in catalytic activity of the effector protein relative to the effector protein of SEQ ID NO: 1.

In certain embodiments, the effector protein described herein can comprise one or more functional domains. In certain embodiments, the effector protein described herein can comprise one or more functional domains comprising a protospacer adjacent motif (PAM)-interacting domain, an oligonucleotide-interacting domain, one or more recognition domains, a non-target strand interacting domain, and a RuvC, domain.

A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of a polypeptide (e.g., effector protein) that interacts with target nucleic acid.

In some embodiments, the effector proteins comprise a RuvC domain. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds. In some embodiments, effector proteins comprise a recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

Effector proteins of the present disclosure, dimers thereof, and multimeric complexes thereof may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region. In some embodiments, the effector protein recognizes a PAM sequence as shown in TABLE 2. In some embodiments, the effector protein recognizes a PAM sequence comprising any of the following nucleotide sequences as set forth in TABLE 2. In some embodiments, a composition comprising an effector protein recognizes a PAM sequence comprising any of the following nucleotide sequences as set forth in TABLE 2.

In some instances, effector proteins described herein recognize a PAM sequence comprising a nucleotide sequence of SEQ ID NO: 2. In some instances, compositions, methods and systems described herein comprises an effector protein that recognizes a PAM sequence comprising a nucleotide sequence of SEQ ID NO: 2. In some instances, effector proteins described herein recognize a PAM sequence comprising a nucleotide sequence of SEQ ID NO: 3. In some instances, compositions, methods and systems described herein comprises an effector protein that recognizes a PAM sequence comprising a nucleotide sequence of SEQ ID NO: 3.

### Engineered Proteins

In some embodiments, effector proteins disclosed herein are engineered proteins. Engineered proteins described herein include modified effector proteins. In some embodiments, the effector proteins described herein can be modified by altering one or more amino acids (e.g., deletion, insertion, or substitution). When describing a mutation that changes an amino acid residue or a nucleotide as described herein, such a change or changes can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation.

When a conservative substitution is described herein, such a substitution refers to the replacement of one amino acid for another such that the replacement takes place within a family of amino acids that are related in their side chains. Alternatively, a non-conservative substitution, when described herein, refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), le (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M)(see, for example, Biochemistry, 4th ed., Ed. by L. Stryer, WH Freeman and Co., 1995, which is incorporated by reference herein in its entirety).

In some embodiments, the effector protein has at least one amino acid residue alteration relative to an amino acid sequence of the naturally-occurring protein, wherein the at least one amino acid residue alteration is a conservative amino acid substitution. In some aspects, such a conservative amino acid sequence is a chemically conservative or an evolutionary conservative amino acid substitution. Methods of identifying conservative amino acids are well known to one of skill in the art, any one of which can be used to generate the effector proteins described herein. In some embodiments, the effector protein has at least one amino acid residue alteration relative to an amino acid sequence of the naturally-occurring protein, wherein the at least one amino acid residue alteration is a non-conservative amino acid substitution.

In some embodiments, the engineered protein has one or more alterations at one or more positions in a region that comprises substrate binding activity, catalytic activity, and/or binding affinity for a substrate such as a target nucleic acid, an engineered guide nucleic acid or a guide nucleic acid-target nucleic acid heteroduplex. In some embodiments, engineered proteins are not identical to a naturally-occurring protein. In some embodiments, the engineered protein may have different nucleic acid-cleaving activity relative to the naturally-occurring protein.

In some embodiments, the effector protein may comprise one or more amino acid changes (e.g., deletion, insertion, or substitution) that reduces the nucleic acid-cleaving activity of the effector protein relative to the naturally-occurring protein. For example, the effector protein can have increased modification activity and/or increased substrate binding activity (e.g., substrate selectivity, specificity, and/or affinity). In some embodiments, the effector protein has one or more activities that are at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over the naturally-occurring protein.

In some embodiments, the effector protein has reduced modification activity (e.g., a catalytically defective effector protein) or no modification activity (e.g., a catalytically inactive effector protein) relative to the naturally-occurring protein. In some embodiments, the effector protein has at least 90%, at least 80%, at least 70%, at least 30%, at least 40%, at least 30%, at least 20%, at least 10%, or 0% one or more activities relative to the naturally-occurring protein. Accordingly, the effector protein as used herein encompasses an effector protein or a variant thereof that does not have nuclease activity. In some embodiments, a variant of an effector protein comprises a form or version of the effector protein that differs from the reference effector protein (e.g., wild-type effector protein). A variant effector protein may have a different function or activity relative to the reference effector protein.

In some embodiments, compositions, systems, and methods described herein comprise an effector protein, or a nucleic acid encoding the effector protein, wherein the effector protein comprises one or more substitutions relative to the amino acid sequence of SEQ ID NO: 1. In some embodiments, the one or more substitutions comprise one or more conservative substitutions, one or more non-conservative substitutions, or combinations thereof.

In some embodiments, compositions, systems, and methods described herein comprise an effector protein, or a nucleic acid encoding the effector protein, wherein the effector protein comprises one or more conservative substitutions relative to the amino acid sequence of SEQ ID NO: 1. In some embodiments, the effector protein comprises at least one, at least two, at least three, at least four, at least five, at least six, at least seven, at least eight, at least nine, at least ten, at least twelve, at least sixteen, at least twenty, or more conservative substitutions relative to the amino acid sequence of SEQ ID NO: 1. In some embodiments, the effector protein comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more conservative substitutions relative to the amino acid sequence of SEQ ID NO: 1. In some embodiments, the effector protein comprises 1-10 conservative substitutions relative to the amino acid sequence of SEQ ID NO: 1. In some embodiments, the effector protein comprises 1-20 conservative substitutions relative to the amino acid sequence of SEQ ID NO: 1. In some embodiments, the effector protein comprises 1-30 conservative substitutions relative to the amino acid sequence of SEQ ID NO: 1. In some embodiments, the effector protein comprises 1-40 conservative substitutions relative to the amino acid sequence of SEQ ID NO: 1. In some embodiments, the effector protein comprises 1-50 conservative substitutions relative to the amino acid sequence of SEQ ID NO: 1. In some embodiments, the effector protein comprises 1-60 conservative substitutions relative to the amino acid sequence of SEQ ID NO: 1. In some embodiments, the effector protein comprises one, two, three, four, five, six, seven, eight, nine, or ten conservative substitutions relative to the amino acid sequence of SEQ ID NO: 1. In some embodiments, the effector protein comprises 1-10, 1-20, 1-30, 1-40, or 1-50 conservative substitutions relative to the amino acid sequence of SEQ ID NO: 1. In some embodiments, compositions, systems, and methods described herein comprise an effector protein, or a nucleic acid encoding the effector protein, wherein the effector protein comprises one or more alterations relative to the amino acid sequence of SEQ ID NO: 1 with the exception of 1, 2, 3, 4, 5, 6, 7, 8, 9 or 10 conservative amino acid substitutions.

In some embodiments, compositions, systems, and methods described herein comprise an effector protein, or a nucleic acid encoding the effector protein, wherein the effector protein comprises one or more non-conservative substitutions relative to the amino acid sequence of SEQ ID NO: 1. In some embodiments, the effector protein comprises at least one, at least two, at least three, at least four, at least five, at least six, at least seven, at least eight, at least nine, at least ten, at least twelve, at least sixteen, at least twenty, or more non-conservative substitutions relative to the amino acid sequence of SEQ ID NO: 1. In some embodiments, the effector protein comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more non-conservative substitutions relative to the amino acid sequence of SEQ ID NO: 1. In some embodiments, the one or more non-conservative substitutions comprise one, two, three, four, five, six, seven, eight, nine, ten non-conservative substitutions relative to the amino acid sequence of SEQ ID NO: 1. In some embodiments, compositions, systems, and methods described herein comprise an effector protein, or a nucleic acid encoding the effector protein, wherein the effector protein comprises one or more alterations relative to the amino acid sequence of SEQ ID NO: 1 with the exception of 1, 2, 3, 4, 5, 6, 7, 8, 9 or 10 non-conservative amino acid alterations.

In some embodiments, the compositions, systems, and methods described herein comprise an effector protein, or a nucleic acid encoding the effector protein, wherein the effector protein comprises an amino acid sequence that is at least 75%, at least 80%, at least 85%, at least 90%, at least 95%, at least 97%, at least 98%, or at least 99% to SEQ ID NO: 1, wherein not more than 1, 2, 3, 4, 5, 6, 7, 8, 9 or 10 amino acids of the amino acid sequence are non-conservative substitutions relative to SEQ ID NO: 1. In some embodiments, the compositions, systems, and methods described herein comprise an effector protein, or a nucleic acid encoding the effector protein, wherein the effector protein comprises an amino acid sequence that is at least 75%, at least 80%, at least 85%, at least 90%, at least 95%, at least 97%, at least 98%, or at least 99% to SEQ ID NO: 1, wherein all but 1, 2, 3, 4, 5, 6, 7, 8, 9 or 10 amino acids of the amino acid sequence are conservative substitutions relative to SEQ ID NO: 1.

Engineered proteins may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also, by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions, methods, and systems described herein may comprise an engineered effector protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

Compositions, methods, and systems may comprise an engineered effector protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions, methods, and systems may comprise an engineered effector protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wild type counterpart protein (e.g., an effector protein). The modified form of the wild type counterpart may comprise an amino acid change (e.g., deletion, insertion, or substitution) that reduces the nucleic acid-cleaving activity of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind (e.g., non-covalently interact) to a nucleic acid but not cleave it. An enzymatically inactive protein may comprise an enzymatically inactive domain (e.g., inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid. In some embodiments, the enzymatically inactive protein is fused with a protein comprising recombinase activity.

### Nuclease-Dead Effector Proteins

In some embodiments, the effector protein can comprise an enzymatically inactive and/or “dead” (abbreviated by “d”) effector protein in combination (e.g., fusion) with a polypeptide comprising recombinase activity. Although an effector protein normally has nuclease activity, in some embodiments, an effector protein does not have nuclease activity. In some embodiments, an effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% identical to the amino acid sequence of SEQ ID NO: 1, wherein the effector protein is a nuclease-dead effector protein. In some embodiments, the effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% identical to the amino acid sequence of SEQ ID NO: 1, wherein the effector protein is modified or engineered to be a nuclease-dead effector protein.

The effector protein can comprise a modified form of a wild type counterpart. The modified form of the wild type counterpart can comprise an amino acid change (e.g., deletion, insertion, or substitution) that reduces the nucleic acid-cleaving activity of the effector protein. For example, a nuclease domain (e.g., HEPN domain) of an effector polypeptide can be deleted or mutated so that it is no longer functional or comprises reduced nuclease activity. The modified form of the effector protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein can have no substantial nucleic acid-cleaving activity. When an effector protein is a modified form that has no substantial nucleic acid-cleaving activity, it can be referred to as enzymatically inactive and/or dead. A dead effector polypeptide can bind to a target sequence but may not cleave the target nucleic acid. A dead effector polypeptide can associate with a guide nucleic acid to activate or repress transcription of a target nucleic acid.

### Fusion Proteins

In some embodiments, an effector protein is a fusion protein, wherein the fusion protein comprises an effector protein and a fusion partner protein. In some embodiments, the effector protein comprises an amino acid sequence that is at least 65%, at least 70%, at least 75%, at least 80%, at least 85%, at least 90%, at least 95%, or 100% identical to the amino acid sequence as set forth in SEQ ID NO: 1. In some embodiments, an effector protein is a fusion protein, wherein the fusion protein comprises an effector protein and a fusion partner protein. In some embodiments, the effector protein comprises an amino acid sequence that is at least 65%, at least 70%, at least 75%, at least 80%, at least 85%, at least 90%, at least 95%, or 100% similar to the amino acid sequence as set forth in SEQ ID NO: 1. In some embodiments, the amino acid of the effector protein is at least 65%, at least 70%, at least 75%, at least 80%, at least 85%, at least 90%, at least 95%, or 100% identical to the amino acid sequence as set forth in SEQ ID NO: 1. In some embodiments, the amino acid of the effector protein is at least 65%, at least 70%, at least 75%, at least 80%, at least 85%, at least 90%, at least 95%, or 100% similar to the amino acid sequence as set forth in SEQ ID NO: 1. Unless otherwise indicated, reference to effector proteins throughout the present disclosure include fusion proteins thereof.

A fusion partner protein is also simply referred to herein as a fusion partner. In some embodiments, the fusion partner promotes the formation of a multimeric complex of the effector protein.

In some embodiments, the fusion partner inhibits the formation of a multimeric complex of the effector protein. By way of a non-limiting example, the fusion protein may comprise an effector protein and a fusion partner comprising a Calcineurin A tag, wherein the fusion protein dimerizes in the presence of Tacrolimus (FK506). Also, by way of non-limiting example, the fusion protein may comprise an effector protein and a SpyTag configured to dimerize or associate with another effector protein in a multimeric complex.

In some embodiments, the effector protein described herein can be modified with one or more modifying heterologous polypeptides (e.g., fusion partner). In some cases, the fusion partner comprises a subcellular localization sequence. In certain embodiments, the subcellular localization sequence can be a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like.

Accordingly, the composition, system and methods described herein may comprise the fusion protein, wherein the fusion partner is the nuclear localization signal (NLS). In some cases, the NLS comprises an entity (e.g., peptide) that facilitates localization of a nucleic acid, protein, or small molecule to the nucleus, when present in a cell that contains a nuclear compartment. An NLS can be located at or near the amino terminus (N-terminus) of the effector protein disclosed herein. An NLS can be located at or near the carboxy terminus (C-terminus) of the effector proteins disclosed herein. In some embodiments, a vector encodes the effector proteins described herein, wherein the vector or vector systems disclosed herein comprises one or more NLSs, such as about or more than about 1, 2, 3, 4, 5, 6, 7, 8, 9, 10, or more NLSs. In some embodiments, the effector protein described herein comprises 1, 2, 3, 4, 5, 6, 7, 8, 9, 10, or more NLSs at or near the N-terminus, 1, 2, 3, 4, 5, 6, 7, 8, 9, 10, or more NLSs at or near the C-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In certain embodiments, an NLS described herein comprises an NLS sequence recited in TABLE 3. Accordingly, in some embodiments, the effector protein described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to the amino acid sequence of SEQ ID NO: 1 and further comprises one or more amino acid sequences set forth in TABLE 3. Accordingly, in some embodiments, the effector protein described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% similar to the amino acid sequence of SEQ ID NO: 1 and further comprises one or more amino acid sequences set forth in TABLE 3. In some cases, an effector protein described herein is not modified with an NLS so that the polypeptide is not targeted to the nucleus, which can be advantageous depending on the circumstance (e.g., when the target nucleic acid is an RNA that is present in the cytosol).

In some cases, the fusion partner comprises a tag. A tag can be a heterologous polypeptide that is detectable for use in tracking and/or purification. Accordingly, in some embodiments, composition, system and methods described herein may comprise a purification tag and/or a fluorescent protein. Non-limiting examples of purification tags include a histidine tag, e.g., a 6×His tag (SEQ ID NO: 508); a hemagglutinin (HA) tag; a FLAG tag; a Myc tag; and maltose binding protein (MBP). Non-limiting examples of fluorescent proteins include green fluorescent protein (GFP), yellow fluorescent protein (YFP), red fluorescent protein (RFP), cyan fluorescent protein (CFP), mCherry, and tdTomato. Accordingly, in some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to the amino acid sequence of SEQ ID NO: 1 and further comprises one or more amino acid sequence of the tag. In some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% similar to the amino acid sequence of SEQ ID NO: 1 and further comprises one or more amino acid sequence of the tag.

In some embodiments, the fusion partner provides activities (e.g., enzymatic activities or transcription modulation activities), wherein the activities are not provided by effector proteins. It is also provided herein that, in some embodiments, such a fusion partner or a nucleic acid encoding the fusion partner can be provided separately, not fused to the effector protein, in the compositions, systems, and methods described herein.

In some embodiments, the fusion partner modulates transcription (e.g., inhibits transcription, increases transcription) of a target nucleic acid. In some embodiments, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some embodiments, the fusion partner is a protein (or a domain from a protein) that increases transcription, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. In some embodiments, the fusion partner is a reverse transcriptase. In some embodiments, the fusion partner is a base editor. In general, a base editor comprises a deaminase that when fused with a protein changes a nucleobase to a different nucleobase, e.g., cytosine to thymine or guanine to adenine. In some embodiments, the base editor comprises a deaminase.

In some embodiments, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity.

In some embodiments, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some embodiments, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof.

In some embodiments, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some embodiments, the fusion partner is a chloroplast transit peptide (CTP), also referred to as a plastid transit peptide. In some embodiments, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some embodiments, the CTP is located at the N-terminus of the fusion protein. Processing efficiency may, however, be affected by the amino acid sequence of the CTP and nearby sequences at the amino terminus (NH2 terminus) of the peptide.

In some embodiments, the fusion partner is an endosomal escape peptide. In some embodiments, an endosomal escape protein comprises the amino acid sequence GLFXALLXLLXSLWXLLLXA (SEQ ID NO: 505), wherein each X is independently selected from lysine, histidine, and arginine. In some embodiments, an endosomal escape protein comprises the amino acid sequence GLFHALLHLLHSLWHLLLHA (SEQ ID NO: 506). In some embodiments, the amino acid sequence of the endosomal escape protein is GLFXALLXLLXSLWXLLLXA (SEQ ID NO: 505) wherein each X is independently selected from lysine, histidine, and arginine or GLFHALLHLLHSLWHLLLHA (SEQ ID NO: 506).

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion proteins are targeted by a guide nucleic acid (guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some embodiments, the modifications are transient (e.g., transcription repression or activation). In some embodiments, the modifications are inheritable. For instance, epigenetic modifications made to a target nucleic acid, or to proteins associated with the target nucleic acid, e.g., nucleosomal histones, in a cell, are observed in cells produced by proliferation of the cell.

Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some embodiments may include a fragment of the protein (e.g., a functional domain). In some embodiments, the functional domain interacts with or binds ssRNA, including intramolecular and/or intermolecular secondary structures thereof, e.g., hairpins, stem-loops, etc.). The functional domain may interact transiently or irreversibly, directly or indirectly. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP Si, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some embodiments, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme fused or linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a target nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. In general, a base editor comprises a deaminase that when fused with a protein changes a nucleobase to a different nucleobase, e.g., cytosine to thymine or guanine to adenine. In some instances, the base editor comprises a deaminase. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3, eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any functional variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as fused to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U•G in DNA is initiated by a UNG, which recognizes a U•G mismatch and cleaves the glyosidic bond between a uracil and a deoxyribose backbone of DNA. In some embodiments, BER results in the reversion of the U•G intermediate created by the first CBE back to a C•G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C•G base pair to a T•A base pair through a U•G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U•G mismatch to favor a U•A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A•T base pair to a G•C base pair. In some embodiments, the ABE converts a target A•T base pair to G•C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, an adenine base editing enzyme of an ABE is an adenosine deaminase. Non-limiting exemplary adenosine base editors suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme fused to TadA by a linker (e.g., wherein the base editing enzyme is fused to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA fused to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene.

### Reverse Transcriptase (RT) Editing

In some embodiments, systems and methods comprise components or uses of an RT editing system to modify a target nucleic acid. In some embodiments, an RT editing system comprises an effector protein that is linked to a fusion partner that comprises an RT editing enzyme. In some embodiments, an RT editing system comprises an effector protein that is linked to a fusion partner that comprises an RT editing enzyme. In some instances, the RT editing enzyme is not linked or otherwise covalently attached to the effector protein, but rather recruited to the target nucleic acid by another means. By way of non-limiting example, the RT editing enzyme may be fused to an aptamer binding protein and the guide nucleic acid may be linked to or comprise a corresponding aptamer. In some embodiments, an RT editing enzyme comprises a polymerase. In some embodiments, an RT editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme. In some instances, systems and methods comprise an RT editing enzyme, wherein the RT editing enzyme is not fused or linked to the effector protein. In some instances, the RT editing enzyme comprises a recruiting moiety that recruits the RT editing enzyme to the target nucleic acid. By way of non-limiting example, the RT editing enzyme may comprise a peptide that binds an aptamer, wherein the aptamer is located on a guide RNA, template RNA, or combination thereof. Also, by way of non-limiting example, the RT editing enzyme may be linked to a protein that binds to (or is bound by) the effector protein or a protein linked/fused to the effector protein.

In some embodiments, an RT editing enzyme may require an RT editing guide RNA (pegRNA) to catalyze editing. Such a pegRNA may be capable of identifying a target nucleotide or target sequence in a target nucleic acid to be edited and encoding a new genetic information that replaces the target nucleotide or target sequence in the target nucleic acid. An RT editing enzyme may require a pegRNA and a single guide RNA to catalyze the editing. In some embodiments, the RT editing system comprises a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule except for at least one nucleotide. In some embodiments, the template RNA is covalently linked to a guide RNA. In some instances, the guide RNA is a single guide RNA. In some embodiments, the template RNA is not covalently linked to a guide RNA. In some embodiments, at least a portion of the template RNA hybridizes to the target nucleic acid. In some embodiments, the target nucleic acid is a dsDNA molecule. In some embodiments, at least a portion of the template RNA hybridizes to a first strand of the target nucleic acid and at least a portion of the single guide RNA hybridizes to a second strand of the target nucleic acid. In some embodiments, the pegRNA comprises a guide RNA comprising a first region that is bound by the effector protein, and a second region comprising a spacer sequence that is complementary to a target sequence of the dsDNA molecule; a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the at least one nucleotide is incorporated into the target nucleic acid by activity of the RT editing enzyme, thereby modifying the target nucleic acid. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### Linkers

In some embodiments, a terminus of the effector protein is linked to a terminus of the fusion partner through an amide bond. In some embodiments, the carboxy terminus of the effector protein is linked to the amino terminus of the fusion partner. In some embodiments, the carboxy terminus of the fusion partner is linked to the amino terminus of the effector protein. In some embodiments, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn (SEQ ID NO: 509), GGSGGSn (SEQ ID NO: 510), and GGGSn (SEQ ID NO: 511), where n is an integer of at least one), glycine-alanine polymers, and alanine-serine polymers. Exemplary linkers may comprise amino acid sequences including, but not limited to, GS, GSGGS (SEQ ID NO: 512), GGSGGS (SEQ ID NO: 513), GGGS (SEQ ID NO: 514), GGSG (SEQ ID NO: 515), GGSGG (SEQ ID NO: 516), GSGSG (SEQ ID NO: 517), GSGGG (SEQ ID NO: 518), GGGSG (SEQ ID NO: 519), and GSSSG (SEQ ID NO: 520).

In some embodiments, a linker may be a peptide linker or a non-peptide linker. In some embodiments, the linker is an XTEN linker. In some embodiments, the XTEN linker is an XTEN20 linker. In some embodiments, the XTEN20 linker has an amino acid sequence of GSGGSPAGSPTSTEEGTSESATPGSG (SEQ ID NO: 507). In some embodiments, the XTEN linker is an XTEN80 linker. In some embodiments, the linker comprises one or more repeats as a GGS tri-peptide. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, linkers do not comprise an amino acid. In some instances, linkers do not comprise a peptide. In some embodiments, linkers comprise a nucleotide, a polynucleotide, a polymer, or a lipid.

### Codon Optimization and Start Codons

In some embodiments, effector proteins described herein may be codon optimized. In some embodiments, effector protein described herein are encoded by a codon optimized nucleic acid. In some embodiments, a nucleic acid sequence encoding an effector protein described herein, is codon optimized. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized Effector protein nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon. Accordingly, in some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a modifying heterologous peptide, such as a fusion protein partner, is located at the N terminus of the effector protein, a start codon for the fusion protein partner serves as a start codon for the effector protein as well. Thus, the natural start codon encoding an amino acid residue sufficient for initiating translation (e.g., Methionine (M) or a Valine (V)) of the effector protein may be removed or absent.

### Synthesis, Isolation, and Assay

Effector proteins of the present disclosure may be produced in vitro or by eukaryotic cells or by prokaryotic cells. When in vitro is described herein, it can be used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

Effector proteins can be further processed by unfolding, e.g. heat denaturation, dithiothreitol reduction, etc. and may be further refolded, using any suitable method. Effector proteins of the present disclosure of the present disclosure may be synthesized, using any suitable method.

Methods of generating and assaying the effector proteins described herein are well known to one of skill in the art. Examples of such methods are described in the Examples provided herein. Any of a variety of methods can be used to generate an effector protein disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art.

In some embodiments, an effector protein provided herein is an isolated effector protein. In some embodiments, effector proteins described herein can be isolated and purified for use in compositions, systems, and/or methods described herein. Methods described here can include the step of isolating effector proteins described herein. An isolated effector protein provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

For example, compositions, methods, and/or systems described herein can further comprise a purification tag that can be attached to an effector protein, or a nucleic acid encoding for a purification tag that can be attached to a nucleic acid encoding for an effector protein as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Examples of purification tags are as described herein.

In some embodiments, effector proteins described herein are isolated from cell lysate. In some embodiments, the compositions, methods, and systems described herein can comprise 20% or more by weight, 75% or more by weight, 95% or more by weight, or 99.5% or more by weight of an effector protein, wherein percentages can be upon total protein content in relation to contaminants. Some aspects related to the method of preparation of compositions described herein and purification thereof, wherein percentages can be upon total protein content in relation to contaminants. Thus, in some cases, an effector protein described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

### V. Multimeric Complexes

Compositions, systems, and methods of the present disclosure may comprise a multimeric complex or uses thereof, wherein the multimeric complex comprises multiple effector proteins that non-covalently interact with one another. A multimeric complex may comprise enhanced activity relative to the activity of any one of its effector proteins alone. For example, a multimeric complex comprising two effector proteins may comprise greater nucleic acid binding affinity, cis-cleavage activity, and/or transcollateral cleavage activity than that of either of the effector proteins provided in monomeric form. A multimeric complex may have an affinity for a target region of a target nucleic acid and is capable of catalytic activity (e.g., cleaving, nicking or modifying the nucleic acid) at or near the target region. Multimeric complexes may be activated when complexed with a guide nucleic acid. Multimeric complexes may be activated when complexed with a guide nucleic acid and a target nucleic acid. In some embodiments, the multimeric complex cleaves the target nucleic acid. In some embodiments, the multimeric complex nicks the target nucleic acid.

Various aspects of the present disclosure include compositions, systems, and methods comprising multiple effector proteins, and uses thereof, respectively. An effector protein comprising an amino acid sequence that is at least 65% identical to the amino acid sequence of SEQ ID NO: 1, wherein the effector protein may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids).

In some embodiments, multimeric complexes comprise at least one effector protein or a fusion protein thereof, wherein the amino acid sequence of the effector protein is at least 65%, at least 70%, at least 75%, at least 80%, at least 85%, at least 90%, at least 95% or 100% identical to the amino acid sequence of SEQ ID NO: 1. In some embodiments, multimeric complexes comprise at least one effector protein or a fusion protein thereof, wherein the amino acid sequence of the effector protein is at least 65%, at least 70%, at least 75%, at least 80%, at least 85%, at least 90%, at least 95% or 100% similar to the amino acid sequence of SEQ ID NO: 1.

In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98%, or at least 99% identical to the amino acid sequence of the second effector protein. In some embodiments, the multimeric complex is a dimer comprising two effector proteins of similar amino acid sequences. In some embodiments, the multimeric complex comprises a first effector protein and a second effector protein, wherein the amino acid sequence of the first effector protein is at least 90%, at least 92%, at least 94%, at least 96%, at least 98%, or at least 99% similar to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% similar to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, the multimeric complex is a homomeric complex. In some embodiments, the multimeric complex is a heteromeric complex. In some embodiments, at least one effector protein of the multimeric complex comprises an amino acid sequence that is at least 65%, at least 70%, at least 75%, at least 80%, at least 85%, at least 90%, at least 95% or 100% identical to the amino acid sequence of SEQ ID NO: 1. In some embodiments, at least one effector protein of the multimeric complex comprises an amino acid sequence that is at least 65%, at least 70%, at least 75%, at least 80%, at least 85%, at least 90%, at least 95% or 100% similar to the amino acid sequence of SEQ ID NO: 1. In some embodiments, each effector protein of the multimeric complex comprises an amino acid sequence that is at least 65%, at least 70%, at least 75%, at least 80%, at least 85%, at least 90%, at least 95% or 100% identical to the amino acid sequence of SEQ ID NO: 1. In some embodiments, each effector protein of the multimeric complex comprises an amino acid sequence that is at least 65%, at least 70%, at least 75%, at least 80%, at least 85%, at least 90%, at least 95% or 100% similar to the amino acid sequence of SEQ ID NO: 1.

### VI. Nucleic Acid Systems

### Guide Nucleic Acids

The compositions, systems, and methods of the present disclosure may comprise a guide nucleic acid or a use thereof. In general, a guide nucleic acid is a nucleic acid molecule that binds (e.g., non-covalently interacts) to an effector protein, thereby forming a ribonucleoprotein complex (RNP). Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein. Guide nucleic acids may comprise DNA, RNA, or a combination thereof (e.g., RNA with a thymine base). Guide nucleic acids may include a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides.

In some embodiments, a guide nucleic acid may comprise a CRISPR RNA (crRNA), a short-complementarity untranslated RNA (scoutRNA), a handle sequence or a combination thereof. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, or handle sequence, or a combination thereof. In some embodiments, the handle sequence may comprise a portion of, or all of, a repeat sequence. A guide nucleic acid may comprise a naturally occurring guide nucleic acid. A guide nucleic acid may comprise a non-naturally occurring guide nucleic acid, including a guide nucleic acid that is designed to contain a chemical or biochemical modification.

A guide RNA can generally comprise a crRNA, at least a portion of which is complementary to a target sequence of a target nucleic acid. In some embodiments, the guide RNA comprises a handle sequence that interacts with the effector protein. In some embodiments, the guide RNA comprises a portion of, or all of a repeat sequence that interacts with the effector protein. In some embodiments, the composition, system or method described herein comprising an effector protein and a guide RNA further comprises a tracrRNA sequence that interacts with the effector protein. In some embodiments, the composition, system, or method described herein does not comprise a tracrRNA. In some embodiments, the guide RNA is a sgRNA. In some embodiments, a crRNA and tracrRNA function as two separate, unlinked molecules. Guide nucleic acids are often referred to as “guide RNA.” However, a guide nucleic acid may comprise deoxyribonucleotides. The term, “guide RNA,” as well as crRNA and tracrRNA, include guide nucleic acids comprising DNA bases and/or RNA bases. The guide RNA may be chemically synthesized or recombinantly produced. The sequence of the guide nucleic acid, or a portion thereof, may be different from the sequence of a naturally occurring nucleic acid.

In some embodiments, effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., modifying the target nucleic acid or modifying a protein associated with the target nucleic acid). The guide RNA may bind/hybridize to a target nucleic acid (e.g., a single strand of a target nucleic acid) or a portion thereof, an amplicon thereof, or a portion thereof. By way of non-limiting example, a guide nucleic acid may bind/hybridize to a target nucleic acid, such as DNA or RNA, from a gene associated with a genetic disorder, or an amplicon thereof, as described herein.

In some embodiments, the compositions, systems, and methods of the present disclosure may comprise an additional guide nucleic acid or a use thereof. An additional guide nucleic acid can target an effector protein to a different location in the target nucleic acid by binding/hybridizing to a different portion of the target nucleic acid from the first guide nucleic acid. For example, a guide nucleic acid can bind/hybridize a portion of the target nucleic acid that is upstream of a premature stop codon of a targeted gene that is formed as a result of an out-of-frame genetic mutation in a cell or subject as described herein (e.g., the dystrophin gene), wherein the additional guide nucleic acid can bind/hybridize to a portion of the target nucleic acid that is located either upstream or downstream of where the first guide RNA has targeted. In such embodiments, the dual-guided compositions, systems, and methods described herein can modify the target nucleic acid in two locations. In some embodiments, the dual-guided compositions, systems, and methods described herein can cleave the target nucleic acid in the two locations targeted by the guide RNAs. In certain embodiments, upon removal of the sequence between the guide nucleic acids, the wild-type reading frame is restored. resulting in at least a partially functional protein. In certain embodiments, upon removal of the sequence between the target sequences of the guide nucleic acids, any desired genomic sequences such as an entire exon or a region of sequences involved in mRNA splicing or multiple exons or certain specific sequences can be deleted. In some embodiments, a donor nucleic acid is inserted in replacement of the deleted sequence. The modification of the target nucleic acid at two different loci is referred to herein as “dual-cutting”. Accordingly, in some embodiments, dual-guide nucleic acid compositions, systems, and methods can comprise two effector proteins, individually corresponding a guide RNA or a single effector protein with two different guide RNA to achieve dual-cutting.

The guide nucleic acid may comprise a first region that is not complementary to a target nucleic acid (FR1) and a second region that is complementary to the target nucleic acid (FR2). In some embodiments, FR1 is located 5′ to FR2 (FR1-FR2). In some embodiments, FR2 is located 5′ to FR1 (FR2-FR1).

In some embodiments, the guide nucleic acid comprises 10, 11, 12, 13, 14, 15, 16, 17, 18, 19, 20, 21, 22, 23, 24, 25, 26, 27, 28, 29, or 30 linked nucleotides. In general, a guide nucleic acid comprises at least linked nucleotides. In some embodiments, a guide nucleic acid comprises at least 25 linked nucleotides. A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid has about 10 to about 60, about 20 to about 50, or about 30 to about 40 linked nucleotides.

In some embodiments, the guide nucleic acid comprises a nucleotide sequence as described herein (e.g., TABLE 4, TABLE 5, or TABLE 6). Such nucleotide sequences described herein (e.g., TABLE 4, TABLE 5, or TABLE 6) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 4, TABLE 5, or TABLE 6) also discloses the complementary nucleotide sequence, the reverse nucleotide sequence, and the reverse complement nucleotide sequence, any one of which can be a nucleotide sequence for use in a guide nucleic acid as described herein.

In some embodiments, the guide nucleic acid comprises a sequence that is at least 70%, at least 80%, at least 85%, at least 90%, at least 95%, or 100% identical to any one of the nucleotide sequences set forth in TABLE 4, TABLE 5, TABLE 6, or any combination thereof.

In some embodiments, the guide nucleic acid comprises a sequence that is at least 70%, at least 75%, at least 80%, at least 85%, at least 90%, at least 95%, or 100% identical to any one of the nucleotide sequences of TABLE 4 and a sequence that is at least 70%, at least 75%, at least 80%, at least 85%, at least 90%, at least 95%, or 100% identical to any one of the nucleotide sequences of TABLE 5. In some embodiments, the guide nucleic acid comprises a spacer sequence and/or a handle sequence. In some embodiments, the guide nucleic acid comprises a spacer sequence that is at least 70%, at least 75%, at least 80%, at least 85%, at least 90%, at least 95%, or 100% identical to any one of the nucleotide sequences of TABLE 4 and a handle sequence that is at least 70%, at least 75%, at least 80%, at least 85%, at least 90%, at least 95%, or 100% identical to any one of the nucleotide sequences of TABLE 5.

In some embodiments, compositions, systems and methods provided herein comprise a nucleotide sequence listed in TABLE 4, TABLE 5, or TABLE 6; and further comprises one or more sequence modification or mutation. Sequence mutation(s) or modification(s) can include, e.g., a substitution, a deletion, an insertion, a chemical modification of one or more nucleobases; or chemical modifications to the phosphate backbone, a nucleotide, a nucleobase, or a nucleoside. Such modifications can be made to the nucleic acid sequence or any sequence disclosed herein (e.g., a vector encoding a guide nucleic acid). Methods of modifying a nucleic acid or amino acid sequence are known. One of ordinary skill in the art will appreciate that the modification(s) may be located at any position(s) of a nucleic acid such that the function of the nucleic acid or synthetic protein is not substantially decreased. For example, software can be used to match identical or similar sequences by assigning degrees of homology to various substitutions, deletions, and/or other modifications. Nucleic acids provided herein can be prepared according to any available technique including, but not limited to chemical synthesis, enzymatic synthesis, which is generally termed in vitro-transcription, cloning, enzymatic, or chemical cleavage, etc. In some cases, the nucleic acids provided herein are not uniformly modified along the entire length of the molecule. Different nucleotide modifications and/or backbone structures can exist at various positions within the nucleic acid.

A person of ordinary skill in the art would appreciate that referring to a nucleotide(s), and/or nucleoside(s), in the context of a nucleic acid molecule having multiple residues, is interchangeable and describe the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a nucleobase, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another nucleotide sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two nucleotide sequences that are complementary to each other and hybridize to form a double stranded RNA duplex (dsRNA duplex). In some embodiments, the two nucleotide sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a nucleotide sequence and the other strand of the stem portion comprises a nucleotide sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of SEQ ID NO: 443 or 504. In some embodiments, the repeat sequence is at least 85% identical to SEQ ID NO: 443 or 504. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of SEQ ID NO: 443 or 504.

In some embodiments, a repeat sequence comprises one or more nucleotide alterations at one or more positions in the sequence recited in SEQ ID NO: 443 or 504. Alternative nucleotides can be any one or more of A, C, G, T or U, or a deletion, or an insertion.

### Spacer Sequence

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a nucleotide sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

The spacer region may comprise complementarity with (e.g., hybridize to) a target sequence of a target nucleic acid. In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleotides that are complementary to the target nucleic acid.

TABLE 4 provides illustrative spacer sequences for use with the compositions and methods of the disclosure. In some embodiments, the spacer sequence comprises a nucleotide sequence that is at least 65%, at least 70%, at least 80%, at least 90%, at least 92%, at least 95%, at least 97%, or at least 99%, or 100% identical to a nucleotide sequence as set forth in TABLE 4.

In some embodiments, the spacer sequence comprises one or more nucleotide alterations at one or more positions in any one of the nucleotide sequences of TABLE 4. Alternative nucleotides can be any one or more of A, C, G, T or U, or a deletion, or an insertion.

It is understood that the sequence of a spacer region need not be 100% complementary to that of a target sequence of a target nucleic acid to hybridize or hybridize specifically to the target sequence. The guide nucleic acid may comprise at least one uracil between nucleic acid residues 5 to 20 of the spacer region that is not complementary to the corresponding nucleotide of the target sequence. The guide nucleic acid may comprise at least one uracil between nucleic acid residues 5 to 9, 10 to 14, or 15 to 20 of the spacer region that is not complementary to the corresponding nucleotide of the target sequence. In some embodiments, the region of the target nucleic acid that is complementary to the spacer region comprises an epigenetic modification or a post-transcriptional modification. In some embodiments, the epigenetic modification comprises an acetylation, methylation, or thiol modification.

It is understood that the spacer sequence of a spacer sequence need not be 100% complementary to that of a target sequence of a target nucleic acid to hybridize or hybridize specifically to the target sequence. For example, the spacer sequence may comprise at least one alteration, such as a substituted or modified nucleotide, that is not complementary to the corresponding nucleotide of the target sequence. Spacer sequences are further described throughout herein.

In some embodiments, the spacer sequence is a truncated version of any of those listed in TABLE 4. In some embodiments, the spacer is shortened at the 5′ end while maintaining the 3′ end (to maintain the PAM) and still long enough to hybridize under physiological conditions. In some embodiments, the spacer sequence comprises at least 5, at least 6, at least 7, at least 8, at least 9, at least 10, at least 11, at least 12, at least 13, at least 14, at least 15, at least 16, at least 17, at least 18, at least 19, or at least 20 contiguous nucleotides of any one of the nucleotide sequences recited in TABLE 4.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first nucleotide sequence, a second nucleotide sequence that is reverse complementary to the first nucleotide sequence, and a stem-loop linking the first nucleotide sequence and the second nucleotide sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to a nucleotide sequence of SEQ ID NO: 441. In some embodiments, an intermediary sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of a nucleotide sequence of SEQ ID NO: 441.

### Handle Sequence

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a nucleotide sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides. In some embodiments, the length of a handle sequence in a sgRNA is 69 nucleotides.

TABLE 5 provides illustrative handle sequence for an sgRNA and exemplary portions of a sgRNA (a handle sequence without a linker or repeat sequence, a linker, and a repeat sequence) for use with the compositions, systems, and methods of the disclosure. In some embodiments, the sgRNA sequence comprises a nucleotide sequence that is at least 65%, at least 70%, at least 80%, at least 90%, at least 92%, at least 95%, at least 97%, or at least 99%, or 100% identical to any one of the nucleotide sequences as set forth in TABLE 5, or a reverse complement thereof.

In some embodiments, the spacer sequence comprises one or more nucleotide alterations at one or more positions in any one of the nucleotide sequences of TABLE 5. Alternative nucleotides can be any one or more of A, C, G, T or U, or a deletion, or an insertion.

In some instances, compositions, systems and methods described herein comprise a nucleotide sequence that is at least 65%, at least 70%, at least 80%, at least 90%, at least 92%, at least 95%, at least 97%, or at least 99%, or 100% identical to any one of the nucleotide sequences as set forth in TABLE 5. In some instances, compositions, systems and methods described herein comprise a guide nucleic acid comprising a nucleotide sequence that is at least 65%, at least 70%, at least 80%, at least 90%, at least 92%, at least 95%, at least 97%, or at least 99%, or 100% identical to any one of the nucleotide sequences as set forth in TABLE 5. In some instances, compositions, systems and methods described herein comprise a single guide nucleic acid comprising a nucleotide sequence that is at least 65%, at least 70%, at least 80%, at least 90%, at least 92%, at least 95%, at least 97%, or at least 99%, or 100% identical to any one of the nucleotide sequences as set forth in TABLE 5.

In some instances, compositions, systems and methods described herein comprise a nucleotide sequence that is at least 65%, at least 70%, at least 80%, at least 90%, at least 92%, at least 95%, at least 97%, at least 99%, or 100% identical to one or more nucleotide sequences as set forth in TABLE 5, wherein the nucleotide sequence is a handle sequence, a handle sequence without a linker or repeat sequence, a linker, or a repeat sequence.

crRNA

Guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpf1, from Type V systems). A crRNA may also be specifically generated outside of processing of a pre-crRNA and individually contacted to an effector protein in vivo or in vitro.

In general, a crRNA can comprise a spacer region that hybridizes to a target sequence of a target nucleic acid, and in some embodiments can further comprise, a repeat region that interacts with the effector protein. In some embodiments, the repeat region may also be referred to as a “protein-binding segment.” Typically, the repeat region is adjacent to the spacer region. For example, a guide RNA that interacts with an effector protein comprises a repeat region that is 5′ of the spacer region.

Accordingly, in some embodiments, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the guide nucleic acid and the effector protein to form an RNP complex.

In some embodiments, the repeat sequence and the spacer sequences are directly connected to each other (e.g., covalent bond (phosphodiester bond)). In some embodiments, the repeat sequence and the spacer sequence are connected by a linker.

In such embodiments, a guide nucleic acid comprises a crRNA wherein, a repeat sequence of the crRNA is capable of connecting the crRNA to an effector protein. In some embodiments, the guide nucleic acid comprising the crRNA is linked to another nucleotide sequence that is capable of being non-covalently bond by an effector protein. In such embodiments, the repeat sequence of the crRNA can be linked to an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a crRNA comprises a nucleotide sequence that is at least 65%, at least 70%, at least 75%, at least 80%, at least 85%, at least 90%, at least 95%, at least 97%, or at least 98%, at least 99%, or 100% identical to any one of the crRNA sequences in TABLE 12. In some embodiments, a crRNA sequence comprises a repeat sequence that is at least 65%, at least 70%, at least 75%, at least 80%, at least 85%, at least 90%, at least 95%, at least 97%, or at least 98%, at least 99%, or 100% identical to SEQ ID NO: 443 or 504, and a spacer sequence that is at least 65%, at least 70%, at least 75%, at least 80%, at least 85%, at least 90%, at least 95%, at least 97%, or at least 98%, at least 99%, or 100% identical to any one of the sequences set forth in TABLE 4. In some embodiments, a crRNA comprises at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous nucleotides of any one of the crRNA sequences recited in TABLE 12. In some embodiments, a crRNA sequence comprises at least 5, at least 6, at least 7, at least 8, at least 9, or at least 10 contiguous nucleotides of any one of the repeat sequences recited in SEQ ID NO: 443 or 504, and at least 5, at least 6, at least 7, at least 8, at least 9, or at least 10 contiguous nucleotides of any one of the spacer sequences recited in TABLE 4.

sgRNA

In some embodiments, the compositions comprising a guide RNA and an effector protein without a tracrRNA (e.g., a single nucleic acid system), wherein the guide RNA is a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding (e.g., non-covalently interacting) of an effector protein to the sgRNA and/or modification activity of an effector protein on a target nucleic acid (e.g., a hairpin region). Such a sequence can be contained within a handle sequence as described herein. A sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

In some embodiments, the nucleotide sequence of the sgRNA comprises distinct regions as described TABLE 5. For example, in some embodiments, the sgRNA comprises a handle sequence, a handle sequence without a linker or repeat sequence, a linker, a repeat sequence, or a combination thereof. In some embodiments, the handle sequence comprises a nucleotide sequence that is at least 65%, at least 70%, at least 80%, at least 90%, at least 92%, at least 95%, at least 97%, or at least 99%, or 100% identical to ACAGCUUAUUUGGAAGCUGAAAUGUGAGGUUUAUAACACUCACAAGAAUCCU (SEQ ID NO: 441). In some embodiments, the sgRNA comprises a linker comprising the nucleotide sequence of GAAA (SEQ ID NO: 442). In some embodiments, the sgRNA comprises a repeat sequence that is at least 70%, at least 80%, at least 90%, at least 92%, at least 95%, at least 97%, at least 99%, or 100% identical to AAGGAUGCCAAAC (SEQ ID NO: 443). In some embodiments, the sgRNA comprises a portion of the handle sequence of TABLE 5 that is a contiguous sequence of nucleotides of one or more of such distinct regions. For example, in some embodiments, the sgRNA comprises at least 9, at least 10, at least 11, at least 12 contiguous nucleotides of SEQ ID NO: 443. In some embodiments, the sgRNA comprises at least 30, at least 35, at least 40, at least 45, at least 50 contiguous nucleotides of the SEQ ID NO: 441.

In some embodiments, compositions, systems, and methods disclosed herein comprise a guide nucleic acid comprising a spacer sequence that is at least 65%, at least 70%, at least 75%, at least 80%, at least 85%, at least 90%, at least 95%, or 100% identical to any one of the nucleotide sequences as set forth in TABLE 4 and comprising a handle sequence that is at least 65%, at least 70%, at least 75%, at least 80%, at least 85%, at least 90%, at least 95%, or 100% identical to the handle sequence of TABLE 5.

In some embodiments, compositions, systems, and methods disclosed herein comprise an effector protein comprising an amino acid sequence that is at least 65%, at least 70%, at least 75%, at least 80%, at least 85%, at least 90%, at least 95%, at least 99%, or 100% identical to the amino acid sequence as set forth in SEQ ID NO: 1; a guide nucleic acid comprising a spacer sequence that is at least 65%, at least 70%, at least 75%, at least 80%, at least 85%, at least 90%, at least 95%, or 100% identical to any one of the nucleotide sequences as set forth in TABLE 4 and comprising a handle sequence that is at least 65%, at least 70%, at least 75%, at least 80%, at least 85%, at least 90%, at least 95%, or 100% identical to the handle sequence of TABLE 5. In some embodiments, compositions, systems, and methods disclosed herein comprise an effector protein comprising an amino acid sequence that is at least 65%, at least 70%, at least 75%, at least 80%, at least 85%, at least 90%, at least 95%, at least 99%, or 100% similar to the amino acid sequence as set forth in SEQ ID NO: 1; a guide nucleic acid comprising a spacer sequence that is at least 65%, at least 70%, at least 75%, at least 80%, at least 85%, at least 90%, at least 95%, or 100% identical to any one of the nucleotide sequences as set forth in TABLE 4 and comprising a handle sequence that is at least 65%, at least 70%, at least 75%, at least 80%, at least 85%, at least 90%, at least 95%, or 100% identical to the handle sequence of TABLE 5.

TABLE 6 provides exemplary gRNA sequences. In some embodiments, the compositions, systems, and methods comprise an effector protein described herein and sgRNAs. Each row in TABLE 6 represents an exemplary gRNAs that can be used in composition, systems, and methods comprising an effector protein as set forth in SEQ ID NO: 1 recognizing a PAM sequence as set forth in TABLE 2 and a guide nucleic acid, wherein the guide nucleic acid is a sgRNA. In some embodiments, the sgRNA comprises a nucleotide sequence of any one of the sgRNA sequences of TABLE 6. In some embodiments, the nucleotide sequence of the guide nucleic acid is at least 65%, at least 70%, at least 75%, at least 80%, at least 85%, at least 90%, at least 95%, at least 97%, or at least 98%, at least 99%, or 100% identical to any one of the sgRNA sequences of TABLE 6.

### A Single Nucleic Acid System

A single nucleic acid system uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein. In a single nucleic system, the guide nucleic acid is not transactivating or transactivated. In a single nucleic acid system, the guide nucleic acid-polypeptide complex (e.g., an RNP complex) is not transactivated or transactivating.

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR1) of the guide nucleic acid non-covalently interacts with the one or more polypeptides described herein. In some embodiments, a second region (FR2) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a sgRNA.

### A Dual Nucleic Acid System

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule. In some embodiments, the dual nucleic acid system comprises a guide nucleic acid and a tracrRNA, wherein the tracrRNA is an additional nucleic acid capable of at least partially hybridizing to the first region of the guide nucleic acid. In some embodiments, the tracrRNA or additional nucleic acid is capable of at least partially hybridizing to the 5′ end of the second region of the guide nucleic acid.

In some embodiments, the compositions comprising a guide RNA and an effector protein (e.g., in a dual nucleic acid system) comprises a tracrRNA. A tracrRNA may include deoxyribonucleosides, ribonucleosides, chemically modified nucleosides, or any combination thereof. A tracrRNA may be separate from, but form a complex with, a guide nucleic acid and an effector protein. A tracrRNA may include a nucleotide sequence that hybridizes with a portion of a guide nucleic acid (e.g., a repeat hybridization region). A tracrRNA may also form a secondary structure (e.g., one or more hairpin loops) that facilitates the binding of an effector protein to a guide nucleic acid and/or modification activity of an effector protein on a target nucleic acid (e.g., a hairpin region). A tracrRNA may include a repeat hybridization region and a hairpin region. The repeat hybridization region may hybridize to all or part of the repeat sequence of a guide nucleic acid. The repeat hybridization region may be positioned 3′ of the hairpin region. The hairpin region may include a first sequence, a second sequence that is reverse complementary to the first sequence, and a stem-loop linking the first sequence and the second sequence.

In some embodiments, tracrRNAs comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the tracrRNA comprises a pseudoknot (e.g., a secondary structure comprising a stem at least partially hybridized to a second stem or half-stem secondary structure). An effector protein may recognize a tracrRNA sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the tracrRNA sequence comprises at least 2, at least 3, at least 4, or at least 5 stem regions.

In some embodiments, the length of a tracrRNA is not greater than 50, 56, 68, 71, 73, 95, or 105 linked nucleotides. In some embodiments, the length of a tracrRNA is about 30 to about 120 linked nucleotides. In some embodiments, the length of a tracrRNA is about 50 to about 105, about 50 to about 95, about 50 to about 73, about 50 to about 71, about 50 to about 68, or about 50 to about 56 linked nucleotides. In some embodiments, the length of a tracrRNA is 56 to 105 linked nucleotides, from 56 to 105 linked nucleotides, 68 to 105 linked nucleotides, 71 to 105 linked nucleotides, 73 to 105 linked nucleotides, or 95 to 105 linked nucleotides. In some embodiments, the length of a tracrRNA is 40 to 60 nucleotides. In some embodiments, the length of a tracrRNA is 50, 56, 68, 71, 73, 95, or 105 linked nucleotides. In some embodiments, the length of a tracrRNA is 50 nucleotides.

An exemplary tracrRNA may comprise, from 5′ to 3′, a 5′ region, a hairpin region, a repeat hybridization region, and a 3′ region. In some embodiments, the 5′ region may hybridize to the 3′ region. In some embodiments, the 5′ region does not hybridize to the 3′ region. In some embodiments, the 3′ region is covalently linked to the crRNA (e.g., through a phosphodiester bond). In some embodiments, a tracrRNA may comprise an un-hybridized region at the 3′ end of the tracrRNA. The un-hybridized region may have a length of about 1, about 2, about 3, about 4, about 5, about 6, about 7, about 8, about 9, about 10, about 12, about 14, about 16, about 18, or about 20 linked nucleotides. In some embodiments, the length of the un-hybridized region is 0 to 20 linked nucleotides.

In some embodiments, the composition comprising an effector protein and a guide RNA does not comprise a tracrRNA sequence. In some embodiments, an effector protein does not require a tracrRNA to locate and/or cleave a target nucleic acid.

### VII. Modifications

Polypeptides (e.g., effector proteins) and nucleic acids (e.g., engineered guide nucleic acids) described herein can be further modified as described throughout and as further described herein. Examples are modifications of interest that do not alter primary sequence, including chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g., D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides (e.g., 2′-O-Methyl (2′OMe) sugar modifications), 2′ Fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VIII. Target Nucleic Acids

Disclosed herein are compositions, systems and methods for detecting and/or editing a target nucleic acid. In certain embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand.

In some embodiments, compositions, systems, and methods described herein comprise a modified target nucleic acid which can describe a target nucleic acid wherein the target nucleic acid has undergone a modification, for example, after contact with an effector protein. In some cases, the modification is an alteration in the nucleotide sequence of the target nucleic acid. In some cases, the modified target nucleic acid comprises an insertion, deletion, or replacement of one or more nucleotides compared to the unmodified target nucleic acid.

In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA. In some embodiments, the target nucleic acid is from a virus, a parasite, or a bacterium described herein.

### PAM

In some embodiments, a target nucleic acid comprises a PAM as described herein that is located on the non-target strand. Such a PAM described herein, in some embodiments, is adjacent (e.g., within 1, 2, 3, 4 or 5 nucleotides) to the 5′ end of the target sequence on the non-target strand of the double stranded DNA molecule. In certain embodiments, such a PAM described herein is directly adjacent to the 5′ end of a target sequence on the non-target strand of the double stranded DNA molecule.

In some embodiments, an effector protein or a multimeric complex thereof recognizes a PAM on a target nucleic acid. In some embodiments, multiple effector proteins of the multimeric complex recognize a PAM on a target nucleic acid. In some embodiments, only one effector protein of the multimeric complex recognizes a PAM on a target nucleic acid. In some embodiments, the PAM is 3′ to the spacer region of the crRNA. In some embodiments, the PAM is directly 3′ to the spacer region of the crRNA. In some embodiments, the PAM sequence comprises a sequence listed in TABLE 2.

An effector protein of the present disclosure, a dimer thereof, or a multimeric complex thereof may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region. In some embodiments, the PAM sequence is read 5′ to 3′ as set forth in TABLE 2.

In some embodiments, the effector protein comprises an amino acid sequence that is at least 65%, at least 70%, at least 75%, at least 80%, at least 85%, at least 90%, at least 95%, at least 98%, at least 99%, or 100% identical to the amino acid sequence of SEQ ID NO: 1, and the target nucleic acid comprises a PAM sequence of any one of the nucleotide sequences as set forth in TABLE 2. In some embodiments, the effector protein comprises an amino acid sequence that is at least 65%, at least 70%, at least 75%, at least 80%, at least 85%, at least 90%, at least 95%, at least 98%, at least 99%, or 100% similar to the amino acid sequence of SEQ ID NO: 1, and the target nucleic acid comprises a PAM sequence of any one of the nucleotide sequences as set forth in TABLE 2.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector system described herein.

In some embodiments, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, the target nucleic acid comprises a portion or a specific region of a nucleic acid from a genomic locus, any DNA amplicon of, a reverse transcribed mRNA, or a cDNA from the gene of TABLE 7.

The terms “dystrophin” and “DMD,” as used herein, refers to the dystrophin from any vertebrate source, including mammals such as primates (e.g., humans), dogs, and rodents (e.g., mice and rats), unless otherwise indicated. Dystrophin is a protein which forms a component of the dystrophin-glycoprotein complex (DGC), which bridges the inner cytoskeleton and the extracellular matrix. The gene encoding human dystrophin, referred to as DMD, contains 79 exons and spans 2.4 Mb, and is located on chromosome X, at cytogenetic location Xp21.2-p21.1. An exemplary amino acid sequence of dystrophin, UniProtKB protein P11532 (DMD_HUMAN), is provided below:

(SEQ ID NO: 448)
MLWWEEVEDCYEREDVQKKTFTKWVNAQFSKFGKQHIENLFSDLQDGRRLLDLLEGLTGQKLP
KEKGSTRVHALNNVNKALRVLQNNNVDLVNIGSTDIVDGNHKLTLGLIWNIILHWQVKNVMKN
IMAGLQQTNSEKILLSWVRQSTRNYPQVNVINFTTSWSDGLALNALIHSHRPDLFDWNSVVCQQ
SATQRLEHAFNIARYQLGIEKLLDPEDVDTTYPDKKSILMYITSLFQVLPQQVSIEAIQEVEMLPRP
PKVTKEEHFQLHHQMHYSQQITVSLAQGYERTSSPKPRFKSYAYTQAAYVTTSDPTRSPFPSQHL
EAPEDKSFGSSLMESEVNLDRYQTALEEVLSWLLSAEDTLQAQGEISNDVEVVKDQFHTHEGYM
MDLTAHQGRVGNILQLGSKLIGTGKLSEDEETEVQEQMNLLNSRWECLRVASMEKQSNLHRVL
MDLQNQKLKELNDWLTKTEERTRKMEEEPLGPDLEDLKRQVQQHKVLQEDLEQEQVRVNSLT
HMVVVVDESSGDHATAALEEQLKVLGDRWANICRWTEDRWVLLQDILLKWQRLTEEQCLFSA
WLSEKEDAVNKIHTTGFKDQNEMLSSLQKLAVLKADLEKKKQSMGKLYSLKQDLLSTLKNKSV
TQKTEAWLDNFARCWDNLVQKLEKSTAQISQAVTTTQPSLTQTTVMETVTTVTTREQILVKHAQ
EELPPPPPQKKRQITVDSEIRKRLDVDITELHSWITRSEAVLQSPEFAIFRKEGNFSDLKEKVNAIER
EKAEKFRKLQDASRSAQALVEQMVNEGVNADSIKQASEQLNSRWIEFCQLLSERLNWLEYQNNI
IAFYNQLQQLEQMTTTAENWLKIQPTTPSEPTAIKSQLKICKDEVNRLSDLQPQIERLKIQSIALKE
KGQGPMFLDADFVAFTNHFKQVFSDVQAREKELQTIFDTLPPMRYQETMSAIRTWVQQSETKLS
IPQLSVTDYEIMEQRLGELQALQSSLQEQQSGLYYLSTTVKEMSKKAPSEISRKYQSEFEEIEGRW
KKLSSQLVEHCQKLEEQMNKLRKIQNHIQTLKKWMAEVDVFLKEEWPALGDSEILKKQLKQCR
LLVSDIQTIQPSLNSVNEGGQKIKNEAEPEFASRLETELKELNTQWDHMCQQVYARKEALKGGL
EKTVSLQKDLSEMHEWMTQAEEEYLERDFEYKTPDELQKAVEEMKRAKEEAQQKEAKVKLLT
ESVNSVIAQAPPVAQEALKKELETLTTNYQWLCTRLNGKCKTLEEVWACWHELLSYLEKANKW
LNEVEFKLKTTENIPGGAEEISEVLDSLENLMRHSEDNPNQIRILAQTLTDGGVMDELINEELETF
NSRWRELHEEAVRRQKLLEQSIQSAQETEKSLHLIQESLTFIDKQLAAYIADKVDAAQMPQEAQK
IQSDLTSHEISLEEMKKHNQGKEAAQRVLSQIDVAQKKLQDVSMKFRLFQKPANFEQRLQESKM
ILDEVKMHLPALETKSVEQEVVQSQLNHCVNLYKSLSEVKSEVEMVIKTGRQIVQKKQTENPKE
LDERVTALKLHYNELGAKVTERKQQLEKCLKLSRKMRKEMNVLTEWLAATDMELTKRSAVEG
MPSNLDSEVAWGKATQKEIEKQKVHLKSITEVGEALKTVLGKKETLVEDKLSLLNSNWIAVTSR
AEEWLNLLLEYQKHMETFDQNVDHITKWIIQADTLLDESEKKKPQQKEDVLKRLKAELNDIRPK
VDSTRDQAANLMANRGDHCRKLVEPQISELNHRFAAISHRIKTGKASIPLKELEQFNSDIQKLLEP
LEAEIQQGVNLKEEDFNKDMNEDNEGTVKELLQRGDNLQQRITDERKREEIKIKQQLLQTKHNA
LKDLRSQRRKKALEISHQWYQYKRQADDLLKCLDDIEKKLASLPEPRDERKIKEIDRELQKKKEE
LNAVRRQAEGLSEDGAAMAVEPTQIQLSKRWREIESKFAQFRRLNFAQIHTVREETMMVMTED
MPLEISYVPSTYLTEITHVSQALLEVEQLLNAPDLCAKDFEDLFKQEESLKNIKDSLQQSSGRIDII
HSKKTAALQSATPVERVKLQEALSQLDFQWEKVNKMYKDRQGRFDRSVEKWRRFHYDIKIFNQ
WLTEAEQFLRKTQIPENWEHAKYKWYLKELQDGIGQRQTVVRTLNATGEEIIQQSSKTDASILQE
KLGSLNLRWQEVCKQLSDRKKRLEEQKNILSEFQRDLNEFVLWLEEADNIASIPLEPGKEQQLKE
KLEQVKLLVEELPLRQGILKQLNETGGPVLVSAPISPEEQDKLENKLKQTNLQWIKVSRALPEKQ
GEIEAQIKDLGQLEKKLEDLEEQLNHLLLWLSPIRNQLEIYNQPNQEGPFDVKETEIAVQAKQPD
VEEILSKGQHLYKEKPATQPVKRKLEDLSSEWKAVNRLLQELRAKQPDLAPGLTTIGASPTQTVT
LVTQPVVTKETAISKLEMPSSLMLEVPALADFNRAWTELTDWLSLLDQVIKSQRVMVGDLEDIN
EMIIKQKATMQDLEQRRPQLEELITAAQNLKNKTSNQEARTIITDRIERIQNQWDEVQEHLQNRR
QQLNEMLKDSTQWLEAKEEAEQVLGQARAKLESWKEGPYTVDAIQKKITETKQLAKDLRQWQ
TNVDVANDLALKLLRDYSADDTRKVHMITENINASWRSIHKRVSEREAALEETHRLLQQFPLDL
EKFLAWLTEAETTANVLQDATRKERLLEDSKGVKELMKQWQDLQGEIEAHTDVYHNLDENSQ
KILRSLEGSDDAVLLQRRLDNMNFKWSELRKKSLNIRSHLEASSDQWKRLHLSLQELLVWLQLK
DDELSRQAPIGGDFPAVQKQNDVHRAFKRELKTKEPVIMSTLETVRIFLTEQPLEGLEKLYQEPRE
LPPEERAQNVTRLLRKQAEEVNTEWEKLNLHSADWQRKIDETLERLQELQEATDELDLKLRQAE
VIKGSWQPVGDLLIDSLQDHLEKVKALRGEIAPLKENVSHVNDLARQLTTLGIQLSPYNLSTLED
LNTRWKLLQVAVEDRVRQLHEAHRDFGPASQHELSTSVQGPWERAISPNKVPYYINHETQTTC
WDHPKMTELYQSLADLNNVRFSAYRTAMKLRRLQKALCLDLLSLSAACDALDQHNLKQNDQP
MDILQIINCLTTIYDRLEQEHNNLVNVPLCVDMCLNWLLNVYDTGRTGRIRVLSFKTGIISLCKAH
LEDKYRYLFKQVASSTGFCDQRRLGLLLHDSIQIPRQLGEVASFGGSNIEPSVRSCFQFANNKPEIE
AALFLDWMRLEPQSMVWLPVLHRVAAAETAKHQAKCNICKECPIIGFRYRSLKHFNYDICQSCF
FSGRVAKGHKMHYPMVEYCTPTTSGEDVRDFAKVLKNKFRTKRYFAKHPRMGYLPVQTVLEG
DNMETPVTLINFWPVDSAPASSPQLSHDDTHSRIEHYASRLAEMENSNGSYLNDSISPNESIDDEH
LLIQHYCQSLNQDSPLSQPRSPAQILISLESEERGELERILADLEEENRNLQAEYDRLKQQHEHKG
LSPLPSPPEMMPTSPQSPRDAELIAEAKLLRQHKGRLEARMQILEDHNKQLESQLHRLRQLLEQP
QAEAKVNGTTVSSPSTSLQRSDSSQPMLLRVVGSQTSDSMGEEDLLSPPQDTSTGLEEVMEQLN
NSFPSSRGRNTPGKPMREDTM

An exemplary encoding nucleic acid sequence of human dystrophin can be found at NCBI Reference Sequence No. NM_004006.3 and is provided below:

(SEQ ID NO: 449)
ATCAGTTACTGTGTTGACTCACTCAGTGTTGGGATCACTCACTTTCCCCCTACAGGACTCAGA
TCTGGGAGGCAATTACCTTCGGAGAAAAACGAATAGGAAAAACTGAAGTGTTACTTTTTTTA
AAGCTGCTGAAGTTTGTTGGTTTCTCATTGTTTTTAAGCCTACTGGAGCAATAAAGTTTGAAG
AACTTTTACCAGGTTTTTTTTATCGCTGCCTTGATATACACTTTTCAAAATGCTTTGGTGGGA
AGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGA
TGGGAGGCGCCTCCTAGACCTCCTCGAAGGCCTGACAGGGCAAAAACTGCCAAAAGAAAAA
GGATCCACAAGAGTTCATGCCCTGAACAATGTCAACAAGGCACTGCGGGTTTTGCAGAACA
ATAATGTTGATTTAGTGAATATTGGAAGTACTGACATCGTAGATGGAAATCATAAACTGACT
CTTGGTTTGATTTGGAATATAATCCTCCACTGGCAGGTCAAAAATGTAATGAAAAATATCAT
GGCTGGATTGCAACAAACCAACAGTGAAAAGATTCTCCTGAGCTGGGTCCGACAATCAACT
CGTAATTATCCACAGGTTAATGTAATCAACTTCACCACCAGCTGGTCTGATGGCCTGGCTTTG
AATGCTCTCATCCATAGTCATAGGCCAGACCTATTTGACTGGAATAGTGTGGTTTGCCAGCA
GTCAGCCACACAACGACTGGAACATGCATTCAACATCGCCAGATATCAATTAGGCATAGAG
AAACTACTCGATCCTGAAGATGTTGATACCACCTATCCAGATAAGAAGTCCATCTTAATGTA
CATCACATCACTCTTCCAAGTTTTGCCTCAACAAGTGAGCATTGAAGCCATCCAGGAAGTGG
AAATGTTGCCAAGGCCACCTAAAGTGACTAAAGAAGAACATTTTCAGTTACATCATCAAATG
CACTATTCTCAACAGATCACGGTCAGTCTAGCACAGGGATATGAGAGAACTTCTTCCCCTAA
GCCTCGATTCAAGAGCTATGCCTACACACAGGCTGCTTATGTCACCACCTCTGACCCTACAC
GGAGCCCATTTCCTTCACAGCATTTGGAAGCTCCTGAAGACAAGTCATTTGGCAGTTCATTG
ATGGAGAGTGAAGTAAACCTGGACCGTTATCAAACAGCTTTAGAAGAAGTATTATCGTGGCT
TCTTTCTGCTGAGGACACATTGCAAGCACAAGGAGAGATTTCTAATGATGTGGAAGTGGTGA
AAGACCAGTTTCATACTCATGAGGGGTACATGATGGATTTGACAGCCCATCAGGGCCGGGTT
GGTAATATTCTACAATTGGGAAGTAAGCTGATTGGAACAGGAAAATTATCAGAAGATGAAG
AAACTGAAGTACAAGAGCAGATGAATCTCCTAAATTCAAGATGGGAATGCCTCAGGGTAGC
TAGCATGGAAAAACAAAGCAATTTACATAGAGTTTTAATGGATCTCCAGAATCAGAAACTG
AAAGAGTTGAATGACTGGCTAACAAAAACAGAAGAAAGAACAAGGAAAATGGAGGAAGAG
CCTCTTGGACCTGATCTTGAAGACCTAAAACGCCAAGTACAACAACATAAGGTGCTTCAAGA
AGATCTAGAACAAGAACAAGTCAGGGTCAATTCTCTCACTCACATGGTGGTGGTAGTTGATG
AATCTAGTGGAGATCACGCAACTGCTGCTTTGGAAGAACAACTTAAGGTATTGGGAGATCG
ATGGGCAAACATCTGTAGATGGACAGAAGACCGCTGGGTTCTTTTACAAGACATCCTTCTCA
AATGGCAACGTCTTACTGAAGAACAGTGCCTTTTTAGTGCATGGCTTTCAGAAAAAGAAGAT
GCAGTGAACAAGATTCACACAACTGGCTTTAAAGATCAAAATGAAATGTTATCAAGTCTTCA
AAAACTGGCCGTTTTAAAAGCGGATCTAGAAAAGAAAAAGCAATCCATGGGCAAACTGTAT
TCACTCAAACAAGATCTTCTTTCAACACTGAAGAATAAGTCAGTGACCCAGAAGACGGAAG
CATGGCTGGATAACTTTGCCCGGTGTTGGGATAATTTAGTCCAAAAACTTGAAAAGAGTACA
GCACAGATTTCACAGGCTGTCACCACCACTCAGCCATCACTAACACAGACAACTGTAATGGA
AACAGTAACTACGGTGACCACAAGGGAACAGATCCTGGTAAAGCATGCTCAAGAGGAACTT
CCACCACCACCTCCCCAAAAGAAGAGGCAGATTACTGTGGATTCTGAAATTAGGAAAAGGT
TGGATGTTGATATAACTGAACTTCACAGCTGGATTACTCGCTCAGAAGCTGTGTTGCAGAGT
CCTGAATTTGCAATCTTTCGGAAGGAAGGCAACTTCTCAGACTTAAAAGAAAAAGTCAATGC
CATAGAGCGAGAAAAAGCTGAGAAGTTCAGAAAACTGCAAGATGCCAGCAGATCAGCTCAG
GCCCTGGTGGAACAGATGGTGAATGAGGGTGTTAATGCAGATAGCATCAAACAAGCCTCAG
AACAACTGAACAGCCGGTGGATCGAATTCTGCCAGTTGCTAAGTGAGAGACTTAACTGGCTG
GAGTATCAGAACAACATCATCGCTTTCTATAATCAGCTACAACAATTGGAGCAGATGACAAC
TACTGCTGAAAACTGGTTGAAAATCCAACCCACCACCCCATCAGAGCCAACAGCAATTAAA
AGTCAGTTAAAAATTTGTAAGGATGAAGTCAACCGGCTATCAGATCTTCAACCTCAAATTGA
ACGATTAAAAATTCAAAGCATAGCCCTGAAAGAGAAAGGACAAGGACCCATGTTCCTGGAT
GCAGACTTTGTGGCCTTTACAAATCATTTTAAGCAAGTCTTTTCTGATGTGCAGGCCAGAGA
GAAAGAGCTACAGACAATTTTTGACACTTTGCCACCAATGCGCTATCAGGAGACCATGAGTG
CCATCAGGACATGGGTCCAGCAGTCAGAAACCAAACTCTCCATACCTCAACTTAGTGTCACC
GACTATGAAATCATGGAGCAGAGACTCGGGGAATTGCAGGCTTTACAAAGTTCTCTGCAAG
AGCAACAAAGTGGCCTATACTATCTCAGCACCACTGTGAAAGAGATGTCGAAGAAAGCGCC
CTCTGAAATTAGCCGGAAATATCAATCAGAATTTGAAGAAATTGAGGGACGCTGGAAGAAG
CTCTCCTCCCAGCTGGTTGAGCATTGTCAAAAGCTAGAGGAGCAAATGAATAAACTCCGAAA
AATTCAGAATCACATACAAACCCTGAAGAAATGGATGGCTGAAGTTGATGTTTTTCTGAAGG
AGGAATGGCCTGCCCTTGGGGATTCAGAAATTCTAAAAAAGCAGCTGAAACAGTGCAGACT
TTTAGTCAGTGATATTCAGACAATTCAGCCCAGTCTAAACAGTGTCAATGAAGGTGGGCAGA
AGATAAAGAATGAAGCAGAGCCAGAGTTTGCTTCGAGACTTGAGACAGAACTCAAAGAACT
TAACACTCAGTGGGATCACATGTGCCAACAGGTCTATGCCAGAAAGGAGGCCTTGAAGGGA
GGTTTGGAGAAAACTGTAAGCCTCCAGAAAGATCTATCAGAGATGCACGAATGGATGACAC
AAGCTGAAGAAGAGTATCTTGAGAGAGATTTTGAATATAAAACTCCAGATGAATTACAGAA
AGCAGTTGAAGAGATGAAGAGAGCTAAAGAAGAGGCCCAACAAAAAGAAGCGAAAGTGAA
ACTCCTTACTGAGTCTGTAAATAGTGTCATAGCTCAAGCTCCACCTGTAGCACAAGAGGCCT
TAAAAAAGGAACTTGAAACTCTAACCACCAACTACCAGTGGCTCTGCACTAGGCTGAATGG
GAAATGCAAGACTTTGGAAGAAGTTTGGGCATGTTGGCATGAGTTATTGTCATACTTGGAGA
AAGCAAACAAGTGGCTAAATGAAGTAGAATTTAAACTTAAAACCACTGAAAACATTCCTGG
CGGAGCTGAGGAAATCTCTGAGGTGCTAGATTCACTTGAAAATTTGATGCGACATTCAGAGG
ATAACCCAAATCAGATTCGCATATTGGCACAGACCCTAACAGATGGCGGAGTCATGGATGA
GCTAATCAATGAGGAACTTGAGACATTTAATTCTCGTTGGAGGGAACTACATGAAGAGGCTG
TAAGGAGGCAAAAGTTGCTTGAACAGAGCATCCAGTCTGCCCAGGAGACTGAAAAATCCTT
ACACTTAATCCAGGAGTCCCTCACATTCATTGACAAGCAGTTGGCAGCTTATATTGCAGACA
AGGTGGACGCAGCTCAAATGCCTCAGGAAGCCCAGAAAATCCAATCTGATTTGACAAGTCA
TGAGATCAGTTTAGAAGAAATGAAGAAACATAATCAGGGGAAGGAGGCTGCCCAAAGAGTC
CTGTCTCAGATTGATGTTGCACAGAAAAAATTACAAGATGTCTCCATGAAGTTTCGATTATT
CCAGAAACCAGCCAATTTTGAGCAGCGTCTACAAGAAAGTAAGATGATTTTAGATGAAGTG
AAGATGCACTTGCCTGCATTGGAAACAAAGAGTGTGGAACAGGAAGTAGTACAGTCACAGC
TAAATCATTGTGTGAACTTGTATAAAAGTCTGAGTGAAGTGAAGTCTGAAGTGGAAATGGTG
ATAAAGACTGGACGTCAGATTGTACAGAAAAAGCAGACGGAAAATCCCAAAGAACTTGATG
AAAGAGTAACAGCTTTGAAATTGCATTATAATGAGCTGGGAGCAAAGGTAACAGAAAGAAA
GCAACAGTTGGAGAAATGCTTGAAATTGTCCCGTAAGATGCGAAAGGAAATGAATGTCTTG
ACAGAATGGCTGGCAGCTACAGATATGGAATTGACAAAGAGATCAGCAGTTGAAGGAATGC
CTAGTAATTTGGATTCTGAAGTTGCCTGGGGAAAGGCTACTCAAAAAGAGATTGAGAAACA
GAAGGTGCACCTGAAGAGTATCACAGAGGTAGGAGAGGCCTTGAAAACAGTTTTGGGCAAG
AAGGAGACGTTGGTGGAAGATAAACTCAGTCTTCTGAATAGTAACTGGATAGCTGTCACCTC
CCGAGCAGAAGAGTGGTTAAATCTTTTGTTGGAATACCAGAAACACATGGAAACTTTTGACC
AGAATGTGGACCACATCACAAAGTGGATCATTCAGGCTGACACACTTTTGGATGAATCAGA
GAAAAAGAAACCCCAGCAAAAAGAAGACGTGCTTAAGCGTTTAAAGGCAGAACTGAATGA
CATACGCCCAAAGGTGGACTCTACACGTGACCAAGCAGCAAACTTGATGGCAAACCGCGGT
GACCACTGCAGGAAATTAGTAGAGCCCCAAATCTCAGAGCTCAACCATCGATTTGCAGCCAT
TTCACACAGAATTAAGACTGGAAAGGCCTCCATTCCTTTGAAGGAATTGGAGCAGTTTAACT
CAGATATACAAAAATTGCTTGAACCACTGGAGGCTGAAATTCAGCAGGGGGTGAATCTGAA
AGAGGAAGACTTCAATAAAGATATGAATGAAGACAATGAGGGTACTGTAAAAGAATTGTTG
CAAAGAGGAGACAACTTACAACAAAGAATCACAGATGAGAGAAAGCGAGAGGAAATAAAG
ATAAAACAGCAGCTGTTACAGACAAAACATAATGCTCTCAAGGATTTGAGGTCTCAAAGAA
GAAAAAAGGCTCTAGAAATTTCTCATCAGTGGTATCAGTACAAGAGGCAGGCTGATGATCTC
CTGAAATGCTTGGATGACATTGAAAAAAAATTAGCCAGCCTACCTGAGCCCAGAGATGAAA
GGAAAATAAAGGAAATTGATCGGGAATTGCAGAAGAAGAAAGAGGAGCTGAATGCAGTGC
GTAGGCAAGCTGAGGGCTTGTCTGAGGATGGGGCCGCAATGGCAGTGGAGCCAACTCAGAT
CCAGCTCAGCAAGCGCTGGCGGGAAATTGAGAGCAAATTTGCTCAGTTTCGAAGACTCAACT
TTGCACAAATTCACACTGTCCGTGAAGAAACGATGATGGTGATGACTGAAGACATGCCTTTG
GAAATTTCTTATGTGCCTTCTACTTATTTGACTGAAATCACTCATGTCTCACAAGCCCTATTA
GAAGTGGAACAACTTCTCAATGCTCCTGACCTCTGTGCTAAGGACTTTGAAGATCTCTTTAA
GCAAGAGGAGTCTCTGAAGAATATAAAAGATAGTCTACAACAAAGCTCAGGTCGGATTGAC
ATTATTCATAGCAAGAAGACAGCAGCATTGCAAAGTGCAACGCCTGTGGAAAGGGTGAAGC
TACAGGAAGCTCTCTCCCAGCTTGATTTCCAATGGGAAAAAGTTAACAAAATGTACAAGGAC
CGACAAGGGCGATTTGACAGATCTGTTGAGAAATGGCGGCGTTTTCATTATGATATAAAGAT
ATTTAATCAGTGGCTAACAGAAGCTGAACAGTTTCTCAGAAAGACACAAATTCCTGAGAATT
GGGAACATGCTAAATACAAATGGTATCTTAAGGAACTCCAGGATGGCATTGGGCAGCGGCA
AACTGTTGTCAGAACATTGAATGCAACTGGGGAAGAAATAATTCAGCAATCCTCAAAAACA
GATGCCAGTATTCTACAGGAAAAATTGGGAAGCCTGAATCTGCGGTGGCAGGAGGTCTGCA
AACAGCTGTCAGACAGAAAAAAGAGGCTAGAAGAACAAAAGAATATCTTGTCAGAATTTCA
AAGAGATTTAAATGAATTTGTTTTATGGTTGGAGGAAGCAGATAACATTGCTAGTATCCCAC
TTGAACCTGGAAAAGAGCAGCAACTAAAAGAAAAGCTTGAGCAAGTCAAGTTACTGGTGGA
AGAGTTGCCCCTGCGCCAGGGAATTCTCAAACAATTAAATGAAACTGGAGGACCCGTGCTTG
TAAGTGCTCCCATAAGCCCAGAAGAGCAAGATAAACTTGAAAATAAGCTCAAGCAGACAAA
TCTCCAGTGGATAAAGGTTTCCAGAGCTTTACCTGAGAAACAAGGAGAAATTGAAGCTCAA
ATAAAAGACCTTGGGCAGCTTGAAAAAAAGCTTGAAGACCTTGAAGAGCAGTTAAATCATC
TGCTGCTGTGGTTATCTCCTATTAGGAATCAGTTGGAAATTTATAACCAACCAAACCAAGAA
GGACCATTTGACGTTAAGGAAACTGAAATAGCAGTTCAAGCTAAACAACCGGATGTGGAAG
AGATTTTGTCTAAAGGGCAGCATTTGTACAAGGAAAAACCAGCCACTCAGCCAGTGAAGAG
GAAGTTAGAAGATCTGAGCTCTGAGTGGAAGGCGGTAAACCGTTTACTTCAAGAGCTGAGG
GCAAAGCAGCCTGACCTAGCTCCTGGACTGACCACTATTGGAGCCTCTCCTACTCAGACTGT
TACTCTGGTGACACAACCTGTGGTTACTAAGGAAACTGCCATCTCCAAACTAGAAATGCCAT
CTTCCTTGATGTTGGAGGTACCTGCTCTGGCAGATTTCAACCGGGCTTGGACAGAACTTACC
GACTGGCTTTCTCTGCTTGATCAAGTTATAAAATCACAGAGGGTGATGGTGGGTGACCTTGA
GGATATCAACGAGATGATCATCAAGCAGAAGGCAACAATGCAGGATTTGGAACAGAGGCGT
CCCCAGTTGGAAGAACTCATTACCGCTGCCCAAAATTTGAAAAACAAGACCAGCAATCAAG
AGGCTAGAACAATCATTACGGATCGAATTGAAAGAATTCAGAATCAGTGGGATGAAGTACA
AGAACACCTTCAGAACCGGAGGCAACAGTTGAATGAAATGTTAAAGGATTCAACACAATGG
CTGGAAGCTAAGGAAGAAGCTGAGCAGGTCTTAGGACAGGCCAGAGCCAAGCTTGAGTCAT
GGAAGGAGGGTCCCTATACAGTAGATGCAATCCAAAAGAAAATCACAGAAACCAAGCAGTT
GGCCAAAGACCTCCGCCAGTGGCAGACAAATGTAGATGTGGCAAATGACTTGGCCCTGAAA
CTTCTCCGGGATTATTCTGCAGATGATACCAGAAAAGTCCACATGATAACAGAGAATATCAA
TGCCTCTTGGAGAAGCATTCATAAAAGGGTGAGTGAGCGAGAGGCTGCTTTGGAAGAAACT
CATAGATTACTGCAACAGTTCCCCCTGGACCTGGAAAAGTTTCTTGCCTGGCTTACAGAAGC
TGAAACAACTGCCAATGTCCTACAGGATGCTACCCGTAAGGAAAGGCTCCTAGAAGACTCC
AAGGGAGTAAAAGAGCTGATGAAACAATGGCAAGACCTCCAAGGTGAAATTGAAGCTCACA
CAGATGTTTATCACAACCTGGATGAAAACAGCCAAAAAATCCTGAGATCCCTGGAAGGTTCC
GATGATGCAGTCCTGTTACAAAGACGTTTGGATAACATGAACTTCAAGTGGAGTGAACTTCG
GAAAAAGTCTCTCAACATTAGGTCCCATTTGGAAGCCAGTTCTGACCAGTGGAAGCGTCTGC
ACCTTTCTCTGCAGGAACTTCTGGTGTGGCTACAGCTGAAAGATGATGAATTAAGCCGGCAG
GCACCTATTGGAGGCGACTTTCCAGCAGTTCAGAAGCAGAACGATGTACATAGGGCCTTCAA
GAGGGAATTGAAAACTAAAGAACCTGTAATCATGAGTACTCTTGAGACTGTACGAATATTTC
TGACAGAGCAGCCTTTGGAAGGACTAGAGAAACTCTACCAGGAGCCCAGAGAGCTGCCTCC
TGAGGAGAGAGCCCAGAATGTCACTCGGCTTCTACGAAAGCAGGCTGAGGAGGTCAATACT
GAGTGGGAAAAATTGAACCTGCACTCCGCTGACTGGCAGAGAAAAATAGATGAGACCCTTG
AAAGACTCCGGGAACTTCAAGAGGCCACGGATGAGCTGGACCTCAAGCTGCGCCAAGCTGA
GGTGATCAAGGGATCCTGGCAGCCCGTGGGCGATCTCCTCATTGACTCTCTCCAAGATCACC
TCGAGAAAGTCAAGGCACTTCGAGGAGAAATTGCGCCTCTGAAAGAGAACGTGAGCCACGT
CAATGACCTTGCTCGCCAGCTTACCACTTTGGGCATTCAGCTCTCACCGTATAACCTCAGCAC
TCTGGAAGACCTGAACACCAGATGGAAGCTTCTGCAGGTGGCCGTCGAGGACCGAGTCAGG
CAGCTGCATGAAGCCCACAGGGACTTTGGTCCAGCATCTCAGCACTTTCTTTCCACGTCTGTC
CAGGGTCCCTGGGAGAGAGCCATCTCGCCAAACAAAGTGCCCTACTATATCAACCACGAGA
CTCAAACAACTTGCTGGGACCATCCCAAAATGACAGAGCTCTACCAGTCTTTAGCTGACCTG
AATAATGTCAGATTCTCAGCTTATAGGACTGCCATGAAACTCCGAAGACTGCAGAAGGCCCT
TTGCTTGGATCTCTTGAGCCTGTCAGCTGCATGTGATGCCTTGGACCAGCACAACCTCAAGC
AAAATGACCAGCCCATGGATATCCTGCAGATTATTAATTGTTTGACCACTATTTATGACCGC
CTGGAGCAAGAGCACAACAATTTGGTCAACGTCCCTCTCTGCGTGGATATGTGTCTGAACTG
GCTGCTGAATGTTTATGATACGGGACGAACAGGGAGGATCCGTGTCCTGTCTTTTAAAACTG
GCATCATTTCCCTGTGTAAAGCACATTTGGAAGACAAGTACAGATACCTTTTCAAGCAAGTG
GCAAGTTCAACAGGATTTTGTGACCAGCGCAGGCTGGGCCTCCTTCTGCATGATTCTATCCA
AATTCCAAGACAGTTGGGTGAAGTTGCATCCTTTGGGGGCAGTAACATTGAGCCAAGTGTCC
GGAGCTGCTTCCAATTTGCTAATAATAAGCCAGAGATCGAAGCGGCCCTCTTCCTAGACTGG
ATGAGACTGGAACCCCAGTCCATGGTGTGGCTGCCCGTCCTGCACAGAGTGGCTGCTGCAGA
AACTGCCAAGCATCAGGCCAAATGTAACATCTGCAAAGAGTGTCCAATCATTGGATTCAGGT
ACAGGAGTCTAAAGCACTTTAATTATGACATCTGCCAAAGCTGCTTTTTTTCTGGTCGAGTTG
CAAAAGGCCATAAAATGCACTATCCCATGGTGGAATATTGCACTCCGACTACATCAGGAGA
AGATGTTCGAGACTTTGCCAAGGTACTAAAAAACAAATTTCGAACCAAAAGGTATTTTGCGA
AGCATCCCCGAATGGGCTACCTGCCAGTGCAGACTGTCTTAGAGGGGGACAACATGGAAAC
TCCCGTTACTCTGATCAACTTCTGGCCAGTAGATTCTGCGCCTGCCTCGTCCCCTCAGCTTTC
ACACGATGATACTCATTCACGCATTGAACATTATGCTAGCAGGCTAGCAGAAATGGAAAAC
AGCAATGGATCTTATCTAAATGATAGCATCTCTCCTAATGAGAGCATAGATGATGAACATTT
GTTAATCCAGCATTACTGCCAAAGTTTGAACCAGGACTCCCCCCTGAGCCAGCCTCGTAGTC
CTGCCCAGATCTTGATTTCCTTAGAGAGTGAGGAAAGAGGGGAGCTAGAGAGAATCCTAGC
AGATCTTGAGGAAGAAAACAGGAATCTGCAAGCAGAATATGACCGTCTAAAGCAGCAGCAC
GAACATAAAGGCCTGTCCCCACTGCCGTCCCCTCCTGAAATGATGCCCACCTCTCCCCAGAG
TCCCCGGGATGCTGAGCTCATTGCTGAGGCCAAGCTACTGCGTCAACACAAAGGCCGCCTGG
AAGCCAGGATGCAAATCCTGGAAGACCACAATAAACAGCTGGAGTCACAGTTACACAGGCT
AAGGCAGCTGCTGGAGCAACCCCAGGCAGAGGCCAAAGTGAATGGCACAACGGTGTCCTCT
CCTTCTACCTCTCTACAGAGGTCCGACAGCAGTCAGCCTATGCTGCTCCGAGTGGTTGGCAG
TCAAACTTCGGACTCCATGGGTGAGGAAGATCTTCTCAGTCCTCCCCAGGACACAAGCACAG
GGTTAGAGGAGGTGATGGAGCAACTCAACAACTCCTTCCCTAGTTCAAGAGGAAGAAATAC
CCCTGGAAAGCCAATGAGAGAGGACACAATGTAGGAAGTCTTTTCCACATGGCAGATGATT
TGGGCAGAGCGATGGAGTCCTTAGTATCAGTCATGACAGATGAAGAAGGAGCAGAATAAAT
GTTTTACAACTCCTGATTCCCGCATGGTTTTTATAATATTCATACAACAAAGAGGATTAGACA
GTAAGAGTTTACAAGAAATAAATCTATATTTTTGTGAAGGGTAGTGGTATTATACTGTAGAT
TTCAGTAGTTTCTAAGTCTGTTATTGTTTTGTTAACAATGGCAGGTTTTACACGTCTATGCAA
TTGTACAAAAAAGTTATAAGAAAACTACATGTAAAATCTTGATAGCTAAATAACTTGCCATT
TCTTTATATGGAACGCATTTTGGGTTGTTTAAAAATTTATAACAGTTATAAAGAAAGATTGTA
AACTAAAGTGTGCTTTATAAAAAAAAGTTGTTTATAAAAACCCCTAAAAACAAAACAAACA
CACACACACACACATACACACACACACACAAAACTTTGAGGCAGCGCATTGTTTTGCATCCT
TTTGGCGTGATATCCATATGAAATTCATGGCTTTTTCTTTTTTTGCATATTAAAGATAAGACT
TCCTCTACCACCACACCAAATGACTACTACACACTGCTCATTTGAGAACTGTCAGCTGAGTG
GGGCAGGCTTGAGTTTTCATTTCATATATCTATATGTCTATAAGTATATAAATACTATAGTTA
TATAGATAAAGAGATACGAATTTCTATAGACTGACTTTTTCCATTTTTTAAATGTTCATGTCA
CATCCTAATAGAAAGAAATTACTTCTAGTCAGTCATCCAGGCTTACCTGCTTGGTCTAGAAT
GGATTTTTCCCGGAGCCGGAAGCCAGGAGGAAACTACACCACACTAAAACATTGTCTACAG
CTCCAGATGTTTCTCATTTTAAACAACTTTCCACTGACAACGAAAGTAAAGTAAAGTATTGG
ATTTTTTTAAAGGGAACATGTGAATGAATACACAGGACTTATTATATCAGAGTGAGTAATCG
GTTGGTTGGTTGATTGATTGATTGATTGATACATTCAGCTTCCTGCTGCTAGCAATGCCACGA
TTTAGATTTAATGATGCTTCAGTGGAAATCAATCAGAAGGTATTCTGACCTTGTGAACATCA
GAAGGTATTTTTTAACTCCCAAGCAGTAGCAGGACGATGATAGGGCTGGAGGGCTATGGATT
CCCAGCCCATCCCTGTGAAGGAGTAGGCCACTCTTTAAGTGAAGGATTGGATGATTGTTCAT
AATACATAAAGTTCTCTGTAATTACAACTAAATTATTATGCCCTCTTCTCACAGTCAAAAGG
AACTGGGTGGTTTGGTTTTTGTTGCTTTTTTAGATTTATTGTCCCATGTGGGATGAGTTTTTAA
ATGCCACAAGACATAATTTAAAATAAATAAACTTTGGGAAAAGGTGTAAAACAGTAGCCCC
ATCACATTTGTGATACTGACAGGTATCAACCCAGAAGCCCATGAACTGTGTTTCCATCCTTTG
CATTTCTCTGCGAGTAGTTCCACACAGGTTTGTAAGTAAGTAAGAAAGAAGGCAAATTGATT
CAAATGTTACAAAAAAACCCTTCTTGGTGGATTAGACAGGTTAAATATATAAACAAACAAA
CAAAAATTGCTCAAAAAAGAGGAGAAAAGCTCAAGAGGAAAAGCTAAGGACTGGTAGGAA
AAAGCTTTACTCTTTCATGCCATTTTATTTCTTTTTGATTTTTAAATCATTCATTCAATAGATA
CCACCGTGTGACCTATAATTTTGCAAATCTGTTACCTCTGACATCAAGTGTAATTAGCTTTTG
GAGAGTGGGCTGACATCAAGTGTAATTAGCTTTTGGAGAGTGGGTTTTGTCCATTATTAATA
ATTAATTAATTAACATCAAACACGGCTTCTCATGCTATTTCTACCTCACTTTGGTTTTGGGGT
GTTCCTGATAATTGTGCACACCTGAGTTCACAGCTTCACCACTTGTCCATTGCGTTATTTTCTT
TTTCCTTTATAATTCTTTCTTTTTCCTTCATAATTTTCAAAAGAAAACCCAAAGCTCTAAGGTA
ACAAATTACCAAATTACATGAAGATTTGGTTTTTGTCTTGCATTTTTTTCCTTTATGTGACGCT
GGACCTTTTCTTTACCCAAGGATTTTTAAAACTCAGATTTAAAACAAGGGGTTACTTTACATC
CTACTAAGAAGTTTAAGTAAGTAAGTTTCATTCTAAAATCAGAGGTAAATAGAGTGCATAAA
TAATTTTGTTTTAATCTTTTTGTTTTTCTTTTAGACACATTAGCTCTGGAGTGAGTCTGTCATA
ATATTTGAACAAAAATTGAGAGCTTTATTGCTGCATTTTAAGCATAATTAATTTGGACATTAT
TTCGTGTTGTGTTCTTTATAACCACCAAGTATTAAACTGTAAATCATAATGTAACTGAAGCAT
AAACATCACATGGCATGTTTTGTCATTGTTTTCAGGTACTGAGTTCTTACTTGAGTATCATAA
TATATTGTGTTTTAACACCAACACTGTAACATTTACGAATTATTTTTTTAAACTTCAGTTTTAC
TGCATTTTCACAACATATCAGACTTCACCAAATATATGCCTTACTATTGTATTATAGTACTGC
TTTACTGTGTATCTCAATAAAGCACGCAGTTATGTTACAAAAAA

The genomic locations of dystrophin, isoform-1, exons can be found at Ensembl No. ENST00000357033.9, Human (GRCh38.p13) and is provided, at least in part in TABLE 8.

In some embodiments, at least partial nucleotide sequences of certain exemplary genomic exons can be found in TABLE 9.

In some embodiments, the target sequence is within the human dystrophin gene. In some embodiments, the target sequence is within an exon of the human dystrophin gene. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within the human dystrophin gene. A targeted exon can mean any portion within, contiguous with, or adjacent to a specified exon of interest can be targeted by the compositions, systems, and methods described herein. In some embodiments, one or more of exons 1 to exon 79, exon 15 to exon 60, exon 20 to exon 55, exon 40 to exon 55, or exon 44 to exon 53 are targeted. In some embodiments, one or more of exon 44, exon 45, exon 50, exon 51, exon 53, or any combination thereof, of the human dystrophin gene are targeted. Accordingly, in some embodiments: exon 44 is targeted; exon 45 is targeted; exon 50 is targeted; exon 51 is targeted; exon 53 is targeted; or any combination thereof.

In some embodiments, the start of an exon is referred to interchangeably herein as the 5′ end of an exon. In certain embodiments, the 5′ region of an exon comprises a sequence about 1 to about 300 nucleotides adjacent to the 5′ end of an exon when moving upstream in the 5′ direction, or a sequence about 1 to about 300 nucleotides adjacent to the 5′ end of an exon when moving downstream in the 3′ direction, or both.

In some embodiments, the end of an exon is referred to interchangeably herein as the 3′ end of an exon. In certain embodiments, the 3′ region of an exon comprises a sequence about 1 to about 300 nucleotides adjacent to the 3′ end of an exon when moving upstream in the 5′ direction, or a sequence about 1 to about 300 nucleotides adjacent to the 3′ end of an exon when moving downstream in the 3′ direction, or both.

Nucleic acids, such as DNA and pre-mRNA, can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In certain embodiments, nucleic acids can contain one or more elements that act as a signal during transcription, splicing, and/or translation. In certain embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs).

In some embodiments, a target sequence that a guide nucleic acid binds/hybridizes is at least partially within a targeted exon within the human dystrophin gene, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds/hybridizes can comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, at least a portion of the target nucleic acid that a guide nucleic acid binds/hybridizes is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: the start of a targeted exon, the end of a targeted exon, or both.

In some embodiments, a target sequence that a guide nucleic acid binds/hybridizes is at least partially within a targeted exon within the human dystrophin gene, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds/hybridizes can comprise a sequence about 1 to about 300 nucleotides, about 10 to about 250, about 20 to about 200, about 30 to about 150, about 40 to about 100, or about 50 nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

In certain embodiments, at least a portion of the target nucleic acid that a guide nucleic acid binds/hybridizes is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

Further description of editing or detecting a target nucleic acid in the foregoing genes can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

In some embodiments, the target nucleic acid is in a cell. In general, the cell is a human cell. In some embodiments, the human cell is a: muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

### Mutations

In some embodiments, target nucleic acids comprise a mutation. In some embodiments, a sequence comprising a mutation may be modified to a wildtype sequence with a composition, system or method described herein. In some embodiments, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, guide nucleic acids described herein hybridize to a region of the target nucleic acid comprising the mutation. The mutation may be located in a non-coding region or a coding region of a gene.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation, SNP, or deletion is associated with a disease such as a genetic disorder. The mutation, such as a single nucleotide mutation, a SNP, or a deletion, may be encoded in the nucleotide sequence of a target nucleic acid from the germline of an organism or may be encoded in a target nucleic acid from a diseased cell.

In some embodiments, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some embodiments, the mutation is selected from the mutations listed in TABLE 10.

In some embodiments, the target nucleic acid comprises a mutation associated with a disease. In some examples, a mutation associated with a disease refers to a mutation whose presence in a subject indicates that the subject is susceptible to, or suffers from, a disease, disorder, or pathological state. In some examples, a mutation associated with a disease, disorder or pathological state refers to a mutation which causes, contributes to the development of, or indicates the existence of the disease, disorder or pathological state. A mutation associated with a disease may also refer to any mutation which generates transcription or translation products at an abnormal level, or in an abnormal form, in cells affected by a disease relative to a control without the disease.

In some embodiments, the disease, disorder or pathological state comprises any one of the diseases, disorders, syndromes, or pathological states as set forth in TABLE 11.

The mutation may cause a disease. The disease may comprise, at least in part, an inherited disorder, a neurological disorder, or both. The disease may comprise, at least in part, an inherited disorder. The disease may comprise, at least in part, a neurological disorder. In some embodiments, the neurological disorder to a neuromuscular disorder. In some embodiments, the neuromuscular disorder comprises: muscular dystrophy; duchenne muscular dystrophy (DMD); muscular dystrophy, pseudohypertrophic progressive, duchenne type; severe dystrophinopathy, duchenne type; muscular dystrophy duchenne type; becker muscular dystrophy (BMD); muscular dystrophy, pseudohypertrophic progressive, becker type; benign congenital myopathy; benign pseudohypertrophic muscular dystrophy; becker dystrophinopathy; muscular dystrophy pseudohypertrophic progressive, becker type; muscular dystrophy becker type; cardiomyopathy; x-linked dilated cardiomyopathy, type 3B (CMD3B); or dystrophinopathies.

### Certain Samples

Various sample types comprising a target nucleic acid of interest are consistent with the present disclosure. These samples may comprise a target nucleic acid for detection. In some embodiments, the detection of the target nucleic indicates an ailment, such as a disease, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, genetic disorder, or any mutation of interest.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unmodified) sample. Raw samples may be applied to a system for detecting or modifying a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some embodiments, the sample is taken from a human. The sample may comprise one or more cells. The sample may be a tissue sample, e.g., a biopsy sample. In some embodiments, the cell is a muscle cell. The sample comprises nucleic acids from a cell lysate from a muscle cell. the sample comprises nucleic acids from a cell lysate from a cardiac muscle cell, smooth or visceral muscle cell, or a skeletal muscle cell. In some embodiments, the sample comprises nucleic acids expressed from a cell.

In some embodiments, samples are used for diagnosing a disease. In some embodiments, the disease is a genetic disorder. The sample used for genetic testing may comprise at least one target nucleic acid that may bind/hybridize to a guide nucleic acid of the reagents described herein. The target nucleic acid, in some embodiments, comprises a portion of a gene comprising a mutation associated with a genetic disease or a gene whose expression is associated with a genetic disease. Sometimes, the target nucleic acid encodes a disease biomarker, such as a gene mutation. In some embodiments, the target nucleic acid is a portion of a nucleic acid from a genomic locus, any DNA amplicon of, a reverse transcribed mRNA, or a cDNA from a locus of at least one of the genes in TABLE 7. Any region of the aforementioned gene loci may be probed for a mutation or deletion using the compositions, systems and methods disclosed herein. For example, in the DMD gene locus, the compositions, systems and methods for detection disclosed herein may be used to detect a single nucleotide polymorphism or a deletion. In some embodiments, the gene is DMD. In some embodiments, the contacting occurs in vitro. In some embodiments, the contacting occurs in vivo. In some embodiments, the contacting occurs ex vivo. In some embodiments, the target nucleic acid comprises a portion of a nucleic acid from a genomic locus, any DNA amplicon of, a reverse transcribed mRNA, or a cDNA from a locus of DMD.

In some embodiments, the genetic disorder is Duchenne muscular dystrophy, Becker Muscular Disorder, or type 3B dilated cardiomyopathy. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is encoded by a gene described in TABLE 7. In some embodiments, the target nucleic acid is encoded by a gene described in TABLE 7 comprising a mutation.

The sample used for phenotyping testing may comprise at least one target nucleic acid that may bind/hybridize to a guide nucleic acid of the reagents described herein. The target nucleic acid, in some embodiments, is a nucleic acid encoding a sequence associated with a phenotypic trait.

The sample used for genotyping testing may comprise at least one target nucleic acid that may bind/hybridize to a guide nucleic acid of the reagents described herein. The target nucleic acid, in some embodiments, is a nucleic acid encoding a sequence associated with a genotype of interest.

The sample used for ancestral testing may comprise at least one target nucleic acid that may bind/hybridize to a guide nucleic acid of the reagents described herein. The target nucleic acid, in some embodiments, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

Any of the above disclosed samples are consistent with the methods, compositions, reagents, enzymes, and systems disclosed herein.

### IX. Vectors and Multiplexed Expression Vectors

In some instances, compositions, systems, and methods provided herein comprise a vector system encoding an effector protein described herein. In some instances, compositions, systems, and methods provided herein comprise a vector system encoding a guide nucleic acid described herein. In some instances, compositions, systems, and provided herein comprise a multi-vector system encoding an effector protein and a guide nucleic acid described herein, wherein the guide nucleic acid and the effector protein are encoded by the same or different vectors. In some instances, the engineered guide and the engineered effector protein are encoded by different vectors of the system. In some embodiments, a nucleic acid encoding an effector protein comprises an expression vector. In some embodiments, a nucleic acid encoding an effector protein is a messenger RNA. In some embodiments, an expression vector comprises or encodes an engineered guide nucleic acid. In some cases, the expression vector encodes the crRNA. In some cases, the expression vector encodes the tracrRNA. In some cases, the expression vector encodes the sgRNA.

In some instances, a vector may encode one or more engineered effector proteins. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 engineered effector proteins. In some instances, a vector can encode one or more engineered effector proteins comprising an amino acid sequence set forth in SEQ ID NO: 1.

In some instances, a vector may encode one or more guide nucleic acids. In some instances, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 different guide nucleic acids. In some instances, a vector can encode one or more guide nucleic acids comprising one or more sequences recited in TABLE 4, TABLE 5, or TABLE 6.

In some embodiments, the vector encoding one or more engineered effector proteins, one or more guide nucleic acids, or combinations thereof are delivered to eukaryotic cells. In some embodiments, the eukaryotic cells comprise induced pluripotent stem cell. In some embodiments, the eukaryotic cells comprise HEK293T cells, cardiomyocytes or myoblasts. In some embodiments, the cardiomyocytes are derived from the induced pluripotent stem cells. In some embodiments, the myoblasts are derived from the induced pluripotent stem cells.

In some instances, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some instances, a vector can comprise or encode for one or more additional elements, such as, for example, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

In some embodiments, vectors described herein comprises two, three, four, or five promoters. In some embodiments, vectors described herein comprises two promoters. In some embodiments, vectors described herein comprises three promoters. In some embodiments, the length of the promoter is less than about 500, less than about 400, or less than about 300 linked nucleotides. In some embodiments, the length of the promoter is at least 100 linked nucleotides.

Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 10 fold, by 100 fold, or by 1000 fold, or more. In addition, vectors used for providing a nucleic acid encoding an engineered guide nucleic acid and/or an effector protein to a cell may include nucleic acid sequences that encode for selectable markers in the target cells, so as to identify cells that have taken up the engineered guide nucleic acid and/or an effector protein.

Other non-limiting examples of promoters include 7SK, EF1a, RPBSA, hPGK, EFS, PGK1, Ubc, human beta actin promoter, CAG, TRE, UAS, Ac5, Polyhedrin, CaMKIIa, GAL1, TEF1, GDS, ADH1, CaMV35S, Ubi, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, some promoters (e.g., U6, enhanced U6, Hl and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein (e.g., TABLE 4, TABLE 5, TABLE 6, TABLE 12, TABLE 13, and TABLE 14) further comprises “g” nucleotide at 5′ end of the guide nucleic acid, wherein the promotor is selected from U6, enhanced U6, Hl and 7SK.

In some embodiments, an effector protein (or a nucleic acid encoding same) and/or an engineered guide nucleic acid (or a nucleic acid encoding same) are co-administered with a donor nucleic acid. Coadministration can be contact with a target nucleic acid, administered to a cell, such as a host cell, or administered as method of nucleic acid detection, editing, and/or treatment as described herein, in a single vehicle, such as a single expression vector. In certain embodiments, an effector protein (or a nucleic acid encoding same) and/or an engineered guide nucleic acid (or a nucleic acid encoding same) are not co-administered with donor nucleic acid in a single vehicle. In certain embodiments, an effector protein (or a nucleic acid encoding same), an engineered guide nucleic acid (or a nucleic acid encoding same), and/or donor nucleic acid are administered in one or more or two or more vehicles, such as one or more, or two or more expression vectors.

### Lipid Particles

In some instances, compositions, systems, and methods provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the effector protein, the sgRNA or crRNA, the nucleic acid encoding the effector protein and/or the DNA molecule encoding the sgRNA or crRNA. LNPs are a non-viral delivery system for gene therapy. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design. In some cases, a method can comprise contacting a cell with an expression vector. In some cases, contacting can comprise electroporation, lipofection, or lipid nanoparticle (LNP) delivery of an expression vector.

### Viral Vectors

An expression vector can be a viral vector. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools can include, but are not limited to, an effector protein, effector protein modifications (e.g., nuclear localization signal (NLS), enhancer, intron, polyA tail), guide nucleic acid(s), respective promoter(s), and a donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In general, viral vectors provided herein comprise at least one promotor or a combination of promoters driving expression or transcription of one or more genome editing tools described herein. In some embodiments, the viral vector comprises a nucleotide sequence of a promoter. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present. e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### X. Pharmaceutical Compositions and Modes of Administration

Disclosed herein, in some aspects, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo, or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds (e.g., non-covalently interacts) to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

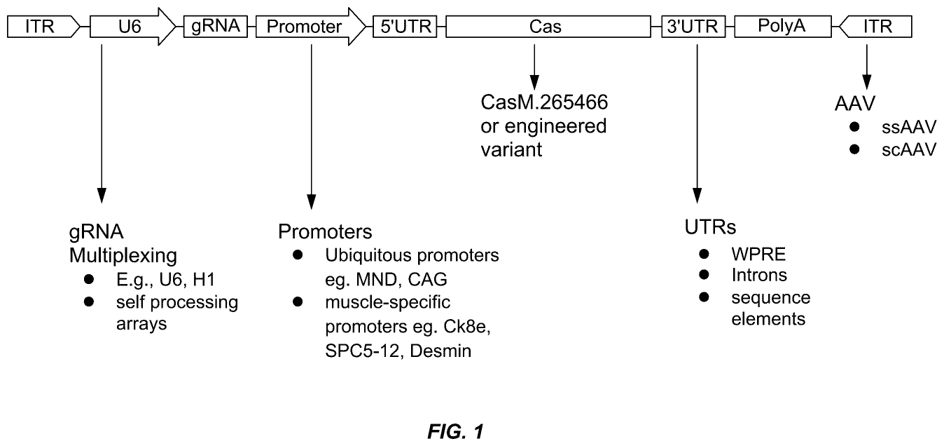

In some embodiments, methods of producing delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments, the AAV vector comprises a sequence encoding a guide nucleic acid. In some embodiments, the guide nucleic acid comprises a crRNA. In some embodiments, the guide nucleic acid is a crRNA. In some embodiments, the guide nucleic acid comprises a sgRNA. In some embodiments, the guide nucleic acid is a sgRNA. In some examples, the AAV vector can package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequence encoding a first guide nucleic acid and a nucleotide sequence encoding a second guide nucleic acid, wherein the first guide nucleic acid and the second guide nucleic acid are the same. In some embodiments, the AAV vector packages a nucleotide sequence encoding a first guide nucleic acid and a nucleotide sequence encoding a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats of ssAAV vector or scAAV vector. In some embodiments, the AAV vector comprises a mutated inverted terminal repeat that lacks a terminal resolution site. FIG. 1 illustrates an exemplary schematic of AAV construct.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises a nucleotide sequence of one or more promoters, enhancers, ribosome binding sites, RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the AAV vector comprises a stuffer sequence. A stuffer sequence can refer to a non-coding sequence of nucleotides that adjusts the length of the viral genome when inserted into a vector to increase packaging efficiency, increase overall viral titer during production, increase transfection efficacy, increase transfection efficiency, and/or decrease vector toxicity. In some embodiments, the stuffer sequence comprises 5′ untranslated region, 3′ untranslated region or combination thereof. In some embodiments, a stuffer sequence serves no other functional purpose than to increase the length of the viral genome. In some embodiments, a stuffer sequence may increase the length of the viral genome as well as have other functional elements.

In some embodiments, the 3′-untranslated region comprises a nucleotide sequence of an intron. In some embodiments, the 3′-untranslated region comprises one or more sequence elements, such as an intron sequence or an enhancer sequence. In some embodiments, the 3′-untranslated region comprises an enhancer.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995). In some embodiments, the enhancer is WPRE.

In some embodiments, the AAV vector comprises one or more polyadenylation (poly A) signal sequences. In some embodiments, the polyadenylation signal sequence comprises hGH poly A signal sequence. In some embodiments, the polyadenylation signal sequence comprises sv40 poly A signal sequence.

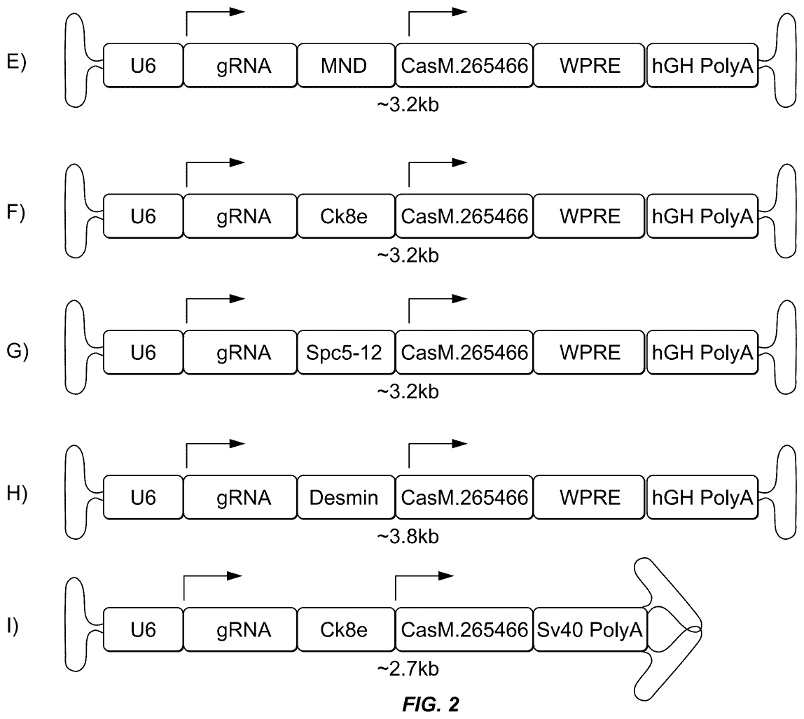

FIG. 2 illustrates exemplary AAV constructs having a nucleic acid encoding one guide nucleic acid. In some embodiments, the guide nucleic acid comprises a guide RNA (e.g., crRNA or sgRNA). Single cutting can be assessed by delivery of a nucleic acid encoding one guide nucleic acid. Accordingly, in some embodiments, any one of AAV construct illustrated in FIG. 2 can be used to modify (e.g., introduce a single cut within or near) a target nucleic acid.

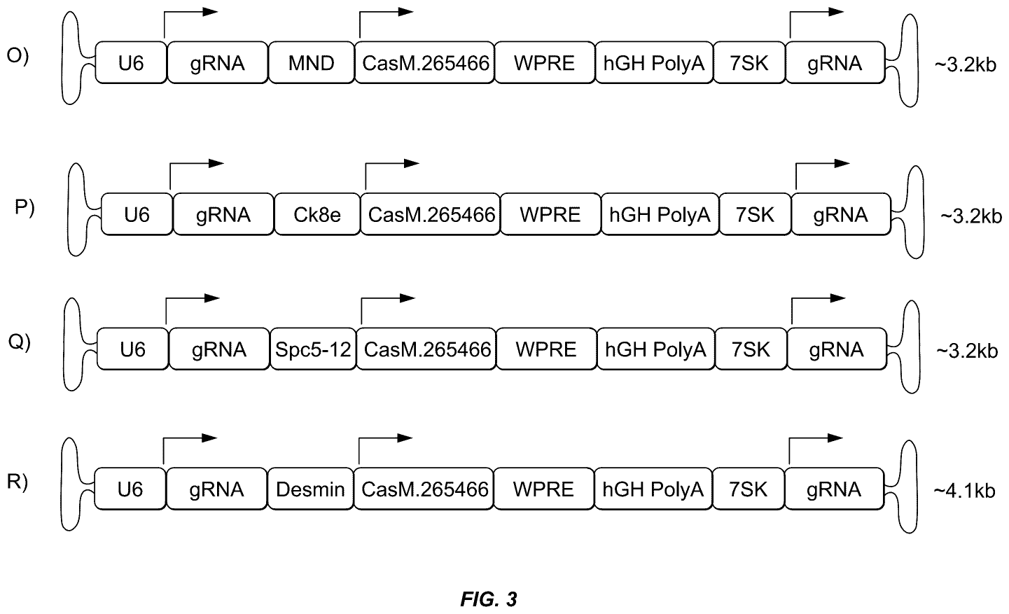

FIG. 3 illustrates exemplary AAV constructs having two nucleic acids encoding two guide nucleic acids. In some embodiments, a first nucleic acid encoding a first guide nucleic acid comprises a first guide RNA and a second nucleic acid encoding a second guide nucleic acid comprises a second guide RNA. In some embodiments, the first nucleic acid and the second nucleic acid are same. Accordingly, in some embodiments, exemplary AAV constructs of FIG. 3 can be used to modify (e.g., introduce a single cut) at a higher rate than the construct having a single copy of a nucleic acid encoding one guide nucleic acid. Alternatively, in some embodiments, the first nucleic acid and the second nucleic acid are different. Dual cutting can be assessed by delivery of two different nucleic acids each encoding guide nucleic acid. Accordingly, in some embodiments, AAV constructs illustrated in FIG. 3 can be used for dual cutting within or near about a target nucleic acid.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent. In some embodiments, guide nucleic acid can be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to the amino acid sequence of SEQ ID NO: 1. In some embodiments, the effector protein comprises a sequence that is at least 65%, at least 70%, at least 75%, at least 80%, at least 85%, at least 90%, at least 95%, at least 97%, or at least 98%, at least 99%, or 100% similar to the amino acid sequence of SEQ ID NO: 1. In some embodiments, the guide nucleic acid comprises a nucleotide sequence of any one of the gRNA sequences of TABLE 6. In some embodiments, the nucleotide sequence of the gRNA is at least 65%, at least 70%, at least 75%, at least 80%, at least 85%, at least 90%, at least 95%, at least 97%, or at least 98%, at least 99%, or 100% identical to any one of the gRNA sequences of TABLE 6.

In combination with a pharmaceutically acceptable carrier or diluent, each row in TABLE 6 can represent an exemplary pharmaceutical composition comprising an effector protein as set forth in SEQ ID NO: 1 recognizing a PAM sequence as set forth in TABLE 2 and a guide nucleic acid wherein the guide nucleic acid is a sgRNA. In some embodiments, the guide nucleic acid comprises a nucleotide sequence of any one of the sgRNA sequences of TABLE 6. In some embodiments, the nucleotide sequence of the gRNA is at least 65%, at least 70%, at least 75%, at least 80%, at least 85%, at least 90%, at least 95%, at least 97%, or at least 98%, at least 99%, or 100% identical to any one of the sgRNA sequences of TABLE 6.

### XI. Methods and Formulations for Introducing Systems and Compositions into a Target Cell

A guide nucleic acid (or a nucleic acid comprising a nucleotide sequence encoding same) and/or an effector protein described herein can be introduced into a host cell by any of a variety of well-known methods. As a non-limiting example, a guide nucleic acid and/or effector protein can be combined with a lipid. As another non-limiting example, a guide RNA and/or effector protein can be combined with a particle, or formulated into a particle.

### Methods for Introducing Systems and Compositions to a Host

Described herein are methods of introducing various components described herein to a host. A host can be any suitable host, such as a host cell. When described herein, a host cell can be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell can be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method can be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some instances, the nucleic acid and/or protein are introduced into a disease cell comprised in a pharmaceutical composition comprising the guide nucleic acid and/or effector protein and a pharmaceutically acceptable excipient.

In certain embodiments, molecules of interest, such as nucleic acids of interest, are introduced to a host. In certain embodiments, polypeptides, such as an effector protein are introduced to a host. In certain embodiments, vectors, such as lipid particles and/or viral vectors can be introduced to a host. Introduction can be for contact with a host or for assimilation into the host, for example, introduction into a host cell.

In some instances, described herein are methods of introducing one or more nucleic acids, such as a nucleic acid encoding an effector protein, a nucleic acid encoding an engineered guide nucleic acid, and/or a donor nucleic acid, or combinations thereof, into a host cell. Any suitable method can be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell can occur in any culture media and under any culture conditions that promote the survival of the cells. Introducing one or more nucleic acids into a host cell can be carried out in vivo or ex vivo. Introducing one or more nucleic acids into a host cell can be carried out in vitro.

In some embodiments, an effector protein can be provided as RNA. The RNA can be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid can be through the use of a vector and/or a vector system, accordingly, in some embodiments, compositions, methods and system described herein comprise a vector and/or a vector system.

Vectors may be introduced directly to a host. In certain embodiments, host cells can be contacted with one or more vectors as described herein, and in certain embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein can also be introduced directly to a host. For example, an engineered guide nucleic acid can be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein can also be introduced directly to a host. In some embodiments, polypeptides described herein can be modified to promote introduction to a host. For example, polypeptides described herein can be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide can be modified to improve stability. For example, the polypeptides may be PEGylated, where the polyethyleneoxy group provides for enhanced lifetime in the blood stream. Polypeptides can also be modified to promote uptake by a host, such as a host cell. For example, a polypeptide described herein can be fused to a polypeptide permeant domain to promote uptake by a host cell. Any suitable permeant domains can be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site can be determined by suitable methods.

### Formulations for Introducing Systems and Compositions to a Host

Described herein are formulations of introducing systems and compositions described herein to a host. In some embodiments, such formulations, systems and compositions described herein comprise an effector protein and a carrier (e.g., excipient, diluent, vehicle, or filling agent).

In some aspects of the present invention the effector protein is provided in a pharmaceutical composition comprising the effector protein and any pharmaceutically acceptable excipient, carrier, or diluent. In some embodiments, a pharmaceutically acceptable excipient, carrier or diluent can describe any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18^{th} edition, A. Gennaro, ed., Mack Publishing Co., Easton, Pa., 1990; and Remington, The Science and Practice of Pharmacy 21^{st} Ed. Mack Publishing, 2005).

### XII. Systems

Disclosed herein, in some aspects, are systems for detecting, modifying, or editing a target nucleic acid, comprising the effector proteins described herein, or a multimeric complex thereof. Systems may be used to detect, modify, or edit a target nucleic acid. Systems may be used to modify the activity or expression of a target nucleic acid. In some embodiments, systems comprise an effector protein described herein, a reagent, support medium, or a combination thereof. In some embodiments, the effector protein comprises an effector protein, or a fusion protein thereof, described herein. In some embodiments, effector proteins comprise an amino acid sequence that is at least 65%, at least 70%, at least 75%, at least 80%, at least 85%, at least 90%, at least 95%, or 100% identical to the amino acid sequence of SEQ ID NO: 1. In some embodiments, the amino acid sequence of the effector protein is at least 65%, at least 70%, at least 75%, at least 80%, at least 85%, at least 90%, at least 95%, or 100% identical to the amino acid sequence of SEQ ID NO: 1. In some embodiments, effector proteins comprise an amino acid sequence that is at least 65%, at least 70%, at least 75%, at least 80%, at least 85%, at least 90%, at least 95%, or 100% similar to the amino acid sequence of SEQ ID NO: 1. In some embodiments, the amino acid sequence of the effector protein is at least 65%, at least 70%, at least 75%, at least 80%, at least 85%, at least 90%, at least 95%, or 100% similar to the amino acid sequence of SEQ ID NO: 1.

In some embodiments, systems comprise an effector protein described herein, a guide nucleic acid described herein, a reagent, support medium, or a combination thereof. In some embodiments, the effector protein comprises an effector protein, or a fusion protein thereof, described herein. In some embodiments, effector protein comprises an amino acid sequence that is at least 65%, at least 70%, at least 75%, at least 80%, at least 85%, at least 90%, at least 95%, or 100% identical to the amino acid sequence of SEQ ID NO: 1. In some embodiments, the amino acid sequence of the effector protein is at least 65%, at least 70%, at least 75%, at least 80%, at least 85%, at least 90%, at least 95%, or 100% identical to the amino acid sequence of SEQ ID NO: 1. In some embodiments, the guide nucleic acid comprises a spacer sequence that is at least 65%, at least 70%, at least 75%, at least 80%, at least 85%, at least 90%, at least 92%, at least 95%, or 100% identical to any one of the nucleotide sequences set forth in TABLE 4 and a handle sequence that is at least 65%, at least 70%, at least 75%, at least 80%, at least 85%, at least 90%, at least 92%, at least 95%, or 100% identical to the handle sequence set forth in TABLE 5. In some embodiments, the nucleotide sequence of the guide nucleic acid is at least 65%, at least 70%, at least 75%, at least 80%, at least 85%, at least 90%, at least 92%, at least 95%, at least 97%, at least 98%, at least 99%, or 100% identical to any one of the sgRNA sequences set forth in TABLE 6.

Systems may be used for detecting the presence or the absence of a target nucleic acid as described herein, for example as set forth in TABLE 7. Systems may be used for detecting the presence or the absence of a mutation of a target nucleic acid as described herein, for example as set forth in TABLE 10. Systems may be used for detecting the presence or the absence of a target nucleic acid associated with or causative of a disease or disorder, such as a genetic disorder. Systems may be used for detecting the presence or the absence of a target nucleic acid associated with or causative of a disease or disorder as described herein, for example as set forth in TABLE 11. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), ß-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

### Reporters

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, Ypet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., or any value 20° C. to 45° C. In some embodiments, the nucleic acid amplification reaction is performed at a temperature of at least 20° C., 25° C., 30° C., 35° C., 37° C., 40° C., or 45° C., or any value 20° C. to 45° C. In some embodiments, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For embodiment, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM via amplification into the target nucleic acid.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, compositions, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, compositions, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl. In some embodiments, the salt is KNO₃. In some embodiments, the salt concentration is less than 150 mM, less than 125 mM, less than 100 mM, less than 75 mM, less than 50 mM, or less than 25 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., or about 50° C.

### XIII. Methods of Nucleic Acid Editing

Provided herein are compositions, methods, and systems for editing target nucleic acids. In general, editing refers to modifying the nucleotide sequence of a target nucleic acid. However, compositions, methods, and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

The target nucleic acid may be a gene or a portion thereof. Methods, systems and compositions may modify a coding portion of a gene, a non-coding portion of a gene, or a combination thereof. Modifying at least one gene using the compositions, systems and methods described herein may reduce or increase expression of one or more genes. In some embodiments, compositions, systems and methods reduce expression of one or more genes by at least 10%, at least 20%, at least 30%, at least 40%, at least 50%, at least 60%, at least 70%, at least 80%, at least 90%, or at least 95%. In some embodiments, compositions, systems and methods remove all expression of a gene, also referred to as genetic knock out. In some embodiments, compositions, systems and methods increase expression of one or more genes by at least 10%, at least 20%, at least 30%, at least 40%, at least 50%, at least 60%, at least 70%, at least 80%, at least 90%, or at least 100%.

In some embodiments, compositions, systems and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some embodiments, effector proteins need not be fused to a partner protein to accomplish the required protein (expression) modification.

In some embodiments, compositions, systems and methods comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions, systems and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of editing may comprise contacting a target nucleic acid with an effector protein described herein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to the amino acid sequence set forth in SEQ ID NO: 1. In some embodiments, the guide nucleic acid comprises a spacer sequence that is at least 65%, at least 70%, at least 75%, at least 80%, at least 85%, at least 90%, at least 92%, at least 95%, or 100% identical to any one of the nucleotide sequences set forth in TABLE 4 and a handle sequence that is at least 65%, at least 70%, at least 75%, at least 80%, at least 85%, at least 90%, at least 92%, at least 95%, or 100% identical to the handle sequence set forth in TABLE 5. In some embodiments, the nucleotide sequence of the guide nucleic acid is at least 65%, at least 70%, at least 75%, at least 80%, at least 85%, at least 90%, at least 92%, at least 95%, at least 97%, at least 98%, at least 99%, or 100% identical to any one of the gRNA sequences set forth in TABLE 6.

Editing may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation.

In some embodiments, wherein the compositions, systems, and methods of the present disclosure comprise an additional guide nucleic acid or a use thereof, the dual-guided compositions, systems, and methods described herein can modify the target nucleic acid in two locations. In some embodiments, dual-guided editing can comprise cleavage of the target nucleic acid in the two locations targeted by the guide RNAs. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some embodiments, the effector protein is fused to a chromatin-modifying enzyme. In some embodiments, the fusion protein chemically modifies the target nucleic acid, for example by methylating, demethylating, or acetylating the target nucleic acid in a sequence specific or non-specific manner.

Methods may comprise use of two or more effector proteins. An illustrative method for introducing a break in a target nucleic acid comprises contacting the target nucleic acid with: (a) a first engineered guide nucleic acid comprising a region that binds to a first effector protein, wherein the effector protein comprises an amino acid sequence that is at least 75% identical to the amino acid sequence of SEQ ID NO: 1; and (b) a second engineered guide nucleic acid comprising a region that binds to a second effector protein, wherein the effector protein comprises an amino acid sequence that is at least 75% identical to the amino acid sequence of SEQ ID NO: 1, wherein the first engineered guide nucleic acid comprises an additional region that binds/hybridizes to the target nucleic acid and wherein the second engineered guide nucleic acid comprises an additional region that binds/hybridizes to the target nucleic acid. In some embodiments, the nucleotide sequence of the guide nucleic acid is at least 65%, at least 70%, at least 75%, at least 80%, at least 85%, at least 90%, at least 92%, at least 95%, at least 97%, at least 98%, at least 99%, or 100% identical to any one of any one of the sgRNA sequences of TABLE 6. In some embodiments, the guide nucleic acid comprises a crRNA sequence comprising a spacer sequence that is at least 65%, at least 70%, at least 75%, at least 80%, at least 85%, at least 90%, at least 92%, at least 95%, or 100% identical to any one of the nucleotide sequences of TABLE 4 and a handle sequence that is at least 65%, at least 70%, at least 75%, at least 80%, at least 85%, at least 90%, at least 92%, at least 95%, or 100% identical to the handle sequence of TABLE 5.

In some embodiments, editing a target nucleic acid comprises genome editing. Genome editing may comprise modifying a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, methods comprise contacting a target nucleic acid with an effector protein comprising an amino acid sequence that is at least 65%, at least 70%, at least 75%, at least 80%, at least 85%, at least 90%, at least 95%, at least 97%, at least 98%, at least 99%, or 100% identical to the amino acid sequence of SEQ ID NO: 1, thereby introducing a single-stranded break in the target nucleic acid; contacting the target nucleic acid with a second effector protein, optionally comprising an amino acid sequence that is at least 65%, at least 70%, at least 75%, at least 80%, at least 85%, at least 90%, at least 95%, at least 97%, at least 98%, or at least 99% identical to the amino acid sequence of SEQ ID NO: 1, to generate a second cleavage site in the target nucleic acid, optionally ligating the regions flanking the first and second cleavage site through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites). In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to the amino acid sequence of SEQ ID NO: 1. In some embodiments, the nucleotide sequence of the guide nucleic acid is at least 65%, at least 70%, at least 75%, at least 80%, at least 85%, at least 90%, at least 92%, at least 95%, at least 97%, at least 98%, at least 99%, or 100% identical to any one of the sgRNA sequences of TABLE 6. In some embodiments, the guide nucleic acid comprises a spacer sequence that is at least 65%, at least 70%, at least 75%, at least 80%, at least 85%, at least 90%, at least 95%, or 100% identical to any one of the nucleotide sequences of TABLE 4 and a handle sequence that is at least 65%, at least 70%, at least 75%, at least 80%, at least 85%, at least 90%, at least 92%, at least 95%, or 100% identical to the handle sequence of TABLE 5.

In some embodiments, editing is achieved by fusing an effector protein to a heterologous sequence. The heterologous sequence may be a suitable fusion partner, e.g., a protein that provides recombinase activity by acting on the target nucleic acid. In some embodiments, the fusion protein comprises an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the effector protein. The heterologous sequence or fusion partner may be fused to the effector protein by a linker. A linker may be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

Methods, systems and compositions described herein can edit or modify a target nucleic acid wherein such editing or modification can effect one or more indels. In some embodiments, where compositions, systems, and/or methods described herein effect one or more indels, then in certain embodiments, the impact on the transcription and/or translation of the target nucleic acid can be predicted depending on: 1) the amount of indels generated; and 2) the location of the indel on the target nucleic acid. For example, as described herein, in certain embodiments, if the amount of indels is not divisible by three, and the indels occur within or along a protein coding region, then the modification or mutation can be a frameshift mutation.

In certain embodiments, if the amount of indels is divisible by three, then a frameshift mutation may not be effected, but a splicing disruption mutation and/or sequence skip mutation may be effected, such as an exon skip mutation. In certain embodiments, if the amount of indels is not evenly divisible by three, then a frameshift mutation may be effected.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain instances, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing or modifications of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof.

A splicing disruption can be a modification that disrupts the splicing of a target nucleic acid or splicing of a sequence that is transcribed from a target nucleic acid relative to a target nucleic acid without the splicing disruption.

A frameshift mutation can be a modification that alters the reading frame of a target nucleic acid relative to a target nucleic acid without the frameshift mutation. In certain embodiments, a frameshift mutation can be a +2 frameshift mutation wherein a reading frame is modified by 2 bases. In certain embodiments, a frameshift mutation can be a +1 frameshift mutation wherein a reading frame is modified by 1 base. In certain embodiments, a frameshift mutation is a modification that alters the number of bases in a target nucleic acid so that it is not divisible by three. In some embodiments, a frameshift mutation can be a modification that is not a splicing disruption.

A sequence as described in reference to a sequence deletion, sequence skipping, sequence reframing, and sequence knock-in can be a DNA sequence, a RNA sequence, a modified DNA or RNA sequence, a mutated sequence, a wild-type sequence, a coding sequence, a non-coding sequence, an exonic sequence (exon), an intronic sequence (intron), or any combination thereof. Such a sequence can be a sequence that is associated with a disease as described herein, such as DMD.

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion can result in or effect a splicing disruption.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can effect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject.

### Donor Nucleic Acids

In some embodiments, a donor nucleic acid comprises a nucleic acid that is incorporated into a target nucleic acid.

In certain embodiments, a donor nucleic acid comprises a transgene. A transgene can be a nucleic acid, such as DNA. In some embodiments, the transgene is inserted or integrated into the target nucleic acid. In some embodiments, the transgene is a nucleotide sequence that is inserted into a cell for expression of the nucleotide sequence in the cell. In some embodiments, the transgene includes (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break-nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

### Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

In some embodiments, upon modification of a target nucleic acid by compositions, systems, and methods described herein, the target nucleic acid can comprise an exon deletion, exon skipping, exon reframing, exon knock-in, or any combination thereof. In certain embodiments, cells and organism described herein can comprise a modified target nucleic acid comprising a splicing disruption, frameshift (e.g., 1+ or 2+ frameshift), sequence deletion, sequence skipping, sequence reframing, sequence knock-in, or any combination thereof, relative to a target nucleic acid that is not modified by the compositions, systems, or methods described herein. In some embodiments, the modified cells can be used to assess the modification of DMD using the compositions, systems, and methods. For example, in some embodiments, an entire exon (e.g., exon 50) can be deleted to generate a model DMD cells. Such cells can be used to assess different repair strategies, such as, introduction of an indel that may result in exon skipping or exon deletion, and thereby reframing the DMD gene.

Methods may comprise contacting a cell with a nucleic acid (e.g., a plasmid or mRNA) comprising a nucleotide sequence encoding an effector protein, wherein the effector protein comprises an amino acid sequence that is at least 65%, at least 70%, at least 75%, at least 80%, at least 85%, at least 90%, at least 95%, at least 97%, at least 98%, or at least 99% identical to the amino acid sequence of SEQ ID NO: 1. In some embodiments, methods may comprise contacting a cell with a nucleic acid (e.g., a plasmid or mRNA) comprising a nucleotide sequence encoding an effector protein, wherein the effector protein comprises an amino acid sequence that is at least 65%, at least 70%, at least 75%, at least 80%, at least 85%, at least 90%, at least 95%, at least 97%, at least 98%, or at least 99% similar to the amino acid sequence of SEQ ID NO: 1.

Methods may comprise contacting cells with a nucleic acid (e.g., a plasmid or mRNA) comprising a nucleotide sequence encoding a guide nucleic acid, sgRNA, a tracrRNA, a crRNA, or any combination thereof. In some embodiments, the nucleotide sequence of the guide nucleic acid is at least 65%, at least 70%, at least 75%, at least 80%, at least 85%, at least 90%, at least 92%, at least 95%, at least 97%, at least 98%, at least 99%, or 100% identical to of any one of the gRNA sequences of TABLE 6. In some embodiments, the guide nucleic acid comprises a crRNA sequence comprising a spacer sequence that is at least 65%, at least 70%, at least 75%, at least 80%, at least 85%, at least 90%, at least 92%, at least 95%, at least 97%, at least 98%, at least 99%, or 100% identical to any one of the nucleotide sequences of TABLE 4, and a handle sequence that is at least 65%, at least 70%, at least 75%, at least 80%, at least 85%, at least 90%, at least 92%, at least 95%, at least 97%, at least 98%, at least 99%, or 100% identical to the handle sequence of TABLE 5. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with an effector protein or a multimeric complex thereof, wherein the effector protein comprises an amino acid sequence that is at least 65%, at least 70%, at least 75%, at least 80%, at least 85%, at least 90%, at least 95%, at least 97%, at least 98%, or at least 99% identical to the amino acid sequence of SEQ ID NO: 1. In some embodiments, methods may comprise contacting a cell with an effector protein or a multimeric complex thereof, wherein the effector protein comprises an amino acid sequence that is at least 65%, at least 70%, at least 75%, at least 80%, at least 85%, at least 90%, at least 95%, at least 97%, at least 98%, or at least 99% similar to the amino acid sequence of SEQ ID NO: 1.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in TABLE 11. A subject may be at risk of developing Duchenne muscular dystrophy. A subject may be suffering from Duchenne muscular dystrophy. A subject may display symptoms of Duchenne muscular dystrophy. The subject may have a mutation associated with the DMD gene. The subject may display symptoms associated with a mutation of the DMD gene. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as set forth in TABLE 10.

Symptoms of muscular dystrophy, including DMD, may vary from mild to severe and may depend on what part of the body is affected, the causative mutation, and the age and overall health of the affected person, can include, e.g., fatigue, learning difficulties, intellectual disability, muscle weakness (e.g., in the legs, pelvis, arms, neck, diaphragm, heart, or other areas of the body), difficulty with motor skills (e.g., running, hopping, or jumping), frequent falls, trouble getting up from a lying position or climbing stairs, progressive difficulty walking, breathing difficulties, heart disease, abnormal heart muscle (e.g., cardiomyopathy), congestive heart failure, irregular heart rhythm (e.g., arrhythmias), deformities of the chest or back (scoliosis), enlarged muscles of the calves, buttocks, or shoulders, pseudohypertrophy, muscle deformities, respiratory disorders (e.g., pneumonia or poor swallowing). Symptoms can be measured for example, by utilizing: electromyography (EMG), genetic tests, muscle biopsy, serum Creatine Kinase (CK) levels, muscular strength tests (e.g., manual muscle testing), or range-of-motion (ROM) tests such as the six minute walk test.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. The tissue may be muscle. The muscle may be skeletal muscle. In certain embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx—inferior, constrictor of pharynx—middle, constrictor of pharynx—superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei—dorsal of hand, interossei—dorsal of foot, interossei—palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani—iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis—multifidus, transversospinalis—rotatores, transversospinalis—semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a: a stem cell, muscle satellite cell, muscle stem cell, myoblast, muscle progenitor cell, a pluripotent stem cell or a cell derived from a pluripotent stem cell.

### XIV. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

In some embodiments, methods of detecting comprise contacting a target nucleic acid, a cell comprising the target nucleic acid, or a sample comprising a target nucleic acid with an effector protein that comprises an amino acid sequence that is at least is at least 65%, at least 70%, at least 75%, at least 80%, at least 85%, at least 90%, at least 92%, at least 95%, at least 97%, at least 98%, at least 99%, or 100% identical to the amino acid sequence of SEQ ID NO: 1. In some embodiments, the amino acid sequence of the effector protein is at least 65%, at least 70%, at least 75%, at least 80%, at least 85%, at least 90%, at least 92%, at least 95%, at least 97%, at least 98%, at least 99%, or 100% identical to the amino acid sequence of SEQ ID NO: 1.

In some embodiments, methods of detecting comprise contacting a target nucleic acid, a cell comprising the target nucleic acid, or a sample comprising a target nucleic acid with an effector protein that comprises an amino acid sequence that is at least is at least 65%, at least 70%, at least 75%, at least 80%, at least 85%, at least 90%, at least 92%, at least 95%, at least 97%, at least 98%, at least 99%, or 100% similar to the amino acid sequence of SEQ ID NO: 1. In some embodiments, the amino acid sequence of the effector protein is at least 65%, at least 70%, at least 75%, at least 80%, at least 85%, at least 90%, at least 92%, at least 95%, at least 97%, at least 98%, at least 99%, or 100% similar to the amino acid sequence of SEQ ID NO: 1.

Methods may comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding/hybridizing to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least about 40° C., at least about 50° C., or at least about 65° C. In some embodiments, the temperature is not greater than 80° C. In some embodiments, the temperature is about 25° C., about 30° C., about 35° C., about 40° C., about 45° C., about 50° C., about 55° C., about 60° C., about 65° C., or about 70° C. In some embodiments, the temperature is about 25° C. to about 45° C., about 35° C. to about 55° C., or about 55° C. to about 65° C.

Methods of detecting may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

### XV. Method of Treating a Disorder

Described herein are methods for treating a disease in a subject by modifying a target nucleic acid associated with a gene or expression of a gene related to the disease. In some embodiments, the disease or disorder comprises one or more of the diseases or disorder set forth in TABLE 11.

In some embodiments, the method for treating a disease comprises modifying at least one gene associated with the disease or modifying expression of the at least one gene such that the disease is treated. In some embodiments, the disease is any one of the diseases or disorders set forth in TABLE 11 and the gene is the gene set forth in TABLE 7. In some embodiments, the disease is Duchenne Muscular Dystrophy and the gene is DMD.

Modifying at least one gene using the compositions, systems and methods described herein can, in some embodiments, induce a reduction or increase in expression of the one or more genes. In some embodiments, the at least one modified gene results in a reduction in expression, also referred to as gene silencing. In some embodiments, the gene silencing reduces expression of one or more genes by at least 10%, at least 20%, at least 30%, at least 40%, at least 50%, at least 60%, at least 70%, at least 80%, at least 90%, or at least 95%. In some embodiments, gene silencing is accomplished by transcriptional silencing, post-transcriptional silencing, or meiotic silencing. In some embodiments, transcriptional silencing is by genomic imprinting, paramutation, transposon silencing, position effect, or RNA-directed DNA methylation. In some embodiments, post-transcriptional silencing is by RNA interference, RNA silencing, or nonsense mediated decay. In some embodiments, meiotic silencing is by transfection or meiotic silencing of unpaired DNA. In some embodiments, the at least one modified gene results in removing all expression, also referred to as the gene being knocked out (KO).

In some embodiments, a gene is modified by repairing or editing a mutation as described herein. In some embodiments, a Cas protein is used to effect the modification. Cas proteins may be fused to transcription activators or transcriptional repressors or deaminases or other nucleic acid modifying proteins. In some embodiments, Cas proteins need not be fused to a partner protein to accomplish the required protein (expression) modification.

In some embodiments, treatment of a disease comprises administration of a gene therapy. “Gene therapy”, as used herein, comprises use of a recombinant nucleic acid (DNA or RNA), administered for the purpose to adjust, repair, replace, add, or remove a gene sequence. In some embodiments, a gene therapy comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Methods of gene therapy are described in more detail in Ingusci et al., “Gene Therapy Tools for Brain Diseases”, Front. Pharmacol. 10:724 (2019) which is hereby incorporated by reference in its entirety.

It is known that CRISPR-Cas9 gene editing techniques may select for p53-mutated cells. Similarly, the presence of KRAS mutations provides a selective advantage during CRISPR-Cas9 gene editing, as further described in Sinha et al., “A systematic genome-wide mapping of oncogenic mutation selection during CRISPR-Cas9 genome editing”, Nature Comm. 12:6512 (2021), which is hereby incorporated by reference in its entirety. In some embodiments, a genome targeted for treatment comprises a wild-type DMD gene or a mutated DMD gene. In some embodiments, the genome comprises a mutated DMD target gene.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, modifying, replacing, transposing, or affecting the regulation of a genomic sequence of a patient in need thereof. In some embodiments, the methods of treating, preventing, or inhibiting a disease or disorder may involve modulating gene expression.

### SEQUENCES AND TABLES

TABLE 1 provides illustrative amino acid sequences of effector proteins that are useful in the compositions, systems and methods described herein.

TABLE 1
Exemplary Amino Acid Sequence of CasM.265466 Effector Protein

| SEQ ID NO: | Amino Acid Sequence |
|---|---|
| 1 | MSVLTRKVQLIPVGDKEERDRVYKYLRDGIEAQNRAMNLYMSGLYFAAINEA |
| | SKEDRKELNQLYSRIATSSKGSAYTTDIEFPTGLASTSTLSMAVRQDFTKSLKD |
| | GLMYGRVSLPTYRKDNPLFVDVRFVALRGTKQKYNGLYHEYKSHTEFLDNL |
| | YSSDLKVYIKFANDITFQVIFGNPRKSSALRSEFQNIFEEYYKVCQSSIQFSGTKI |
| | ILNMAMDIPDKEIELDEDVCVGVDLGIAIPAVCALNKNRYSRVSIGSKEDFLRV |
| | RTKIRNQRKRLQTNLKSSNGGHGRKKKMKPMDRFRDYEANWVQNYNHYVS |
| | RQVVDFAVKNKAKYINLENLEGIRDDVKNEWLLSNWSYYQLQQYITYKAKT |
| | YGIEVRKINPYHTSQRCSCCGYEDAGNRPKKEKGQAYFKCLKCGEEMNADFN |
| | AARNIAMSTEFQSGKKTKKQKKEQHENK |

TABLE 2 provides illustrative PAM sequences that are useful in the compositions, systems and methods described herein.

TABLE 2
PAM Sequences

| SEQ ID NO: | PAM Sequence (5′ to 3′)* |
|---|---|
| 2 | NNTNTR |
| 3 | TNTR |
| 487 | NNTN |
| 488 | ANTR |
| 489 | CNTR |
| 490 | GNTR |
| 491 | TNAR |
| 492 | TNCR |
| 493 | TNGR |
| 494 | TNTC |
| 495 | TNTT |
| 496 | VNTY |
| 497 | TNVY |
| 500 | TCTG |
| 501 | TATG |
| 502 | TTTG |
| 503 | TGTG |

*wherein each N is independently selected from any nucleotide; wherein each V is independently selected from adenine, cytosine and guanine; and wherein each R is independently selected from adenine and guanine.

TABLE 3 provides illustrative amino acid sequences of exemplary heterologous polypeptide modifications of effector protein(s) that are useful in the compositions, systems and methods described herein.

TABLE 3
Exemplary Amino Acid Sequences of Nuclear
Localization Signals

| SEQ ID NO: | Sequence* |
|---|---|
| 466 | KR(K/R)R |
| 467 | (P/R)XXKR(^{∧}DE)(K/R) |
| 468 | KRX(W/F/Y)XXAF |
| 469 | (R/P)XXKR(K/R)(^{∧}DE) |
| 470 | LGKR(K/R)(W/F/Y) |
| 471 | KRX₁₀₋₁₂K(KR)(KR) |
| 472 | APKKKRKVGIHGVPAA |
| 473 | KRPAATKKAGQAKKKKEF |
| 474 | K(K/R)RK |
| 475 | KRX₁₀₋₁₂K(KR)X(K/R) |

*X is any amino-acid; and ^{∧}D/E is any amino-acid except Asp or Glu

TABLE 4 provides illustrative spacer sequences for use with the compositions, systems and methods of the disclosure.

TABLE 4
Spacer Sequences of gRNAs

| Spacer sequence (5′-to-3′), | |
|---|---|
| shown as RNA | SEQ ID NO |
| AUACUAACCUUGGUUUCUGU | 5 |
| CUGUUCAUUUCAGCUUUAAC | 6 |
| CCACUGCACUUUAGCCUGGG | 7 |
| UCAAAUGUAACCAGUAUUUU | 8 |
| GCCUGGGUGACAGUGAGACU | 9 |
| AAAAGGUAUCUUUGAUACUA | 10 |
| ACGUGAUUUUCUGUUAAUAA | 11 |
| CAAAGUCUACUGUUCAUUUC | 12 |
| AUUUUAUCAAAUGUAACCAG | 13 |
| UUUUCUUAGAGACAGAGUCU | 14 |
| CCAUAGAUUGUAAUUUAAUG | 15 |
| UUUAUUUUCUUAGAGACAGA | 16 |
| GCUAGGAUGAUGAACAACAG | 17 |
| GUAGUAAAUGCUAGUCUGGA | 18 |
| AUGGCAAAUAUUAGUUUCUG | 19 |
| UAGUAGUAAAUGCUAGUCUG | 20 |
| UUAUGGCUAGGAUGAUGAAC | 21 |
| GAGGAGACAUUUUAAAUGUA | 22 |
| AAUGUAACUUCCAAACGUUA | 23 |
| ACUUCCAAACGUUAUCUCAC | 24 |
| CUUUUUUGAUGGCAAAUAUU | 25 |
| AGAUAACGUUUGGAAGUUAC | 26 |
| CCAUCAAAAAAGCAAAGAAU | 27 |
| AAUAAGCAACAUAAAUGUGA | 28 |
| GUUGAAAGAAUUCAGAAUCA | 29 |
| GAAGUUACAUUUAAAAUGUC | 30 |
| ACAGAAACUAAUAUUUGCCA | 31 |
| AAAUGUCUCCUCCAGACUAG | 32 |
| UUCUAGUUGAAAGAAUUCAG | 33 |
| CCAACUUUUAUCAUUUUUUC | 34 |
| UUUGCUGAGAGAGAAACAGU | 35 |
| CUUAGGCUGAAUAGUGAGAG | 36 |
| UCAUUUUUUCUCAUACCUUC | 37 |
| CUUGAUGAUCAUCUCGUUGA | 38 |
| CUGAGAGAGAAACAGUUGCC | 39 |
| GCUACUUUUGUUAUUUGCAU | 40 |
| GGAGAGUAAAGUGAUUGGUG | 41 |
| UGGCUACUUUUGUUAUUUGC | 42 |
| AAGAAAAACUUCUGCCAACU | 43 |
| UAUCCUUGAUUAUACUUAGG | 44 |
| UCCUUGAUUAUACUUAGGCU | 45 |
| AAAUGAAGAUUUUCCACCAA | 46 |
| CUCUCCUAGACCAUUUCCCA | 47 |
| AGUAGGAGCUAAAAUAUUUU | 48 |
| GAUACUUUGUUUAGCAAUAC | 49 |
| GGUUUUUGCAAAAAGGAAAA | 50 |
| UCUUUUUCUAACAAUGUGGA | 51 |
| ACAAUGUGGAUACUUUGUUU | 52 |
| AGCCAAACUCUUAUUCAUGA | 53 |
| AUUGAAGAGUAACAAUUUGA | 54 |
| GCAAUACAUGGUAGAAAAUG | 55 |
| CAAAAAGGAAAAAAGAAGAA | 56 |
| UUUAGCAAUACAUGGUAGAA | 57 |
| UAUCUUUUUCUAACAAUGUG | 58 |
| AUGUCAUGAAUAAGAGUUUG | 59 |
| GCUCCUACUCAGACUGUUAC | 60 |
| GCUUGUGUUUCUAAUUUUUC | 61 |
| UUGCUAAACAAAGUAUCCAC | 62 |
| UAAUGUCAUGAAUAAGAGUU | 63 |
| CCAUGUAUUGCUAAACAAAG | 64 |
| GCUCAAAUUGUUACUCUUCA | 65 |
| AGACUUUUUGCACAGUCAAU | 66 |
| CUUACAGGCUCCAAUAGUGG | 67 |
| CACAGUCAAUAACACAAAGG | 68 |
| GAAUUGAAACAAAUUUUCUC | 69 |
| UCUCUAUCUUUAGAAUUGAA | 70 |
| AACAAAUAGCUAGAGCCAAA | 71 |
| AAUCAGAGUCAAUUUCCAAG | 72 |
| CUCUAAGACUUUUUGCACAG | 73 |
| UUCAAAAGUGCAACUAUGAA | 74 |
| GCUCUAGCUAUUUGUUCAAA | 75 |
| GCUAUUUGUUCAAAAGUGCA | 76 |
| AGUAUACUGGAUCCCAUUCU | 77 |
| UGUUAUUGACUGUGCAAAAA | 78 |
| AUUCAAAGUGUUGCAUGACA | 79 |
| UUUCAAUUCUAAAGAUAGAG | 80 |
| AAGAUAGAGAUAAACCUUUG | 81 |
| UUAUUGACUGUGCAAAAAGU | 82 |
| AAGUGAUGACUGGGUGAGAG | 83 |
| CUGGAUCCCAUUCUCUUUGG | 84 |
| CAAAAAGUCUUAGAGUACAU | 85 |
| AAGAUAAUUCAUGAACAUCU | 86 |
| AUUAUUUUAGCCAACCACCC | 87 |
| CUUCUAAAUUAACUUUAGUG | 88 |
| AAUUAACUUUAGUGGGUAGA | 89 |
| GCCAACCACCCUACAAAUAU | 90 |
| GUGGGUAGAAUUUCUUUUAA | 91 |
| ACAGAAAAGCAUACACAUUA | 92 |
| ACUUCCUCUUUAACAGAAAA | 93 |
| AAAGAAAUUCUACCCACUAA | 94 |
| UAGGGUGGUUGGCUAAAAUA | 95 |
| UAUGCUUUUCUGUUAAAGAG | 96 |
| CCCACUAAAGUUAAUUUAGA | 97 |
| UUAAAGAGGAAGUUAGAAGA | 98 |
| UGCUUUUCUGUUAAAGAGGA | 99 |
| UUCACCAAAUGGAUUAAGAU | 100 |
| GGGUGGUUGGCUAAAAUAAU | 101 |
| CUUUUCUGUUAAAGAGGAAG | 102 |
| CUAAAAUGUUUUCAUUCCUA | 103 |
| CUGCUGUUGAUUAAUGGUUG | 104 |
| AUCUCUCAUGAAAUAUUCUU | 105 |
| AAGAAAGCUUAAAAAGUCUG | 106 |
| UCGCCCUACCUCUUUUUUCU | 107 |
| AUGUUAGUGCCUUUCACCCU | 108 |
| AGCAGGGUGAAAGGCACUAA | 109 |
| GAAGAAUAUUUCAUGAGAGA | 110 |
| AGCUUUCUUUAGAAGAAUAU | 111 |
| CUAAAAUAUAUACUUGUGGC | 112 |
| GCAGACUUUUUAAGCUUUCU | 113 |
| AAAAUUUCCCUAUGAAACUG | 114 |
| UUUGAGAAAAGAUUAAACAG | 115 |
| AGAAAAGAUUAAACAGUGUG | 116 |
| AGAUACCAAAAAGGCAAAAC | 117 |
| CUGGCAAAGAAAGAAAUACA | 118 |
| CUACCACAUGCAGUUGUACU | 119 |
| AAACUGACAUGCCCAUAUCC | 120 |
| UUUUGCCUUUUUGGUAUCUU | 121 |
| GUAGCACACUGUUUAAUCUU | 122 |
| UCCUUUGGAUAUGGGCAUGU | 123 |
| UAUUUCUUUCUUUGCCAGUA | 124 |
| UCUUGUAUCCUUUGGAUAUG | 125 |
| CCUUUUUGGUAUCUUACAGG | 126 |
| GAUAUGGGCAUGUCAGUUUC | 127 |
| GUAUCUUACAGGAACUCCAG | 128 |
| UUUCUUUCUUUGCCAGUACA | 129 |
| CCAGUACAACUGCAUGUGGU | 130 |
| GGCAUGUCAGUUUCAUAGGG | 131 |
| GUAAUAUAAUGAUGACAACA | 132 |
| AGAAGUUAAAGAGUCCAGAU | 133 |
| AUGAUGACAACAACAGUCAA | 134 |
| CUGAAGAUAAAUACAAUUUC | 135 |
| UUUAUCUUCAGCACAUCUGG | 136 |
| AAGGGUGAUGGAAAUUACUU | 137 |
| ACUGUUGUUGUCAUCAUUAU | 138 |
| ACUUCUUAAAGAUCAGGUUC | 139 |
| UCUUCAGCACAUCUGGACUC | 140 |
| GACUUUUUGUGUCAGGAUGA | 141 |
| GACUCUUUAACUUCUUAAAG | 142 |
| GUUAUACUGACAAAGAUAUC | 143 |
| UUUUUUGGUUAUACUGACAA | 144 |
| CUGACAAAGAUAUCACUCUG | 145 |
| GCGUAUAUUUUUUGGUUAUA | 146 |
| CAGAGUUUAGUUUCAAGUAA | 147 |
| AUAGUUUCCUGCAUUUGCAG | 148 |
| UCAAAUCGCCUGCAGGUAAA | 149 |
| GAUCAAGAAAAAUAGAUGGA | 150 |
| CACACCUAGCAUGUACACAC | 151 |
| GAGAUAUAGCGUAUAUUUUU | 152 |
| CCUGCAGGCGAUUUGACAGA | 153 |
| CGCUAUAUCUCUAUAAUCUG | 154 |
| UUUUUCUUGAUCCAUAUGCU | 155 |
| CAAAUGCAGGAAACUAUCAG | 156 |
| UUUGUUACUUGAAACUAAAC | 157 |
| UACAUGCUAGGUGUGUAUAU | 158 |
| UCUCUAUAAUCUGUUUUACA | 159 |
| UGUACAUGCUAGGUGUGUAU | 160 |
| CUUUUACCUGCAGGCGAUUU | 161 |
| UUACUUGAAACUAAACUCUG | 162 |
| ACAGAUCUGUUGAGAAAUGG | 163 |
| AAAUGUUGUGUGUACAUGCU | 164 |
| UAAUCUGUUUUACAUAAUCC | 165 |
| CAUGCUAGGUGUGUAUAUUA | 166 |
| CAUAAUCCAUCUAUUUUUCU | 167 |
| AUCUGUUUUACAUAAUCCAU | 168 |
| ACCAAAAAAUAUACGCUAUA | 169 |
| AUUUUCUUUUGGAUUGCAUC | 170 |
| UUGAAUCCUUUAACAUUUCA | 171 |
| GAUUGCAUCUACUGUAUAGG | 172 |
| ACAUUUCAUUCAACUGUUGC | 173 |
| UAGGGACCCUCCUUCCAUGA | 174 |
| CUUCAUCCCACUGAUUCUGA | 175 |
| AAGGUGUUCUUGUACUUCAU | 176 |
| UGAUUUUCUUUUGGAUUGCA | 177 |
| GGGACCCUCCUUCCAUGACU | 178 |
| CUGUAUAGGGACCCUCCUUC | 179 |
| GCCUGUCCUAAGACCUGCUC | 180 |
| CAGUAGAUGCAAUCCAAAAG | 181 |
| UCCAAGCCCGGUUGAAAUCU | 182 |
| GUUUGGAGAUGGCAGUUUCC | 183 |
| UCACCAGAGUAACAGUCUGA | 184 |
| AUUUUAUAACUUGAUCAAGC | 185 |
| UAACUUGAUCAAGCAGAGAA | 186 |
| ACUUGAUCAAGCAGAGAAAG | 187 |
| CCAGAGCAGGUACCUCCAAC | 188 |
| GAGAUGGCAGUUUCCUUAGU | 189 |
| GUUACUAAGGAAACUGCCAU | 190 |
| GCAGAUUUCAACCGGGCUUG | 191 |
| GUGACACAACCUGUGGUUAC | 192 |
| AAAUCACAGAGGGUGAUGGU | 193 |
| CUUGAUCAAGUUAUAAAAUC | 194 |
| CCGCCUUCCACUCAGAGCUC | 195 |
| CCCUCAGCUCUUGAAGUAAA | 196 |
| AGUGGAAGGCGGUAAACCGU | 197 |
| CUUCAAGAGCUGAGGGCAAA | 198 |
| AGCUCUGAGUGGAAGGCGGU | 199 |
| ACAGCUGUUUGCAGACCUCC | 200 |
| UUUUUGAGGAUUGCUGAAUU | 201 |
| CAGACCUCCUGCCACCGCAG | 202 |
| AGGAUUGCUGAAUUAUUUCU | 203 |
| GAAUACUGGCAUCUGUUUUU | 204 |
| UCUGACAGCUGUUUGCAGAC | 205 |
| ACAACAGUUUGCCGCUGCCC | 206 |
| CCGCUGCCCAAUGCCAUCCU | 207 |
| CAAACAGCUGUCAGACAGAA | 208 |
| CAGGAAAAAUUGGGAAGCCU | 209 |
| CGGUGGCAGGAGGUCUGCAA | 210 |
| GCAUGUUCCCAAUUCUCAGG | 211 |
| UCAUAAUGAAAACGCCGCCA | 212 |
| UCUUUCUGAGAAACUGUUCA | 213 |
| UUAGCCACUGAUUAAAUAUC | 214 |
| AGAAACUGUUCAGCUUCUGU | 215 |
| UAUCAUAAUGAAAACGCCGC | 216 |
| UUUAGCAUGUUCCCAAUUCU | 217 |
| UAUUUAGCAUGUUCCCAAUU | 218 |
| UGUCUUUCUGAGAAACUGUU | 219 |
| AUCAGUGGCUAACAGAAGCU | 220 |
| UUGAGAAAUGGCGGCGUUUU | 221 |
| AAGAUAUUUAAUCAGUGGCU | 222 |

TABLE 5 provides illustrative handle sequences for use in guide nucleic acids that are useful in the compositions, systems and methods described herein.

TABLE 5
Exemplary Handle Sequence and Portions Thereof

| Effector | sgRNA Sequences (5′-to-3′), | |
|---|---|---|
| Protein | shown as RNA | Sequence Description |
| CasM.265466 | ACAGCUUAUUUGGAAGCUGAAAUGUG | Handle sequence |
| | AGGUUUAUAACACUCACAAGAAUCCU | |
| | GAAAaaggaugccaaac (SEQ ID NO: 4) | |
| CasM.265466 | ACAGCUUAUUUGGAAGCUGAAAUGUG | Handle sequence without Linker or |
| | AGGUUUAUAACACUCACAAGAAUCCU | Repeat sequence OR intermediary |
| | (SEQ ID NO: 441) | sequence |
| CasM.265466 | GAAA (SEQ ID NO: 442) | Linker |
| CasM.265466 | aaggaugccaaac (SEQ ID NO: 443) | Repeat sequence |
| CasM.265466 | GUUUGAGAACCUUAUGAAAUUACA | Repeat sequence |
| | AGGAUGCCAAAC (SEQ ID NO: 504) | |

TABLE 6 provides illustrative guide nucleic acid sequences that are useful in the compositions, systems and methods described herein.

TABLE 6
Exemplary gRNAs

| sgRNA Sequence (5′-to-3′), shown as RNA | SEQ ID NO: |
|---|---|
| ACAGCUUAUUUGGAAGCUGAAAUGUGAGGUUUAUAACACUCACAAGAAUCC | 223 |
| UGAAAaaggaugccaaacAUACUAACCUUGGUUUCUGU | |
| ACAGCUUAUUUGGAAGCUGAAAUGUGAGGUUUAUAACACUCACAAGAAUCC | 224 |
| UGAAAaaggaugccaaacCUGUUCAUUUCAGCUUUAAC | |
| ACAGCUUAUUUGGAAGCUGAAAUGUGAGGUUUAUAACACUCACAAGAAUCC | 225 |
| UGAAAaaggaugccaaacCCACUGCACUUUAGCCUGGG | |
| ACAGCUUAUUUGGAAGCUGAAAUGUGAGGUUUAUAACACUCACAAGAAUCC | 226 |
| UGAAAaaggaugccaaacUCAAAUGUAACCAGUAUUUU | |
| ACAGCUUAUUUGGAAGCUGAAAUGUGAGGUUUAUAACACUCACAAGAAUCC | 227 |
| UGAAAaaggaugccaaacGCCUGGGUGACAGUGAGACU | |
| ACAGCUUAUUUGGAAGCUGAAAUGUGAGGUUUAUAACACUCACAAGAAUCC | 228 |
| UGAAAaaggaugccaaacAAAAGGUAUCUUUGAUACUA | |
| ACAGCUUAUUUGGAAGCUGAAAUGUGAGGUUUAUAACACUCACAAGAAUCC | 229 |
| UGAAAaaggaugccaaacACGUGAUUUUCUGUUAAUAA | |
| ACAGCUUAUUUGGAAGCUGAAAUGUGAGGUUUAUAACACUCACAAGAAUCC | 230 |
| UGAAAaaggaugccaaacCAAAGUCUACUGUUCAUUUC | |
| ACAGCUUAUUUGGAAGCUGAAAUGUGAGGUUUAUAACACUCACAAGAAUCC | 231 |
| UGAAAaaggaugccaaacAUUUUAUCAAAUGUAACCAG | |
| ACAGCUUAUUUGGAAGCUGAAAUGUGAGGUUUAUAACACUCACAAGAAUCC | 232 |
| UGAAAaaggaugccaaacUUUUCUUAGAGACAGAGUCU | |
| ACAGCUUAUUUGGAAGCUGAAAUGUGAGGUUUAUAACACUCACAAGAAUCC | 233 |
| UGAAAaaggaugccaaacCCAUAGAUUGUAAUUUAAUG | |
| ACAGCUUAUUUGGAAGCUGAAAUGUGAGGUUUAUAACACUCACAAGAAUCC | 234 |
| UGAAAaaggaugccaaacUUUAUUUUCUUAGAGACAGA | |
| ACAGCUUAUUUGGAAGCUGAAAUGUGAGGUUUAUAACACUCACAAGAAUCC | 235 |
| UGAAAaaggaugccaaacGCUAGGAUGAUGAACAACAG | |
| ACAGCUUAUUUGGAAGCUGAAAUGUGAGGUUUAUAACACUCACAAGAAUCC | 236 |
| UGAAAaaggaugccaaacGUAGUAAAUGCUAGUCUGGA | |
| ACAGCUUAUUUGGAAGCUGAAAUGUGAGGUUUAUAACACUCACAAGAAUCC | 237 |
| UGAAAaaggaugccaaacAUGGCAAAUAUUAGUUUCUG | |
| ACAGCUUAUUUGGAAGCUGAAAUGUGAGGUUUAUAACACUCACAAGAAUCC | 238 |
| UGAAAaaggaugccaaacUAGUAGUAAAUGCUAGUCUG | |
| ACAGCUUAUUUGGAAGCUGAAAUGUGAGGUUUAUAACACUCACAAGAAUCC | 239 |
| UGAAAaaggaugccaaacUUAUGGCUAGGAUGAUGAAC | |
| ACAGCUUAUUUGGAAGCUGAAAUGUGAGGUUUAUAACACUCACAAGAAUCC | 240 |
| UGAAAaaggaugccaaacGAGGAGACAUUUUAAAUGUA | |
| ACAGCUUAUUUGGAAGCUGAAAUGUGAGGUUUAUAACACUCACAAGAAUCC | 241 |
| UGAAAaaggaugccaaacAAUGUAACUUCCAAACGUUA | |
| ACAGCUUAUUUGGAAGCUGAAAUGUGAGGUUUAUAACACUCACAAGAAUCC | 242 |
| UGAAAaaggaugccaaacACUUCCAAACGUUAUCUCAC | |
| ACAGCUUAUUUGGAAGCUGAAAUGUGAGGUUUAUAACACUCACAAGAAUCC | 243 |
| UGAAAaaggaugccaaacCUUUUUUGAUGGCAAAUAUU | |
| ACAGCUUAUUUGGAAGCUGAAAUGUGAGGUUUAUAACACUCACAAGAAUCC | 244 |
| UGAAAaaggaugccaaacAGAUAACGUUUGGAAGUUAC | |
| ACAGCUUAUUUGGAAGCUGAAAUGUGAGGUUUAUAACACUCACAAGAAUCC | 245 |
| UGAAAaaggaugccaaacCCAUCAAAAAAGCAAAGAAU | |
| ACAGCUUAUUUGGAAGCUGAAAUGUGAGGUUUAUAACACUCACAAGAAUCC | 246 |
| UGAAAaaggaugccaaacAAUAAGCAACAUAAAUGUGA | |
| ACAGCUUAUUUGGAAGCUGAAAUGUGAGGUUUAUAACACUCACAAGAAUCC | 247 |
| UGAAAaaggaugccaaacGUUGAAAGAAUUCAGAAUCA | |
| ACAGCUUAUUUGGAAGCUGAAAUGUGAGGUUUAUAACACUCACAAGAAUCC | 248 |
| UGAAAaaggaugccaaacGAAGUUACAUUUAAAAUGUC | |
| ACAGCUUAUUUGGAAGCUGAAAUGUGAGGUUUAUAACACUCACAAGAAUCC | 249 |
| UGAAAaaggaugccaaacACAGAAACUAAUAUUUGCCA | |
| ACAGCUUAUUUGGAAGCUGAAAUGUGAGGUUUAUAACACUCACAAGAAUCC | 250 |
| UGAAAaaggaugccaaacAAAUGUCUCCUCCAGACUAG | |
| ACAGCUUAUUUGGAAGCUGAAAUGUGAGGUUUAUAACACUCACAAGAAUCC | 251 |
| UGAAAaaggaugccaaacUUCUAGUUGAAAGAAUUCAG | |
| ACAGCUUAUUUGGAAGCUGAAAUGUGAGGUUUAUAACACUCACAAGAAUCC | 252 |
| UGAAAaaggaugccaaacCCAACUUUUAUCAUUUUUUC | |
| ACAGCUUAUUUGGAAGCUGAAAUGUGAGGUUUAUAACACUCACAAGAAUCC | 253 |
| UGAAAaaggaugccaaacUUUGCUGAGAGAGAAACAGU | |
| ACAGCUUAUUUGGAAGCUGAAAUGUGAGGUUUAUAACACUCACAAGAAUCC | 254 |
| UGAAAaaggaugccaaacCUUAGGCUGAAUAGUGAGAG | |
| ACAGCUUAUUUGGAAGCUGAAAUGUGAGGUUUAUAACACUCACAAGAAUCC | 255 |
| UGAAAaaggaugccaaacUCAUUUUUUCUCAUACCUUC | |
| ACAGCUUAUUUGGAAGCUGAAAUGUGAGGUUUAUAACACUCACAAGAAUCC | 256 |
| UGAAAaaggaugccaaacCUUGAUGAUCAUCUCGUUGA | |
| ACAGCUUAUUUGGAAGCUGAAAUGUGAGGUUUAUAACACUCACAAGAAUCC | 257 |
| UGAAAaaggaugccaaacCUGAGAGAGAAACAGUUGCC | |
| ACAGCUUAUUUGGAAGCUGAAAUGUGAGGUUUAUAACACUCACAAGAAUCC | 258 |
| UGAAAaaggaugccaaacGCUACUUUUGUUAUUUGCAU | |
| ACAGCUUAUUUGGAAGCUGAAAUGUGAGGUUUAUAACACUCACAAGAAUCC | 259 |
| UGAAAaaggaugccaaacGGAGAGUAAAGUGAUUGGUG | |
| ACAGCUUAUUUGGAAGCUGAAAUGUGAGGUUUAUAACACUCACAAGAAUCC | 260 |
| UGAAAaaggaugccaaacUGGCUACUUUUGUUAUUUGC | |
| ACAGCUUAUUUGGAAGCUGAAAUGUGAGGUUUAUAACACUCACAAGAAUCC | 261 |
| UGAAAaaggaugccaaacAAGAAAAACUUCUGCCAACU | |
| ACAGCUUAUUUGGAAGCUGAAAUGUGAGGUUUAUAACACUCACAAGAAUCC | 262 |
| UGAAAaaggaugccaaacUAUCCUUGAUUAUACUUAGG | |
| ACAGCUUAUUUGGAAGCUGAAAUGUGAGGUUUAUAACACUCACAAGAAUCC | 263 |
| UGAAAaaggaugccaaacUCCUUGAUUAUACUUAGGCU | |
| ACAGCUUAUUUGGAAGCUGAAAUGUGAGGUUUAUAACACUCACAAGAAUCC | 264 |
| UGAAAaaggaugccaaacAAAUGAAGAUUUUCCACCAA | |
| ACAGCUUAUUUGGAAGCUGAAAUGUGAGGUUUAUAACACUCACAAGAAUCC | 265 |
| UGAAAaaggaugccaaacCUCUCCUAGACCAUUUCCCA | |
| ACAGCUUAUUUGGAAGCUGAAAUGUGAGGUUUAUAACACUCACAAGAAUCC | 266 |
| UGAAAaaggaugccaaacAGUAGGAGCUAAAAUAUUUU | |
| ACAGCUUAUUUGGAAGCUGAAAUGUGAGGUUUAUAACACUCACAAGAAUCC | 267 |
| UGAAAaaggaugccaaacGAUACUUUGUUUAGCAAUAC | |
| ACAGCUUAUUUGGAAGCUGAAAUGUGAGGUUUAUAACACUCACAAGAAUCC | 268 |
| UGAAAaaggaugccaaacGGUUUUUGCAAAAAGGAAAA | |
| ACAGCUUAUUUGGAAGCUGAAAUGUGAGGUUUAUAACACUCACAAGAAUCC | 269 |
| UGAAAaaggaugccaaacUCUUUUUCUAACAAUGUGGA | |
| ACAGCUUAUUUGGAAGCUGAAAUGUGAGGUUUAUAACACUCACAAGAAUCC | 270 |
| UGAAAaaggaugccaaacACAAUGUGGAUACUUUGUUU | |
| ACAGCUUAUUUGGAAGCUGAAAUGUGAGGUUUAUAACACUCACAAGAAUCC | 271 |
| UGAAAaaggaugccaaacAGCCAAACUCUUAUUCAUGA | |
| ACAGCUUAUUUGGAAGCUGAAAUGUGAGGUUUAUAACACUCACAAGAAUCC | 272 |
| UGAAAaaggaugccaaacAUUGAAGAGUAACAAUUUGA | |
| ACAGCUUAUUUGGAAGCUGAAAUGUGAGGUUUAUAACACUCACAAGAAUCC | 273 |
| UGAAAaaggaugccaaacGCAAUACAUGGUAGAAAAUG | |
| ACAGCUUAUUUGGAAGCUGAAAUGUGAGGUUUAUAACACUCACAAGAAUCC | 274 |
| UGAAAaaggaugccaaacCAAAAAGGAAAAAAGAAGAA | |
| ACAGCUUAUUUGGAAGCUGAAAUGUGAGGUUUAUAACACUCACAAGAAUCC | 275 |
| UGAAAaaggaugccaaacUUUAGCAAUACAUGGUAGAA | |
| ACAGCUUAUUUGGAAGCUGAAAUGUGAGGUUUAUAACACUCACAAGAAUCC | 276 |
| UGAAAaaggaugccaaacUAUCUUUUUCUAACAAUGUG | |
| ACAGCUUAUUUGGAAGCUGAAAUGUGAGGUUUAUAACACUCACAAGAAUCC | 277 |
| UGAAAaaggaugccaaacAUGUCAUGAAUAAGAGUUUG | |
| ACAGCUUAUUUGGAAGCUGAAAUGUGAGGUUUAUAACACUCACAAGAAUCC | 278 |
| UGAAAaaggaugccaaacGCUCCUACUCAGACUGUUAC | |
| ACAGCUUAUUUGGAAGCUGAAAUGUGAGGUUUAUAACACUCACAAGAAUCC | 279 |
| UGAAAaaggaugccaaacGCUUGUGUUUCUAAUUUUUC | |
| ACAGCUUAUUUGGAAGCUGAAAUGUGAGGUUUAUAACACUCACAAGAAUCC | 280 |
| UGAAAaaggaugccaaacUUGCUAAACAAAGUAUCCAC | |
| ACAGCUUAUUUGGAAGCUGAAAUGUGAGGUUUAUAACACUCACAAGAAUCC | 281 |
| UGAAAaaggaugccaaacUAAUGUCAUGAAUAAGAGUU | |
| ACAGCUUAUUUGGAAGCUGAAAUGUGAGGUUUAUAACACUCACAAGAAUCC | 282 |
| UGAAAaaggaugccaaacCCAUGUAUUGCUAAACAAAG | |
| ACAGCUUAUUUGGAAGCUGAAAUGUGAGGUUUAUAACACUCACAAGAAUCC | 283 |
| UGAAAaaggaugccaaacGCUCAAAUUGUUACUCUUCA | |
| ACAGCUUAUUUGGAAGCUGAAAUGUGAGGUUUAUAACACUCACAAGAAUCC | 284 |
| UGAAAaaggaugccaaacAGACUUUUUGCACAGUCAAU | |
| ACAGCUUAUUUGGAAGCUGAAAUGUGAGGUUUAUAACACUCACAAGAAUCC | 285 |
| UGAAAaaggaugccaaacCUUACAGGCUCCAAUAGUGG | |
| ACAGCUUAUUUGGAAGCUGAAAUGUGAGGUUUAUAACACUCACAAGAAUCC | 286 |
| UGAAAaaggaugccaaacCACAGUCAAUAACACAAAGG | |
| ACAGCUUAUUUGGAAGCUGAAAUGUGAGGUUUAUAACACUCACAAGAAUCC | 287 |
| UGAAAaaggaugccaaacGAAUUGAAACAAAUUUUCUC | |
| ACAGCUUAUUUGGAAGCUGAAAUGUGAGGUUUAUAACACUCACAAGAAUCC | 288 |
| UGAAAaaggaugccaaacUCUCUAUCUUUAGAAUUGAA | |
| ACAGCUUAUUUGGAAGCUGAAAUGUGAGGUUUAUAACACUCACAAGAAUCC | 289 |
| UGAAAaaggaugccaaacAACAAAUAGCUAGAGCCAAA | |
| ACAGCUUAUUUGGAAGCUGAAAUGUGAGGUUUAUAACACUCACAAGAAUCC | 290 |
| UGAAAaaggaugccaaacAAUCAGAGUCAAUUUCCAAG | |
| ACAGCUUAUUUGGAAGCUGAAAUGUGAGGUUUAUAACACUCACAAGAAUCC | 291 |
| UGAAAaaggaugccaaacCUCUAAGACUUUUUGCACAG | |
| ACAGCUUAUUUGGAAGCUGAAAUGUGAGGUUUAUAACACUCACAAGAAUCC | 292 |
| UGAAAaaggaugccaaacUUCAAAAGUGCAACUAUGAA | |
| ACAGCUUAUUUGGAAGCUGAAAUGUGAGGUUUAUAACACUCACAAGAAUCC | 293 |
| UGAAAaaggaugccaaacGCUCUAGCUAUUUGUUCAAA | |
| ACAGCUUAUUUGGAAGCUGAAAUGUGAGGUUUAUAACACUCACAAGAAUCC | 294 |
| UGAAAaaggaugccaaacGCUAUUUGUUCAAAAGUGCA | |
| ACAGCUUAUUUGGAAGCUGAAAUGUGAGGUUUAUAACACUCACAAGAAUCC | 295 |
| UGAAAaaggaugccaaacAGUAUACUGGAUCCCAUUCU | |
| ACAGCUUAUUUGGAAGCUGAAAUGUGAGGUUUAUAACACUCACAAGAAUCC | 296 |
| UGAAAaaggaugccaaacUGUUAUUGACUGUGCAAAAA | |
| ACAGCUUAUUUGGAAGCUGAAAUGUGAGGUUUAUAACACUCACAAGAAUCC | 297 |
| UGAAAaaggaugccaaacAUUCAAAGUGUUGCAUGACA | |
| ACAGCUUAUUUGGAAGCUGAAAUGUGAGGUUUAUAACACUCACAAGAAUCC | 298 |
| UGAAAaaggaugccaaacUUUCAAUUCUAAAGAUAGAG | |
| ACAGCUUAUUUGGAAGCUGAAAUGUGAGGUUUAUAACACUCACAAGAAUCC | 299 |
| UGAAAaaggaugccaaacAAGAUAGAGAUAAACCUUUG | |
| ACAGCUUAUUUGGAAGCUGAAAUGUGAGGUUUAUAACACUCACAAGAAUCC | 300 |
| UGAAAaaggaugccaaacUUAUUGACUGUGCAAAAAGU | |
| ACAGCUUAUUUGGAAGCUGAAAUGUGAGGUUUAUAACACUCACAAGAAUCC | 301 |
| UGAAAaaggaugccaaacAAGUGAUGACUGGGUGAGAG | |
| ACAGCUUAUUUGGAAGCUGAAAUGUGAGGUUUAUAACACUCACAAGAAUCC | 302 |
| UGAAAaaggaugccaaacCUGGAUCCCAUUCUCUUUGG | |
| ACAGCUUAUUUGGAAGCUGAAAUGUGAGGUUUAUAACACUCACAAGAAUCC | 303 |
| UGAAAaaggaugccaaacCAAAAAGUCUUAGAGUACAU | |
| ACAGCUUAUUUGGAAGCUGAAAUGUGAGGUUUAUAACACUCACAAGAAUCC | 304 |
| UGAAAaaggaugccaaacAAGAUAAUUCAUGAACAUCU | |
| ACAGCUUAUUUGGAAGCUGAAAUGUGAGGUUUAUAACACUCACAAGAAUCC | 305 |
| UGAAAaaggaugccaaacAUUAUUUUAGCCAACCACCC | |
| ACAGCUUAUUUGGAAGCUGAAAUGUGAGGUUUAUAACACUCACAAGAAUCC | 306 |
| UGAAAaaggaugccaaacCUUCUAAAUUAACUUUAGUG | |
| ACAGCUUAUUUGGAAGCUGAAAUGUGAGGUUUAUAACACUCACAAGAAUCC | 307 |
| UGAAAaaggaugccaaacAAUUAACUUUAGUGGGUAGA | |
| ACAGCUUAUUUGGAAGCUGAAAUGUGAGGUUUAUAACACUCACAAGAAUCC | 308 |
| UGAAAaaggaugccaaacGCCAACCACCCUACAAAUAU | |
| ACAGCUUAUUUGGAAGCUGAAAUGUGAGGUUUAUAACACUCACAAGAAUCC | 309 |
| UGAAAaaggaugccaaacGUGGGUAGAAUUUCUUUUAA | |
| ACAGCUUAUUUGGAAGCUGAAAUGUGAGGUUUAUAACACUCACAAGAAUCC | 310 |
| UGAAAaaggaugccaaacACAGAAAAGCAUACACAUUA | |
| ACAGCUUAUUUGGAAGCUGAAAUGUGAGGUUUAUAACACUCACAAGAAUCC | 311 |
| UGAAAaaggaugccaaacACUUCCUCUUUAACAGAAAA | |
| ACAGCUUAUUUGGAAGCUGAAAUGUGAGGUUUAUAACACUCACAAGAAUCC | 312 |
| UGAAAaaggaugccaaacAAAGAAAUUCUACCCACUAA | |
| ACAGCUUAUUUGGAAGCUGAAAUGUGAGGUUUAUAACACUCACAAGAAUCC | 313 |
| UGAAAaaggaugccaaacUAGGGUGGUUGGCUAAAAUA | |
| ACAGCUUAUUUGGAAGCUGAAAUGUGAGGUUUAUAACACUCACAAGAAUCC | 314 |
| UGAAAaaggaugccaaacUAUGCUUUUCUGUUAAAGAG | |
| ACAGCUUAUUUGGAAGCUGAAAUGUGAGGUUUAUAACACUCACAAGAAUCC | 315 |
| UGAAAaaggaugccaaacCCCACUAAAGUUAAUUUAGA | |
| ACAGCUUAUUUGGAAGCUGAAAUGUGAGGUUUAUAACACUCACAAGAAUCC | 316 |
| UGAAAaaggaugccaaacUUAAAGAGGAAGUUAGAAGA | |
| ACAGCUUAUUUGGAAGCUGAAAUGUGAGGUUUAUAACACUCACAAGAAUCC | 317 |
| UGAAAaaggaugccaaacUGCUUUUCUGUUAAAGAGGA | |
| ACAGCUUAUUUGGAAGCUGAAAUGUGAGGUUUAUAACACUCACAAGAAUCC | 318 |
| UGAAAaaggaugccaaacUUCACCAAAUGGAUUAAGAU | |
| ACAGCUUAUUUGGAAGCUGAAAUGUGAGGUUUAUAACACUCACAAGAAUCC | 319 |
| UGAAAaaggaugccaaacGGGUGGUUGGCUAAAAUAAU | |
| ACAGCUUAUUUGGAAGCUGAAAUGUGAGGUUUAUAACACUCACAAGAAUCC | 320 |
| UGAAAaaggaugccaaacCUUUUCUGUUAAAGAGGAAG | |
| ACAGCUUAUUUGGAAGCUGAAAUGUGAGGUUUAUAACACUCACAAGAAUCC | 321 |
| UGAAAaaggaugccaaacCUAAAAUGUUUUCAUUCCUA | |
| ACAGCUUAUUUGGAAGCUGAAAUGUGAGGUUUAUAACACUCACAAGAAUCC | 322 |
| UGAAAaaggaugccaaacCUGCUGUUGAUUAAUGGUUG | |
| ACAGCUUAUUUGGAAGCUGAAAUGUGAGGUUUAUAACACUCACAAGAAUCC | 323 |
| UGAAAaaggaugccaaacAUCUCUCAUGAAAUAUUCUU | |
| ACAGCUUAUUUGGAAGCUGAAAUGUGAGGUUUAUAACACUCACAAGAAUCC | 324 |
| UGAAAaaggaugccaaacAAGAAAGCUUAAAAAGUCUG | |
| ACAGCUUAUUUGGAAGCUGAAAUGUGAGGUUUAUAACACUCACAAGAAUCC | 325 |
| UGAAAaaggaugccaaacUCGCCCUACCUCUUUUUUCU | |
| ACAGCUUAUUUGGAAGCUGAAAUGUGAGGUUUAUAACACUCACAAGAAUCC | 326 |
| UGAAAaaggaugccaaacAUGUUAGUGCCUUUCACCCU | |
| ACAGCUUAUUUGGAAGCUGAAAUGUGAGGUUUAUAACACUCACAAGAAUCC | 327 |
| UGAAAaaggaugccaaacAGCAGGGUGAAAGGCACUAA | |
| ACAGCUUAUUUGGAAGCUGAAAUGUGAGGUUUAUAACACUCACAAGAAUCC | 328 |
| UGAAAaaggaugccaaacGAAGAAUAUUUCAUGAGAGA | |
| ACAGCUUAUUUGGAAGCUGAAAUGUGAGGUUUAUAACACUCACAAGAAUCC | 329 |
| UGAAAaaggaugccaaacAGCUUUCUUUAGAAGAAUAU | |
| ACAGCUUAUUUGGAAGCUGAAAUGUGAGGUUUAUAACACUCACAAGAAUCC | 330 |
| UGAAAaaggaugccaaacCUAAAAUAUAUACUUGUGGC | |
| ACAGCUUAUUUGGAAGCUGAAAUGUGAGGUUUAUAACACUCACAAGAAUCC | 331 |
| UGAAAaaggaugccaaacGCAGACUUUUUAAGCUUUCU | |
| ACAGCUUAUUUGGAAGCUGAAAUGUGAGGUUUAUAACACUCACAAGAAUCC | 332 |
| UGAAAaaggaugccaaacAAAAUUUCCCUAUGAAACUG | |
| ACAGCUUAUUUGGAAGCUGAAAUGUGAGGUUUAUAACACUCACAAGAAUCC | 333 |
| UGAAAaaggaugccaaacUUUGAGAAAAGAUUAAACAG | |
| ACAGCUUAUUUGGAAGCUGAAAUGUGAGGUUUAUAACACUCACAAGAAUCC | 334 |
| UGAAAaaggaugccaaacAGAAAAGAUUAAACAGUGUG | |
| ACAGCUUAUUUGGAAGCUGAAAUGUGAGGUUUAUAACACUCACAAGAAUCC | 335 |
| UGAAAaaggaugccaaacAGAUACCAAAAAGGCAAAAC | |
| ACAGCUUAUUUGGAAGCUGAAAUGUGAGGUUUAUAACACUCACAAGAAUCC | 336 |
| UGAAAaaggaugccaaacCUGGCAAAGAAAGAAAUACA | |
| ACAGCUUAUUUGGAAGCUGAAAUGUGAGGUUUAUAACACUCACAAGAAUCC | 337 |
| UGAAAaaggaugccaaacCUACCACAUGCAGUUGUACU | |
| ACAGCUUAUUUGGAAGCUGAAAUGUGAGGUUUAUAACACUCACAAGAAUCC | 338 |
| UGAAAaaggaugccaaacAAACUGACAUGCCCAUAUCC | |
| ACAGCUUAUUUGGAAGCUGAAAUGUGAGGUUUAUAACACUCACAAGAAUCC | 339 |
| UGAAAaaggaugccaaacUUUUGCCUUUUUGGUAUCUU | |
| ACAGCUUAUUUGGAAGCUGAAAUGUGAGGUUUAUAACACUCACAAGAAUCC | 340 |
| UGAAAaaggaugccaaacGUAGCACACUGUUUAAUCUU | |
| ACAGCUUAUUUGGAAGCUGAAAUGUGAGGUUUAUAACACUCACAAGAAUCC | 341 |
| UGAAAaaggaugccaaacUCCUUUGGAUAUGGGCAUGU | |
| ACAGCUUAUUUGGAAGCUGAAAUGUGAGGUUUAUAACACUCACAAGAAUCC | 342 |
| UGAAAaaggaugccaaacUAUUUCUUUCUUUGCCAGUA | |
| ACAGCUUAUUUGGAAGCUGAAAUGUGAGGUUUAUAACACUCACAAGAAUCC | 343 |
| UGAAAaaggaugccaaacUCUUGUAUCCUUUGGAUAUG | |
| ACAGCUUAUUUGGAAGCUGAAAUGUGAGGUUUAUAACACUCACAAGAAUCC | 344 |
| UGAAAaaggaugccaaacCCUUUUUGGUAUCUUACAGG | |
| ACAGCUUAUUUGGAAGCUGAAAUGUGAGGUUUAUAACACUCACAAGAAUCC | 345 |
| UGAAAaaggaugccaaacGAUAUGGGCAUGUCAGUUUC | |
| ACAGCUUAUUUGGAAGCUGAAAUGUGAGGUUUAUAACACUCACAAGAAUCC | 346 |
| UGAAAaaggaugccaaacGUAUCUUACAGGAACUCCAG | |
| ACAGCUUAUUUGGAAGCUGAAAUGUGAGGUUUAUAACACUCACAAGAAUCC | 347 |
| UGAAAaaggaugccaaacUUUCUUUCUUUGCCAGUACA | |
| ACAGCUUAUUUGGAAGCUGAAAUGUGAGGUUUAUAACACUCACAAGAAUCC | 348 |
| UGAAAaaggaugccaaacCCAGUACAACUGCAUGUGGU | |
| ACAGCUUAUUUGGAAGCUGAAAUGUGAGGUUUAUAACACUCACAAGAAUCC | 349 |
| UGAAAaaggaugccaaacGGCAUGUCAGUUUCAUAGGG | |
| ACAGCUUAUUUGGAAGCUGAAAUGUGAGGUUUAUAACACUCACAAGAAUCC | 350 |
| UGAAAaaggaugccaaacGUAAUAUAAUGAUGACAACA | |
| ACAGCUUAUUUGGAAGCUGAAAUGUGAGGUUUAUAACACUCACAAGAAUCC | 351 |
| UGAAAaaggaugccaaacAGAAGUUAAAGAGUCCAGAU | |
| ACAGCUUAUUUGGAAGCUGAAAUGUGAGGUUUAUAACACUCACAAGAAUCC | 352 |
| UGAAAaaggaugccaaacAUGAUGACAACAACAGUCAA | |
| ACAGCUUAUUUGGAAGCUGAAAUGUGAGGUUUAUAACACUCACAAGAAUCC | 353 |
| UGAAAaaggaugccaaacCUGAAGAUAAAUACAAUUUC | |
| ACAGCUUAUUUGGAAGCUGAAAUGUGAGGUUUAUAACACUCACAAGAAUCC | 354 |
| UGAAAaaggaugccaaacUUUAUCUUCAGCACAUCUGG | |
| ACAGCUUAUUUGGAAGCUGAAAUGUGAGGUUUAUAACACUCACAAGAAUCC | 355 |
| UGAAAaaggaugccaaacAAGGGUGAUGGAAAUUACUU | |
| ACAGCUUAUUUGGAAGCUGAAAUGUGAGGUUUAUAACACUCACAAGAAUCC | 356 |
| UGAAAaaggaugccaaacACUGUUGUUGUCAUCAUUAU | |
| ACAGCUUAUUUGGAAGCUGAAAUGUGAGGUUUAUAACACUCACAAGAAUCC | 357 |
| UGAAAaaggaugccaaacACUUCUUAAAGAUCAGGUUC | |
| ACAGCUUAUUUGGAAGCUGAAAUGUGAGGUUUAUAACACUCACAAGAAUCC | 358 |
| UGAAAaaggaugccaaacUCUUCAGCACAUCUGGACUC | |
| ACAGCUUAUUUGGAAGCUGAAAUGUGAGGUUUAUAACACUCACAAGAAUCC | 359 |
| UGAAAaaggaugccaaacGACUUUUUGUGUCAGGAUGA | |
| ACAGCUUAUUUGGAAGCUGAAAUGUGAGGUUUAUAACACUCACAAGAAUCC | 360 |
| UGAAAaaggaugccaaacGACUCUUUAACUUCUUAAAG | |
| ACAGCUUAUUUGGAAGCUGAAAUGUGAGGUUUAUAACACUCACAAGAAUCC | 361 |
| UGAAAaaggaugccaaacGUUAUACUGACAAAGAUAUC | |
| ACAGCUUAUUUGGAAGCUGAAAUGUGAGGUUUAUAACACUCACAAGAAUCC | 362 |
| UGAAAaaggaugccaaacUUUUUUGGUUAUACUGACAA | |
| ACAGCUUAUUUGGAAGCUGAAAUGUGAGGUUUAUAACACUCACAAGAAUCC | 363 |
| UGAAAaaggaugccaaacCUGACAAAGAUAUCACUCUG | |
| ACAGCUUAUUUGGAAGCUGAAAUGUGAGGUUUAUAACACUCACAAGAAUCC | 364 |
| UGAAAaaggaugccaaacGCGUAUAUUUUUUGGUUAUA | |
| ACAGCUUAUUUGGAAGCUGAAAUGUGAGGUUUAUAACACUCACAAGAAUCC | 365 |
| UGAAAaaggaugccaaacCAGAGUUUAGUUUCAAGUAA | |
| ACAGCUUAUUUGGAAGCUGAAAUGUGAGGUUUAUAACACUCACAAGAAUCC | 366 |
| UGAAAaaggaugccaaacAUAGUUUCCUGCAUUUGCAG | |
| ACAGCUUAUUUGGAAGCUGAAAUGUGAGGUUUAUAACACUCACAAGAAUCC | 367 |
| UGAAAaaggaugccaaacUCAAAUCGCCUGCAGGUAAA | |
| ACAGCUUAUUUGGAAGCUGAAAUGUGAGGUUUAUAACACUCACAAGAAUCC | 368 |
| UGAAAaaggaugccaaacGAUCAAGAAAAAUAGAUGGA | |
| ACAGCUUAUUUGGAAGCUGAAAUGUGAGGUUUAUAACACUCACAAGAAUCC | 369 |
| UGAAAaaggaugccaaacCACACCUAGCAUGUACACAC | |
| ACAGCUUAUUUGGAAGCUGAAAUGUGAGGUUUAUAACACUCACAAGAAUCC | 370 |
| UGAAAaaggaugccaaacGAGAUAUAGCGUAUAUUUUU | |
| ACAGCUUAUUUGGAAGCUGAAAUGUGAGGUUUAUAACACUCACAAGAAUCC | 371 |
| UGAAAaaggaugccaaacCCUGCAGGCGAUUUGACAGA | |
| ACAGCUUAUUUGGAAGCUGAAAUGUGAGGUUUAUAACACUCACAAGAAUCC | 372 |
| UGAAAaaggaugccaaacCGCUAUAUCUCUAUAAUCUG | |
| ACAGCUUAUUUGGAAGCUGAAAUGUGAGGUUUAUAACACUCACAAGAAUCC | 373 |
| UGAAAaaggaugccaaacUUUUUCUUGAUCCAUAUGCU | |
| ACAGCUUAUUUGGAAGCUGAAAUGUGAGGUUUAUAACACUCACAAGAAUCC | 374 |
| UGAAAaaggaugccaaacCAAAUGCAGGAAACUAUCAG | |
| ACAGCUUAUUUGGAAGCUGAAAUGUGAGGUUUAUAACACUCACAAGAAUCC | 375 |
| UGAAAaaggaugccaaacUUUGUUACUUGAAACUAAAC | |
| ACAGCUUAUUUGGAAGCUGAAAUGUGAGGUUUAUAACACUCACAAGAAUCC | 376 |
| UGAAAaaggaugccaaacUACAUGCUAGGUGUGUAUAU | |
| ACAGCUUAUUUGGAAGCUGAAAUGUGAGGUUUAUAACACUCACAAGAAUCC | 377 |
| UGAAAaaggaugccaaacUCUCUAUAAUCUGUUUUACA | |
| ACAGCUUAUUUGGAAGCUGAAAUGUGAGGUUUAUAACACUCACAAGAAUCC | 378 |
| UGAAAaaggaugccaaacUGUACAUGCUAGGUGUGUAU | |
| ACAGCUUAUUUGGAAGCUGAAAUGUGAGGUUUAUAACACUCACAAGAAUCC | 379 |
| UGAAAaaggaugccaaacCUUUUACCUGCAGGCGAUUU | |
| ACAGCUUAUUUGGAAGCUGAAAUGUGAGGUUUAUAACACUCACAAGAAUCC | 380 |
| UGAAAaaggaugccaaacUUACUUGAAACUAAACUCUG | |
| ACAGCUUAUUUGGAAGCUGAAAUGUGAGGUUUAUAACACUCACAAGAAUCC | 381 |
| UGAAAaaggaugccaaacACAGAUCUGUUGAGAAAUGG | |
| ACAGCUUAUUUGGAAGCUGAAAUGUGAGGUUUAUAACACUCACAAGAAUCC | 382 |
| UGAAAaaggaugccaaacAAAUGUUGUGUGUACAUGCU | |
| ACAGCUUAUUUGGAAGCUGAAAUGUGAGGUUUAUAACACUCACAAGAAUCC | 383 |
| UGAAAaaggaugccaaacUAAUCUGUUUUACAUAAUCC | |
| ACAGCUUAUUUGGAAGCUGAAAUGUGAGGUUUAUAACACUCACAAGAAUCC | 384 |
| UGAAAaaggaugccaaacCAUGCUAGGUGUGUAUAUUA | |
| ACAGCUUAUUUGGAAGCUGAAAUGUGAGGUUUAUAACACUCACAAGAAUCC | 385 |
| UGAAAaaggaugccaaacCAUAAUCCAUCUAUUUUUCU | |
| ACAGCUUAUUUGGAAGCUGAAAUGUGAGGUUUAUAACACUCACAAGAAUCC | 386 |
| UGAAAaaggaugccaaacAUCUGUUUUACAUAAUCCAU | |
| ACAGCUUAUUUGGAAGCUGAAAUGUGAGGUUUAUAACACUCACAAGAAUCC | 387 |
| UGAAAaaggaugccaaacACCAAAAAAUAUACGCUAUA | |
| ACAGCUUAUUUGGAAGCUGAAAUGUGAGGUUUAUAACACUCACAAGAAUCC | 388 |
| UGAAAaaggaugccaaacAUUUUCUUUUGGAUUGCAUC | |
| ACAGCUUAUUUGGAAGCUGAAAUGUGAGGUUUAUAACACUCACAAGAAUCC | 389 |
| UGAAAaaggaugccaaacUUGAAUCCUUUAACAUUUCA | |
| ACAGCUUAUUUGGAAGCUGAAAUGUGAGGUUUAUAACACUCACAAGAAUCC | 390 |
| UGAAAaaggaugccaaacGAUUGCAUCUACUGUAUAGG | |
| ACAGCUUAUUUGGAAGCUGAAAUGUGAGGUUUAUAACACUCACAAGAAUCC | 391 |
| UGAAAaaggaugccaaacACAUUUCAUUCAACUGUUGC | |
| ACAGCUUAUUUGGAAGCUGAAAUGUGAGGUUUAUAACACUCACAAGAAUCC | 392 |
| UGAAAaaggaugccaaacUAGGGACCCUCCUUCCAUGA | |
| ACAGCUUAUUUGGAAGCUGAAAUGUGAGGUUUAUAACACUCACAAGAAUCC | 393 |
| UGAAAaaggaugccaaacCUUCAUCCCACUGAUUCUGA | |
| ACAGCUUAUUUGGAAGCUGAAAUGUGAGGUUUAUAACACUCACAAGAAUCC | 394 |
| UGAAAaaggaugccaaacAAGGUGUUCUUGUACUUCAU | |
| ACAGCUUAUUUGGAAGCUGAAAUGUGAGGUUUAUAACACUCACAAGAAUCC | 395 |
| UGAAAaaggaugccaaacUGAUUUUCUUUUGGAUUGCA | |
| ACAGCUUAUUUGGAAGCUGAAAUGUGAGGUUUAUAACACUCACAAGAAUCC | 396 |
| UGAAAaaggaugccaaacGGGACCCUCCUUCCAUGACU | |
| ACAGCUUAUUUGGAAGCUGAAAUGUGAGGUUUAUAACACUCACAAGAAUCC | 397 |
| UGAAAaaggaugccaaacCUGUAUAGGGACCCUCCUUC | |
| ACAGCUUAUUUGGAAGCUGAAAUGUGAGGUUUAUAACACUCACAAGAAUCC | 398 |
| UGAAAaaggaugccaaacGCCUGUCCUAAGACCUGCUC | |
| ACAGCUUAUUUGGAAGCUGAAAUGUGAGGUUUAUAACACUCACAAGAAUCC | 399 |
| UGAAAaaggaugccaaacCAGUAGAUGCAAUCCAAAAG | |
| ACAGCUUAUUUGGAAGCUGAAAUGUGAGGUUUAUAACACUCACAAGAAUCC | 400 |
| UGAAAaaggaugccaaacUCCAAGCCCGGUUGAAAUCU | |
| ACAGCUUAUUUGGAAGCUGAAAUGUGAGGUUUAUAACACUCACAAGAAUCC | 401 |
| UGAAAaaggaugccaaacGUUUGGAGAUGGCAGUUUCC | |
| ACAGCUUAUUUGGAAGCUGAAAUGUGAGGUUUAUAACACUCACAAGAAUCC | 402 |
| UGAAAaaggaugccaaacUCACCAGAGUAACAGUCUGA | |
| ACAGCUUAUUUGGAAGCUGAAAUGUGAGGUUUAUAACACUCACAAGAAUCC | 403 |
| UGAAAaaggaugccaaacAUUUUAUAACUUGAUCAAGC | |
| ACAGCUUAUUUGGAAGCUGAAAUGUGAGGUUUAUAACACUCACAAGAAUCC | 404 |
| UGAAAaaggaugccaaacUAACUUGAUCAAGCAGAGAA | |
| ACAGCUUAUUUGGAAGCUGAAAUGUGAGGUUUAUAACACUCACAAGAAUCC | 405 |
| UGAAAaaggaugccaaacACUUGAUCAAGCAGAGAAAG | |
| ACAGCUUAUUUGGAAGCUGAAAUGUGAGGUUUAUAACACUCACAAGAAUCC | 406 |
| UGAAAaaggaugccaaacCCAGAGCAGGUACCUCCAAC | |
| ACAGCUUAUUUGGAAGCUGAAAUGUGAGGUUUAUAACACUCACAAGAAUCC | 407 |
| UGAAAaaggaugccaaacGAGAUGGCAGUUUCCUUAGU | |
| ACAGCUUAUUUGGAAGCUGAAAUGUGAGGUUUAUAACACUCACAAGAAUCC | 408 |
| UGAAAaaggaugccaaacGUUACUAAGGAAACUGCCAU | |
| ACAGCUUAUUUGGAAGCUGAAAUGUGAGGUUUAUAACACUCACAAGAAUCC | 409 |
| UGAAAaaggaugccaaacGCAGAUUUCAACCGGGCUUG | |
| ACAGCUUAUUUGGAAGCUGAAAUGUGAGGUUUAUAACACUCACAAGAAUCC | 410 |
| UGAAAaaggaugccaaacGUGACACAACCUGUGGUUAC | |
| ACAGCUUAUUUGGAAGCUGAAAUGUGAGGUUUAUAACACUCACAAGAAUCC | 411 |
| UGAAAaaggaugccaaacAAAUCACAGAGGGUGAUGGU | |
| ACAGCUUAUUUGGAAGCUGAAAUGUGAGGUUUAUAACACUCACAAGAAUCC | 412 |
| UGAAAaaggaugccaaacCUUGAUCAAGUUAUAAAAUC | |
| ACAGCUUAUUUGGAAGCUGAAAUGUGAGGUUUAUAACACUCACAAGAAUCC | 413 |
| UGAAAaaggaugccaaacCCGCCUUCCACUCAGAGCUC | |
| ACAGCUUAUUUGGAAGCUGAAAUGUGAGGUUUAUAACACUCACAAGAAUCC | 414 |
| UGAAAaaggaugccaaacCCCUCAGCUCUUGAAGUAAA | |
| ACAGCUUAUUUGGAAGCUGAAAUGUGAGGUUUAUAACACUCACAAGAAUCC | 415 |
| UGAAAaaggaugccaaacAGUGGAAGGCGGUAAACCGU | |
| ACAGCUUAUUUGGAAGCUGAAAUGUGAGGUUUAUAACACUCACAAGAAUCC | 416 |
| UGAAAaaggaugccaaacCUUCAAGAGCUGAGGGCAAA | |
| ACAGCUUAUUUGGAAGCUGAAAUGUGAGGUUUAUAACACUCACAAGAAUCC | 417 |
| UGAAAaaggaugccaaacAGCUCUGAGUGGAAGGCGGU | |
| ACAGCUUAUUUGGAAGCUGAAAUGUGAGGUUUAUAACACUCACAAGAAUCC | 418 |
| UGAAAaaggaugccaaacACAGCUGUUUGCAGACCUCC | |
| ACAGCUUAUUUGGAAGCUGAAAUGUGAGGUUUAUAACACUCACAAGAAUCC | 419 |
| UGAAAaaggaugccaaacUUUUUGAGGAUUGCUGAAUU | |
| ACAGCUUAUUUGGAAGCUGAAAUGUGAGGUUUAUAACACUCACAAGAAUCC | 420 |
| UGAAAaaggaugccaaacCAGACCUCCUGCCACCGCAG | |
| ACAGCUUAUUUGGAAGCUGAAAUGUGAGGUUUAUAACACUCACAAGAAUCC | 421 |
| UGAAAaaggaugccaaacAGGAUUGCUGAAUUAUUUCU | |
| ACAGCUUAUUUGGAAGCUGAAAUGUGAGGUUUAUAACACUCACAAGAAUCC | 422 |
| UGAAAaaggaugccaaacGAAUACUGGCAUCUGUUUUU | |
| ACAGCUUAUUUGGAAGCUGAAAUGUGAGGUUUAUAACACUCACAAGAAUCC | 423 |
| UGAAAaaggaugccaaacUCUGACAGCUGUUUGCAGAC | |
| ACAGCUUAUUUGGAAGCUGAAAUGUGAGGUUUAUAACACUCACAAGAAUCC | 424 |
| UGAAAaaggaugccaaacACAACAGUUUGCCGCUGCCC | |
| ACAGCUUAUUUGGAAGCUGAAAUGUGAGGUUUAUAACACUCACAAGAAUCC | 425 |
| UGAAAaaggaugccaaacCCGCUGCCCAAUGCCAUCCU | |
| ACAGCUUAUUUGGAAGCUGAAAUGUGAGGUUUAUAACACUCACAAGAAUCC | 426 |
| UGAAAaaggaugccaaacCAAACAGCUGUCAGACAGAA | |
| ACAGCUUAUUUGGAAGCUGAAAUGUGAGGUUUAUAACACUCACAAGAAUCC | 427 |
| UGAAAaaggaugccaaacCAGGAAAAAUUGGGAAGCCU | |
| ACAGCUUAUUUGGAAGCUGAAAUGUGAGGUUUAUAACACUCACAAGAAUCC | 428 |
| UGAAAaaggaugccaaacCGGUGGCAGGAGGUCUGCAA | |
| ACAGCUUAUUUGGAAGCUGAAAUGUGAGGUUUAUAACACUCACAAGAAUCC | 429 |
| UGAAAaaggaugccaaacGCAUGUUCCCAAUUCUCAGG | |
| ACAGCUUAUUUGGAAGCUGAAAUGUGAGGUUUAUAACACUCACAAGAAUCC | 430 |
| UGAAAaaggaugccaaacUCAUAAUGAAAACGCCGCCA | |
| ACAGCUUAUUUGGAAGCUGAAAUGUGAGGUUUAUAACACUCACAAGAAUCC | 431 |
| UGAAAaaggaugccaaacUCUUUCUGAGAAACUGUUCA | |
| ACAGCUUAUUUGGAAGCUGAAAUGUGAGGUUUAUAACACUCACAAGAAUCC | 432 |
| UGAAAaaggaugccaaacUUAGCCACUGAUUAAAUAUC | |
| ACAGCUUAUUUGGAAGCUGAAAUGUGAGGUUUAUAACACUCACAAGAAUCC | 433 |
| UGAAAaaggaugccaaacAGAAACUGUUCAGCUUCUGU | |
| ACAGCUUAUUUGGAAGCUGAAAUGUGAGGUUUAUAACACUCACAAGAAUCC | 434 |
| UGAAAaaggaugccaaacUAUCAUAAUGAAAACGCCGC | |
| ACAGCUUAUUUGGAAGCUGAAAUGUGAGGUUUAUAACACUCACAAGAAUCC | 435 |
| UGAAAaaggaugccaaacUUUAGCAUGUUCCCAAUUCU | |
| ACAGCUUAUUUGGAAGCUGAAAUGUGAGGUUUAUAACACUCACAAGAAUCC | 436 |
| UGAAAaaggaugccaaacUAUUUAGCAUGUUCCCAAUU | |
| ACAGCUUAUUUGGAAGCUGAAAUGUGAGGUUUAUAACACUCACAAGAAUCC | 437 |
| UGAAAaaggaugccaaacUGUCUUUCUGAGAAACUGUU | |
| ACAGCUUAUUUGGAAGCUGAAAUGUGAGGUUUAUAACACUCACAAGAAUCC | 438 |
| UGAAAaaggaugccaaacAUCAGUGGCUAACAGAAGCU | |
| ACAGCUUAUUUGGAAGCUGAAAUGUGAGGUUUAUAACACUCACAAGAAUCC | 439 |
| UGAAAaaggaugccaaacUUGAGAAAUGGCGGCGUUUU | |
| ACAGCUUAUUUGGAAGCUGAAAUGUGAGGUUUAUAACACUCACAAGAAUCC | 440 |
| UGAAAaaggaugccaaacAAGAUAUUUAAUCAGUGGCU | |

*In italics is a handle sequence without a linker or repeat sequence, in bold is a linker, lowercase is a repeat sequence, and no formatting is a spacer sequence.

TABLE 7 provides exemplary genes that are useful in the compositions, systems and methods described herein.

TABLE 7
Certain exemplary genes
Certain exemplary genes
DMD (also known as: BMD, CMD3B, MRX85, DXS142, DXS164,
DXS206, DXS230, DXS239, DXS268, DXS269, DXS270, DXS272)

TABLE 8 provides exemplary exons that are useful in the compositions, systems and methods described herein.

TABLE 8
Certain exemplary exons

| Exon No. | Start | End |
|---|---|---|
| 1 | 33,211,549 | 33,211,282 |
| 2 | 33,020,200 | 33,020,139 |
| 3 | 32,849,820 | 32,849,728 |
| 4 | 32,844,860 | 32,844,783 |
| 5 | 32,823,387 | 32,823,295 |
| 6 | 32,816,640 | 32,816,468 |
| 7 | 32,809,611 | 32,809,493 |
| 8 | 32,699,293 | 32,699,112 |
| 9 | 32,697,998 | 32,697,870 |
| 10 | 32,645,152 | 32,644,964 |
| 11 | 32,644,313 | 32,644,132 |
| 12 | 32,614,453 | 32,614,303 |
| 13 | 32,595,876 | 32,595,757 |
| 14 | 32,573,846 | 32,573,745 |
| 15 | 32,573,637 | 32,573,530 |
| 16 | 32,565,881 | 32,565,702 |
| 17 | 32,545,334 | 32,545,159 |
| 18 | 32,518,131 | 32,518,008 |
| 19 | 32,501,842 | 32,501,755 |
| 20 | 32,491,518 | 32,491,277 |
| 21 | 32,485,099 | 32,484,919 |
| 22 | 32,472,309 | 32,472,164 |
| 23 | 32,468,710 | 32,468,498 |
| 24 | 32,464,699 | 32,464,586 |
| 25 | 32,463,594 | 32,463,439 |
| 26 | 32,454,832 | 32,454,662 |
| 27 | 32,448,638 | 32,448,456 |
| 28 | 32,441,314 | 32,441,180 |
| 29 | 32,438,390 | 32,438,241 |
| 30 | 32,411,913 | 32,411,752 |
| 31 | 32,390,181 | 32,390,071 |
| 32 | 32,389,674 | 32,389,501 |
| 33 | 32,386,465 | 32,386,310 |
| 34 | 32,380,680 | 32,380,510 |
| 35 | 32,365,199 | 32,365,020 |
| 36 | 32,364,710 | 32,364,582 |
| 37 | 32,362,958 | 32,362,788 |
| 38 | 32,348,528 | 32,348,406 |
| 39 | 32,346,080 | 32,345,943 |
| 40 | 32,343,286 | 32,343,134 |
| 41 | 32,342,282 | 32,342,100 |
| 42 | 32,310,276 | 32,310,082 |
| 43 | 32,287,701 | 32,287,529 |
| 44 | 32,217,063 | 32,216,916 |
| 45 | 31,968,514 | 31,968,339 |
| 46 | 31,932,227 | 31,932,080 |
| 47 | 31,929,745 | 31,929,596 |
| 48 | 31,875,373 | 31,875,188 |
| 49 | 31,836,819 | 31,836,718 |
| 50 | 31,820,083 | 31,819,975 |
| 51 | 31,774,192 | 31,773,960 |
| 52 | 31,729,748 | 31,729,631 |
| 53 | 31,679,586 | 31,679,375 |
| 54 | 31,658,144 | 31,657,990 |
| 55 | 31,627,862 | 31,627,673 |
| 56 | 31,507,453 | 31,507,281 |
| 57 | 31,496,944 | 31,496,788 |
| 58 | 31,479,103 | 31,478,983 |
| 59 | 31,478,374 | 31,478,106 |
| 60 | 31,444,627 | 31,444,481 |
| 61 | 31,348,634 | 31,348,556 |
| 62 | 31,323,658 | 31,323,598 |
| 63 | 31,261,016 | 31,260,955 |
| 64 | 31,223,121 | 31,223,047 |
| 65 | 31,209,699 | 31,209,498 |
| 66 | 31,206,667 | 31,206,582 |
| 67 | 31,204,118 | 31,203,961 |
| 68 | 31,182,904 | 31,182,738 |
| 69 | 31,180,481 | 31,180,370 |
| 70 | 31,178,805 | 31,178,669 |
| 71 | 31,177,970 | 31,177,932 |
| 72 | 31,173,604 | 31,173,539 |
| 73 | 31,172,413 | 31,172,348 |
| 74 | 31,169,601 | 31,169,443 |
| 75 | 31,147,518 | 31,147,275 |
| 76 | 31,146,414 | 31,146,291 |
| 77 | 31,134,194 | 31,134,102 |
| 78 | 31,126,673 | 31,126,642 |
| 79 | 31,121,930 | 31,119,222 |

TABLE 9 provides exemplary genomic exons that are useful in the compositions, systems and methods described herein.

TABLE 9
Certain Exemplary Genomic Exon Sequences

| Exon | | SEQ ID |
|---|---|---|
| No. | Exon Sequence | NO: |
| 44 | CTTAAGATACCATTTGTATTTAGCATGTTCCCAATTCTCAGGAATTTGTG | 451 |
| | TCTTTCTGAGAAACTGTTCAGCTTCTGTTAGCCACTGATTAAATATCTTT | |
| | ATATCATAATGAAAACGCCGCCATTTCTCAACAGATCTGTCAAATCGC | |
| 45 | GAACTCCAGGATGGCATTGGGCAGCGGCAAACTGTTGTCAGAACATTGA | 452 |
| | ATGCAACTGGGGAAGAAATAATTCAGCAATCCTCAAAAACAGATGCCA | |
| | GTATTCTACAGGAAAAATTGGGAAGCCTGAATCTGCGGTGGCAGGAGGT | |
| | CTGCAAACAGCTGTCAGACAGAAAAAAGAG | |
| 50 | AGGAAGTTAGAAGATCTGAGCTCTGAGTGGAAGGCGGTAAACCGTTTAC | 453 |
| | TTCAAGAGCTGAGGGCAAAGCAGCCTGACCTAGCTCCTGGACTGACCAC | |
| | TATTGGAGCCT | |
| 51 | CTCCTACTCAGACTGTTACTCTGGTGACACAACCTGTGGTTACTAAGGA | 454 |
| | AACTGCCATCTCCAAACTAGAAATGCCATCTTCCTTGATGTTGGAGGTA | |
| | CCTGCTCTGGCAGATTTCAACCGGGCTTGGACAGAACTTACCGACTGGC | |
| | TTTCTCTGCTTGATCAAGTTATAAAATCACAGAGGGTGATGGTGGGTGA | |
| | CCTTGAGGATATCAACGAGATGATCATCAAGCAGAAG | |
| 53 | TTGAAAGAATTCAGAATCAGTGGGATGAAGTACAAGAACACCTTCAGA | 455 |
| | ACCGGAGGCAACAGTTGAATGAAATGTTAAAGGATTCAACACAATGGC | |
| | TGGAAGCTAAGGAAGAAGCTGAGCAGGTCTTAGGACAGGCCAGAGCCA | |
| | AGCTTGAGTCATGGAAGGAGGGTCCCTATACAGTAGATGCAATCCAAAA | |
| | GAAAATCACAGAAACCAAG | |

TABLE 10 provides exemplary mutations that can be targeted by the compositions, systems and methods described herein.

TABLE 10
Certain exemplary mutations

| Exemplary Mutation Expression | Exemplary Target Gene Region |
|---|---|
| DMD exon 48-50 deletions | DMD exon 51 |
| and DMD nonsense mutation in | |
| exon 51 | |
| DMD exon 45-52 deletions | DMD exon 53 |
| DMD exon 44 deletion | DMD exon 45 |
| DMD nonsense mutation in exon 23 | DMD exon 23 |
| DMD exon 48-50 deletions DMD | DMD exon 51 |
| nonsense mutation in exon 51 | |
| DMD nonsense mutation in exon 23 | DMD intron 22 and 23 |
| DMD exon 46-51 deletions DMD | DMD intron 44 and 55 |
| exon 46-47 deletions | |
| DMD nonsense mutation in exon 23 | DMD intron 22 and 23 |
| DMD nonsense mutation in exon 23 | DMD exon 23 |
| DMD point mutation in intron 47, | DMD exon 47A, exon 51 |
| exon 51 | |
| DMD exon 44 deletion | DMD splice site of exon 43 or |
| | exon 45 |
| DMD nonsense mutation in exon 53 | DMD splice acceptor site of |
| | exon 53 |
| DMD exon 50 deletion | DMD splice acceptor site of |
| | exon 51 |
| DMD exon 50 deletion | DMD splice acceptor site of |
| | exon 51 |
| DMD nonsense mutation in exon 23 | DMD exon 23 |
| DMD nonsense mutation in exon 23 | DMD exon 23 |
| DMD nonsense mutation in exon 53 | DMD exon 53 |
| DMD exon 44 deletion | DMD exon 44 |
| DMD exon 7 skipping | DMD splice acceptor site of |
| | intron 6 and exon 7 boundary |
| DMD nonsense mutation in exon 23 | DMD exon 23 |
| DMD exon 51 deletion | DMD splice site of exon 50 |
| DMD nonsense mutation in exon 20 | DMD exon 20 |
| DMD exon 45-52 deletions | UTRN A, B promoter |
| DMD nonsense mutation in exon 23 | Lamal promoter |
| DMD nonsense mutation in exon 23 | klotho and Utrn |
| DMD exon 46-51 deletions | 3' UTR of UTRN inhibitory |
| | miRNA target region |
| epigenetic dysregulation of DUX4 | DUX4 promoter or DUX4 exon 1 |

TABLE 11 provides exemplary diseases that can be targeted by the compositions, systems and methods described herein.

TABLE 11
Exemplary diseases

Muscular Dystrophy (MD); Muscular Dystrophy, Duchenne Type (DMD); Dilated Cardiomyopathy
(DCM) Type 3B; Muscular Dystrophy; Muscular Dystrophy, Becker Type (BMD); Dystrophinopathies;
Familial Isolated Dilated Cardiomyopathy; Dilated Cardiomyopathy; Myopathy; Colorectal Cancer;
Isolated Elevated Serum Creatine Phosphokinase Levels; Atrial Standstill 1; Creatine Phosphokinase,
Elevated Serum; Neuromuscular Disease; Atrial Heart Septal Defect; Arrhythmogenic Right Ventricular
Cardiomyopathy; Heart Disease; Glycerol Kinase Deficiency; Non-Syndromic X-Linked Intellectual
Disability; Respiratory Failure; Rhabdomyosarcoma; Miyoshi Muscular Dystrophy; Scoliosis;
Facioscapulohumeral Muscular Dystrophy 1; Ptosis; Hypertrophic Cardiomyopathy; Schizophrenia;
Myositis; Autism; Myocarditis; Muscular Dystrophy, Limb-Girdle, Autosomal Recessive 2; Adrenal
Hypoplasia, Congenital; Autosomal Recessive Limb-Girdle Muscular Dystrophy; Restrictive
Cardiomyopathy; Walker-Warburg Syndrome; Muscular Dystrophy, Congenital, Lmna-Related;
Centronuclear Myopathy; Cataract; Spinal Muscular Atrophy; Long Qt Syndrome; Emery-Dreifuss
Muscular Dystrophy; Retinitis Pigmentosa; Malignant Hyperthermia; Pectus Excavatum; Brugada
Syndrome; Myoglobinuria; Muscular Dystrophy-Dystroglycanopathy, Type a, 4; Muscle Hypertrophy;
Cardiomyopathy, Familial Hypertrophic, 1; Batten-Turner Congenital Myopathy; Bethlem Myopathy 1;
Eye Disease; Aland Island Eye Disease; Glycogen Storage Disease II; Left Ventricular Noncompaction;
Glycogen Storage Disease; Brody Myopathy; Myofibrillar Myopathy; Beckwith-Wiedemann Syndrome;
Polyglucosan Body Myopathy 1 with or Without Immunodeficiency; Interatrial Communication;
Congenital Fiber-Type Disproportion; Chromosome Xp21 Deletion Syndrome; Multiple Pterygium
Syndrome, Escobar Variant; Lissencephaly; Rigid Spine Muscular Dystrophy 1; Hypertrophic Pyloric
Stenosis; Progressive Muscular Dystrophy; X-Linked Recessive Disease; Muscular Disease; Disease of
Mental Health; Exophthalmos; Gas Gangrene; Symptomatic Form of Muscular Dystrophy of Duchenne
and Becker in Female Carriers; Muscular Atrophy; Qualitative or Quantitative Defects of Dystrophin;
Muscular Dystrophy, Congenital Merosin-Deficient, 1a; Autosomal Recessive Limb-Girdle Muscular
Dystrophy Type 2c; Mcleod Syndrome; Muscular Dystrophy, Duchenne and Becker Type; Autosomal
Recessive Limb-Girdle Muscular Dystrophy Type 2d; Ullrich Congenital Muscular Dystrophy 1; Nr0b1-
Related Adrenal Hypoplasia Congenita; Waardenburg Syndrome, Type 4b; Nonaka Myopathy; Intrinsic
Cardiomyopathy; Autosomal Recessive Limb-Girdle Muscular Dystrophy Type 2b; Myotonic
Dystrophy 1; Muscular Dystrophy, Limb-Girdle, Autosomal Recessive 6; Emery-Dreifuss Muscular
Dystrophy 2, Autosomal Dominant; Muscular Dystrophy, Limb-Girdle, Autosomal Recessive 7;
Myopathy, Myofibrillar, 3; Myopathy, Myofibrillar, 5; Myopathy, Myofibrillar, 1; Peripheral Nervous
System Disease; Muscle Eye Brain Disease; Cardiomyopathy, Familial Hypertrophic, 4; Microcolon;
Hemophagocytic Lymphohistiocytosis, Familial, 1; Autosomal Recessive Limb-Girdle Muscular
Dystrophy Type 2a; Tibial Muscular Dystrophy; Congenital Muscular Dystrophy-Dystroglycanopathy
Type a; Bone Structure Disease; Autosomal Recessive Limb-Girdle Muscular Dystrophy Type 2f;
Immunodeficiency 26; Oculomedin; Cardioneuromyopathy with Hyaline Masses and Nemaline Rods;
Keratosis Follicularis Spinulosa Decalvans, X-Linked; Myoglobinuria, Recurrent; Muscular Dystrophy-
Dystroglycanopathy; X-Linked Monogenic Disease; Muscle Tissue Disease; Fundus Dystrophy;
Interstitial Myocarditis; Localized Lipodystrophy; Extracardiac Rhabdomyoma; Cytoplasmic Body
Myopathy; Autosomal Dominant Distal Myopathy; Reducing Body Myopathy; Cobblestone
Lissencephaly; Multiple Sclerosis; Barrett Esophagus; Gastric Cancer, Hereditary Diffuse; Colorectal
Cancer 12; Polyposis Syndrome, Hereditary Mixed, 1; Small Intestine Adenocarcinoma; Esophageal
Cancer; Esophagus Squamous Cell Carcinoma; Cardiomyopathy, Dilated, 1b; Limb-Girdle Muscular
Dystrophy; 48, xyyy; 48, xxxy; 48, xxyy; Alacrima, Achalasia, and Mental Retardation Syndrome;
49, xyyyy; 49, xxxxx; 49, xxxxy; 47, xyy; Lmna-Related Dilated Cardiomyopathy; Lung Combined Type
Small Cell Carcinoma; Lung Occult Small Cell Carcinoma; Lung Non-Squamous Non-Small Cell
Carcinoma; Cardiomyopathy, Dilated, 1a; Cardiomyopathy, Dilated, 1h; Cardiac Conduction Defect;
Meningioma, Familial; Congestive Heart Failure; Myotonic Dystrophy; Breast Cancer; Severe
Combined Immunodeficiency; Dysphagia; Fibrosis of Extraocular Muscles, Congenital, 1; Muscular
Dystrophy, Limb-Girdle, Autosomal Recessive 5; Autism Spectrum Disorder; Polykaryocytosis Inducer;
Progressive Familial Heart Block; Heart Conduction Disease; Relapsing-Remitting Multiple Sclerosis;
Colon Adenocarcinoma; Glioblastoma; Encephalopathy, Progressive, Early-Onset, with Episodic
Rhabdomyolysis; Metabolic Crises, Recurrent, with Rhabdomyolysis, Cardiac Arrhythmias, and
Neurodegeneration; Attention Deficit-Hyperactivity Disorder; Neuroretinitis; Retinitis; Learning
Disability; Secondary Progressive Multiple Sclerosis; Myotonia Congenita, Autosomal Recessive;
Endomyocardial Fibrosis; Pik3ca-Related Overgrowth Syndrome; Genetic Neuromuscular Disease;
Short Stature-Obesity Syndrome; Hypoadrenocorticism, Familial; Cleft Palate, Isolated; Osteoporosis;
Bone Mineral Density Quantitative Trait Locus 8; Bone Mineral Density Quantitative Trait Locus 15;
Gonadal Dysgenesis; Turner Syndrome; Hypoxia; Phenylketonuria; Brugada Syndrome 1; Neuronal
Migration Disorders; Cardiac Arrest; Hypotonia; Amyotrophic Lateral Sclerosis 1; Glioma Susceptibility
1; Lateral Sclerosis; Corneal Edema; Polymyositis; Sleep Disorder; Cleft Lip; Muscular Dystrophy,
Limb-Girdle, Autosomal Recessive 1; Glioma Susceptibility 9; Glioma Susceptibility 2; Glioma
Susceptibility 3; Pilocytic Astrocytoma; Diarrhea; Hemophilia; Chronic Granulomatous Disease; Cleft
Lip with or Without Cleft Palate; Type 2 Diabetes Mellitus; Triiodothyronine Receptor Auxiliary
Protein; Macroglossia; Melanoma; Orofaciodigital Syndrome I; Orofaciodigital Syndrome; Aging;
Rapidly Involuting Congenital Hemangioma; Sensorineural Hearing Loss; Yemenite Deaf-Blind
Hypopigmentation Syndrome; Toxic Encephalopathy; West Syndrome; Gastrointestinal Stromal Tumor;
Osteogenic Sarcoma; Skeletal Muscle Disease; Intracranial Meningioma; Muscular Dystrophy-
Dystroglycanopathy, Type C, 5; Ataxia, Combined Cerebellar and Peripheral, with Hearing Loss and
Diabetes Mellitus; Branchiootic Syndrome 1; Deafness, X-Linked 3; Secretory Meningioma;
Lymphoplasmacyte-Rich Meningioma; Factor Viii Deficiency; Hemophilia a; Dermatomyositis;
Calpain-3-Related Limb-Girdle Muscular Dystrophy R1; Qualitative or Quantitative Defects of Alpha-
Dystroglycan; Congenital Muscular Dystrophy Due to Dystroglycanopathy; Growth Hormone
Deficiency; Cleft Lip/palate; Parkinsonism; Microcephaly; Cerebral Palsy; Osteomalacia; Bosma
Arhinia Microphthalmia Syndrome; Intraocular Pressure Quantitative Trait Locus; Combined
Immunodeficiency; Maple Syrup Urine Disease; Papillomatosis, Confluent and Reticulated; Peutz-
Jeghers Syndrome; Rippling Muscle Disease 2; Muscular Dystrophy-Dystroglycanopathy, Type B, 5;
Nonsyndromic 46, xx Testicular Disorders of Sex Development; Hand Skill, Relative; Orofacial Cleft;
Retinal Detachment; Constipation; Sarcoma; Spindle Cell Sarcoma; Premature Menopause; Sleep
Apnea; Dysferlinopathy; Qualitative or Quantitative Defects of Dysferlin; Autonomic Dysfunction;
Graft-Versus-Host Disease; Microphthalmia, Syndromic 10; Chondroblastoma; Bone Mineral Density
Quantitative Trait Locus 3; Leukemia, Acute Lymphoblastic; Methane Production; Ischemia; Idiopathic
Scoliosis; Alcohol Dependence; Premature Ovarian Failure 1; Demyelinating Disease; Qualitative or
Quantitative Defects of Sarcoglycan; Mitral Valve Insufficiency; Myopathy, X-Linked, with Excessive
Autophagy; Mucositis; Inclusion Body Myositis; Dystonia; Bone Resorption Disease; Body Mass Index
Quantitative Trait Locus 1; Neuritis; Cystic Fibrosis; Polycystic Kidney Disease; Charcot-Marie-Tooth
Disease; Myasthenia Gravis; Helix Syndrome; Hyperinsulinism; Lipid Metabolism Disorder; Tooth
Disease; Lung Disease; Muscular Dystrophy, Limb-Girdle, Autosomal Recessive 3; Miyoshi Muscular
Dystrophy 1; Pseudohyperkalemia, Familial, 2, Due to Red Cell Leak; Urinary Tract Infection; Early-
Onset Generalized Limb-Onset Dystonia; Multinucleated Neurons, Anhydramnios, Renal Dysplasia,
Cerebellar Hypoplasia, and Hydranencephaly; Agenesis of Corpus Callosum, Cardiac, Ocular, and
Genital Syndrome; Pneumothorax; Proteasome-Associated Autoinflammatory Syndrome 1; Pancreatic
Cancer; Gastric Cancer; Neuromyelitis Optica; Open-Angle Glaucoma; Disease by Infectious Agent;
Resting Heart Rate, Variation in; Poliomyelitis; Childhood Type Dermatomyositis; Progressive
Multifocal Leukoencephalopathy; Swallowing Disorders; Premature Aging; Rickets; Hirschsprung
Disease, Cardiac Defects, and Autonomic Dysfunction; Optic Neuritis; Progressive Muscular Atrophy;
Spinal Muscular Atrophy, Type Ii; Glucose Intolerance; Nephrolithiasis; Hypogonadism; Motor Neuron
Disease; Congenital Muscular Dystrophy Type 1a; Autoimmune Disease; Atrioventricular Block;
Glucocorticoid-Induced Osteoporosis; Epilepsy; Back Pain; Fragile X Syndrome; B-Lymphoblastic
Leukemia/lymphoma; Mitochondrial Myopathy; Dyslexia; Ataxia-Telangiectasia; Obsessive-
Compulsive Disorder; Torticollis; Proteinuria, Chronic Benign; Pulmonary Fibrosis; Myotonia;
Metabolic Acidosis; Brittle Bone Disorder; Scoliosis, Isolated 1; Vascular Disease; Bilirubin Metabolic
Disorder; Night Blindness; Chromosomal Triplication; Dentinogenesis Imperfecta Type 2; Astigmatism;
Severe Acute Respiratory Syndrome; Telangiectasis; Skin Disease; Microphthalmia; Myopathy, Distal,
with Anterior Tibial Onset; Hyperhomocysteinemia; Congenital Stationary Night Blindness;
Hypothyroidism; Mitral Valve Disease; Leiomyosarcoma; Nemaline Myopathy; Distal Muscular
Dystrophy with Anterior Tibial Onset; Diabetes Mellitus; Influenza; Herpes Simplex; Juvenile
Rheumatoid Arthritis; Central Sleep Apnea; Homocysteinemia; Mitochondrial Disorders; Nervous
System Disease; Keratoconus; Nasopharyngitis; Glaucoma, Primary Open Angle; Central Centrifugal
Cicatricial Alopecia; Anxiety; Dermatitis, Atopic; Aphasia; Sexual Disorder; Acute Cystitis; Dermatitis;
Kidney Disease; Tetanus; Myopia; Hypokalemia; Spinal Cord Injury; Cyanide Poisoning; Cardiogenic
Shock; Huntington Disease; Spinal Muscular Atrophy, Type I; Colitis; Down Syndrome; Hair Whorl;
Achondroplasia; Apnea, Obstructive Sleep; Barth Syndrome; Autosomal Recessive Disease; Hepatitis
B; Movement Disease; Hemosiderosis; Hepatitis; Gastric Dilatation; Teratoma; Gastroparesis;
Progressive Familial Heart Block, Type Ia; Chondroma; Neurofibromatosis; Hyperthyroidism;
Enchondroma; Pulmonary Embolism; Hypoglycemia; Rare Hereditary Hemochromatosis; Paresthesia;
Charcot-Marie-Tooth Disease, Demyelinating, Type 1a; Keratitis, Hereditary; Insulin-Like Growth
Factor I; Drug Allergy; Body Mass Index Quantitative Trait Locus 8; Body Mass Index Quantitative
Trait Locus 14; Body Mass Index Quantitative Trait Locus 18; Body Mass Index Quantitative Trait
Locus 7; Body Mass Index Quantitative Trait Locus 4; Body Mass Index Quantitative Trait Locus 10;
Orthostatic Intolerance; Body Mass Index Quantitative Trait Locus 12; Vitreoretinopathy, Neovascular
Inflammatory; Abetalipoproteinemia; Body Mass Index Quantitative Trait Locus 11; Body Mass Index
Quantitative Trait Locus 9; Ichthyosis, X-Linked; Lymphoma; Ocular Albinism; Cervical Dystonia;
Uveitis; Hydrocephalus; Liver Cirrhosis; Acute Myocarditis; Skin Carcinoma; Ichthyosis; Hypertensive
Heart Disease; Hypogonadotropic Hypogonadism; Hepatocellular Carcinoma; Body Mass Index
Quantitative Trait Locus 19; Pulmonary Hypertension; Albinism; B-Cell Lymphoma; Allergic
Encephalomyelitis; Cytokine Deficiency; Vitreoretinopathy; Prader-Willi Syndrome; Oculopharyngeal
Muscular Dystrophy; Neurodegeneration with Brain Iron Accumulation 2a; Spondylometaphyseal
Dysplasia, Sedaghatian Type; Aspiration Pneumonia; Human Immunodeficiency Virus Type 1; Arcus
Corneae; Taqi Polymorphism; Inflammatory Bowel Disease; Epidermolysis Bullosa; Temporal Lobe
Epilepsy; Blepharospasm; Corneal Neovascularization; Neuroaxonal Dystrophy; Neurodevelopmental,
Jaw, Eye, and Digital Syndrome; Blistering, Acantholytic, of Oral and Laryngeal Mucosa; Collagen Vi-
Related Dystrophies; Waardenburg's Syndrome; Fasting Hypoglycemia; X-Linked Congenital
Stationary Night Blindness; Malignant Hyperthermia Susceptibility; 48, Xxxx; Tremor; Aneurysm;
Chronic Pain; Thalassemia; Leukemia, Chronic Lymphocytic; Netherton Syndrome; Sjogren Syndrome;
Perlman Syndrome; Rothmund-Thomson Syndrome, Type 2; Clostridium Difficile Colitis; Covid-19;
Pulmonary Fibrosis, Idiopathic; Muscular Dystrophy, Limb-Girdle, Autosomal Recessive 4;
Poikiloderma with Neutropenia; Myopathy, Proximal, with Ophthalmoplegia; Glucocorticoid
Resistance, Generalized; Alstrom Syndrome; Intellectual Developmental Disorder, X-Linked 21;
Cognitive Function 1, Social; Medulloblastoma; Danon Disease; Limb Ischemia; Dementia;
Conjunctivitis; Neutropenia; Ehlers-Danlos Syndrome; Polyneuropathy; Phaeohyphomycosis; Vaginal
Discharge; Hyperglycemia; Bullous Keratopathy; Keratopathy; Acute Disseminated Encephalomyelitis;
Thyroiditis; Soft Tissue Sarcoma; Pathologic Nystagmus; Pachygyria; Depression; Overgrowth
Syndrome; Optic Atrophy 1; Muscular Dystrophy-Dystroglycanopathy, Type a, 1; Hypertriglyceridemia
1; Schwartz-Jampel Syndrome, Type 1; Moyamoya Disease 1; Left Bundle Branch Hemiblock; Usher
Syndrome; Thrombotic Thrombocytopenia Purpura; Neural Tube Defects; Fibrodysplasia Ossificans
Progressiva; Gastroesophageal Reflux; Meniere Disease; Diamond-Blackfan Anemia 2; Night
Blindness, Congenital Stationary, Type 1a; Retinoschisis 1, X-Linked, Juvenile; Retinitis Pigmentosa-
Deafness Syndrome; Type 1 Diabetes Mellitus; Acute Kidney Failure; Leukemia; Epidermolysis Bullosa
Dystrophica; Pneumonia; Osteochondrodysplasia; Purpura; Interstitial Lung Disease; Heart Septal
Defect; Compartment Syndrome; Acne; Adenoma; Ileus; Chronic Kidney Disease; Mitochondrial
Encephalomyopathy; Measles; Ltbp4-Related Cutis Laxa; Spinocerebellar Degeneration; Nonsyndromic
Hearing Loss; Primary Adrenal Insufficiency; Leukemia, Chronic Lymphocytic 2;
Pseudoachondroplasia; Blood Group -- Kell System; Cardiomyopathy, Familial Hypertrophic, 2; Central
Core Disease of Muscle; Rhabdomyosarcoma 2; Human Cytomegalovirus Infection; Frontometaphyseal
Dysplasia; Cholelithiasis; Congenital Hypothyroidism; Hypophosphatemia; Juvenile Glaucoma;
Epicanthus; Exudative Vitreoretinopathy 1; Charcot-Marie-Tooth Disease, Axonal, Type 2e; Progressive
Familial Heart Block, Type Ib; Chorea, Childhood-Onset, with Psychomotor Retardation; Myopathy,
Distal, with Rimmed Vacuoles; Deafness, X-Linked 1; Frontometaphyseal Dysplasia 1; Budd-Chiari
Syndrome; Myocardial Infarction; Ectodermal Dysplasia-Syndactyly Syndrome 2; Alpha/beta T-Cell
Lymphopenia with Gamma/delta T-Cell Expansion, Severe Cytomegalovirus Infection, and
Autoimmunity; Cardiomyopathy, Dilated, 1x; Apnea, Central Sleep; Adrenal Hyperplasia, Congenital,
Due to 21-Hydroxylase Deficiency; Thyroid Carcinoma, Familial Medullary; Intellectual Developmental
Disorder, X-Linked 29; Orofaciodigital Syndrome Viii; Myopathy, Centronuclear, X-Linked;
Progressive Relapsing Multiple Sclerosis; Duane Retraction Syndrome; Choreatic Disease;
Hemopericardium; Cardiac Tamponade; Right Bundle Branch Block; Candidiasis;
Pseudohermaphroditism; Laryngitis; Multiple Endocrine Neoplasia; Tricuspid Valve Insufficiency;
Mouth Disease; Superior Mesenteric Artery Syndrome; Histiocytosis; Pericardial Effusion; Clubfoot;
Testicular Disease; Thyroid Gland Medullary Carcinoma; Cerebrovascular Disease; Mitochondrial
Metabolism Disease; Hypertrichosis; Acute Myocardial Infarction; Skin Melanoma; Dyskinesia of
Esophagus; Dysautonomia; Congenital Hydrocephalus; Athetosis; Progeroid Syndrome; Muscular
Lipidosis; Laminin Subunit Alpha 2-Related Congenital Muscular Dystrophy; Cerebrofacial
Arteriovenous Metameric Syndrome; Isolated Duane Retraction Syndrome; Thyroid Carcinoma;
Arteries, Anomalies of; Lipoid Congenital Adrenal Hyperplasia; Multiple System Atrophy 1; Chediak-
Higashi Syndrome; Pneumothorax, Primary Spontaneous; Lymphoproliferative Syndrome; Pre-
Eclampsia; Myelodysplastic Syndrome; Familial Adenomatous Polyposis; Myopathy, Lactic Acidosis,
and Sideroblastic Anemia; Hashimoto Thyroiditis; Muscular Dystrophy-Dystroglycanopathy, Type C,
4; 46, xy Sex Reversal 2; Cardiomyopathy, Dilated, 1g; Sickle Cell Anemia; Muscular Dystrophy, Limb-
Girdle, Autosomal Recessive 10; Ataxia and Polyneuropathy, Adult-Onset; Leukemia, Acute Myeloid;
Macular Degeneration, Age-Related, 1; Muscular Dystrophy, Limb-Girdle, Autosomal Dominant 3;
Aspergillosis; Muscular Dystrophy, Limb-Girdle, Autosomal Dominant 1; Polydactyly; Methylmalonic
Acidemia and Homocysteinemia, Cblx Type; Rett Syndrome; Adenomyosis; Myotonic Dystrophy 2;
Intellectual Developmental Disorder, X-Linked 41; Incontinentia Pigmenti; Ornithine Transcarbamylase
Deficiency, Hyperammonemia Due to; Coffin-Lowry Syndrome; Mucopolysaccharidosis, Type Ii;
Paralytic Poliomyelitis; Primary Progressive Multiple Sclerosis; Neuronal Ceroid Lipofuscinosis; Mood
Disorder; Postpoliomyelitis Syndrome; Avoidant Personality Disorder; Personality Disorder; Dysentery;
Guillain-Barre Syndrome; Basilar Artery Occlusion; Squamous Cell Papilloma; Megacolon;
Hyperuricemia; Lactic Acidosis; Hermaphroditism; Toxic Megacolon; T Cell Deficiency; Goiter;
Retinal Ischemia; Inguinal Hernia; Thrombosis; Craniosynostosis with Fibular Aplasia; Retinal Vascular
Disease; Papilloma; Sensory Peripheral Neuropathy; Lipoprotein Quantitative Trait Locus;
Fibromyalgia; Overnutrition; Liver Disease; Peptic Ulcer Disease; Interstitial Keratitis; Sideroblastic
Anemia; Juvenile Retinoschisis; Limb-Girdle Muscular Dystrophy Type 1a; Pattern Dystrophy; Pellucid
Marginal Degeneration; X-Linked Congenital Retinoschisis; 46, Xy Disorders of Sexual Development;
Limb-Girdle Muscular Dystrophy Type 1b; Limb-Girdle Muscular Dystrophy Type 1c; Genetic Skeletal
Muscle Disease; Ventilator-Induced Diaphragmatic Dysfunction; Mesial Temporal Lobe Epilepsy with
Hippocampal Sclerosis; Acute Adrenal Insufficiency; Atherosclerosis Susceptibility; Noonan Syndrome
1; Ovarian Cancer; Dowling-Degos Disease 1; Lymphatic Malformation 5; Antigen Defined by
Monoclonal Antibody Aj9; Myopathy, Congenital, with Fiber-Type Disproportion; Obesity-
Hypoventilation Syndrome; Ocular Motor Apraxia; Mitochondrial Complex I Deficiency, Nuclear Type
1; Muscular Dystrophy, Limb-Girdle, Autosomal Recessive 8; Respiratory Distress Syndrome in
Premature Infants; Bacterial Infectious Disease; Actn3 Deficiency; Tetralogy of Fallot; Sarcoidosis 1;
Parkinson Disease, Late-Onset; Progressive External Ophthalmoplegia with Mitochondrial Dna
Deletions, Autosomal Dominant 1; Myopathy, Tubular Aggregate, 1; Chromosome 3q29 Deletion
Syndrome; Progressive External Ophthalmoplegia with Mitochondrial Dna Deletions, Autosomal
Dominant 4; Salih Myopathy; Major Depressive Disorder; Methylmalonic Aciduria and
Homocystinuria, Cblc Type; Retinitis Pigmentosa 3; Progressive External Ophthalmoplegia with
Mitochondrial Dna Deletions, Autosomal Dominant 2; Nemaline Myopathy 1; Glut1 Deficiency
Syndrome 2; Nasopharyngeal Carcinoma; Dengue Virus; Peripartum Cardiomyopathy; Impaired
Intellectual Development and Distinctive Facial Features with or Without Cardiac Defects; Hemophilia
B; Malaria; Hamamy Syndrome; Night Blindness, Congenital Stationary, Type 1e;
Mucopolysaccharidosis-Plus Syndrome; Mental Retardation, Autosomal Dominant 7; Beta-
Thalassemia; Breasts and/or Nipples, Aplasia or Hypoplasia of, 1; Kearns-Sayre Syndrome; Retinitis
Pigmentosa 11; Stroke, Ischemic; Menkes Disease; Nemaline Myopathy 3; Linear Skin Defects with
Multiple Congenital Anomalies 1; Third-Degree Atrioventricular Block; Tracheomalacia; Ifap
Syndrome 2; Severe Pre-Eclampsia; Adenocarcinoma; Squamous Cell Carcinoma; Childhood Absence
Epilepsy; Dysthymic Disorder; Cholera; Anal Squamous Cell Carcinoma; Adenosine Deaminase
Deficiency; Posterior Myocardial Infarction; Lymphopenia; Thrombocytopenia; Graves' Disease;
Chronic Progressive External Ophthalmoplegia; Newborn Respiratory Distress Syndrome; Primary
Biliary Cholangitis; Olivopontocerebellar Atrophy; Gastroenteritis; Optic Nerve Disease; Enthesopathy;
Focal Epilepsy; Mental Depression; Fibrosarcoma; Placental Insufficiency; Cystic Lymphangioma; Egg
Allergy; Rhinitis; Intracranial Embolism; Neurilemmoma; Mesenchymal Cell Neoplasm; Middle East
Respiratory Syndrome; Eclampsia; Autosomal Dominant Non-Syndromic Intellectual Disability 5;
Epidermolysis Bullosa Simplex with Muscular Dystrophy; Peripheral Vascular Disease; Angina
Pectoris; Prion Disease; Neuroblastoma; Viral Infectious Disease; Cholangitis; in Situ Carcinoma;
Collagen Disease; Polyhydramnios; Atp7a-Related Copper Transport Disorders; Dyrkla Syndrome;
Grin1-Related Neurodevelopmental Disorder; Cap Myopathy; Splenomegaly; Gigantism; Iqsec2;
Pseudo-Turner Syndrome; Neuropathy; Chilaiditi Syndrome; Childhood-Onset Nemaline Myopathy;
Broken Heart Syndrome; Syngap1-Related Intellectual Disability; Homologous Wasting Disease;
Necrotizing Autoimmune Myopathy; Methylmalonic Acidemia with Homocystinuria; Encephalitis;
Intermediate Congenital Nemaline Myopathy; Paroxysmal Exertion-Induced Dyskinesia; Pediatric
Multiple Sclerosis; Hypereosinophilic Syndrome; Specific Language Disorder; Periodic Paralysis;
Univentricular Heart; Qualitative or Quantitative Defects of Beta-Sarcoglycan; Acute Generalized
Exanthematous Pustulosis; Disorder of Copper Metabolism; Headache; Tracheobronchomalacia; Seizure
Disorder; Metabolic Myopathy; Inclusion Body Myopathy with Early-Onset Paget Disease with or
Without Frontotemporal Dementia 1; Prostate Cancer; Volvulus of Midgut; Acyl-Coa Dehydrogenase,
Very Long-Chain, Deficiency of; Arachnoid Cysts, Intracranial; Bladder Cancer; Candidiasis, Familial,
1; Cone-Rod Dystrophy 2; Jalili Syndrome; Schopf-Schulz-Passarge Syndrome; Carpal Tunnel
Syndrome; Clubfoot, Congenital, with or Without Deficiency of Long Bones and/or Mirror-Image
Polydactyly; Retinoblastoma; Kabuki Syndrome 1; Migraine with or Without Aura 1; Leprosy 3;
Myosclerosis, Autosomal Recessive; Nemaline Myopathy 2; Sialuria; Fryns Syndrome;
Dihydrolipoamide Dehydrogenase Deficiency; D-Bifunctional Protein Deficiency; Periodontitis,
Chronic; Exanthem; Peripheral Artery Disease; Pontocerebellar Hypoplasia; 3-Methylglutaconic
Aciduria; Mitochondrial Dna Depletion Syndrome; Neurofibromatosis, Type I; Hemophagocytic
Lymphohistiocytosis; Chromosome 16p11.2 Deletion Syndrome; Syndromic X-Linked Intellectual
Disability Snyder Type; Intestinal Pseudo-Obstruction; Leber Plus Disease; Non-Syndromic X-Linked
Intellectual Disability 2; Strabismus; Exostoses, Multiple, Type I; Gnathodiaphyseal Dysplasia;
Nondisjunction; Hypercholesterolemia, Familial, 1; Cholestasis, Intrahepatic, of Pregnancy, 1;
Leiomyoma, Uterine; Frontotemporal Dementia and/or Amyotrophic Lateral Sclerosis 1; Facial Spasm;
Kleefstra Syndrome 1; Muscular Dystrophy, Limb-Girdle, Autosomal Recessive 12; Cavitary Optic Disc
Anomalies; Wilson Disease; Night Blindness, Congenital Stationary, Type 2a; Thiamine Metabolism
Dysfunction Syndrome 2; Nemaline Myopathy 4; Meningioma, Radiation-Induced; Accelerated Tumor
Formation; Arthrogryposis, Mental Retardation, and Seizures; Muscular Dystrophy-Dystroglycanopathy,
Type C, 7; Tremor, Hereditary Essential, 5; Developmental and Epileptic Encephalopathy 75; Neuronal
Ceroid-Lipofuscinoses; Mandibular Hypoplasia, Deafness, Progeroid Features, and Lipodystrophy
Syndrome; Encephalopathy, Progressive, Early-Onset, with Brain Edema and/or Leukoencephalopathy,
1; Mycobacterium Tuberculosis 1; Hypophosphatemic Rickets, X-Linked Recessive; Fragile X
Tremor/ataxia Syndrome; Pigmentary Disorder, Reticulate, with Systemic Manifestations, X-Linked;
Nance-Horan Syndrome; Chondrodysplasia Punctata 2, X-Linked Dominant; Choroideremia; Paget
Disease of Bone 2, Early-Onset; Aromatic L-Amino Acid Decarboxylase Deficiency; Cervical Cancer;
Muscular Dystrophy-Dystroglycanopathy, Type a, 7; Cardiomyopathy, Dilated, lii; Mitochondrial Dna
Depletion Syndrome 13; 3-Methylglutaconic Aciduria, Type Vii; Muscular Dystrophy-
Dystroglycanopathy, Type B, 2; Premature Ovarian Failure 7; Muscular Dystrophy-Dystroglycanopathy,
Type C, 2; Frontotemporal Dementia and/or Amyotrophic Lateral Sclerosis 6; Muscular Dystrophy-
Dystroglycanopathy, Type a, 2; Canavan Disease; Thymoma, Familial; Citrullinemia, Type Ii, Adult-
Onset; Cardiomyopathy, Familial Restrictive, 3; Lesch-Nyhan Syndrome; Severe Combined
Immunodeficiency, X-Linked; Intellectual Developmental Disorder, X-Linked, Syndromic, Snyder-
Robinson Type; Pyruvate Dehydrogenase E1-Alpha Deficiency; Chondrosarcoma; Perrault Syndrome
1; Cholestasis, Progressive Familial Intrahepatic, 2; Fryns Microphthalmia Syndrome; Cardiomyopathy,
Dilated, 1d; Macular Degeneration, X-Linked Atrophic; Developmental and Epileptic Encephalopathy
1; Renpenning Syndrome 1; Prostatic Hyperplasia, Benign; X Inactivation, Familial Skewed, 1;
Adrenoleukodystrophy; Pettigrew Syndrome; Intellectual Developmental Disorder, X-Linked,
Syndromic, Lujan-Fryns Type; Ubiquitin-Activating Enzyme, Y-Linked; Tooth Agenesis; Coenzyme
Q10 Deficiency Disease; Perrault Syndrome; Tongue Squamous Cell Carcinoma; Oral Squamous Cell
Carcinoma; Syndromic X-Linked Intellectual Disability 14; Progressive Familial Intrahepatic
Cholestasis; Gynecomastia; Leiomyoma; Color Blindness; Prostatic Adenoma; Paraplegia;
Demyelinating Polyneuropathy; Essential Tremor; Chronic Inflammatory Demyelinating
Polyradiculoneuropathy; Detrusor Sphincter Dyssynergia; Familial Hypercholesterolemia; Angioedema;
Cerebral Degeneration; Gaucher's Disease; Amelogenesis Imperfecta; Thymoma; Olfactory
Neuroblastoma; Transient Cerebral Ischemia; Aortic Aneurysm; Junctional Epidermolysis Bullosa;
Malignant Astrocytoma; Complex Regional Pain Syndrome; Neuromuscular Junction Disease;
Rectosigmoid Cancer; Macular Retinal Edema; Systemic Scleroderma; Skull Base Meningioma; Corneal
Dystrophy; Kidney Cancer; Prostatic Hypertrophy; Adult Respiratory Distress Syndrome; Pulmonary
Edema; Hemiplegia; Infant Gynecomastia; Congenital Muscular Dystrophy-Dystroglycanopathy A7;
Congenital Muscular Dystrophy-Dystroglycanopathy Type A2; Non-Alcoholic Steatohepatitis;
Cystinosis; Osteopetrosis; Achromatopsia; Allergic Disease; Atrial Fibrillation; Lung Cancer;
Panniculitis; Urticaria; Dental Caries; Cholestasis; Gastritis; Axonal Neuropathy; Clear Cell
Meningioma; Acquired Immunodeficiency Syndrome; Retinal Degeneration; Vasculitis; Mesenchymal
Chondrosarcoma; Biotin-Thiamine-Responsive Basal Ganglia Disease; Free Sialic Acid Storage
Disorders; Giant Axonal Neuropathy; Chronic Fatigue Syndrome; Amyloidosis; Periodontitis;
Stenotrophomonas Maltophilia Infection; Encephalopathy; Congenital Nystagmus; Hereditary
Neuropathies; Coccygodynia; Glioma; Anca-Associated Vasculitis; Auriculo-Condylar Syndrome;
Chromosome Xp Deletion; Corticobasal Degeneration; Mechanical Strabismus; Trichorhinophalangeal
Syndrome; Adrenomyeloneuropathy; Exencephaly; Hansen's Disease; Precocious Puberty; Madelung
Deformity; Ocular Albinism, X-Linked; Rrm2b Mitochondrial Dna Maintenance Defects; Diabetic
Neuropathy; Glial Tumor; Familial Intrahepatic Cholestasis; Spastic Paraplegia-Paget Disease of Bone
Syndrome; Rigid Spine Muscular Dystrophy; Traumatic Brain Injury; Laminin Subunit Alpha 2-Related
Muscular Dystrophy; Ring Chromosome; Homozygous Familial Hypercholesterolemia; Cerebral
Aneurysms; Anoxia; Cerebral Hypoxia; Color Vision Deficiency; Laminopathy; Familial Isolated
Restrictive Cardiomyopathy; Polyploidy; Argyria; or Non-Syndromic Pontocerebellar Hypoplasia

### EXAMPLES

The following examples are included for illustrative purposes only and are not intended to limit the scope of the invention.

### Example 1: PAM Screening for D2S Effector Protein CasM.265466

D2S effector protein CasM.265466 and guide RNA combinations represented in TABLE 12 and TABLE 13 were screened by in vitro enrichment (IVE) for PAM recognition. Specifically, compositions included in the PAM screening assay included Comp. Nos.: 1, 2, and 3 as shown in TABLE 12 and TABLE 13. TABLE 12 and TABLE 13 show the components of each effector protein-guide RNA complex assayed for PAM recognition. The amino acid sequence of CasM.265466 is shown in TABLE 1 herein. The nucleotide sequences of the guide components are shown in TABLE 12 and TABLE 13 herein. For example, as shown in TABLE 12, an effector protein comprising an amino acid sequence of SEQ ID NO: 1 complexed with a guide comprising a crRNA of SEQ ID NO: 444 and a tracrRNA of SEQ ID NO: 441 was screened for PAM recognition.

Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a 5′ PAM library in 1× Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Next generation sequencing was performed on cut sequences to identify enriched PAM sequence for CasM.265466 using crRNA and tracrRNA sequences as shown in TABLE 12. Cis cleavage by each complex was confirmed by gel electrophoresis. The most enriched PAM was represented by the sequence 5′-TNTR-3′ (SEQ ID NO: 3), wherein N is any nucleotide and R is adenine or guanine.

TABLE 12
Exemplary Compositions of D2S Effector Protein,
crRNA and tracrRNA of CasM.265466

| Comp. | | | |
|---|---|---|---|
| No. | Protein | crRNA | tracrRNA |
| 1 | CasM.265466 | GUUUGAGAACCUUAUGA | ACAGCUUAUUUGGAAGCUGAA |
| | (SEQ ID | AAUUACAAGGAUGCCAA | AUGUGAGGUUUAUAACACUCA |
| | NO: 1) | ACUAUUAAAUACUCGUA | CAAGAAUCCU |
| | | UUGCU | (SEQ ID NO: 441) |
| | | (SEQ ID NO: 444) | |
| 2 | CasM.265466 | GUUUGAGAACCUUAUGA | UAUAUUUGAUAAAAAUAUACA |
| | (SEQ ID | AAUUACAAGGAUGCCAA | GCUUAUUUGGAAGCUGAAAUG |
| | NO: 1) | ACUAUUAAAUACUCGUA | UGAGGUUUAUAACACUCACAA |
| | | UUGCU | GAAUCC |
| | | (SEQ ID NO: 445) | (SEQ ID NO: 446) |

TABLE 13
Exemplary Compositions of D2S
Effector Protein, sgRNA

| Comp. No. | Protein | sgRNA |
|---|---|---|
| 3 | CasM.265466 | ACAGCUUAUUUGGAAGCUGA |
| | (SEQ ID | AAUGUGAGGUUUAUAACACU |
| | NO: 1) | CACAAGAAUCCUGAAAaagg |
| | | augccaaacUAUUAAAUACU |
| | | CGUAUUGCU |
| | | (SEQ ID NO: 447) |

*In italics is a handle sequence without a linker or repeat sequence, in bold is a linker, lowercase is a repeat sequence, and no formatting is a spacer sequence.

### Example 2: Indel Activity of Effector Protein

Combinations of the effector protein (as set forth in SEQ ID NO: 1) and guide nucleic acids having spacer sequences as set forth in TABLE 4 to target various exons (loci) of the DMD gene, as represented in TABLE 14, were tested for their ability to produce indels in HEK293T cells. Nucleotide sequences of targeted exons are as set forth in TABLE 9. Some indels are predicted to result in exon skipping.

Briefly, 300ng of plasmids expressing the effector protein (as set forth in SEQ ID NO: 1) and transcribing targeting gRNA were delivered by lipofection to HEK293T cells in 96 well plates. TransIT-293 reagent was diluted with warmed up OPTIMEM and mixed with the plasmid DNA at the ratio of 2:1 lipid:DNA. Lipid:DNA mixture were incubated for 10 minutes at room temperature before adding 20 μL of the lipid:DNA optimem mixture to each well. Cells were incubated for 3 days before being lysed and subjected to PCR amplification. Each composition was assayed in two replicate batches. Indels were detected by next generation sequencing of PCR amplicons at the targeted loci and indel percentage was calculated as the fraction of sequencing reads containing insertions or deletions relative to the unedited DMD gene sequence, and are provided in TABLE 15.

TABLE 14
Effector Protein (SEQ ID NO: 1) and Guide Nucleic Acid (sgRNA)
Combinations and their Target Exon

| Effector Protein (SEQ ID NO: 1) and Guide Nucleic Acid | SEQ ID | Exon |
|---|---|---|
| Combinations, shown as RNA | NO: | No. |
| ACAGCUUAUUUGGAAGCUGAAAUGUGAGGUUUAUAACACUCACAAGAAUCCUGAAA | 223 | 53 |
| aaggaugccaaacAUACUAACCUUGGUUUCUGU | | |
| ACAGCUUAUUUGGAAGCUGAAAUGUGAGGUUUAUAACACUCACAAGAAUCCUGAAA | 224 | 53 |
| aaggaugccaaacCUGUUCAUUUCAGCUUUAAC | | |
| ACAGCUUAUUUGGAAGCUGAAAUGUGAGGUUUAUAACACUCACAAGAAUCCUGAAA | 225 | 53 |
| aaggaugccaaacCCACUGCACUUUAGCCUGGG | | |
| ACAGCUUAUUUGGAAGCUGAAAUGUGAGGUUUAUAACACUCACAAGAAUCCUGAAA | 226 | 53 |
| aaggaugccaaacUCAAAUGUAACCAGUAUUUU | | |
| ACAGCUUAUUUGGAAGCUGAAAUGUGAGGUUUAUAACACUCACAAGAAUCCUGAAA | 227 | 53 |
| aaggaugccaaacGCCUGGGUGACAGUGAGACU | | |
| ACAGCUUAUUUGGAAGCUGAAAUGUGAGGUUUAUAACACUCACAAGAAUCCUGAAA | 228 | 53 |
| aaggaugccaaacAAAAGGUAUCUUUGAUACUA | | |
| ACAGCUUAUUUGGAAGCUGAAAUGUGAGGUUUAUAACACUCACAAGAAUCCUGAAA | 229 | 53 |
| aaggaugccaaacACGUGAUUUUCUGUUAAUAA | | |
| ACAGCUUAUUUGGAAGCUGAAAUGUGAGGUUUAUAACACUCACAAGAAUCCUGAAA | 230 | 53 |
| aaggaugccaaacCAAAGUCUACUGUUCAUUUC | | |
| ACAGCUUAUUUGGAAGCUGAAAUGUGAGGUUUAUAACACUCACAAGAAUCCUGAAA | 231 | 53 |
| aaggaugccaaacAUUUUAUCAAAUGUAACCAG | | |
| ACAGCUUAUUUGGAAGCUGAAAUGUGAGGUUUAUAACACUCACAAGAAUCCUGAAA | 232 | 53 |
| aaggaugccaaacUUUUCUUAGAGACAGAGUCU | | |
| ACAGCUUAUUUGGAAGCUGAAAUGUGAGGUUUAUAACACUCACAAGAAUCCUGAAA | 233 | 53 |
| aaggaugccaaacCCAUAGAUUGUAAUUUAAUG | | |
| ACAGCUUAUUUGGAAGCUGAAAUGUGAGGUUUAUAACACUCACAAGAAUCCUGAAA | 234 | 53 |
| aaggaugccaaacUUUAUUUUCUUAGAGACAGA | | |
| ACAGCUUAUUUGGAAGCUGAAAUGUGAGGUUUAUAACACUCACAAGAAUCCUGAAA | 235 | 53 |
| aaggaugccaaacGCUAGGAUGAUGAACAACAG | | |
| ACAGCUUAUUUGGAAGCUGAAAUGUGAGGUUUAUAACACUCACAAGAAUCCUGAAA | 236 | 53 |
| aaggaugccaaacGUAGUAAAUGCUAGUCUGGA | | |
| ACAGCUUAUUUGGAAGCUGAAAUGUGAGGUUUAUAACACUCACAAGAAUCCUGAAA | 23 | 53 |
| aaggaugccaaacAUGGCAAAUAUUAGUUUCUG | | |
| ACAGCUUAUUUGGAAGCUGAAAUGUGAGGUUUAUAACACUCACAAGAAUCCUGAAA | 238 | 53 |
| aaggaugccaaacUAGUAGUAAAUGCUAGUCUG | | |
| ACAGCUUAUUUGGAAGCUGAAAUGUGAGGUUUAUAACACUCACAAGAAUCCUGAAA | 239 | 53 |
| aaggaugccaaacUUAUGGCUAGGAUGAUGAAC | | |
| ACAGCUUAUUUGGAAGCUGAAAUGUGAGGUUUAUAACACUCACAAGAAUCCUGAAA | 240 | 53 |
| aaggaugccaaacGAGGAGACAUUUUAAAUGUA | | |
| ACAGCUUAUUUGGAAGCUGAAAUGUGAGGUUUAUAACACUCACAAGAAUCCUGAAA | 241 | 53 |
| aaggaugccaaacAAUGUAACUUCCAAACGUUA | | |
| ACAGCUUAUUUGGAAGCUGAAAUGUGAGGUUUAUAACACUCACAAGAAUCCUGAAA | 242 | 53 |
| aaggaugccaaacACUUCCAAACGUUAUCUCAC | | |
| ACAGCUUAUUUGGAAGCUGAAAUGUGAGGUUUAUAACACUCACAAGAAUCCUGAAA | 243 | 53 |
| aaggaugccaaacCUUUUUUGAUGGCAAAUAUU | | |
| ACAGCUUAUUUGGAAGCUGAAAUGUGAGGUUUAUAACACUCACAAGAAUCCUGAAA | 244 | 53 |
| aaggaugccaaacAGAUAACGUUUGGAAGUUAC | | |
| ACAGCUUAUUUGGAAGCUGAAAUGUGAGGUUUAUAACACUCACAAGAAUCCUGAAA | 245 | 53 |
| aaggaugccaaacCCAUCAAAAAAGCAAAGAAU | | |
| ACAGCUUAUUUGGAAGCUGAAAUGUGAGGUUUAUAACACUCACAAGAAUCCUGAAA | 246 | 53 |
| aaggaugccaaacAAUAAGCAACAUAAAUGUGA | | |
| ACAGCUUAUUUGGAAGCUGAAAUGUGAGGUUUAUAACACUCACAAGAAUCCUGAAA | 247 | 53 |
| aaggaugccaaacGUUGAAAGAAUUCAGAAUCA | | |
| ACAGCUUAUUUGGAAGCUGAAAUGUGAGGUUUAUAACACUCACAAGAAUCCUGAAA | 248 | 53 |
| aaggaugccaaacGAAGUUACAUUUAAAAUGUC | | |
| ACAGCUUAUUUGGAAGCUGAAAUGUGAGGUUUAUAACACUCACAAGAAUCCUGAAA | 249 | 53 |
| aaggaugccaaacACAGAAACUAAUAUUUGCCA | | |
| ACAGCUUAUUUGGAAGCUGAAAUGUGAGGUUUAUAACACUCACAAGAAUCCUGAAA | 250 | 53 |
| aaggaugccaaacAAAUGUCUCCUCCAGACUAG | | |
| ACAGCUUAUUUGGAAGCUGAAAUGUGAGGUUUAUAACACUCACAAGAAUCCUGAAA | 251 | 53 |
| aaggaugccaaacUUCUAGUUGAAAGAAUUCAG | | |
| ACAGCUUAUUUGGAAGCUGAAAUGUGAGGUUUAUAACACUCACAAGAAUCCUGAAA | 252 | 51 |
| aaggaugccaaacCCAACUUUUAUCAUUUUUUC | | |
| ACAGCUUAUUUGGAAGCUGAAAUGUGAGGUUUAUAACACUCACAAGAAUCCUGAAA | 253 | 51 |
| aaggaugccaaacUUUGCUGAGAGAGAAACAGU | | |
| ACAGCUUAUUUGGAAGCUGAAAUGUGAGGUUUAUAACACUCACAAGAAUCCUGAAA | 254 | 51 |
| aaggaugccaaacCUUAGGCUGAAUAGUGAGAG | | |
| ACAGCUUAUUUGGAAGCUGAAAUGUGAGGUUUAUAACACUCACAAGAAUCCUGAAA | 255 | 51 |
| aaggaugccaaacUCAUUUUUUCUCAUACCUUC | | |
| ACAGCUUAUUUGGAAGCUGAAAUGUGAGGUUUAUAACACUCACAAGAAUCCUGAAA | 256 | 51 |
| aaggaugccaaacCUUGAUGAUCAUCUCGUUGA | | |
| ACAGCUUAUUUGGAAGCUGAAAUGUGAGGUUUAUAACACUCACAAGAAUCCUGAAA | 257 | 51 |
| aaggaugccaaacCUGAGAGAGAAACAGUUGCC | | |
| ACAGCUUAUUUGGAAGCUGAAAUGUGAGGUUUAUAACACUCACAAGAAUCCUGAAA | 258 | 51 |
| aaggaugccaaacGCUACUUUUGUUAUUUGCAU | | |
| ACAGCUUAUUUGGAAGCUGAAAUGUGAGGUUUAUAACACUCACAAGAAUCCUGAAA | 259 | 51 |
| aaggaugccaaacGGAGAGUAAAGUGAUUGGUG | | |
| ACAGCUUAUUUGGAAGCUGAAAUGUGAGGUUUAUAACACUCACAAGAAUCCUGAAA | 260 | 51 |
| aaggaugccaaacUGGCUACUUUUGUUAUUUGC | | |
| ACAGCUUAUUUGGAAGCUGAAAUGUGAGGUUUAUAACACUCACAAGAAUCCUGAAA | 261 | 51 |
| aaggaugccaaacAAGAAAAACUUCUGCCAACU | | |
| ACAGCUUAUUUGGAAGCUGAAAUGUGAGGUUUAUAACACUCACAAGAAUCCUGAAA | 262 | 51 |
| aaggaugccaaacUAUCCUUGAUUAUACUUAGG | | |
| ACAGCUUAUUUGGAAGCUGAAAUGUGAGGUUUAUAACACUCACAAGAAUCCUGAAA | 263 | 51 |
| aaggaugccaaacUCCUUGAUUAUACUUAGGCU | | |
| ACAGCUUAUUUGGAAGCUGAAAUGUGAGGUUUAUAACACUCACAAGAAUCCUGAAA | 264 | 51 |
| aaggaugccaaacAAAUGAAGAUUUUCCACCAA | | |
| ACAGCUUAUUUGGAAGCUGAAAUGUGAGGUUUAUAACACUCACAAGAAUCCUGAAA | 265 | 51 |
| aaggaugccaaacCUCUCCUAGACCAUUUCCCA | | |
| ACAGCUUAUUUGGAAGCUGAAAUGUGAGGUUUAUAACACUCACAAGAAUCCUGAAA | 266 | 51 |
| aaggaugccaaacAGUAGGAGCUAAAAUAUUUU | | |
| ACAGCUUAUUUGGAAGCUGAAAUGUGAGGUUUAUAACACUCACAAGAAUCCUGAAA | 267 | 51 |
| aaggaugccaaacGAUACUUUGUUUAGCAAUAC | | |
| ACAGCUUAUUUGGAAGCUGAAAUGUGAGGUUUAUAACACUCACAAGAAUCCUGAAA | 268 | 51 |
| aaggaugccaaacGGUUUUUGCAAAAAGGAAAA | | |
| ACAGCUUAUUUGGAAGCUGAAAUGUGAGGUUUAUAACACUCACAAGAAUCCUGAAA | 269 | 51 |
| aaggaugccaaacUCUUUUUCUAACAAUGUGGA | | |
| ACAGCUUAUUUGGAAGCUGAAAUGUGAGGUUUAUAACACUCACAAGAAUCCUGAAA | 270 | 51 |
| aaggaugccaaacACAAUGUGGAUACUUUGUUU | | |
| ACAGCUUAUUUGGAAGCUGAAAUGUGAGGUUUAUAACACUCACAAGAAUCCUGAAA | 271 | 51 |
| aaggaugccaaacAGCCAAACUCUUAUUCAUGA | | |
| ACAGCUUAUUUGGAAGCUGAAAUGUGAGGUUUAUAACACUCACAAGAAUCCUGAAA | 272 | 51 |
| aaggaugccaaacAUUGAAGAGUAACAAUUUGA | | |
| ACAGCUUAUUUGGAAGCUGAAAUGUGAGGUUUAUAACACUCACAAGAAUCCUGAAA | 273 | 51 |
| aaggaugccaaacGCAAUACAUGGUAGAAAAUG | | |
| ACAGCUUAUUUGGAAGCUGAAAUGUGAGGUUUAUAACACUCACAAGAAUCCUGAAA | 274 | 51 |
| aaggaugccaaacCAAAAAGGAAAAAAGAAGAA | | |
| ACAGCUUAUUUGGAAGCUGAAAUGUGAGGUUUAUAACACUCACAAGAAUCCUGAAA | 275 | 51 |
| aaggaugccaaacUUUAGCAAUACAUGGUAGAA | | |
| ACAGCUUAUUUGGAAGCUGAAAUGUGAGGUUUAUAACACUCACAAGAAUCCUGAAA | 276 | 51 |
| aaggaugccaaacUAUCUUUUUCUAACAAUGUG | | |
| ACAGCUUAUUUGGAAGCUGAAAUGUGAGGUUUAUAACACUCACAAGAAUCCUGAAA | 277 | 51 |
| aaggaugccaaacAUGUCAUGAAUAAGAGUUUG | | |
| ACAGCUUAUUUGGAAGCUGAAAUGUGAGGUUUAUAACACUCACAAGAAUCCUGAAA | 278 | 51 |
| aaggaugccaaacGCUCCUACUCAGACUGUUAC | | |
| ACAGCUUAUUUGGAAGCUGAAAUGUGAGGUUUAUAACACUCACAAGAAUCCUGAAA | 279 | 51 |
| aaggaugccaaacGCUUGUGUUUCUAAUUUUUC | | |
| ACAGCUUAUUUGGAAGCUGAAAUGUGAGGUUUAUAACACUCACAAGAAUCCUGAAA | 280 | 51 |
| aaggaugccaaacUUGCUAAACAAAGUAUCCAC | | |
| ACAGCUUAUUUGGAAGCUGAAAUGUGAGGUUUAUAACACUCACAAGAAUCCUGAAA | 281 | 51 |
| aaggaugccaaacUAAUGUCAUGAAUAAGAGUU | | |
| ACAGCUUAUUUGGAAGCUGAAAUGUGAGGUUUAUAACACUCACAAGAAUCCUGAAA | 282 | 51 |
| aaggaugccaaacCCAUGUAUUGCUAAACAAAG | | |
| ACAGCUUAUUUGGAAGCUGAAAUGUGAGGUUUAUAACACUCACAAGAAUCCUGAAA | 283 | 51 |
| aaggaugccaaacGCUCAAAUUGUUACUCUUCA | | |
| ACAGCUUAUUUGGAAGCUGAAAUGUGAGGUUUAUAACACUCACAAGAAUCCUGAAA | 284 | 50 |
| aaggaugccaaacAGACUUUUUGCACAGUCAAU | | |
| ACAGCUUAUUUGGAAGCUGAAAUGUGAGGUUUAUAACACUCACAAGAAUCCUGAAA | 285 | 50 |
| aaggaugccaaacCUUACAGGCUCCAAUAGUGG | | |
| ACAGCUUAUUUGGAAGCUGAAAUGUGAGGUUUAUAACACUCACAAGAAUCCUGAAA | 286 | 50 |
| aaggaugccaaacCACAGUCAAUAACACAAAGG | | |
| ACAGCUUAUUUGGAAGCUGAAAUGUGAGGUUUAUAACACUCACAAGAAUCCUGAAA | 287 | 50 |
| aaggaugccaaacGAAUUGAAACAAAUUUUCUC | | |
| ACAGCUUAUUUGGAAGCUGAAAUGUGAGGUUUAUAACACUCACAAGAAUCCUGAAA | 288 | 50 |
| aaggaugccaaacUCUCUAUCUUUAGAAUUGAA | | |
| ACAGCUUAUUUGGAAGCUGAAAUGUGAGGUUUAUAACACUCACAAGAAUCCUGAAA | 289 | 50 |
| aaggaugccaaacAACAAAUAGCUAGAGCCAAA | | |
| ACAGCUUAUUUGGAAGCUGAAAUGUGAGGUUUAUAACACUCACAAGAAUCCUGAAA | 290 | 50 |
| aaggaugccaaacAAUCAGAGUCAAUUUCCAAG | | |
| ACAGCUUAUUUGGAAGCUGAAAUGUGAGGUUUAUAACACUCACAAGAAUCCUGAAA | 291 | 50 |
| aaggaugccaaacCUCUAAGACUUUUUGCACAG | | |
| ACAGCUUAUUUGGAAGCUGAAAUGUGAGGUUUAUAACACUCACAAGAAUCCUGAAA | 292 | 50 |
| aaggaugccaaacUUCAAAAGUGCAACUAUGAA | | |
| ACAGCUUAUUUGGAAGCUGAAAUGUGAGGUUUAUAACACUCACAAGAAUCCUGAAA | 293 | 50 |
| aaggaugccaaacGCUCUAGCUAUUUGUUCAAA | | |
| ACAGCUUAUUUGGAAGCUGAAAUGUGAGGUUUAUAACACUCACAAGAAUCCUGAAA | 294 | 50 |
| aaggaugccaaacGCUAUUUGUUCAAAAGUGCA | | |
| ACAGCUUAUUUGGAAGCUGAAAUGUGAGGUUUAUAACACUCACAAGAAUCCUGAAA | 295 | 50 |
| aaggaugccaaacAGUAUACUGGAUCCCAUUCU | | |
| ACAGCUUAUUUGGAAGCUGAAAUGUGAGGUUUAUAACACUCACAAGAAUCCUGAAA | 296 | 50 |
| aaggaugccaaacUGUUAUUGACUGUGCAAAAA | | |
| ACAGCUUAUUUGGAAGCUGAAAUGUGAGGUUUAUAACACUCACAAGAAUCCUGAAA | 297 | 50 |
| aaggaugccaaacAUUCAAAGUGUUGCAUGACA | | |
| ACAGCUUAUUUGGAAGCUGAAAUGUGAGGUUUAUAACACUCACAAGAAUCCUGAAA | 298 | 50 |
| aaggaugccaaacUUUCAAUUCUAAAGAUAGAG | | |
| ACAGCUUAUUUGGAAGCUGAAAUGUGAGGUUUAUAACACUCACAAGAAUCCUGAAA | 299 | 50 |
| aaggaugccaaacAAGAUAGAGAUAAACCUUUG | | |
| ACAGCUUAUUUGGAAGCUGAAAUGUGAGGUUUAUAACACUCACAAGAAUCCUGAAA | 300 | 50 |
| aaggaugccaaacUUAUUGACUGUGCAAAAAGU | | |
| ACAGCUUAUUUGGAAGCUGAAAUGUGAGGUUUAUAACACUCACAAGAAUCCUGAAA | 301 | 50 |
| aaggaugccaaacAAGUGAUGACUGGGUGAGAG | | |
| ACAGCUUAUUUGGAAGCUGAAAUGUGAGGUUUAUAACACUCACAAGAAUCCUGAAA | 302 | 50 |
| aaggaugccaaacCUGGAUCCCAUUCUCUUUGG | | |
| ACAGCUUAUUUGGAAGCUGAAAUGUGAGGUUUAUAACACUCACAAGAAUCCUGAAA | 303 | 50 |
| aaggaugccaaacCAAAAAGUCUUAGAGUACAU | | |
| ACAGCUUAUUUGGAAGCUGAAAUGUGAGGUUUAUAACACUCACAAGAAUCCUGAAA | 304 | 50 |
| aaggaugccaaacAAGAUAAUUCAUGAACAUCU | | |
| ACAGCUUAUUUGGAAGCUGAAAUGUGAGGUUUAUAACACUCACAAGAAUCCUGAAA | 305 | 50 |
| aaggaugccaaacAUUAUUUUAGCCAACCACCC | | |
| ACAGCUUAUUUGGAAGCUGAAAUGUGAGGUUUAUAACACUCACAAGAAUCCUGAAA | 306 | 50 |
| aaggaugccaaacCUUCUAAAUUAACUUUAGUG | | |
| ACAGCUUAUUUGGAAGCUGAAAUGUGAGGUUUAUAACACUCACAAGAAUCCUGAAA | 307 | 50 |
| aaggaugccaaacAAUUAACUUUAGUGGGUAGA | | |
| ACAGCUUAUUUGGAAGCUGAAAUGUGAGGUUUAUAACACUCACAAGAAUCCUGAAA | 308 | 50 |
| aaggaugccaaacGCCAACCACCCUACAAAUAU | | |
| ACAGCUUAUUUGGAAGCUGAAAUGUGAGGUUUAUAACACUCACAAGAAUCCUGAAA | 309 | 50 |
| aaggaugccaaacGUGGGUAGAAUUUCUUUUAA | | |
| ACAGCUUAUUUGGAAGCUGAAAUGUGAGGUUUAUAACACUCACAAGAAUCCUGAAA | 310 | 50 |
| aaggaugccaaacACAGAAAAGCAUACACAUUA | | |
| ACAGCUUAUUUGGAAGCUGAAAUGUGAGGUUUAUAACACUCACAAGAAUCCUGAAA | 311 | 50 |
| aaggaugccaaacACUUCCUCUUUAACAGAAAA | | |
| ACAGCUUAUUUGGAAGCUGAAAUGUGAGGUUUAUAACACUCACAAGAAUCCUGAAA | 312 | 50 |
| aaggaugccaaacAAAGAAAUUCUACCCACUAA | | |
| ACAGCUUAUUUGGAAGCUGAAAUGUGAGGUUUAUAACACUCACAAGAAUCCUGAAA | 313 | 50 |
| aaggaugccaaacUAGGGUGGUUGGCUAAAAUA | | |
| ACAGCUUAUUUGGAAGCUGAAAUGUGAGGUUUAUAACACUCACAAGAAUCCUGAAA | 314 | 50 |
| aaggaugccaaacUAUGCUUUUCUGUUAAAGAG | | |
| ACAGCUUAUUUGGAAGCUGAAAUGUGAGGUUUAUAACACUCACAAGAAUCCUGAAA | 315 | 50 |
| aaggaugccaaacCCCACUAAAGUUAAUUUAGA | | |
| ACAGCUUAUUUGGAAGCUGAAAUGUGAGGUUUAUAACACUCACAAGAAUCCUGAAA | 316 | 50 |
| aaggaugccaaacUUAAAGAGGAAGUUAGAAGA | | |
| ACAGCUUAUUUGGAAGCUGAAAUGUGAGGUUUAUAACACUCACAAGAAUCCUGAAA | 317 | 50 |
| aaggaugccaaacUGCUUUUCUGUUAAAGAGGA | | |
| ACAGCUUAUUUGGAAGCUGAAAUGUGAGGUUUAUAACACUCACAAGAAUCCUGAAA | 318 | 50 |
| aaggaugccaaacUUCACCAAAUGGAUUAAGAU | | |
| ACAGCUUAUUUGGAAGCUGAAAUGUGAGGUUUAUAACACUCACAAGAAUCCUGAAA | 319 | 50 |
| aaggaugccaaacGGGUGGUUGGCUAAAAUAAU | | |
| ACAGCUUAUUUGGAAGCUGAAAUGUGAGGUUUAUAACACUCACAAGAAUCCUGAAA | 320 | 50 |
| aaggaugccaaacCUUUUCUGUUAAAGAGGAAG | | |
| ACAGCUUAUUUGGAAGCUGAAAUGUGAGGUUUAUAACACUCACAAGAAUCCUGAAA | 321 | 45 |
| aaggaugccaaacCUAAAAUGUUUUCAUUCCUA | | |
| ACAGCUUAUUUGGAAGCUGAAAUGUGAGGUUUAUAACACUCACAAGAAUCCUGAAA | 322 | 45 |
| aaggaugccaaacCUGCUGUUGAUUAAUGGUUG | | |
| ACAGCUUAUUUGGAAGCUGAAAUGUGAGGUUUAUAACACUCACAAGAAUCCUGAAA | 323 | 45 |
| aaggaugccaaacAUCUCUCAUGAAAUAUUCUU | | |
| ACAGCUUAUUUGGAAGCUGAAAUGUGAGGUUUAUAACACUCACAAGAAUCCUGAAA | 324 | 45 |
| aaggaugccaaacAAGAAAGCUUAAAAAGUCUG | | |
| ACAGCUUAUUUGGAAGCUGAAAUGUGAGGUUUAUAACACUCACAAGAAUCCUGAAA | 325 | 45 |
| aaggaugccaaacUCGCCCUACCUCUUUUUUCU | | |
| ACAGCUUAUUUGGAAGCUGAAAUGUGAGGUUUAUAACACUCACAAGAAUCCUGAAA | 326 | 45 |
| aaggaugccaaacAUGUUAGUGCCUUUCACCCU | | |
| ACAGCUUAUUUGGAAGCUGAAAUGUGAGGUUUAUAACACUCACAAGAAUCCUGAAA | 327 | 45 |
| aaggaugccaaacAGCAGGGUGAAAGGCACUAA | | |
| ACAGCUUAUUUGGAAGCUGAAAUGUGAGGUUUAUAACACUCACAAGAAUCCUGAAA | 328 | 45 |
| aaggaugccaaacGAAGAAUAUUUCAUGAGAGA | | |
| ACAGCUUAUUUGGAAGCUGAAAUGUGAGGUUUAUAACACUCACAAGAAUCCUGAAA | 329 | 45 |
| aaggaugccaaacAGCUUUCUUUAGAAGAAUAU | | |
| ACAGCUUAUUUGGAAGCUGAAAUGUGAGGUUUAUAACACUCACAAGAAUCCUGAAA | 330 | 45 |
| aaggaugccaaacCUAAAAUAUAUACUUGUGGC | | |
| ACAGCUUAUUUGGAAGCUGAAAUGUGAGGUUUAUAACACUCACAAGAAUCCUGAAA | 331 | 45 |
| aaggaugccaaacGCAGACUUUUUAAGCUUUCU | | |
| ACAGCUUAUUUGGAAGCUGAAAUGUGAGGUUUAUAACACUCACAAGAAUCCUGAAA | 332 | 45 |
| aaggaugccaaacAAAAUUUCCCUAUGAAACUG | | |
| ACAGCUUAUUUGGAAGCUGAAAUGUGAGGUUUAUAACACUCACAAGAAUCCUGAAA | 333 | 45 |
| aaggaugccaaacUUUGAGAAAAGAUUAAACAG | | |
| ACAGCUUAUUUGGAAGCUGAAAUGUGAGGUUUAUAACACUCACAAGAAUCCUGAAA | 334 | 45 |
| aaggaugccaaacAGAAAAGAUUAAACAGUGUG | | |
| ACAGCUUAUUUGGAAGCUGAAAUGUGAGGUUUAUAACACUCACAAGAAUCCUGAAA | 335 | 45 |
| aaggaugccaaacAGAUACCAAAAAGGCAAAAC | | |
| ACAGCUUAUUUGGAAGCUGAAAUGUGAGGUUUAUAACACUCACAAGAAUCCUGAAA | 336 | 45 |
| aaggaugccaaacCUGGCAAAGAAAGAAAUACA | | |
| ACAGCUUAUUUGGAAGCUGAAAUGUGAGGUUUAUAACACUCACAAGAAUCCUGAAA | 337 | 45 |
| aaggaugccaaacCUACCACAUGCAGUUGUACU | | |
| ACAGCUUAUUUGGAAGCUGAAAUGUGAGGUUUAUAACACUCACAAGAAUCCUGAAA | 338 | 45 |
| aaggaugccaaacAAACUGACAUGCCCAUAUCC | | |
| ACAGCUUAUUUGGAAGCUGAAAUGUGAGGUUUAUAACACUCACAAGAAUCCUGAAA | 339 | 45 |
| aaggaugccaaacUUUUGCCUUUUUGGUAUCUU | | |
| ACAGCUUAUUUGGAAGCUGAAAUGUGAGGUUUAUAACACUCACAAGAAUCCUGAAA | 340 | 45 |
| aaggaugccaaacGUAGCACACUGUUUAAUCUU | | |
| ACAGCUUAUUUGGAAGCUGAAAUGUGAGGUUUAUAACACUCACAAGAAUCCUGAAA | 341 | 45 |
| aaggaugccaaacUCCUUUGGAUAUGGGCAUGU | | |
| ACAGCUUAUUUGGAAGCUGAAAUGUGAGGUUUAUAACACUCACAAGAAUCCUGAAA | 342 | 45 |
| aaggaugccaaacUAUUUCUUUCUUUGCCAGUA | | |
| ACAGCUUAUUUGGAAGCUGAAAUGUGAGGUUUAUAACACUCACAAGAAUCCUGAAA | 343 | 45 |
| aaggaugccaaacUCUUGUAUCCUUUGGAUAUG | | |
| ACAGCUUAUUUGGAAGCUGAAAUGUGAGGUUUAUAACACUCACAAGAAUCCUGAAA | 344 | 45 |
| aaggaugccaaacCCUUUUUGGUAUCUUACAGG | | |
| ACAGCUUAUUUGGAAGCUGAAAUGUGAGGUUUAUAACACUCACAAGAAUCCUGAAA | 345 | 45 |
| aaggaugccaaacGAUAUGGGCAUGUCAGUUUC | | |
| ACAGCUUAUUUGGAAGCUGAAAUGUGAGGUUUAUAACACUCACAAGAAUCCUGAAA | 346 | 45 |
| aaggaugccaaacGUAUCUUACAGGAACUCCAG | | |
| ACAGCUUAUUUGGAAGCUGAAAUGUGAGGUUUAUAACACUCACAAGAAUCCUGAAA | 347 | 45 |
| aaggaugccaaacUUUCUUUCUUUGCCAGUACA | | |
| ACAGCUUAUUUGGAAGCUGAAAUGUGAGGUUUAUAACACUCACAAGAAUCCUGAAA | 348 | 45 |
| aaggaugccaaacCCAGUACAACUGCAUGUGGU | | |
| ACAGCUUAUUUGGAAGCUGAAAUGUGAGGUUUAUAACACUCACAAGAAUCCUGAAA | 349 | 45 |
| aaggaugccaaacGGCAUGUCAGUUUCAUAGGG | | |
| ACAGCUUAUUUGGAAGCUGAAAUGUGAGGUUUAUAACACUCACAAGAAUCCUGAAA | 350 | 44 |
| aaggaugccaaacGUAAUAUAAUGAUGACAACA | | |
| ACAGCUUAUUUGGAAGCUGAAAUGUGAGGUUUAUAACACUCACAAGAAUCCUGAAA | 351 | 44 |
| aaggaugccaaacAGAAGUUAAAGAGUCCAGAU | | |
| ACAGCUUAUUUGGAAGCUGAAAUGUGAGGUUUAUAACACUCACAAGAAUCCUGAAA | 352 | 44 |
| aaggaugccaaacAUGAUGACAACAACAGUCAA | | |
| ACAGCUUAUUUGGAAGCUGAAAUGUGAGGUUUAUAACACUCACAAGAAUCCUGAAA | 353 | 44 |
| aaggaugccaaacCUGAAGAUAAAUACAAUUUC | | |
| ACAGCUUAUUUGGAAGCUGAAAUGUGAGGUUUAUAACACUCACAAGAAUCCUGAAA | 354 | 44 |
| aaggaugccaaacUUUAUCUUCAGCACAUCUGG | | |
| ACAGCUUAUUUGGAAGCUGAAAUGUGAGGUUUAUAACACUCACAAGAAUCCUGAAA | 355 | 44 |
| aaggaugccaaacAAGGGUGAUGGAAAUUACUU | | |
| ACAGCUUAUUUGGAAGCUGAAAUGUGAGGUUUAUAACACUCACAAGAAUCCUGAAA | 356 | 44 |
| aaggaugccaaacACUGUUGUUGUCAUCAUUAU | | |
| ACAGCUUAUUUGGAAGCUGAAAUGUGAGGUUUAUAACACUCACAAGAAUCCUGAAA | 357 | 44 |
| aaggaugccaaacACUUCUUAAAGAUCAGGUUC | | |
| ACAGCUUAUUUGGAAGCUGAAAUGUGAGGUUUAUAACACUCACAAGAAUCCUGAAA | 358 | 44 |
| aaggaugccaaacUCUUCAGCACAUCUGGACUC | | |
| ACAGCUUAUUUGGAAGCUGAAAUGUGAGGUUUAUAACACUCACAAGAAUCCUGAAA | 359 | 44 |
| aaggaugccaaacGACUUUUUGUGUCAGGAUGA | | |
| ACAGCUUAUUUGGAAGCUGAAAUGUGAGGUUUAUAACACUCACAAGAAUCCUGAAA | 360 | 44 |
| aaggaugccaaacGACUCUUUAACUUCUUAAAG | | |
| ACAGCUUAUUUGGAAGCUGAAAUGUGAGGUUUAUAACACUCACAAGAAUCCUGAAA | 361 | 44 |
| aaggaugccaaacGUUAUACUGACAAAGAUAUC | | |
| ACAGCUUAUUUGGAAGCUGAAAUGUGAGGUUUAUAACACUCACAAGAAUCCUGAAA | 362 | 44 |
| aaggaugccaaacUUUUUUGGUUAUACUGACAA | | |
| ACAGCUUAUUUGGAAGCUGAAAUGUGAGGUUUAUAACACUCACAAGAAUCCUGAAA | 363 | 44 |
| aaggaugccaaacCUGACAAAGAUAUCACUCUG | | |
| ACAGCUUAUUUGGAAGCUGAAAUGUGAGGUUUAUAACACUCACAAGAAUCCUGAAA | 364 | 44 |
| aaggaugccaaacGCGUAUAUUUUUUGGUUAUA | | |
| ACAGCUUAUUUGGAAGCUGAAAUGUGAGGUUUAUAACACUCACAAGAAUCCUGAAA | 365 | 44 |
| aaggaugccaaacCAGAGUUUAGUUUCAAGUAA | | |
| ACAGCUUAUUUGGAAGCUGAAAUGUGAGGUUUAUAACACUCACAAGAAUCCUGAAA | 366 | 44 |
| aaggaugccaaacAUAGUUUCCUGCAUUUGCAG | | |
| ACAGCUUAUUUGGAAGCUGAAAUGUGAGGUUUAUAACACUCACAAGAAUCCUGAAA | 367 | 44 |
| aaggaugccaaacUCAAAUCGCCUGCAGGUAAA | | |
| ACAGCUUAUUUGGAAGCUGAAAUGUGAGGUUUAUAACACUCACAAGAAUCCUGAAA | 368 | 44 |
| aaggaugccaaacGAUCAAGAAAAAUAGAUGGA | | |
| ACAGCUUAUUUGGAAGCUGAAAUGUGAGGUUUAUAACACUCACAAGAAUCCUGAAA | 369 | 44 |
| aaggaugccaaacCACACCUAGCAUGUACACAC | | |
| ACAGCUUAUUUGGAAGCUGAAAUGUGAGGUUUAUAACACUCACAAGAAUCCUGAAA | 370 | 44 |
| aaggaugccaaacGAGAUAUAGCGUAUAUUUUU | | |
| ACAGCUUAUUUGGAAGCUGAAAUGUGAGGUUUAUAACACUCACAAGAAUCCUGAAA | 371 | 44 |
| aaggaugccaaacCCUGCAGGCGAUUUGACAGA | | |
| ACAGCUUAUUUGGAAGCUGAAAUGUGAGGUUUAUAACACUCACAAGAAUCCUGAAA | 372 | 44 |
| aaggaugccaaacCGCUAUAUCUCUAUAAUCUG | | |
| ACAGCUUAUUUGGAAGCUGAAAUGUGAGGUUUAUAACACUCACAAGAAUCCUGAAA | 373 | 44 |
| aaggaugccaaacUUUUUCUUGAUCCAUAUGCU | | |
| ACAGCUUAUUUGGAAGCUGAAAUGUGAGGUUUAUAACACUCACAAGAAUCCUGAAA | 374 | 44 |
| aaggaugccaaacCAAAUGCAGGAAACUAUCAG | | |
| ACAGCUUAUUUGGAAGCUGAAAUGUGAGGUUUAUAACACUCACAAGAAUCCUGAAA | 375 | 44 |
| aaggaugccaaacUUUGUUACUUGAAACUAAAC | | |
| ACAGCUUAUUUGGAAGCUGAAAUGUGAGGUUUAUAACACUCACAAGAAUCCUGAAA | 376 | 44 |
| aaggaugccaaacUACAUGCUAGGUGUGUAUAU | | |
| ACAGCUUAUUUGGAAGCUGAAAUGUGAGGUUUAUAACACUCACAAGAAUCCUGAAA | 377 | 44 |
| aaggaugccaaacUCUCUAUAAUCUGUUUUACA | | |
| ACAGCUUAUUUGGAAGCUGAAAUGUGAGGUUUAUAACACUCACAAGAAUCCUGAAA | 378 | 44 |
| aaggaugccaaacUGUACAUGCUAGGUGUGUAU | | |
| ACAGCUUAUUUGGAAGCUGAAAUGUGAGGUUUAUAACACUCACAAGAAUCCUGAAA | 379 | 44 |
| aaggaugccaaacCUUUUACCUGCAGGCGAUUU | | |
| ACAGCUUAUUUGGAAGCUGAAAUGUGAGGUUUAUAACACUCACAAGAAUCCUGAAA | 380 | 44 |
| aaggaugccaaacUUACUUGAAACUAAACUCUG | | |
| ACAGCUUAUUUGGAAGCUGAAAUGUGAGGUUUAUAACACUCACAAGAAUCCUGAAA | 381 | 44 |
| aaggaugccaaacACAGAUCUGUUGAGAAAUGG | | |
| ACAGCUUAUUUGGAAGCUGAAAUGUGAGGUUUAUAACACUCACAAGAAUCCUGAAA | 382 | 44 |
| aaggaugccaaacAAAUGUUGUGUGUACAUGCU | | |
| ACAGCUUAUUUGGAAGCUGAAAUGUGAGGUUUAUAACACUCACAAGAAUCCUGAAA | 383 | 44 |
| aaggaugccaaacUAAUCUGUUUUACAUAAUCC | | |
| ACAGCUUAUUUGGAAGCUGAAAUGUGAGGUUUAUAACACUCACAAGAAUCCUGAAA | 384 | 44 |
| aaggaugccaaacCAUGCUAGGUGUGUAUAUUA | | |
| ACAGCUUAUUUGGAAGCUGAAAUGUGAGGUUUAUAACACUCACAAGAAUCCUGAAA | 385 | 44 |
| aaggaugccaaacCAUAAUCCAUCUAUUUUUCU | | |
| ACAGCUUAUUUGGAAGCUGAAAUGUGAGGUUUAUAACACUCACAAGAAUCCUGAAA | 386 | 44 |
| aaggaugccaaacAUCUGUUUUACAUAAUCCAU | | |
| ACAGCUUAUUUGGAAGCUGAAAUGUGAGGUUUAUAACACUCACAAGAAUCCUGAAA | 387 | 44 |
| aaggaugccaaacACCAAAAAAUAUACGCUAUA | | |
| ACAGCUUAUUUGGAAGCUGAAAUGUGAGGUUUAUAACACUCACAAGAAUCCUGAAA | 388 | 53 |
| aaggaugccaaacAUUUUCUUUUGGAUUGCAUC | | |
| ACAGCUUAUUUGGAAGCUGAAAUGUGAGGUUUAUAACACUCACAAGAAUCCUGAAA | 389 | 53 |
| aaggaugccaaacUUGAAUCCUUUAACAUUUCA | | |
| ACAGCUUAUUUGGAAGCUGAAAUGUGAGGUUUAUAACACUCACAAGAAUCCUGAAA | 390 | 53 |
| aaggaugccaaacGAUUGCAUCUACUGUAUAGG | | |
| ACAGCUUAUUUGGAAGCUGAAAUGUGAGGUUUAUAACACUCACAAGAAUCCUGAAA | 391 | 53 |
| aaggaugccaaacACAUUUCAUUCAACUGUUGC | | |
| ACAGCUUAUUUGGAAGCUGAAAUGUGAGGUUUAUAACACUCACAAGAAUCCUGAAA | 392 | 53 |
| aaggaugccaaacUAGGGACCCUCCUUCCAUGA | | |
| ACAGCUUAUUUGGAAGCUGAAAUGUGAGGUUUAUAACACUCACAAGAAUCCUGAAA | 393 | 53 |
| aaggaugccaaacCUUCAUCCCACUGAUUCUGA | | |
| ACAGCUUAUUUGGAAGCUGAAAUGUGAGGUUUAUAACACUCACAAGAAUCCUGAAA | 394 | 53 |
| aaggaugccaaacAAGGUGUUCUUGUACUUCAU | | |
| ACAGCUUAUUUGGAAGCUGAAAUGUGAGGUUUAUAACACUCACAAGAAUCCUGAAA | 395 | 53 |
| aaggaugccaaacUGAUUUUCUUUUGGAUUGCA | | |
| ACAGCUUAUUUGGAAGCUGAAAUGUGAGGUUUAUAACACUCACAAGAAUCCUGAAA | 396 | 53 |
| aaggaugccaaacGGGACCCUCCUUCCAUGACU | | |
| ACAGCUUAUUUGGAAGCUGAAAUGUGAGGUUUAUAACACUCACAAGAAUCCUGAAA | 397 | 53 |
| aaggaugccaaacCUGUAUAGGGACCCUCCUUC | | |
| ACAGCUUAUUUGGAAGCUGAAAUGUGAGGUUUAUAACACUCACAAGAAUCCUGAAA | 398 | 53 |
| aaggaugccaaacGCCUGUCCUAAGACCUGCUC | | |
| ACAGCUUAUUUGGAAGCUGAAAUGUGAGGUUUAUAACACUCACAAGAAUCCUGAAA | 399 | 53 |
| aaggaugccaaacCAGUAGAUGCAAUCCAAAAG | | |
| ACAGCUUAUUUGGAAGCUGAAAUGUGAGGUUUAUAACACUCACAAGAAUCCUGAAA | 400 | 51 |
| aaggaugccaaacUCCAAGCCCGGUUGAAAUCU | | |
| ACAGCUUAUUUGGAAGCUGAAAUGUGAGGUUUAUAACACUCACAAGAAUCCUGAAA | 401 | 51 |
| aaggaugccaaacGUUUGGAGAUGGCAGUUUCC | | |
| ACAGCUUAUUUGGAAGCUGAAAUGUGAGGUUUAUAACACUCACAAGAAUCCUGAAA | 402 | 51 |
| aaggaugccaaacUCACCAGAGUAACAGUCUGA | | |
| ACAGCUUAUUUGGAAGCUGAAAUGUGAGGUUUAUAACACUCACAAGAAUCCUGAAA | 403 | 51 |
| aaggaugccaaacAUUUUAUAACUUGAUCAAGC | | |
| ACAGCUUAUUUGGAAGCUGAAAUGUGAGGUUUAUAACACUCACAAGAAUCCUGAAA | 404 | 51 |
| aaggaugccaaacUAACUUGAUCAAGCAGAGAA | | |
| ACAGCUUAUUUGGAAGCUGAAAUGUGAGGUUUAUAACACUCACAAGAAUCCUGAAA | 405 | 51 |
| aaggaugccaaacACUUGAUCAAGCAGAGAAAG | | |
| ACAGCUUAUUUGGAAGCUGAAAUGUGAGGUUUAUAACACUCACAAGAAUCCUGAAA | 406 | 51 |
| aaggaugccaaacCCAGAGCAGGUACCUCCAAC | | |
| ACAGCUUAUUUGGAAGCUGAAAUGUGAGGUUUAUAACACUCACAAGAAUCCUGAAA | 407 | 51 |
| aaggaugccaaacGAGAUGGCAGUUUCCUUAGU | | |
| ACAGCUUAUUUGGAAGCUGAAAUGUGAGGUUUAUAACACUCACAAGAAUCCUGAAA | 408 | 51 |
| aaggaugccaaacGUUACUAAGGAAACUGCCAU | | |
| ACAGCUUAUUUGGAAGCUGAAAUGUGAGGUUUAUAACACUCACAAGAAUCCUGAAA | 409 | 51 |
| aaggaugccaaacGCAGAUUUCAACCGGGCUUG | | |
| ACAGCUUAUUUGGAAGCUGAAAUGUGAGGUUUAUAACACUCACAAGAAUCCUGAAA | 410 | 51 |
| aaggaugccaaacGUGACACAACCUGUGGUUAC | | |
| ACAGCUUAUUUGGAAGCUGAAAUGUGAGGUUUAUAACACUCACAAGAAUCCUGAAA | 411 | 51 |
| aaggaugccaaacAAAUCACAGAGGGUGAUGGU | | |
| ACAGCUUAUUUGGAAGCUGAAAUGUGAGGUUUAUAACACUCACAAGAAUCCUGAAA | 412 | 51 |
| aaggaugccaaacCUUGAUCAAGUUAUAAAAUC | | |
| ACAGCUUAUUUGGAAGCUGAAAUGUGAGGUUUAUAACACUCACAAGAAUCCUGAAA | 413 | 50 |
| aaggaugccaaacCCGCCUUCCACUCAGAGCUC | | |
| ACAGCUUAUUUGGAAGCUGAAAUGUGAGGUUUAUAACACUCACAAGAAUCCUGAAA | 414 | 50 |
| aaggaugccaaacCCCUCAGCUCUUGAAGUAAA | | |
| ACAGCUUAUUUGGAAGCUGAAAUGUGAGGUUUAUAACACUCACAAGAAUCCUGAAA | 415 | 50 |
| aaggaugccaaacAGUGGAAGGCGGUAAACCGU | | |
| ACAGCUUAUUUGGAAGCUGAAAUGUGAGGUUUAUAACACUCACAAGAAUCCUGAAA | 416 | 50 |
| aaggaugccaaacCUUCAAGAGCUGAGGGCAAA | | |
| ACAGCUUAUUUGGAAGCUGAAAUGUGAGGUUUAUAACACUCACAAGAAUCCUGAAA | 417 | 50 |
| aaggaugccaaacAGCUCUGAGUGGAAGGCGGU | | |
| ACAGCUUAUUUGGAAGCUGAAAUGUGAGGUUUAUAACACUCACAAGAAUCCUGAAA | 418 | 45 |
| aaggaugccaaacACAGCUGUUUGCAGACCUCC | | |
| ACAGCUUAUUUGGAAGCUGAAAUGUGAGGUUUAUAACACUCACAAGAAUCCUGAAA | 419 | 45 |
| aaggaugccaaacUUUUUGAGGAUUGCUGAAUU | | |
| ACAGCUUAUUUGGAAGCUGAAAUGUGAGGUUUAUAACACUCACAAGAAUCCUGAAA | 420 | 45 |
| aaggaugccaaacCAGACCUCCUGCCACCGCAG | | |
| ACAGCUUAUUUGGAAGCUGAAAUGUGAGGUUUAUAACACUCACAAGAAUCCUGAAA | 421 | 45 |
| aaggaugccaaacAGGAUUGCUGAAUUAUUUCU | | |
| ACAGCUUAUUUGGAAGCUGAAAUGUGAGGUUUAUAACACUCACAAGAAUCCUGAAA | 422 | 45 |
| aaggaugccaaacGAAUACUGGCAUCUGUUUUU | | |
| ACAGCUUAUUUGGAAGCUGAAAUGUGAGGUUUAUAACACUCACAAGAAUCCUGAAA | 423 | 45 |
| aaggaugccaaacUCUGACAGCUGUUUGCAGAC | | |
| ACAGCUUAUUUGGAAGCUGAAAUGUGAGGUUUAUAACACUCACAAGAAUCCUGAAA | 424 | 45 |
| aaggaugccaaacACAACAGUUUGCCGCUGCCC | | |
| ACAGCUUAUUUGGAAGCUGAAAUGUGAGGUUUAUAACACUCACAAGAAUCCUGAAA | 425 | 45 |
| aaggaugccaaacCCGCUGCCCAAUGCCAUCCU | | |
| ACAGCUUAUUUGGAAGCUGAAAUGUGAGGUUUAUAACACUCACAAGAAUCCUGAAA | 426 | 45 |
| aaggaugccaaacCAAACAGCUGUCAGACAGAA | | |
| ACAGCUUAUUUGGAAGCUGAAAUGUGAGGUUUAUAACACUCACAAGAAUCCUGAAA | 427 | 45 |
| aaggaugccaaacCAGGAAAAAUUGGGAAGCCU | | |
| ACAGCUUAUUUGGAAGCUGAAAUGUGAGGUUUAUAACACUCACAAGAAUCCUGAAA | 428 | 45 |
| aaggaugccaaacCGGUGGCAGGAGGUCUGCAA | | |
| ACAGCUUAUUUGGAAGCUGAAAUGUGAGGUUUAUAACACUCACAAGAAUCCUGAAA | 429 | 44 |
| aaggaugccaaacGCAUGUUCCCAAUUCUCAGG | | |
| ACAGCUUAUUUGGAAGCUGAAAUGUGAGGUUUAUAACACUCACAAGAAUCCUGAAA | 430 | 44 |
| aaggaugccaaacUCAUAAUGAAAACGCCGCCA | | |
| ACAGCUUAUUUGGAAGCUGAAAUGUGAGGUUUAUAACACUCACAAGAAUCCUGAAA | 431 | 44 |
| aaggaugccaaacUCUUUCUGAGAAACUGUUCA | | |
| ACAGCUUAUUUGGAAGCUGAAAUGUGAGGUUUAUAACACUCACAAGAAUCCUGAAA | 432 | 44 |
| aaggaugccaaacUUAGCCACUGAUUAAAUAUC | | |
| ACAGCUUAUUUGGAAGCUGAAAUGUGAGGUUUAUAACACUCACAAGAAUCCUGAAA | 433 | 44 |
| aaggaugccaaacAGAAACUGUUCAGCUUCUGU | | |
| ACAGCUUAUUUGGAAGCUGAAAUGUGAGGUUUAUAACACUCACAAGAAUCCUGAAA | 434 | 44 |
| aaggaugccaaacUAUCAUAAUGAAAACGCCGC | | |
| ACAGCUUAUUUGGAAGCUGAAAUGUGAGGUUUAUAACACUCACAAGAAUCCUGAAA | 435 | 44 |
| aaggaugccaaacUUUAGCAUGUUCCCAAUUCU | | |
| ACAGCUUAUUUGGAAGCUGAAAUGUGAGGUUUAUAACACUCACAAGAAUCCUGAAA | 436 | 44 |
| aaggaugccaaacUAUUUAGCAUGUUCCCAAUU | | |
| ACAGCUUAUUUGGAAGCUGAAAUGUGAGGUUUAUAACACUCACAAGAAUCCUGAAA | 437 | 44 |
| aaggaugccaaacUGUCUUUCUGAGAAACUGUU | | |
| ACAGCUUAUUUGGAAGCUGAAAUGUGAGGUUUAUAACACUCACAAGAAUCCUGAAA | 438 | 44 |
| aaggaugccaaacAUCAGUGGCUAACAGAAGCU | | |
| ACAGCUUAUUUGGAAGCUGAAAUGUGAGGUUUAUAACACUCACAAGAAUCCUGAAA | 439 | 44 |
| aaggaugccaaacUUGAGAAAUGGCGGCGUUUU | | |
| ACAGCUUAUUUGGAAGCUGAAAUGUGAGGUUUAUAACACUCACAAGAAUCCUGAAA | 440 | 44 |
| aaggaugccaaacAAGAUAUUUAAUCAGUGGCU | | |

*In italics is a handle sequence without a linker or repeat sequence, in bold is a linker, lowercase is a repeat sequence, and no formatting is a spacer sequence.

TABLE 15
Indel activity of Effector Protein (SEQ ID NO: 1) and
Guide Nucleic Acid Combinations at Target Exons

| Spacer | sgRNA | | | |
|---|---|---|---|---|
| SEQ ID | SEQ ID | % INDEL | % INDEL | |
| NO: | NO: | REP1 | REP2 | EXON |
| 5 | 223 | 23.61995754 | 28.01176965 | 53 |
| 6 | 224 | 2.638048411 | 2.392092257 | 53 |
| 7 | 225 | 3.819145004 | 4.936770428 | 53 |
| 8 | 226 | 26.12584503 | 19.75439954 | 53 |
| 9 | 227 | 3.795977532 | 5.17471835 | 53 |
| 10 | 228 | 9.493192133 | 9.873276702 | 53 |
| 11 | 229 | 1.929797903 | 2.619366003 | 53 |
| 12 | 230 | 10.87478802 | 14.41530829 | 53 |
| 13 | 231 | 0.361933149 | 0.44251051 | 53 |
| 14 | 232 | 9.802673456 | 8.692604276 | 53 |
| 15 | 233 | 7.354593987 | 9.635651912 | 53 |
| 16 | 234 | 0 | 0 | 53 |
| 17 | 235 | 20.28317419 | 15.78315663 | 53 |
| 18 | 236 | 24.51086304 | 28.39372634 | 53 |
| 19 | 237 | 14.2396561 | 11.42857143 | 53 |
| 20 | 238 | 22.46527223 | 21.06015485 | 53 |
| 21 | 239 | 8.003822721 | 7.531472359 | 53 |
| 22 | 240 | 5.034013605 | 5.813953488 | 53 |
| 23 | 241 | 20.12229923 | 18.35788325 | 53 |
| 24 | 242 | 38.05126272 | 46.97030767 | 53 |
| 25 | 243 | 0 | 0 | 53 |
| 26 | 244 | 44.44585341 | 50.10243802 | 53 |
| 27 | 245 | 0.164925783 | 0.592885375 | 53 |
| 28 | 246 | 26.26648824 | 28.24489796 | 53 |
| 29 | 247 | 12.92918028 | 11.87330147 | 53 |
| 30 | 248 | 7.093410051 | 8.80671339 | 53 |
| 31 | 249 | 33.73835083 | 40.3687344 | 53 |
| 32 | 250 | 18.28652776 | 21.3036566 | 53 |
| 33 | 251 | 7.888065523 | 7.756770567 | 53 |
| 34 | 252 | 0.059824515 | 0 | 51 |
| 35 | 253 | 25.65177885 | 33.41127923 | 51 |
| 36 | 254 | 10.23438754 | 16.74389867 | 51 |
| 37 | 255 | 0 | 0.008216927 | 51 |
| 38 | 256 | 1.139000211 | 1.897112788 | 51 |
| 39 | 257 | 0 | 0 | 51 |
| 40 | 258 | 0.088864584 | 0.111873508 | 51 |
| 41 | 259 | 50.11990408 | 42.72502591 | 51 |
| 42 | 260 | 0.327208658 | 0.220794862 | 51 |
| 43 | 261 | 35.99516945 | 36.06216755 | 51 |
| 44 | 262 | 29.89436348 | 29.74344516 | 51 |
| 45 | 263 | 13.84764364 | 16.53330852 | 51 |
| 46 | 264 | 28.79448363 | 32.45961356 | 51 |
| 47 | 265 | 26.99039249 | 27.17571419 | 51 |
| 48 | 266 | 31.50049986 | 30.93789341 | 51 |
| 49 | 267 | 5.252404869 | 5.872613946 | 51 |
| 50 | 268 | 0.006578515 | 0 | 51 |
| 51 | 269 | 3.325137572 | 3.487358326 | 51 |
| 52 | 270 | 43.55739773 | 50.51090438 | 51 |
| 53 | 271 | 7.061286639 | 7.556626506 | 51 |
| 54 | 272 | 37.74017275 | 36.3926649 | 51 |
| 55 | 273 | 11.36732705 | 13.73254338 | 51 |
| 56 | 274 | 8.576341621 | 8.593503627 | 51 |
| 57 | 275 | 13.28252586 | 14.4140547 | 51 |
| 58 | 276 | 0.328744607 | 0.217737208 | 51 |
| 59 | 277 | 40.76778405 | 43.74037404 | 51 |
| 60 | 278 | 18.60111505 | 20.2645346 | 51 |
| 61 | 279 | 0.01592864 | 0.01772107 | 51 |
| 62 | 280 | 37.9160243 | 44.56223007 | 51 |
| 63 | 281 | 9.258900929 | 9.472285497 | 51 |
| 64 | 282 | 8.414419424 | 9.648533692 | 51 |
| 65 | 283 | 37.54007086 | 27.58913413 | 51 |
| 66 | 284 | 0.293050516 | 0.249429875 | 50 |
| 67 | 285 | 22.97531202 | 24.0800151 | 50 |
| 68 | 286 | 26.04305865 | 24.52532644 | 50 |
| 69 | 287 | 18.26977803 | 20.89901811 | 50 |
| 70 | 288 | 28.13031415 | 27.26699685 | 50 |
| 71 | 289 | 37.03826587 | 37.60559775 | 50 |
| 72 | 290 | 16.50099404 | 17.79319917 | 50 |
| 73 | 291 | 3.501678887 | 4.634968177 | 50 |
| 74 | 292 | 0.040594301 | 0 | 50 |
| 75 | 293 | 32.45206521 | 35.52957359 | 50 |
| 76 | 294 | 0.321802092 | 0.53998253 | 50 |
| 77 | 295 | 22.0463501 | 22.9641919 | 50 |
| 78 | 296 | 17.81462585 | 17.9298207 | 50 |
| 79 | 297 | 99.57519116 | 100 | 50 |
| 80 | 298 | 16.74498396 | 20.01243008 | 50 |
| 81 | 299 | 50.10969724 | 59.87529111 | 50 |
| 82 | 300 | 85.40076336 | 86.25504189 | 50 |
| 83 | 301 | 0 | 0.007770612 | 50 |
| 84 | 302 | 36.26391353 | 34.84024473 | 50 |
| 85 | 303 | 95.88414634 | 96.30952381 | 50 |
| 86 | 304 | 9.972268441 | 9.723302589 | 50 |
| 87 | 305 | 0.061297985 | 0.049146949 | 50 |
| 88 | 306 | 7.384898711 | 7.744360902 | 50 |
| 89 | 307 | 2.298850575 | 0.832626629 | 50 |
| 90 | 308 | 12.23214286 | 10.0430018 | 50 |
| 91 | 309 | 14.41213654 | 12.31696813 | 50 |
| 92 | 310 | 35.41716 | 29.11864407 | 50 |
| 93 | 311 | 10.4481535 | 7.693931398 | 50 |
| 94 | 312 | 18.11267606 | 17.34883345 | 50 |
| 95 | 313 | 34.15958658 | 21.83196793 | 50 |
| 96 | 314 | 0.238641579 | 0.249783841 | 50 |
| 97 | 315 | 11.31324616 | 8.035242291 | 50 |
| 98 | 316 | 36.95875807 | 25.76005826 | 50 |
| 99 | 317 | 5.301228696 | 3.833260458 | 50 |
| 100 | 318 | 0.05806077 | 0 | 50 |
| 101 | 319 | 22.07878627 | 20.69654126 | 50 |
| 102 | 320 | 1.239468568 | 1.306679668 | 50 |
| 103 | 321 | 0.747088552 | 0.814520267 | 45 |
| 104 | 322 | 29.60853355 | 24.21937095 | 45 |
| 105 | 323 | 14.31869054 | 9.854503258 | 45 |
| 106 | 324 | 1.769995267 | 1.739835933 | 45 |
| 107 | 325 | 0.435624395 | 0.606449772 | 45 |
| 108 | 326 | 43.20284698 | 44.65734101 | 45 |
| 109 | 327 | 0.043155533 | 0.036169636 | 45 |
| 110 | 328 | 38.60697492 | 35.88639274 | 45 |
| 111 | 329 | 0.054966627 | 0.326095602 | 45 |
| 112 | 330 | 3.705583756 | 2.731451083 | 45 |
| 113 | 331 | 1.588310038 | 1.965625297 | 45 |
| 114 | 332 | 0.767443498 | 1.261004045 | 45 |
| 115 | 333 | 14.69059318 | 17.66553024 | 45 |
| 116 | 334 | 31.20067428 | 31.40407288 | 45 |
| 117 | 335 | 34.04039379 | 34.8565356 | 45 |
| 118 | 336 | 25.99582463 | 29.40226171 | 45 |
| 119 | 337 | 0.219431915 | 0.419869058 | 45 |
| 120 | 338 | 0 | 0 | 45 |
| 121 | 339 | 0.009436633 | 0.017750954 | 45 |
| 122 | 340 | 17.94871795 | 21.18150685 | 45 |
| 123 | 341 | 11.86304821 | 13.42361863 | 45 |
| 124 | 342 | 1.12302195 | 0.822368421 | 45 |
| 125 | 343 | 1.956712723 | 4.945543454 | 45 |
| 126 | 344 | 0 | 0.057527942 | 45 |
| 127 | 345 | 9.565928348 | 12.44846039 | 45 |
| 128 | 346 | 9.976247031 | 11.70955882 | 45 |
| 129 | 347 | 26.29117131 | 32.12337156 | 45 |
| 130 | 348 | 31.46872937 | 36.41762452 | 45 |
| 131 | 349 | 5.794643488 | 5.653164145 | 45 |
| 132 | 350 | 10.63069296 | 14.84184915 | 44 |
| 133 | 351 | 38.40498315 | 47.11611434 | 44 |
| 134 | 352 | 0.142662201 | 0.097738062 | 44 |
| 135 | 353 | 9.961997828 | 12.18886567 | 14 |
| 136 | 354 | 0.887353519 | 0.777279522 | 44 |
| 137 | 355 | 8.723307587 | 9.683203825 | 44 |
| 138 | 356 | 0 | 0 | 44 |
| 139 | 357 | 3.140531585 | 3.660273055 | 44 |
| 140 | 358 | 0 | 0 | 44 |
| 141 | 359 | 0 | 0 | 44 |
| 142 | 360 | 0.213561132 | 0.355926958 | 44 |
| 143 | 361 | 0.024931439 | 0.008218953 | 44 |
| 144 | 362 | 0.214789813 | 0.205409106 | 44 |
| 145 | 363 | 47.84385857 | 54.12429379 | 44 |
| 146 | 364 | 1.284373456 | 1.566579634 | 44 |
| 147 | 365 | 0.798091163 | 1.029846477 | 44 |
| 148 | 366 | 0 | 0 | 44 |
| 149 | 367 | 3.761830598 | 2.868635546 | 44 |
| 150 | 368 | 16.22790523 | 16.73387097 | 44 |
| 151 | 369 | 25.28669725 | 26.73450509 | 44 |
| 152 | 370 | 39.7138117 | 47.06325301 | 44 |
| 153 | 371 | 0.01754386 | 0.01312336 | 44 |
| 154 | 372 | 27.61527654 | 31.01811907 | 44 |
| 155 | 373 | 0.138365747 | 0.250066982 | 44 |
| 156 | 374 | 16.23097113 | 16.15668295 | 44 |
| 157 | 375 | 27.13236077 | 30.36927257 | 44 |
| 158 | 376 | 2.544192962 | 2.813556883 | 44 |
| 159 | 377 | 5.931720036 | 6.277268275 | 44 |
| 160 | 378 | 3.607298293 | 4.324870816 | 44 |
| 161 | 379 | 0.113994501 | 0.148367953 | 44 |
| 162 | 380 | 23.93154129 | 26.43246626 | 44 |
| 163 | 381 | 15.54796859 | 15.4852931 | 44 |
| 164 | 382 | 51.76140497 | 53.47078199 | 44 |
| 165 | 383 | 0.390320062 | 0.235118093 | 44 |
| 166 | 384 | 21.20779838 | 20.10540184 | 44 |
| 167 | 385 | 0.170205868 | 0.2236909 | 44 |
| 168 | 386 | 0.305387915 | 0.231998625 | 44 |
| 169 | 387 | 57.07070707 | 62.90007513 | 44 |
| 170 | 388 | 0 | 0 | 53 |
| 171 | 389 | 5.690862783 | 6.091758709 | 53 |
| 172 | 390 | 22.30419279 | 21.29781421 | 53 |
| 173 | 391 | 11.79009016 | 12.19205367 | 53 |
| 174 | 392 | 30.34825871 | 30.33740975 | 53 |
| 175 | 393 | 11.91565546 | 13.43570058 | 53 |
| 176 | 394 | 0.028887124 | 0.019946145 | 53 |
| 177 | 395 | 0.053177346 | 0.010186411 | 53 |
| 178 | 396 | 38.22965196 | 36.59087487 | 53 |
| 179 | 397 | 14.55825153 | 14.07195161 | 53 |
| 180 | 398 | 17.40871614 | 19.21377419 | 53 |
| 181 | 399 | 0.544810678 | 0.318910533 | 53 |
| 182 | 400 | 6.66014909 | 8.732246186 | 51 |
| 183 | 401 | 8.287371965 | 12.15900933 | 51 |
| 184 | 402 | 39.34018052 | 43.21138663 | 51 |
| 185 | 403 | 0.141894289 | 0.060933811 | 51 |
| 186 | 404 | 6.831728565 | 5.945851833 | 51 |
| 187 | 405 | 12.21783741 | 10.46691106 | 51 |
| 188 | 406 | 29.2476934 | 24.42951996 | 51 |
| 189 | 407 | 2.089059923 | 2.590909091 | 51 |
| 190 | 408 | 6.65066633 | 7.026394677 | 51 |
| 191 | 409 | 1.296540248 | 1.328098897 | 51 |
| 192 | 410 | 18.77411435 | 16.04627297 | 51 |
| 193 | 411 | 0.042007982 | 0.029469548 | 51 |
| 194 | 412 | 0 | 0 | 51 |
| 195 | 413 | 0.752914508 | 0.868268725 | 50 |
| 196 | 414 | 0.431137725 | 0.406791652 | 50 |
| 197 | 415 | 0 | 0 | 50 |
| 198 | 416 | 3.918941551 | 3.283502403 | 50 |
| 199 | 417 | 2.383863081 | 2.96239052 | 50 |
| 200 | 418 | 0.523265175 | 0.325243933 | 45 |
| 201 | 419 | 0.008017317 | 0.350433174 | 45 |
| 202 | 420 | 9.445981058 | 7.432762836 | 45 |
| 203 | 421 | 22.55677469 | 20.02229122 | 45 |
| 204 | 422 | 14.72246958 | 13.11384445 | 45 |
| 205 | 423 | 16.57385309 | 15.2514427 | 45 |
| 206 | 424 | 1.228389445 | 2.139737991 | 45 |
| 207 | 425 | 3.116666667 | 1.759425494 | 45 |
| 208 | 426 | 8.816656044 | 10.06737623 | 45 |
| 209 | 427 | 4.859652473 | 5.258268302 | 45 |
| 210 | 428 | 0 | 0 | 45 |
| 211 | 429 | 40.94212302 | 42.50884782 | 44 |
| 212 | 430 | 5.103196029 | 11.47360704 | 44 |
| 213 | 431 | 16.68339691 | 24.14692685 | 44 |
| 214 | 432 | 3.392580574 | 7.101563058 | 44 |
| 215 | 433 | 1.350383632 | 3.72960373 | 44 |
| 216 | 434 | 30.75262628 | 37.65018338 | 44 |
| 217 | 435 | 37.05688764 | 26.53993334 | 44 |
| 218 | 436 | 7.792741008 | 9.932024169 | 44 |
| 219 | 437 | 27.1852212 | 28.4099723 | 44 |
| 220 | 438 | 0 | 0 | 44 |
| 221 | 439 | 0 | 0 | 44 |
| 222 | 440 | 20.21107129 | 24.08042038 | 44 |

### Example 3: Indel Activity and Splicing Disruptions and Frameshifts Analysis

Indel activity can be used to predict frameshift and splicing interruptions. Specifically, upon NGS sequencing, the location and number of indels (“reads”) can be used to predict exon-specific frameshifts, splicing interruptions, and other mutations.

Splicing interruption: Briefly, splicing interruptions can be predicted based on the location of the coding sequence overlaid on an amplicon, by counting the number of reads where there is an indel on the first 2 bases before or after the coding sequence. When indel activity reaches the edge of a coding sequence (i.e., the end or start of the coding sequence), indel counting for splice disruption analysis would not begin until after or before the end or start of the coding sequence, respectively.

Frameshifts: Frameshifts are predicted by counting all reads that are modified but not predicted for splicing interruption, and have a specific indel size.

A specific indel size depends on the frame shift and can be calculated by a modulo operation. A modulo operation is an action that given two positive integers, the operation returns the remainder after one integer is divided by another. Generally, a modulo operation can be represented by the formula (a mod n) where a is the dividend and n is the divisor. For example, the expression “5 mod 2” would evaluate to 1, because 5 divided by 2 has a quotient of 2 and a remainder of 1, while “9 mod 3” would evaluate to 0, because the division of 9 by 3 has a quotient of 3 and a remainder of 0; there is nothing to subtract from 9 after multiplying 3 times 3.

Here, frameshifts can be predicted by using the following formula:

$$x mod 3 = y , where x is the number of modified reads , \qquad Equation 1$$

$$3 is divisor and the remainder y gives the frameshift ( i . e . , 2 , 1 or 0 ) .$$

Per equation 1, the number of modified reads is divided by 3 if the remainder is 2, then a 2 frameshift mutation is predicted; if the remainder is 1, then a 1 frameshift mutation is predict, and if the remainder is 0, then an inframe mutation is predict. Inframe mutations also include where there are 0 modified reasons.

Modified reads are changes that are done in the quantification window. The quantification window is a window defined based on the effector's splicing position. It is used by the tool to define a real modification vs NGS errors. If a modification is done within the window the read is counted as modified, otherwise it is considered unmodified. An example could be an amplicon with a poly T region far from predicted splicing site, those regions can often show deletions but are actually an NGS artifact.

Other Mutations: Other mutations can also be predicted based on the location and number of indels, or based on other factors.

Indel Patterns: Analysis of splicing disruptions and frameshift mutations is used to pattern mutations as a function of indel % range for each targeted exon. Hypothetical ranges for exon-specific indel cutting patterns can be seen in TABLE 16 below.

TABLE 16
Hypothetical Exon-Specific Indel Cutting Patterns

| | % indel, | % indel, | % indel, | % indel, | % indel, |
|---|---|---|---|---|---|
| Mutation | exon A | exon B | exon C | exon D | exon E |
| 2 frameshift | 0 to 2.5% | 0 to 5% | 0 to 10% | 0 to 15% | 0 to 20% or |
| | | | | | more |
| 1 frameshift | 2.5% or less | 5% to 15% | 10% to 20% | 15% to 25% | 20% to 25% |
| | to 3.5% | | | | or more |
| splice | 3.5% or less | 15% to 17% | 20% to 25% | 25% to 27% | 25% to 30% |
| disruption | to 15% | | | | or more |
| Other | n/a | 17% to 25% | n/a | n/a | n/a |

### Example 4: Indel Activity of Effector Protein in Cardiomyocytes: Lipofection, Viability and Expression of eGFP (Plasmid and mRNA Delivery), and Indel Activity in iPSC-Derived Cardiomyocytes

Lipofection, Viability and Expression of eGFP (Plasmid and mRNA Delivery) in iPSC-Derived Cardiomyocytes

Lipofection of iPSC derived cardiomyocytes: Briefly, iPSC derived cardiomyocytes is purchased and cultured according to Takara Bio Europe AB, Cellartis® Cardiomyocytes User Manual, Cat. No. Y10075, pp. 1-6 (2018). Plasmid or mRNA encoding GFP are delivered by lipofection as described in ThermoFisher Scientific, Lipofectamine™ Stem Transfection Reagent, Pub. No. MAN0017080, pp. 1-2 (2017) and in TAN et al., “Non-viral vector based gene transfection with human induced pluripotent stem cells derived cardiomyocytes,” Sci. Reports, 9:14404 (2019) (modifying ThermoFischer Scientific, 2017 in terms of kit and lipid to DNA ratio). Results will demonstrate successful lipofection of iPSC derived cardiomyocytes.

Cardiomyocytes GFP mRNA and plasmid expression after 48h: GFP positivity of mRNA and plasmid delivered cardiomyocytes are measured 48 hours after lipofection by flow cytometry to establish the incidence of GFP expression. Mean fluorescence intensity (MFI) is measured 48 hours after lipofection by flow cytometry to establish the level of GFP expression. Results will demonstrate successful integration lipofection delivery of GFP in cardiomyocytes.

### Indel Activity of Effector Protein in Cardiomyocytes Compared to a GFP Control

Plasmids expressing effector protein/guide nucleic acid combinations and eGFP targeting the DMD gene are delivered by lipofection to iPSC derived cardiomyocytes as set forth in above Example 4. Effector protein (SEQ ID NO: 1) and guide nucleic acid combinations (TABLE 4 and TABLE 5) are delivered on the same vector.

Single and Dual cutting is assessed by delivery of one or two guides, respectively. GFP expression and indel activity is assessed 72 hours post lipofection. Results indicate indel activity. Prediction of indels are made based on NGS data as described in Example 3. Results will demonstrate that effector protein and guide nucleic acid combinations can be predicted to effect in-frame, +1 frameshift, +2 frameshift mutations, splicing disruption, and/or full sequence deletion/dual cutting. Splice disruptions mutations and 1+ frameshift mutations are predicted to be the most helpful for DMD gene editing.

### Example 5: Indel Activity of Effector Protein in Myoblasts: Lipofection, Viability, Expression of eGFP (Plasmid and mRNA Delivery), and Activity in iPSC-Derived Myoblasts

Lipofection, Viability and Expression of eGFP (Plasmid and mRNA Delivery) in iPSC-Derived Myoblasts

Lipofection of iPSC derived myoblasts: iPSC derived myoblasts are purchased and cultured according to Life Technologies Corporation, HSkM-S, Cat. No. A12555, pp. 1-2(2010). Plasmid or mRNA encoding GFP are delivered by lipofection as described in ThermoFisher Scientific, Lipofectamine™ Stem Transfection Reagent. Pub. No. MAN0017080, pp. 1-2 (2017) and in TAN et al., “Non-viral vector based gene transfection with human induced pluripotent stem cells derived cardiomyocytes,” Sci. Reports, 9:14404 (2019) as described for cardiomyocytes above. Results will demonstrate successful lipofection of iPSC derived myoblasts.

Myoblasts GFP mRNA and plasmid expression after 48h: GFP positivity of mRNA and plasmid delivered myoblasts are measured 48 hours after lipofection by flow cytometry to establish the incidence of GFP expression. Mean fluorescence intensity (MFI) is measured 48 hours after lipofection by flow cytometry to establish the level of GFP expression. Results will demonstrate successful integration lipofection delivery of GFP in cardiomyocytes.

### Indel Activity of Effector Protein in Myoblasts Compared to a GFP Control

Single and Dual cutting is assessed by delivery of one or two guides, respectively. GFP expression and indel activity are assessed 72 hours post lipofection. Results will indicate indel activity.

Prediction of mutations are made based on NGS data as described in Example 3. Results will demonstrate that effector protein (SEQ ID NO: 1) and guide nucleic acid combinations (e.g., TABLE 4 and TABLE 5) can be predicted to effect in-frame and +1 frameshift mutations.

### Example 6. CasM.265466 DMD Locus Dual Cut Pair Screening in HEK293T Cells

Guide pairs targeting DMD were screened in HEK293T cells for the identification and selection of guides for exon skipping, exon deletion/forced exon skipping and reframing therapeutic strategies. Plasmids co-expressing CasM.265466 and gRNA (1 plasmid/target) were tested in pairs for dual cut deletions of DMD locus targeting intronic and exonic regions of multiple exons (45, 50, 51 and 53). Plasmid pairs were co-transfected in HEK293T cells via lipofection with a total of 150ng of each plasmid (300ng total). Cells were incubated for 48 hours before being harvested for DNA, PCR amplified and sequenced via NGS. The sequencing data were then analyzed using CRISPRESSO to detect/quantify % indel.

Guide sequences used an sgRNA handle represented by the sequence: ACAGCUUAUUUGGAAGCUGAAAUGUGAGGUUUAUAACACUCACAAGAAUCCUGAAAAA GGAUGCCAAAC (SEQ ID NO: 4), wherein ACAGCUUAUUUGGAAGCUGAAAUGUGAGGUUUAUAACACUCACAAGAAUCCU (SEQ ID NO: 441) is a portion of a CasM.265466 tracrRNA, GAAA is the linker, and AAGGAUGCCAAAC (SEQ ID NO: 443) is the repeat. Spacer sequences were located 3′ of the sgRNA handle.

The full sequence of the polypeptide used in this experiment is: MDYKDHDGDYKDHDIDYKDDDDKMAPKKKRKVgihgvpaaMSVLTRKVQLIPVGDKEERDRVY KYLRDGIEAQNRAMNLYMSGLYFAAINEASKEDRKELNQLYSRIATSSKGSAYTTDIEFPTGLAS TSTLSMAVRQDFTKSLKDGLMYGRVSLPTYRKDNPLFVDVRFVALRGTKQKYNGLYHEYKSHT EFLDNLYSSDLKVYIKFANDITFQVIFGNPRKSSALRSEFQNIFEEYYKVCQSSIQFSGTKIILNMA MDIPDKEIELDEDVCVGVDLGIAIPAVCALNKNRYSRVSIGSKEDFLRVRTKIRNQRKRLQTNLKS SNGGHGRKKKMKPMDRFRDYEANWVQNYNHYVSRQVVDFAVKNKAKYINLENLEGIRDDVK NEWLLSNWSYYQLQQYITYKAKTYGIEVRKINPYHTSQRCSCCGYEDAGNRPKKEKGQAYFKC LKCGEEMNADFNAARNIAMSTEFQSGKKTKKQKKEQHENKKRPAATKKAGQAKKKKEFGSG EGRGSLLTCGDVEENPGPmakplsgeestlieratatinsipisedysvasaalssdgriftgvnvyhftggpcaelvvlgtaaaaaagnltc ivaignenrgilspcgrcrqvlldlhpgikaivkdsdgqptavgirellpsgyvweg* (SEQ ID NO: 476) wherein MDYKDHDGDYKDHDIDYKDDDDK (SEQ ID NO: 477) is a FLAG tag; MAPKKKRKV (SEQ ID NO: 478) is a nuclear localization signal (NLS) (SV40); gihgvpaa (SEQ ID NO: 479) is a linker; KRPAATKKAGQAKKKK (SEQ ID NO: 480) is nucleoplasmin NLS; EFGSGEGRGSLLTCGDVEENPGP (SEQ ID NO: 481) is a linker+T2A sequence (self cleaving peptide); makplsgeestlieratatinsipisedysvasaalssdgriftgvnvyhftggpcaelvvlgtaaaaaagnltcivaignenrgilspcgrcrqvlldlhp gikaivkdsdgqptavgirellpsgyvweg (SEQ ID NO: 482) confers blasticidin resistance; and the remainder of the sequence represents the 265466 protein.

Total indel and break down of different mutation patterns are provided in TABLE 17 (obtained from NGS data). Results demonstrated that combinations of nuclease and gRNA pairs (dual cut) can enhance overall target activity when compared to single cut. Additionally, pairing allows for deletion of relatively small, but also large fragments, although optimal distance between each guide must be further investigated. Dual pair cutting may have resulted in deletion of an exon/intron junction, thereby resulting in skipping of that exon.

TABLE 17
Indel Activity and Mutation Patterns

| GUIDE RNA | GUIDE RNA | | | | | | | | |
|---|---|---|---|---|---|---|---|---|---|
| PAIR | PAIR | | | | | | | | |
| SPACER - 1 | SPACER - 2 | | | | | | | | |
| SEQ ID NO: | SEQ ID NO: | A | B | C | D | E | F | G | H |
| 94 | 92 | 50 | 81.32 | 7.90 | 0.00 | 0.00 | 0.00 | 70.26 | 11.06 |
| 101 | 92 | 50 | 83.97 | 4.85 | 0.00 | 0.00 | 0.00 | 62.40 | 21.57 |
| 94 | 92 | 50 | 68.50 | 6.64 | 0.00 | 0.00 | 0.00 | 55.78 | 12.72 |
| 98 | 71 | 50 | 65.36 | 58.37 | 1.94 | 1.71 | 2.24 | 53.65 | 11.70 |
| 101 | 92 | 50 | 68.52 | 3.30 | 0.00 | 0.00 | 0.00 | 44.29 | 24.23 |
| 98 | 71 | 50 | 53.48 | 45.57 | 1.83 | 2.12 | 2.11 | 41.07 | 12.41 |
| 98 | 195 | 50 | 42.54 | 13.45 | 10.81 | 8.63 | 9.65 | 39.98 | 2.56 |
| 98 | 67 | 50 | 57.87 | 29.77 | 9.31 | 9.41 | 9.38 | 37.20 | 20.67 |
| 98 | 67 | 50 | 55.42 | 27.03 | 9.31 | 9.79 | 9.29 | 34.71 | 20.71 |
| 97 | 195 | 45 | 46.03 | 35.43 | 3.02 | 2.65 | 2.87 | 32.17 | 13.86 |
| 101 | 93 | 50 | 43.34 | 7.71 | 0.00 | 0.00 | 0.00 | 29.52 | 13.82 |
| 75 | 67 | 50 | 37.25 | 29.07 | 1.32 | 2.05 | 1.64 | 27.37 | 9.88 |
| 97 | 195 | 45 | 41.25 | 28.98 | 3.96 | 2.96 | 3.04 | 25.75 | 15.50 |
| 75 | 67 | 50 | 36.35 | 25.97 | 1.71 | 2.65 | 2.01 | 24.23 | 12.11 |
| 97 | 92 | 45 | 52.89 | 7.12 | 0.00 | 0.00 | 0.00 | 22.69 | 30.20 |
| 97 | 92 | 45 | 51.97 | 6.16 | 0.00 | 0.00 | 0.00 | 21.80 | 30.17 |
| 101 | 93 | 50 | 32.03 | 3.55 | 0.00 | 0.00 | 0.00 | 19.04 | 12.99 |
| 98 | 92 | 50 | 62.27 | 24.61 | 9.71 | 8.24 | 8.63 | 18.36 | 43.91 |
| 198 | 67 | 50 | 32.38 | 4.72 | 8.32 | 9.26 | 10.07 | 17.46 | 14.92 |
| 198 | 67 | 50 | 31.74 | 4.51 | 8.08 | 8.83 | 10.32 | 16.38 | 15.36 |
| 98 | 92 | 50 | 54.40 | 20.67 | 8.29 | 7.39 | 7.38 | 15.27 | 39.13 |
| 97 | 93 | 45 | 24.11 | 14.94 | 0.00 | 0.00 | 0.00 | 14.93 | 9.18 |
| 184 | 190 | 51 | 38.14 | 29.53 | 3.21 | 3.21 | 2.18 | 13.45 | 24.68 |
| 184 | 191 | 51 | 41.62 | 33.40 | 3.27 | 3.04 | 1.90 | 12.95 | 28.67 |
| 184 | 190 | 51 | 38.12 | 29.85 | 2.95 | 3.33 | 2.00 | 12.82 | 25.30 |
| 97 | 93 | 45 | 20.31 | 11.42 | 0.00 | 0.00 | 0.00 | 12.24 | 8.07 |
| 29 | 178 | 53 | 34.21 | 2.47 | 9.49 | 11.48 | 10.77 | 12.16 | 22.05 |
| 184 | 191 | 51 | 40.16 | 31.97 | 3.40 | 3.02 | 1.77 | 11.73 | 28.43 |
| 29 | 178 | 53 | 35.63 | 2.96 | 10.11 | 12.55 | 10.01 | 11.69 | 23.94 |
| 94 | 93 | 50 | 35.60 | 4.75 | 0.00 | 0.00 | 0.00 | 9.07 | 26.52 |
| 94 | 88 | 50 | 32.65 | 0.77 | 0.00 | 0.00 | 0.00 | 8.74 | 23.90 |
| 98 | 93 | 50 | 40.96 | 14.71 | 9.65 | 8.12 | 8.36 | 8.69 | 32.27 |
| 100 | 67 | 50 | 29.21 | 13.65 | 4.66 | 5.48 | 5.08 | 8.08 | 21.12 |
| 184 | 192 | 51 | 34.62 | 27.59 | 2.64 | 2.56 | 1.83 | 7.58 | 27.04 |
| 95 | 88 | 50 | 12.59 | 0.01 | 0.00 | 0.00 | 0.00 | 7.56 | 5.03 |
| 94 | 93 | 50 | 31.31 | 3.80 | 0.00 | 0.00 | 0.00 | 7.26 | 24.05 |
| 184 | 192 | 51 | 35.24 | 28.20 | 2.65 | 2.59 | 1.81 | 7.22 | 28.03 |
| 97 | 88 | 45 | 31.71 | 24.30 | 0.00 | 0.00 | 0.00 | 6.83 | 24.88 |
| 95 | 88 | 50 | 11.65 | 0.01 | 0.00 | 0.00 | 0.00 | 6.82 | 4.83 |
| 98 | 93 | 50 | 38.91 | 11.90 | 8.95 | 8.72 | 9.27 | 6.77 | 32.13 |
| 184 | 60 | 51 | 35.52 | 15.59 | 8.17 | 6.26 | 5.50 | 5.67 | 29.84 |
| 100 | 67 | 50 | 26.11 | 11.02 | 4.51 | 5.13 | 5.12 | 5.60 | 20.51 |
| 33 | 179 | 53 | 11.49 | 0.87 | 3.45 | 3.27 | 3.90 | 5.56 | 5.93 |
| 94 | 88 | 50 | 23.46 | 0.14 | 0.00 | 0.00 | 0.00 | 5.49 | 17.96 |
| 97 | 196 | 45 | 57.30 | 47.36 | 0.18 | 0.29 | 0.27 | 5.23 | 52.06 |
| 184 | 60 | 51 | 32.19 | 14.12 | 7.73 | 5.44 | 4.91 | 5.08 | 27.11 |
| 33 | 173 | 53 | 13.85 | 1.26 | 4.20 | 4.67 | 3.72 | 4.60 | 9.25 |
| 199 | 196 | 50 | 4.55 | 3.66 | 0.22 | 0.34 | 0.32 | 4.55 | 0.00 |
| 33 | 173 | 53 | 12.99 | 1.08 | 4.26 | 4.43 | 3.21 | 4.51 | 8.48 |
| 97 | 196 | 45 | 47.84 | 36.85 | 0.24 | 0.24 | 0.25 | 4.46 | 43.38 |
| 33 | 179 | 53 | 9.16 | 0.54 | 3.10 | 2.69 | 2.82 | 4.17 | 4.99 |
| 199 | 196 | 50 | 4.14 | 3.49 | 0.23 | 0.20 | 0.23 | 4.14 | 0.00 |
| 95 | 90 | 50 | 11.59 | 0.00 | 0.00 | 0.00 | 0.00 | 4.09 | 7.50 |
| 97 | 88 | 45 | 25.89 | 19.68 | 0.00 | 0.00 | 0.00 | 3.88 | 22.01 |
| 96 | 71 | 50 | 20.66 | 9.86 | 0.05 | 0.19 | 0.16 | 3.81 | 16.85 |
| 95 | 90 | 50 | 11.97 | 0.00 | 0.00 | 0.00 | 0.00 | 3.57 | 8.39 |
| 102 | 196 | 50 | 36.35 | 21.78 | 5.27 | 4.48 | 4.81 | 3.51 | 32.84 |
| 33 | 171 | 53 | 12.16 | 1.04 | 3.21 | 4.07 | 3.83 | 3.42 | 8.74 |
| 96 | 71 | 50 | 21.93 | 10.04 | 0.20 | 0.18 | 0.17 | 3.34 | 18.59 |
| 29 | 175 | 53 | 9.73 | 4.51 | 1.59 | 1.91 | 1.73 | 3.03 | 6.71 |
| 94 | 89 | 50 | 21.21 | 0.49 | 0.00 | 0.00 | 0.00 | 2.92 | 18.29 |
| 94 | 89 | 50 | 22.09 | 0.25 | 0.00 | 0.00 | 0.00 | 2.78 | 19.31 |
| 94 | 195 | 50 | 32.84 | 3.20 | 0.64 | 0.76 | 0.60 | 2.66 | 30.17 |
| 102 | 196 | 50 | 37.32 | 22.09 | 6.09 | 4.55 | 4.58 | 2.65 | 34.66 |
| 94 | 196 | 50 | 66.54 | 56.55 | 0.02 | 0.04 | 0.07 | 2.42 | 64.13 |
| 95 | 89 | 50 | 3.97 | 0.00 | 0.00 | 0.00 | 0.00 | 2.40 | 1.57 |
| 33 | 171 | 53 | 9.08 | 0.54 | 2.25 | 3.39 | 2.90 | 2.37 | 6.71 |
| 128 | 203 | 45 | 30.12 | 20.70 | 2.67 | 3.37 | 3.38 | 2.34 | 27.78 |
| 29 | 175 | 53 | 7.17 | 3.47 | 0.90 | 1.43 | 1.37 | 2.17 | 5.00 |
| 198 | 71 | 50 | 23.77 | 8.51 | 0.03 | 0.01 | 0.00 | 2.13 | 21.64 |
| 198 | 71 | 50 | 22.84 | 8.29 | 0.01 | 0.00 | 0.01 | 2.12 | 20.72 |
| 95 | 89 | 50 | 4.29 | 0.00 | 0.00 | 0.00 | 0.00 | 2.11 | 2.18 |
| 96 | 67 | 50 | 19.58 | 4.49 | 4.66 | 5.54 | 4.88 | 2.10 | 17.47 |
| 96 | 92 | 50 | 60.05 | 3.59 | 0.17 | 0.23 | 0.19 | 2.09 | 57.96 |
| 128 | 203 | 45 | 28.93 | 21.28 | 2.22 | 2.83 | 2.59 | 1.97 | 26.96 |
| 94 | 195 | 50 | 29.32 | 2.31 | 0.62 | 0.70 | 0.58 | 1.85 | 27.47 |
| 96 | 67 | 50 | 22.38 | 4.96 | 5.09 | 6.59 | 5.73 | 1.83 | 20.55 |
| 96 | 92 | 50 | 45.95 | 2.80 | 0.19 | 0.25 | 0.22 | 1.54 | 44.41 |
| 94 | 196 | 50 | 53.94 | 44.86 | 0.01 | 0.06 | 0.04 | 1.53 | 52.41 |
| 198 | 196 | 50 | 3.24 | 2.61 | 0.23 | 0.23 | 0.16 | 1.45 | 1.78 |
| 198 | 196 | 50 | 2.73 | 2.29 | 0.12 | 0.18 | 0.14 | 1.28 | 1.44 |
| 102 | 88 | 50 | 3.74 | 1.27 | 0.05 | 0.13 | 0.15 | 1.23 | 2.51 |
| 128 | 201 | 45 | 23.34 | 15.89 | 2.06 | 2.73 | 2.65 | 1.18 | 22.15 |
| 99 | 91 | 50 | 19.12 | 1.29 | 0.17 | 0.34 | 0.44 | 1.13 | 17.99 |
| 75 | 71 | 50 | 15.73 | 1.88 | 0.00 | 0.00 | 0.00 | 1.12 | 14.62 |
| 102 | 88 | 50 | 4.15 | 1.21 | 0.14 | 0.20 | 0.15 | 1.11 | 3.05 |
| 99 | 91 | 50 | 23.24 | 1.69 | 0.36 | 0.73 | 0.40 | 1.10 | 22.14 |
| 128 | 201 | 45 | 22.34 | 14.40 | 2.34 | 3.06 | 2.54 | 1.02 | 21.32 |

A = exon targeted
B = % of target nucleic acids modified (% indel) (modified/unmodified × 100%)
C = % of modifications predicted to result in a splice disruption
D = % of modifications predicted to result in an in frame deletion
E = % of modifications predicted to result in a +1 nucleotide frameshift
F = % of modifications predicted to result in a +2 nucleotide frameshift
G = % of target nucleic acids having a full region deletion
H = % of target nucleic acids modified (% indel) minus % full region deletion

### Example 7: Gene Editing of iPSC-Derived Cardiomyocytes with AAV Vector Encoding Effector Protein and Guide Nucleic Acid

An AAV vector was constructed to contain a transgene between its ITRs, the transgene providing or encoding, in a 5′ to 3′ direction, a U6 promoter, a guide nucleic acid, a MND promoter, a sequence encoding CasM.265466, a WPRE enhancer, and an hGH-poly A signal was packaged into an AAV vector, as illustrated in FIG. 2, construct E). The effector protein has an amino acid sequence of SEQ ID NO: 1. The guide nucleic acid comprised a sequence that is represented by:

(SEQ ID NO: 450)
ACAGCUUAUUUGGAAGCUGAAAUGUGAGGUUUAUAACACUCACA
AGAAUCCGAAAAAGGAUGCCAAACUCACCAGAGUAACAGUCUGA.

The AAV vector was expressed with supporting plasmids to produce an adeno-associated virus (AAV). iPSC-derived cardiomyocytes were contacted with the AAV. After about 72 hours, DNA was isolated from the infected cells. Indels generated with this AAV system were detected and quantified by sequencing. As much as 12% indel were observed.

### Example 8: AAV Vectors for Gene Editing by a Single Cut

An AAV vector is constructed to contain a transgene between its ITRs, the transgene providing or encoding, in a 5′ to 3′ direction, a nucleotide sequence of a first promoter, a nucleotide sequence encoding a guide nucleic acid, a nucleotide sequence of a second promoter, a nucleotide sequence encoding an effector protein, optionally an enhancer, and a poly A signal sequence as illustrated in FIG. 2 are packaged into an AAV vector. The effector protein has an amino acid sequence that has at least 80% identical to the amino acid sequence of SEQ ID NO: 1. The guide nucleic acid comprises a nucleotide sequence that has at least 90% identical to any one of the nucleotide sequences recited in TABLE 4, TABLE 5 and TABLE 6. As illustrated in FIG. 2, the effector protein can be expressed either ubiquitously or in a specific muscle based on the promotor the AAV vector is engineered to have. The AAV vector has a second promotor U6, and WPRE enhancer. The AAV vector may have hGH Poly A signal sequence or sv40 Poly A signal sequence. The AAV vector is expressed with supporting plasmids to produce an adeno-associated virus (AAV).

### Example 9: AAV Vectors for Gene Editing by a Dual Cut

An AAV vector is constructed to contain a transgene between its ITRs, the transgene providing or encoding, in a 5′ to 3′ direction, a first promoter, a first guide nucleic acid, a second promoter, an effector protein, an enhancer, a poly A signal sequence, a third promotor, and a second guide nucleic acid as illustrated in FIG. 3 are packaged into an AAV vector. The effector protein has an amino acid sequence that has at least 80% identical to the amino acid sequence of SEQ ID NO: 1. The first guide nucleic acid and the second guide nucleic acid comprise different spacer sequences targeting them to different target sequences of DMD. The first guide nucleic acid and the second guide nucleic acid independently comprises a nucleotide sequence that is at least 90% identical to any one of the nucleotide sequences recited in TABLE 4, TABLE 5, and TABLE 6. In some examples, the first guide nucleic acid and the second guide nucleic acid are complementary to sequences 5′ and 3′ of a given exon, respectively. Therefore, the dual cut can remove the exon. As illustrated in FIG. 3, the effector protein can be expressed either ubiquitously or in a specific muscle based on the promotor the AAV vector is engineered to have. The AAV vector also has U6 first promotor, 7SK second promotor, WPRE enhancer, and hGH Poly A signal sequence. The AAV vector is expressed with supporting plasmids to produce an adeno-associated virus (AAV).

### Example 10: Gene Editing of iPSC-Derived Cardiomyocytes with AAV Vector Encoding Effector Protein and Guide Nucleic Acid

An AAV vector is constructed to contain a transgene between its ITRs according to any one of the constructs described in Example 8 and 9. The AAV vector is expressed with supporting plasmids to produce an adeno-associated virus (AAV). iPSC-derived cardiomyocytes are contacted with the AAV. After about 48-96 hours, DNA or RNA is isolated from the infected cells. An indel caused by the guide nucleic acid is confirmed by sequencing and/or Q-PCR.

### Example 11: In Vivo Gene Editing in a Mammalian Model for Treating Muscular Dystrophy Mutation(s) by AAV

An AAV vector is constructed to contain a transgene between its ITRs according to any one of the constructs described in Example 8 and 9. The AAV vector is expressed with supporting plasmids to produce an adeno-associated virus (AAV). A mouse with muscular dystrophy is administered an effective dose of the AAV. About four weeks post administration, a sample muscle is extracted for analysis of dystrophin restoration. The sample muscle can be chosen based on the promotor used for expressing the effector protein. The analysis can be performed by any technique known to a skillful artisan, which includes but are not limited to immunohistochemistry, western blot analysis and deep-sequencing analysis. Similarly, rescue of pathological phenotypes can be determined by performing any technique known to a skillful artisan, which includes but are not limited to hematoxylin and eosin (H&E) staining, Masson's trichrome staining, grip-strength analysis, muscular electrophysiological analysis, and serum creatine kinase (CK).

### Example 12. CasM.265466 DMD Exon Deletion in HEK293T Cells

Guide pairs targeting DMD were screened in HEK293T cells for the identification and selection of guides for exon deletion therapeutic strategies. Plasmids co-expressing CasM.265466 and gRNA (1 plasmid/target) were tested in pairs for dual cut deletions of certain DMD locus exons (44, 45, 50, 51, or 53). Plasmid pairs were co-transfected in HEK293T cells via lipofection. Cells were incubated for 72 hours before being harvested for DNA, PCR amplified and sequenced via NGS. The sequencing data were then analyzed using CRISPRESSO to detect/quantify % indel and exon deletions.

Guide nucleic acids used an sgRNA handle sequence represented by the sequence: ACAGCUUAUUUGGAAGCUGAAAUGUGAGGUUUAUAACACUCACAAGAAUCCUGAAAAA GGAUGCCAAAC (SEQ ID NO: 4), wherein ACAGCUUAUUUGGAAGCUGAAAUGUGAGGUUUAUAACACUCACAAGAAUCCU (SEQ ID NO: 441) is a portion of a CasM.265466 tracrRNA, GAAA is the linker, and AAGGAUGCCAAAC (SEQ ID NO: 443) is the repeat sequence. Spacer sequences were located 3′ of the sgRNA handle.

The full sequence of the polypeptide used in this experiment is: MDYKDHDGDYKDHDIDYKDDDDKMAPKKKRKVgihgvpaaMSVLTRKVQLIPVGDKEERDRVY KYLRDGIEAQNRAMNLYMSGLYFAAINEASKEDRKELNQLYSRIATSSKGSAYTTDIEFPTGLAS TSTLSMAVRQDFTKSLKDGLMYGRVSLPTYRKDNPLFVDVRFVALRGTKQKYNGLYHEYKSHT EFLDNLYSSDLKVYIKFANDITFQVIFGNPRKSSALRSEFQNIFEEYYKVCQSSIQFSGTKIILNMA MDIPDKEIELDEDVCVGVDLGIAIPAVCALNKNRYSRVSIGSKEDFLRVRTKIRNQRKRLQTNLKS SNGGHGRKKKMKPMDRFRDYEANWVQNYNHYVSRQVVDFAVKNKAKYINLENLEGIRDDVK NEWLLSNWSYYQLQQYITYKAKTYGIEVRKINPYHTSQRCSCCGYEDAGNRPKKEKGQAYFKC LKCGEEMNADFNAARNIAMSTEFQSGKKTKKQKKEQHENKKRPAATKKAGQAKKKKEFGSG EGRGSLLTCGDVEENPGPmakpLsgeestlieratatinsipisedysvasaalssdgriftgvnvyhftggpcaelvvlgtaaaaaagnltc ivaignenrgilspcgrcrqvlldlhpgikaivkdsdgqptavgirellpsgyvweg* (SEQ ID NO: 476) wherein MDYKDHDGDYKDHDIDYKDDDDK (SEQ ID NO: 477) is a FLAG tag; MAPKKKRKV (SEQ ID NO: 478) is a nuclear localization signal (NLS) (SV40); gihgvpaa (SEQ ID NO: 479) is a linker; KRPAATKKAGQAKKKK (SEQ ID NO: 480) is nucleoplasmin NLS; EFGSGEGRGSLLTCGDVEENPGP (SEQ ID NO: 481) is a linker+T2A sequence (self cleaving peptide); makplsgeestlieratatinsipisedysvasaalssdgriftgvnvyhftggpcaelvvlgtaaaaaagnltcivaignenrgilspcgrcrqvldlhp gikaivkdsdgqptavgirellpsgyvweg (SEQ ID NO: 482) confers blasticidin resistance; and the remainder of the sequence represents the 265466 protein.

Total indel of different exon deletions are provided in TABLE 18 (obtained from NGS data). Results demonstrated that combinations of nuclease and gRNA pairs (dual cut) can be used to delete an entire exon (44, 45, 50, 51, or 53), thereby resulting in skipping of that exon during translation and protein production.

TABLE 18
Indel Activity and Exon Deletion

| | | | | | FULL | FULL | |
|---|---|---|---|---|---|---|---|
| | Spacer-1 | Spacer-2 | % | % | REGION | REGION | |
| | SEQ ID | SEQ ID | INDEL | INDEL | DELETION | DELETION | |
| No. | NO: | NO: | REP1 | REP2 | REP1 | REP2 | EXON |
| 1 | 155 | 218 | + | + | * | * | 44 |
| 2 | 155 | 217 | + | + | * | * | 44 |
| 3 | 155 | 211 | + | + | * | * | 44 |
| 4 | 161 | 218 | ++ | ++ | * | * | 44 |
| 5 | 161 | 217 | + | + | * | * | 44 |
| 6 | 161 | 211 | +++ | +++ | ** | * | 44 |
| 7 | 153 | 218 | ++ | +++ | ** | ** | 44 |
| 8 | 153 | 217 | ++ | + | ** | ** | 44 |
| 9 | 153 | 211 | +++ | +++ | ** | ** | 44 |
| 10 | 150 | 136 | + | + | ND | ND | 44 |
| 11 | 150 | 140 | ++ | ++ | ND | ND | 44 |
| 12 | 149 | 136 | + | + | ND | ND | 44 |
| 13 | 149 | 140 | ++ | ++ | ND | ND | 44 |
| 14 | 122 | 103 | ++ | ++ | ND | ND | 45 |
| 15 | 122 | 107 | ++ | ++ | ND | ND | 45 |
| 16 | 121 | 103 | + | + | ND | ND | 45 |
| 17 | 121 | 107 | + | + | * | ND | 45 |
| 18 | 126 | 103 | + | + | ND | ND | 45 |
| 19 | 126 | 107 | + | + | ND | ND | 45 |
| 20 | 128 | 103 | +++ | +++ | * | * | 45 |
| 21 | 128 | 107 | +++ | +++ | * | * | 45 |
| 22 | 117 | 208 | +++ | +++ | ND | ND | 45 |
| 23 | 117 | 113 | +++ | +++ | ND | ND | 45 |
| 24 | 207 | 208 | + | + | * | * | 45 |
| 25 | 207 | 113 | ++ | ++ | ND | ND | 45 |
| 26 | 206 | 208 | + | + | * | * | 45 |
| 27 | 206 | 113 | + | + | ND | ND | 45 |
| 28 | 96 | 67 | +++ | +++ | * | * | 50 |
| 29 | 96 | 71 | +++ | +++ | * | | 50 |
| 30 | 99 | 67 | +++ | +++ | ** | ** | 50 |
| 31 | 99 | 71 | +++ | +++ | ** | | 50 |
| 32 | 102 | 67 | +++ | +++ | * | * | 50 |
| 33 | 102 | 71 | +++ | +++ | * | * | 50 |
| 34 | 98 | 67 | +++ | +++ | ** | ** | 50 |
| 35 | 98 | 71 | +++ | +++ | ** | ** | 50 |
| 36 | 86 | 77 | ++ | ++ | ND | ND | 50 |
| 37 | 86 | 84 | ++ | ++ | ND | ND | 50 |
| 38 | 86 | 75 | ++ | ++ | ND | ND | 50 |
| 39 | 86 | 76 | + | + | ND | ND | 50 |
| 40 | 86 | 74 | + | + | ND | ND | 50 |
| 41 | 86 | 83 | + | + | ND | ND | 50 |
| 42 | 92 | 77 | +++ | +++ | ** | ** | 50 |
| 43 | 92 | 84 | +++ | +++ | ** | ** | 50 |
| 44 | 92 | 75 | ++ | ++ | * | * | 50 |
| 45 | 92 | 76 | +++ | +++ | ND | ND | 50 |
| 46 | 92 | 74 | +++ | +++ | ND | ND | 50 |
| 47 | 92 | 83 | +++ | +++ | ND | ND | 50 |
| 48 | 93 | 77 | ++ | ++ | ** | ** | 50 |
| 49 | 93 | 84 | ++ | +++ | * | ** | 50 |
| 50 | 93 | 75 | ++ | +++ | * | * | 50 |
| 51 | 93 | 76 | + | + | * | ND | 50 |
| 52 | 93 | 74 | + | + | ND | ND | 50 |
| 53 | 93 | 83 | + | ++ | ND | ND | 50 |
| 54 | 195 | 77 | ++ | +++ | * | * | 50 |
| 55 | 195 | 84 | +++ | +++ | * | * | 50 |
| 56 | 195 | 75 | +++ | +++ | * | * | 50 |
| 57 | 195 | 76 | + | + | * | * | 50 |
| 58 | 195 | 74 | + | + | * | * | 50 |
| 59 | 195 | 83 | + | + | * | * | 50 |
| 60 | 60 | 37 | ++ | ++ | ND | ND | 51 |
| 61 | 60 | 34 | ++ | ++ | ND | ND | 51 |
| 62 | 60 | 43 | ++ | ++ | ND | ND | 51 |
| 63 | 48 | 46 | ++ | ++ | ND | ND | 51 |
| 64 | 48 | 193 | + | + | ND | ND | 51 |
| 65 | 184 | 46 | ++ | ++ | ND | ND | 51 |
| 66 | 184 | 193 | +++ | +++ | ND | ND | 51 |
| 67 | 189 | 46 | ++ | ++ | ND | ND | 51 |
| 68 | 189 | 193 | ++ | ++ | * | * | 51 |
| 69 | 33 | 177 | ++ | ++ | * | * | 53 |
| 70 | 33 | 5 | +++ | +++ | * | * | 53 |
| 71 | 33 | 10 | + | + | * | * | 53 |
| 72 | 33 | 8 | ++ | ++ | ND | ND | 53 |
| 73 | 29 | 177 | + | + | * | * | 53 |
| 74 | 29 | 5 | ++ | ++ | * | * | 53 |
| 75 | 29 | 10 | + | + | * | * | 53 |
| 76 | 29 | 8 | ++ | ++ | ND | ND | 53 |
| 77 | 18 | 181 | ++ | ++ | ND | ND | 53 |
| 78 | 18 | 5 | ++ | ++ | ND | ND | 53 |
| 79 | 20 | 181 | ++ | ++ | ND | ND | 53 |
| 80 | 20 | 5 | ++ | ++ | ND | ND | 53 |
| 81 | 175 | 181 | ++ | ++ | ** | * | 53 |
| 82 | 175 | 5 | +++ | +++ | * | * | 53 |
| 83 | 176 | 181 | ++ | ++ | * | * | 53 |
| 84 | 176 | 5 | ++ | ++ | * | * | 53 |

“+” indicates <15% indel; “++” indicates ≥15% to <35% indel; “+++” indicates ≥35% indel; ND = Not detected; “*” indicates >0 to <5% exon deletion; “**” indicates ≥5% exon deletion.

The data was further confirmed for exon deletion by sequencing. TABLE 19 provides nucleotide sequences of primers that were used to confirm exon deletion.

TABLE 19
Sequencing Primers for Exon Deletion Confirmation

| | SEQ ID | Primer | |
|---|---|---|---|
| Exon | NO: | Type | Primer Sequence (5′ to 3′) |
| 44 | 456 | Forward | TCGTCGGCAGCGTCAGATGTGTATAAGAGACAGCTCTG |
| | | Primer | CAAATGCAGGAAACTATCAGAG |
| | 457 | Reverse | GTCTCGTGGGCTCGGAGATGTGTATAAGAGACAGTAAA |
| | | Primer | CCAGCTCCGTCCAGGC |
| 45 | 458 | Forward | TCGTCGGCAGCGTCAGATGTGTATAAGAGACAGCTGTC |
| | | Primer | TTGTATCCTTTGGATATGGGC |
| | 459 | Reverse | GTCTCGTGGGCTCGGAGATGTGTATAAGAGACAGCTGC |
| | | Primer | TGTTGATTAATGGTTGATAGGTTC |
| 50 | 460 | Forward | TCGTCGGCAGCGTCAGATGTGTATAAGAGACAGGCAAT |
| | | Primer | TGATAAATATTTGTAGGGTGGTTGG |
| | 461 | Reverse | GTCTCGTGGGCTCGGAGATGTGTATAAGAGACAGAGTC |
| | | Primer | AATTTCCAAGGAATGTACTCTAAGAC |
| 51 | 462 | Forward | TCGTCGGCAGCGTCAGATGTGTATAAGAGACAGATTGA |
| | | Primer | AATTGGCTCTTTAGCTTGTGTTTCT |
| | 463 | Reverse | GTCTCGTGGGCTCGGAGATGTGTATAAGAGACAGCAGT |
| | | Primer | TGCCTAAGAACTGGTGGGA |
| 53 | 464 | Forward | TCGTCGGCAGCGTCAGATGTGTATAAGAGACAGTTGTT |
| | | Primer | CATCATCCTAGCCATAACACAAT |
| | 465 | Reverse | GTCTCGTGGGCTCGGAGATGTGTATAAGAGACAGGTCT |
| | | Primer | ACTGTTCATTTCAGCTTTAACGTG |

An analysis of sequencing data confirmed that CasM.265466 can be used to delete a whole exon of interest to correct the frame of DMD in patients.

### Example 13: In Vitro Enrichment in Mammalian Cells

In this experiment CasM.265466 was expressed in HEK293T cells, and cell lysate was tested for nucleic acid cleaving activity. Purified CasM.265466 from HEK293T cells was also tested for nucleic acid cleaving activity. In both cases, cis cleavage activity was detected by the presence of bands. The PAM requirements were determined to be TNTR (SEQ ID NO: 3) by NGS after in vitro enrichment of DNA fragments.

In vitro enrichment involved the amplification of DNA fragments excised by potential CRISPR-Cas candidates. The method began with a cis cleavage assay, which was then followed by dA end repair, ligation, and multiple rounds of PCR. Magnetic bead purification was also performed after interference, ligation, and both rounds of PCR. The final purified PCR product was then sequenced on a MiSeq instrument. Details of these steps are provided as follows.

### HEK293 Lipofection and Lysis

Opti-MEM media was warmed to 37° C. and transfection reagent was equilibrated at room temperature. Final transfection ratio was prepared at pDNA:Lipid—300 ng pDNA: 0.6 μl tx reagent per transfection. A first solution was prepared by diluting pDNA with Opti-MEM—360 ng pDNA diluted with media to final volume of 12 μL. A second solution was prepared by diluting the transfection reagent with Opti-MEM—0.72 μl tx reagent diluted with media to final volume of 12 μL. 12 μL of the first solution and 12 μL of the second solution were mixed and incubated at room temperature for 15 minutes, and then a 20 μL aliquot of the mixture was dispensed over the cells followed by incubation at 37° C. for approximately 72 hours before harvesting.

### Interference Assay

Purified CRISPR effector protein CasM.265466 (50 μM) was added to a reaction containing 10 μl 10× Cutsmart buffer, and a plasmid (1000 ng per reaction). Additionally, prepared in parallel was a solution containing 3 μL of EcoRI and 7 μL dH₂O as a positive control. Dilutions and volumes of the prepared reactions for 3′ PAM and 5′ PAM are shown in TABLE 20. The reaction was incubated at 37° C. for 30 minutes, 5 μL EDTA+1 μL Proteinase K solution were then added, and the reaction was further incubated at 37° C. for 30 minutes. NGS was subsequently performed, and the required PAM determined was 5′-TNTR-3′ (SEQ ID NO: 3).

TABLE 20
Interference Assay Reaction

| | 3′PAM | | 5′PAM | |
|---|---|---|---|---|
| | 1x Volume (μL) | 50 | 1X Volume (uL) | 20 |
| 10x CutSmart Buffer | 10 | 500 | 10 | 200 |
| Plasmid (1000 ng/rxn) | 0.9496676163 | 47.4833808167 | 3.0303030303 | 60.6060606061 |
| dH2O | | 3852.5166191833 | | 1499.3939393939 |
| Protein (50 μM) | 12 | 600 | 12 | 240 |
| Total | 100 | 5000 | 100 | 2000 |

### Magnetic Bead Purification I

SPRIselect beads for resuspension in solution were prepared. 60 μL of each bead solution was added to each interference assay reaction and incubated for 5 minutes at 25° C. The reactions were then placed on a magnetic stand. After 1 minute, clear liquid was aspirated from each reaction without disturbing the magnetic beads. To each reaction still containing the magnetic beads, 190 μL of 80% ethanol was added. The ethanol was then removed from each well without disturbing the magnetic beads. The addition and removal of ethanol was repeated with 200 μL of 80% ethanol to each reaction. The magnetic beads in each reaction vessel were then resuspended in 55 μL nuclease free 1×TE buffer or dH₂O. The resuspension solutions were incubated for 1 minute at 25° C. and returned to the magnetic stand. 50 μL of each resuspension solution were then transferred into new reaction plates.

End Repair—dA-Tailing

Reactions containing purified DNA were expose to 7 μL of Ultra II EP buffer and diluted to 180 μL. An additional 3 μL of Ultra II End Prep Enzyme Mix was added to each reaction. The reactions were then mixed thoroughly and then placed in a thermocycler according to the timeline in TABLE 21.

TABLE 21
Thermocycler Programming

| Steps | Time (minutes) | Temperature (° C.) |
|---|---|---|
| 1 | 30 | 20 |
| 2 | 30 | 65 |
| 3 | ∞ | 4 |

### Adapter Ligation

To the end prepared reactions described above, the following components described in TABLE 22 were added at 0° C. An adapter sequence of ILM8-UDI-UMI was used and the reactions were mixed thoroughly and incubated at 20° C. for 15 minutes in a thermocycler with the heated lid removed from the apparatus.

TABLE 22
Adapter Ligation Reaction Components

| | Volume (μL) |
|---|---|
| EndRepair Reaction | 60 |
| NEBNext Ultra II Ligation Master Mix | 30 |
| NEBNext Ligation Enhancer | 1 |
| ILM8-UDI-UMI adaptor (1.5 μM) | 2.5 |
| dH2O | 6.5 |
| Total | 100 |

### Magnetic Bead Purification II

SPRIselect Beads were mixed to resuspend the beads in solution. To each ligation reaction described above, 60 μL of the SPRIselect Bead suspension were added and then 25 μL of nuclease-free water was added.

### PCR for Target Enrichment

PCR for target enrichment was conducted by preparing reactions with various IVE primers possessing different overhang sequences as shown in TABLE 23 and TABLE 24.

TABLE 23
PCR for Target Enrichment Reaction Components

| Reagent | 1x Volume (μL) | 96 |
|---|---|---|
| 2X Q5 NEBNext | 12.5 | 1200 |
| IVE F pool (100 μM) | 0.125 | 12 |
| P7 Reverse Primer (100 μM) | 0.125 | 12 |
| Water | 2.25 | 216 |
| Post Clean up Ligation Reaction | 10 | |
| Total Volume | 25 | 2400 |

TABLE 24
IVE Primer Sequences

| Name | Sequence |
|---|---|
| IVE longF-A | ACACTCTTTCCCTACACGACGCTCTTCC |
| AJD001 | GATCtNcctttcgtctcgcgcgtttcgg |
| | (SEQ ID NO: 483) |
| IVE longF-B | ACACTCTTTCCCTACACGACGCTCTTCC |
| AJD001 | GATCtNNNcctttcgtctcgcgcgtttc |
| | gg (SEQ ID NO: 484) |
| IVE longF-C | ACACTCTTTCCCTACACGACGCTCTTCC |
| AJD001 | GATCtNNNNNcctttcgtctcgcgcgtt |
| | tcgg (SEQ ID NO: 485) |
| IVE longF-D | ACACTCTTTCCCTACACGACGCTCTTCC |
| AJD001 | GATCtNNNNNNNcctttcgtctcgc |
| | gcgtttcgg (SEQ ID NO: 486) |

### Example 14: Additional PAM Screening for CasM.265466

Prior in vitro screening as described in Example 1 for effector protein CasM.265466 (SEQ ID NO: 1) PAM recognition demonstrated that the most enriched PAM sequence for CasM.265466 (SEQ ID NO: 1) was a TNTR (SEQ ID NO: 3) PAM sequence, but also indicated that the effector protein may tolerate a more flexible PAM sequences beyond TNTR (SEQ ID NO: 3) without significantly compromising nuclease activity. Effector protein and flexible PAM group combinations as set forth in TABLE 25 were screened to confirm that chromosomal DNA may be efficiently targeted in mammalian cells (HEK293T) using a more flexible PAM sequence.

Single and double point mutations were made along TNTR (SEQ ID NO: 3).

TABLE 25
PAM SEQUENCES

| SEQ ID NO: | PAM Group* |
|---|---|
| 487 | NNTN |
| 488 | ANTR |
| 489 | CNTR |
| 490 | GNTR |
| 491 | TNAR |
| 492 | TNCR |
| 493 | TNGR |
| 494 | TNTC |
| 495 | TNTT |
| 496 | VNTY |
| 497 | TNVY |

*wherein each N is any nucleotide, each R is A or G, and each V is A, C or G.

At least six spacers that previously showed >3% indel rate were selected for each PAM group identified in TABLE 25.

Single guide nucleic acids (sgRNA) comprising a handle sequence of SEQ ID NO: 4 and a spacer sequence comprising 20 nucleotides was used.

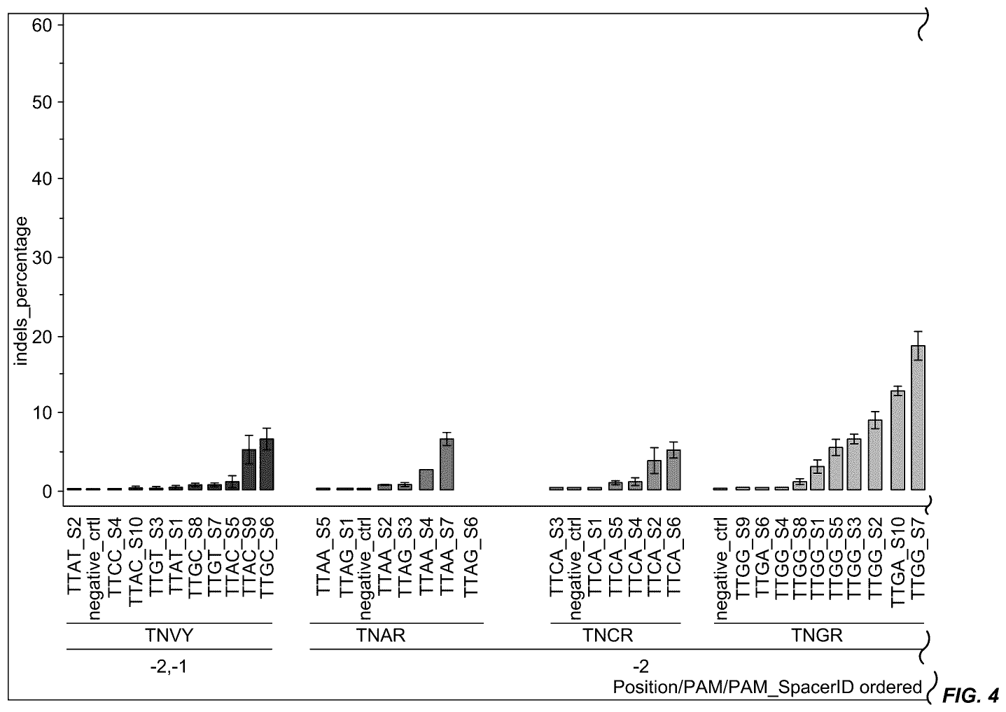

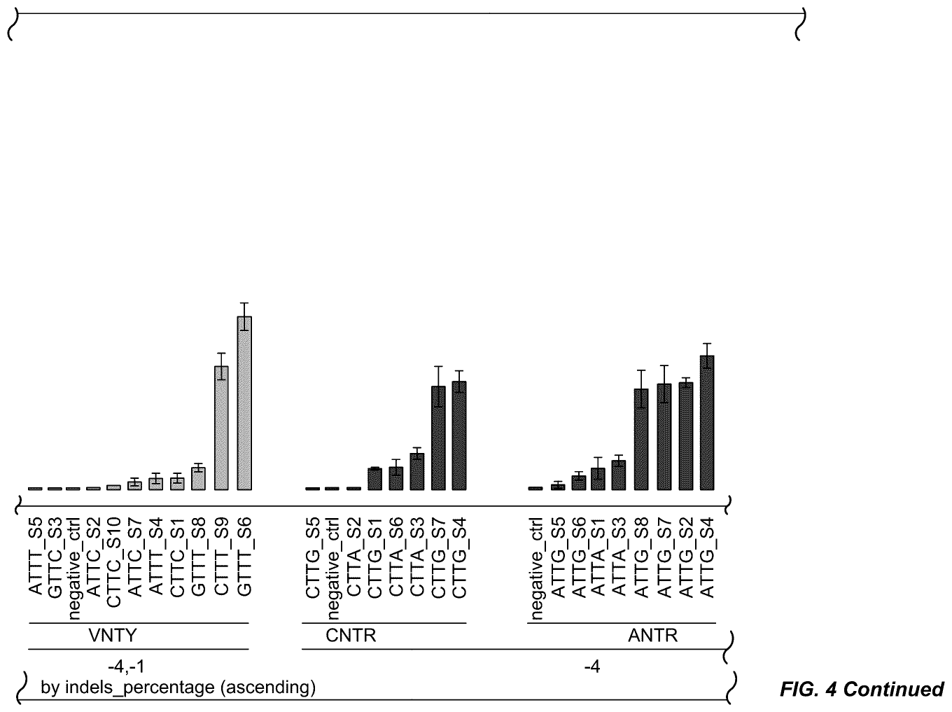

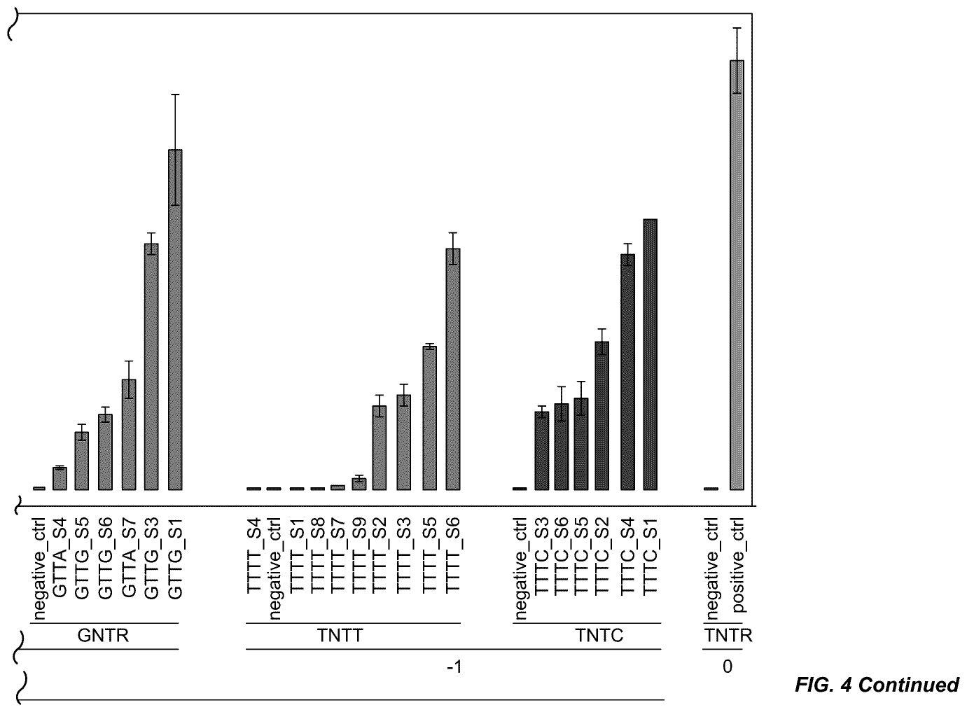

Plasmids encoding CasM.265466 effector protein (SEQ ID NO: 1) and plasmids encoding the sgRNAs were delivered via lipofection to HEK293T cells and permitted to grow to allow for indel formation. Cells were lysed and indels were detected by next generation sequencing. Indel percentage was calculated and plotted as shown in FIG. 4.

Some complexes were found to produce up to or greater than 30% indel. Data also demonstrated that single and double point mutations at −4 and −1 were the most permissive for allowing nuclease activity. Furthermore, the CasM.265466 effector protein (SEQ ID NO: 1) complexed with two different sgRNAs having different spacer sequences generated 20% indel at targeted sequences adjacent to an NNTN (SEQ ID NO: 487) PAM. Therefore, these results further confirm the results of Example 1 and demonstrate that the CasM.265466 effector protein (SEQ ID NO: 1) recognizes a flexible NNTN (SEQ ID NO: 487) PAM sequence.

### Example 15: In Vitro Testing of Exon Skipping Approach in iPSC

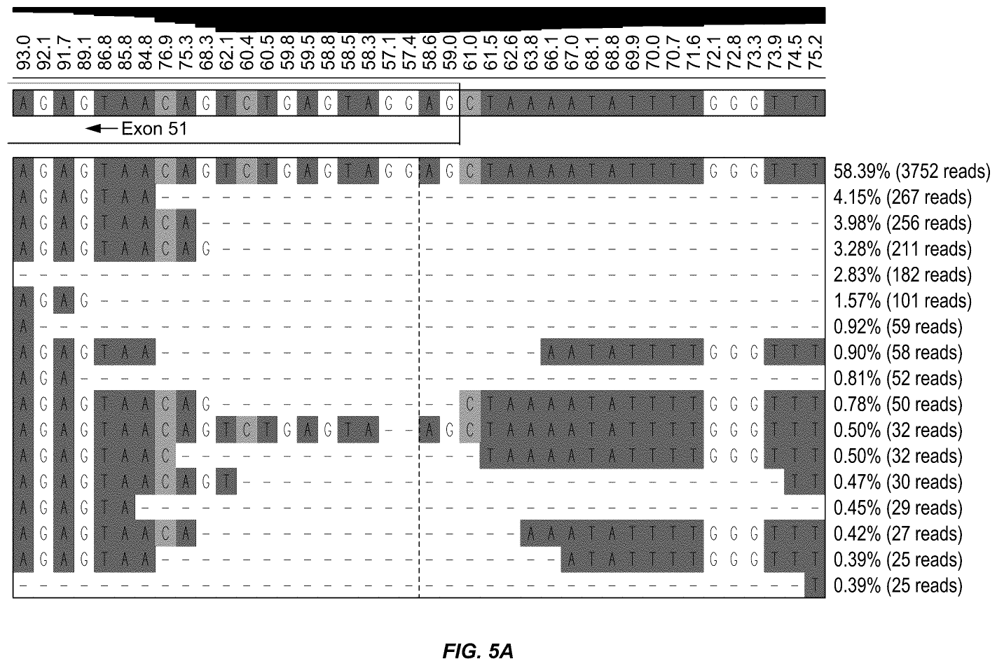

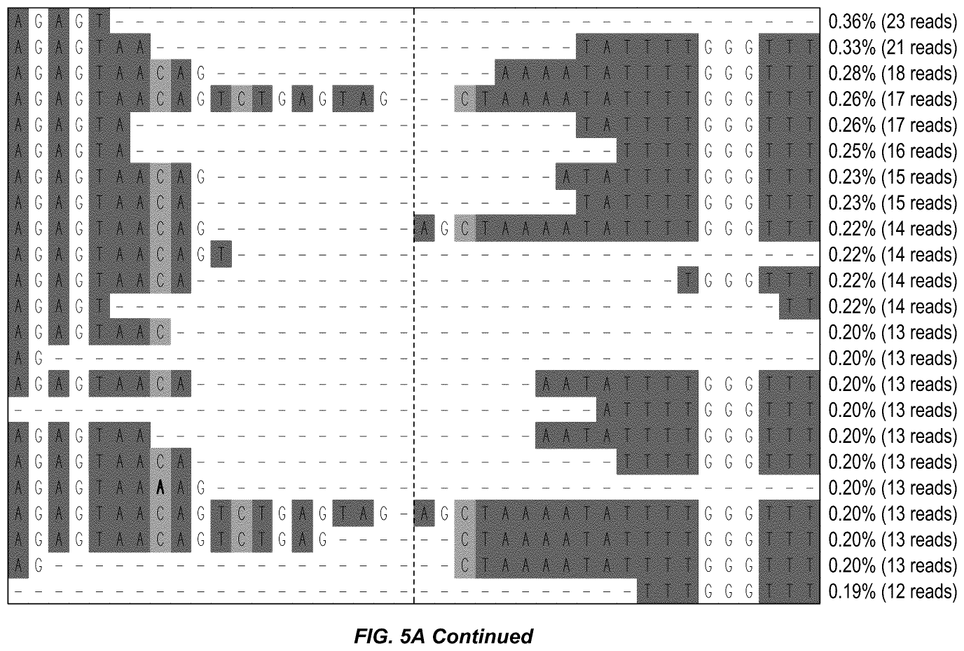

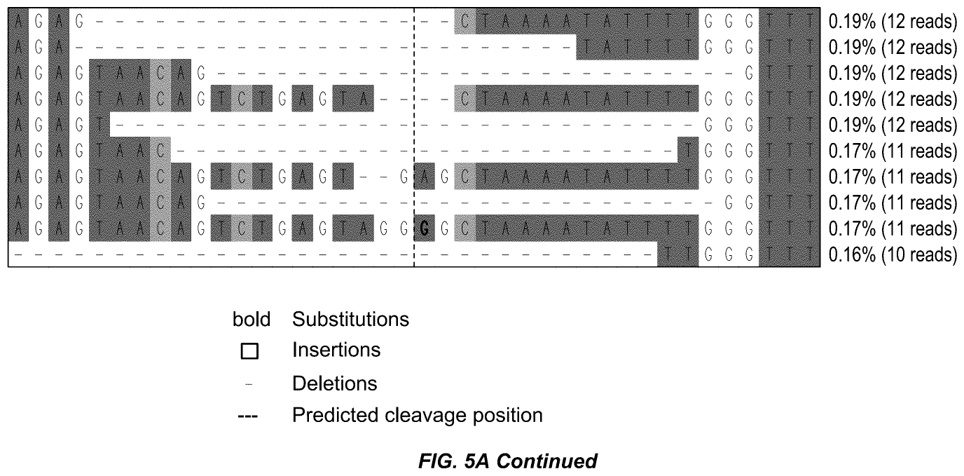

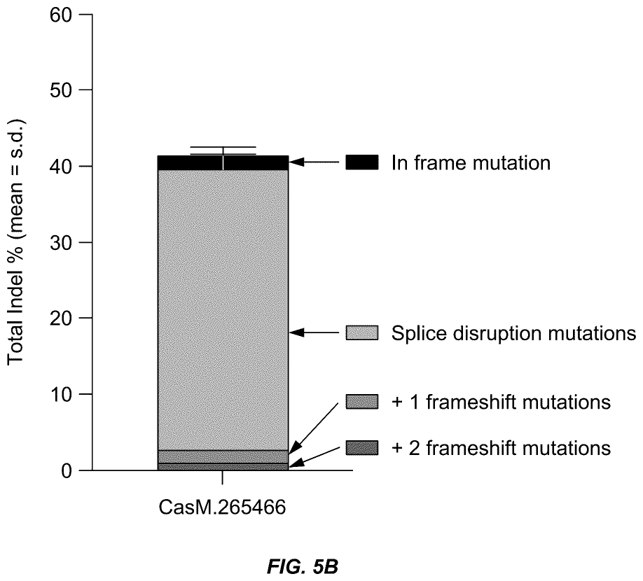

From Example 2, a candidate guide nucleic acid (e.g., SEQ ID NO: 402) for an exon skipping approach to reframe the DMD gene in del50 patients by targeting the splicing acceptor site of exon 51 was identified. FIG. 5A shows a deletion pattern of CasM265466 and guide nucleic acid represented by SEQ ID NO: 402, which results in ablation of splicing acceptor of exon 51 (see FIG. 5B).

### Example 16: In Vitro Testing of Exon Deletion Approach in iPSC

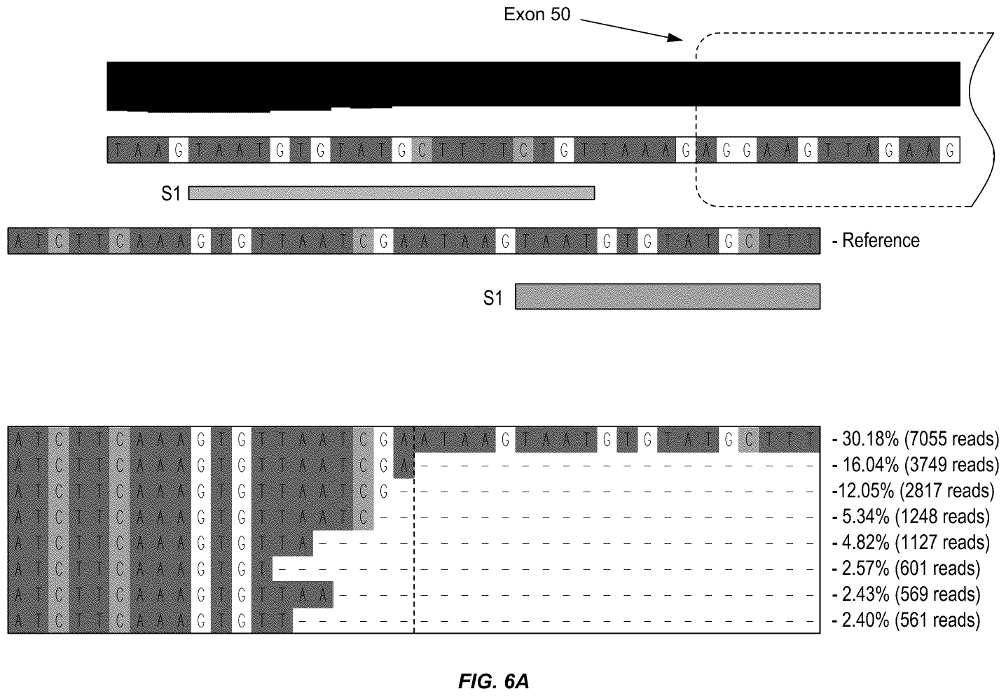

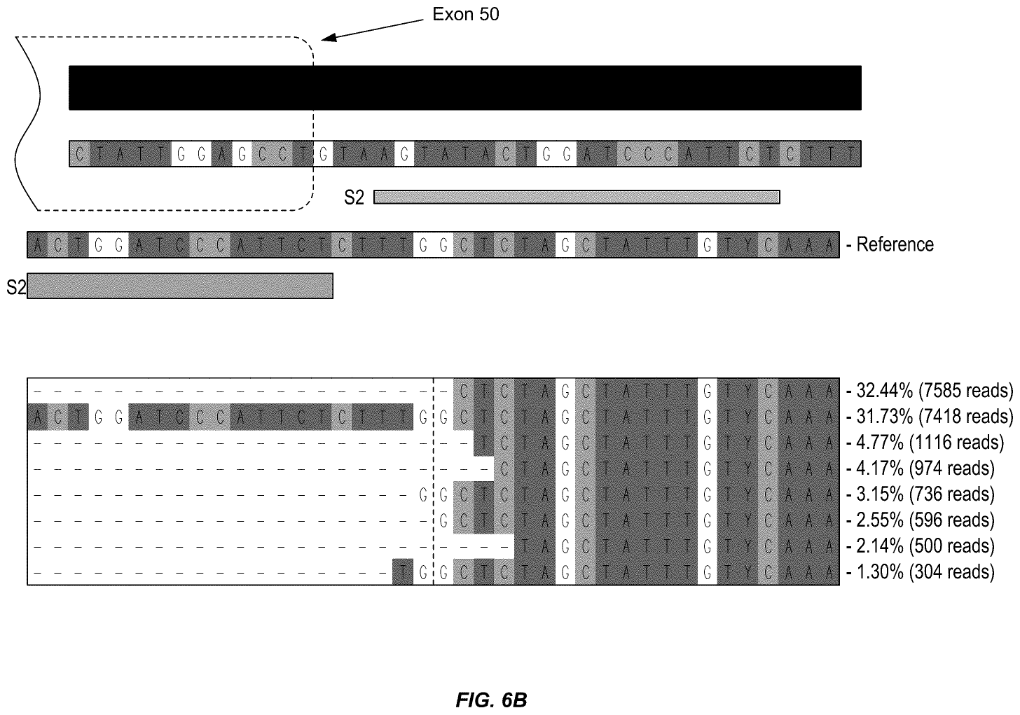

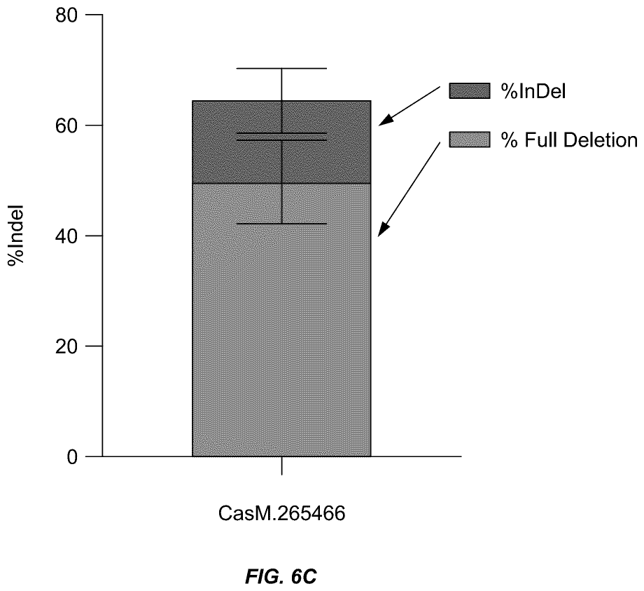

From Example 2, guide nucleic acids, R13445 (SEQ ID NO: 295, PL16669) and R13444 (SEQ ID NO: 310, PL16684), which collectively flank exon 50 of DMD, were identified for generating a deletion of exon 50 (exon Δ50). FIG. 6A and FIG. 6B show cutting patterns on the 5′ and 3′ sides of exon 50, respectively. FIG. 6C shows % indel and % full deletion achieved with these guide pairs in Example 2.

### Example 17: iPSC Models of Duchenne's Muscular Dystrophy to Test Exon Skipping Approach and/or Exon Deletion Approach

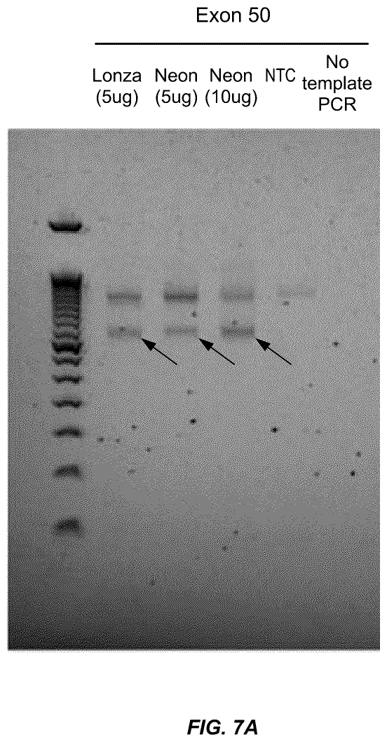

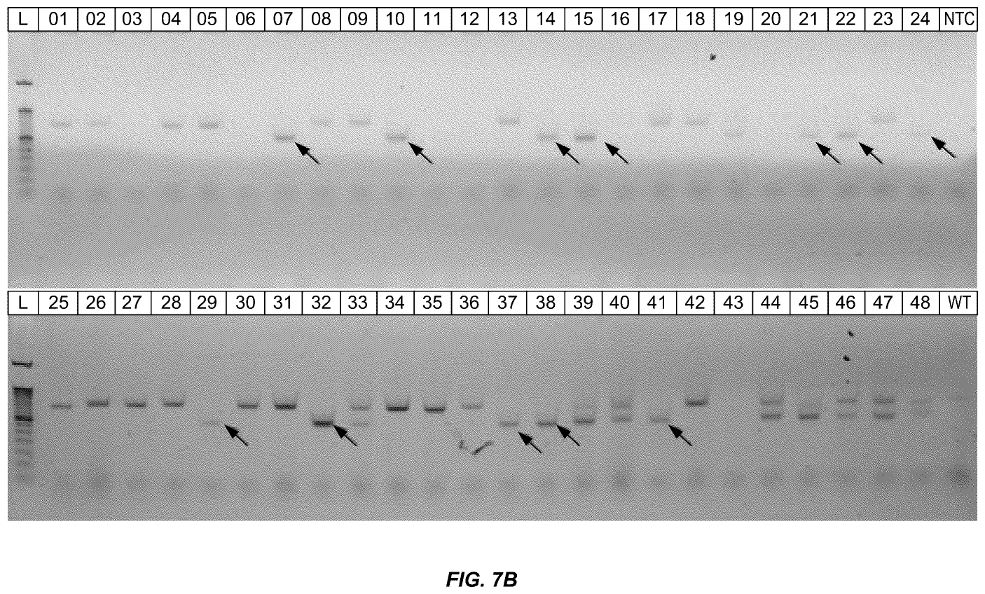

In order to test exon skipping candidate guide nucleic acids (e.g., SEQ ID NO: 402), a cell model was generated to investigate the potential of CasM.265466 as a potential drug to treat DMD, in both exon skipping and exon deletion approaches. Generation of a del50 iPSC cell line was achieved by nucleofecting iPSC with CasM.265466 mRNA and guide nucleic acids (R13444 (SEQ ID NO: 310, PL16684), R13445 (SEQ ID NO: 295, PL16669)), which induced double-stranded DNA breaks between the two guides, causing a whole exon deletion as confirmed by PCR and gel electrophoresis (see FIG. 7A). The gel in FIG. 7A shows that cells electroporated with CasM265466 and the guides flanking exon 50 have two bands, one for the unedited cells (larger ˜550-600 bp) and one for the cells with deleted exon 50 (smaller, about ˜400 bp). Single cell isolation was performed to obtain a clonal population with exon Δ50, and exon 50 deletion was confirmed in individual clones, see FIG. 7B.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1. A composition that comprises:
(a) an effector protein comprising an amino acid sequence that is at least 90% identical to SEQ ID NO: 1, or a nucleic acid encoding the same, and
(b) a guide nucleic acid that comprises a spacer sequence that hybridizes to a target sequence of a human dystrophin (DMD) locus, or a nucleic acid encoding the same.

2. The composition of claim 1, wherein the guide nucleic acid comprises a nucleotide sequence that is at least 90% identical to SEO ID NO: 441 or a nucleotide sequence that is at least 90% identical to SEO ID NO: 443.

3.-7. (canceled)

8. The composition of claim 1, wherein the spacer sequence comprises a nucleotide sequence that is at least 90% identical to any one of SEQ ID NOs: 5-222.

9. The composition of claim 1, wherein the guide nucleic acid comprises a nucleotide sequence that is at least 90% identical to any one of SEQ ID NOs: 223-440.

10. The composition of claim 1, wherein the effector protein comprises an amino acid sequence that is at least 95% identical to SEO ID NO: 1.

11. The composition of claim 10, wherein the effector protein recognizes a protospacer adjacent motif (PAM) sequence recited in TABLE 2 or TABLE 25.

12.-15. (canceled)

16. The composition of claim 1, comprising a partner protein or a nucleic acid encoding the same.

17. The composition of claim 16, wherein partner protein is fused or linked to the effector protein.

18. The composition of claim 16, wherein the partner protein comprises a reverse transcriptase (RT) or a functional domain thereof.

19.-38. (canceled)

39. The composition of claim 18, wherein the RT is not fused or linked to the effector protein.

40. The composition of claim 19, wherein the RT is fused or linked to an aptamer binding protein and wherein the guide nucleic acid comprises an aptamer.

41. The composition of claim 11, comprising a template RNA.

42. The composition of claim 1, wherein the nucleic acid encoding the effector protein and the nucleic acid encoding the guide nucleic acid are located in a nucleic acid expression vector.

43. The composition of claim 42, wherein the nucleic acid expression vector is an adeno associated viral (AAV) vector.

44. A method of modifying a human dystrophin gene (DMD), the method comprising contacting DMD with the composition of claim 1.

45. A cell comprising the composition of claim 1 or modified by the composition of claim 1.

46. The cell of claim 45, wherein the cell is a cardiac muscle cell, a cardiomyocyte, a myocyte, a smooth muscle cell, a skeletal muscle cell, or a visceral muscle cell.

47. A method of treating a disease associated with a mutation or aberrant expression of DMD in a human subject in need thereof, the method comprising administering to the human subject the composition of claim 1.
